(19) United States
(12) Patent Application Publication
(10) Pub. No.: US 2005/0100554 A1
(43) Pub. Date: May 12, 2005

(54) Complexes and methods of using same

(76) Inventors: Amanda Jackson, Paris (FR); Chean Eng Ooi, Branford, CT (US); David Lewin, New Haven, CT (US); Scott Cuthill, Oxfordshire (GB)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/367,057

(22) Filed: Feb. 14, 2003

Related U.S. Application Data
(60) Provisional application No. 60/356,911, filed on Feb. 14, 2002.

(51) Int. Cl. C07H 21/04; C12P 21/04; A61K 39/00; C07K 14/47; C07K 16/18

(52) U.S. Cl. 424/192.1; 435/69.7; 435/320.1; 435/325; 530/350; 530/388.25; 536/23.5

(57) ABSTRACT

The invention provides complexes of at least two polypeptides, and methods of using the same. Purified complexes of two polypeptides are provided, including chimeric complexes, and chimeric polypeptides and complexes thereof are also provided, as are nucleic acids encoding chimeric polypeptides and vectors and cells containing the same. Also provided are methods of identifying agents that disrupt polypeptide complexes, methods of identifying complex or polypeptide in a sample, and for removing the same, methods of determining altered expression of a polypeptide in a subject, and methods of treating/preventing disorders involving altered levels of complex or polypeptide.

### RELATED APPLICATIONS

This application claims priority from U.S. Ser. No. 60/256,911 filed Feb. 14, 2002 the contents of which are incorporated by reference in their entirety.

### FIELD OF THE INVENTION

The invention relates generally to polypeptides and complexes of two or more polypeptides, as well as to methods of use thereof.

### BACKGROUND OF THE INVENTION

Greater than seventy types of human papilloma virus (HPV) are recognized, each type associated with a specific clinical manifestation. (Principles of Internal Medicine, Fauci et al, pp. 190-1100, 14^{th} edition, McGraw Hill). It is predicted that as many as 1-2% of sexually active individuals have genital warts induced predominantly by various types of HPV. Infection may results in a spectrum of epithelial proliferative disorders ranging from common warts through genital warts to invasive cervical cancer. Currently, HPV is the most common sexually transmitted disease and is the etiological agent for 99.7% of all cervical cancers including a number of other cancers such as cancer of the anogenital tract, cancer of the oral cavity, head, neck, and larynx (Walboomers, et al., J Pathol 189 (1): 12-9 (1999); Munoz, J Clin Virol 19 1-2): 1-5 (2000); Gillison, et. al., Curr Opin Oncol 13 (3): 183-8 (2001)). Cervical cancer has a very high survival rate (95% for Stage IA and 80% for Stage IIA) of five-years and is highly tractable to treatment when caught early. The progression of disease is relatively slow compared to many cancers. In later stages, such as IIB, III, or IVA, the five-year survival rate drops off precipitously (65, 40, and 20 percent, respectively) (Cannistra, et al., N Engl J Med 334 (16): 1030-8 (1996)).

HPV genomes are highly conserved and express only eight proteins two of which are capsid proteins (L1 and L2). The other proteins (E1, E2 and E4) are nonstructural and are involved in the replication and transcription of the HPV genome. E1 is a helicase and represents the only enzyme function that encodes HPV. The remaining proteins function via protein-protein or protein-DNA interactions within the infected cell.

HPV has to facilitate its own replication within terminally differentiating (non-replicating) host cells. Important to the viral proliferation is the expression of early genes from the viral genome, E5, E6, and E7. HPV pathogenesis requires the availability of epidermal and mucosal epithelial cells that are still capable of proliferation. E5, E6 and E7 proteins are involved in the aberrant proliferation of the epithelial basal layer such that there is enhanced proliferation and lateral expansion of the basal layer (zur Hausen, Biochim Biophys Acta 1288 (2): F55-78 (1996)).

Cancer caused by HPV is due to the integration, and subsequent expression, of viral E6 and E7 proteins. E6 interacts with p53 (Werness, et al., Science 248 (4951): 76-9 (1990)). and affects the expression of the catalytic subunit of telomerase (hTERT) and Rb (Dyson et al. Science 243(4893): 934-7 (1989); Kiyono et al., Natural 396(6706): 84-8 (1998)). E6 and E7 work synergistically to transform cells (Munger, et al. J Virol 63 (10): 4417-21 (1989); McDougall, Curr Top Microbiol Immunol 186: 101-19 (1994)). E5 is not required for long-term transformation, however it is known to interact with growth factor receptors that mediate cellular proliferation, such as the epidermal growth factor receptor, the platelet-derived growth factor-β receptor, and the colony-stimulating factor-1 receptor (Hwang, et al., Virology 211(1): 227-33 (1995)). The E6 and E7 proteins of HPV maintain infected keratinocytes in a cycling undifferentiated state by blocking p53- and RB-mediated cell-cycle control pathways. U.S. Pat. No. 5,532,348 describes the purification of the E6 protein and methods of blocking the inhibitory effect of E6 on tumor suppressor protein, p53. In oncogenic indications, HPV causes alteration in cyclin-cyclin-dependent kinase complexes, as a prelude to loss of genomic stability, predisposing the cell to neoplastic transformation. E6 and E7 proteins must persist in the cell for it to remain transformed (Tindle, Nature Rev Cancer 2 (1): 59-65 (2002)).

Existing diagnostic methodologies have been described in detail in Wright, et al., Jama 287 (16): 2120-9 (2002). Papanicolaou testing (Pap smear) is the front line screen for cervical cancer testing. However this methodology is limited to reproductive and anogenital screening and does not apply for screening patients for head and neck/larynx cancers. Furthermore, the methodology is not conclusive, ambiguous Pap smear results must be clarified by PCR testing for the presence of high-risk HPV strains (Schiffman, et al., J Clin Microbiol 33 (3): 545-50 (1995)). Following a positive result from PCR testing or multiple ASCUS (Atypical Squamous Cells of Undetermined Significance) results, colposcopy (visual exam of the cervical region) and possibly a biopsy are performed to determine if premalignant or malignant lesions are present (Wright, et al., Jama 287 (16): 2120-9 (2002)).

Current therapies for HPV infection are largely ablative, ineffective and limited. Immunomodulatory cytokines show promise for the suppression of HPV transcription, for example transforming growth factor-β and interleukin-1. Retinoic acid can suppress HPV infection and may have some effect on premalignant and malignant cervical lesions. Cidovovir™, an acyclic nuycleoside phosphonate, with a broad specificity for DNA viruses can suppress HPV infection as can the immunomodulatory drug Imiquimod™. Both drugs seem to act through the stimulation of cytokines (zur Hausen, Nat Rev Cancer 2 (5): 342-50 (2002)).

Other than the avoidance of contact, resolution of the disease has not been successful. Despite the current understanding of HPV infection and replication, the detection and prevention of HPV has been difficult because the nature of replication, expression, proliferation and host protein interactions are incredibly complex. Host defense responses to HPV infection are not largely understood. The ability to identify critical HPV-host protein interactions would not only help clarify how these complicated processes are regulated but could potentially identify important new therapeutic targets and diagnostic markers.

There remains a need to elucidate biochemical pathways that specifically affect the survival or condition of host cells and to determine what molecules and/or functional elements of such molecules are responsible for regulating such pathways. There is also a need for products and processes that permit the effective regulation of specific steps in such biochemical pathways. The identification of protein complexes associated with specific biological activities can be used to identify or prevent conditions associated with the absence or presence of these complexes.

### SUMMARY OF THE INVENTION

The invention is based, in part, upon the identification of protein-protein interactions in and humans. Interacting proteins present in complexes according to the invention are shown in, e.g., Tables 1-7, in the second and fifth columns thereof (i.e., columns 2 and 5).

In one aspect, the invention provides a purified complex including a first interactor listed in Tables 1-7 column 2, and a second interactor recited in column 5 of Tables 1-7.

In another aspect, the invention provides an antibody which specifically binds polypeptide complexes according to the invention. The antibody preferably binds to a complex comprising one or more polypeptides with greater affinity than its affinity for either polypeptide that is not present in the complex.

Also provided by the invention are kits containing reagents that can specifically detect the complexes of the invention. In one embodiment, the reagent is a complex-specific antibody, while in other embodiments the reagent is an antibody specific for the first or second polypeptides of the complex.

In another aspect, the invention provides pharmaceutical compositions including the complexes described herein. Such compositions are formulated to be suitable for therapeutic administration in the treatment of deficiencies or diseases involving altered levels of the complexes of the invention.

In still another aspect, the invention provides methods of identifying an agent that disrupts a polypeptide complex by providing a complex described herein, contacting the complex with a test agent, and detecting the presence or amount of a polypeptide in the complex.

In a further aspect, the invention provides a method for inhibiting the interaction of a protein with a ligand by contacting a complex of the protein and ligand with an agent that blocks formation of the complex.

In yet another aspect, the invention provides a method of identifying a polypeptide complex in a subject by providing a biological sample from the subject and detecting, if present, the level of a complex, described herein, in the subject. In a specific embodiment, the invention encompasses a method to monitor protein interactions or formation of the protein complexes as an indicator of specific state or condition in response to treatment with a drug or pharmaceutical. An aspect of this embodiment includes the use of antibodies, specific for the protein complex, as a reagent in a method to determine the relative abundance of the complex under various conditions or in specific tissues. Recombinant proteins may be expressed with “epitope” tags in order to easily monitor their expression and interactions.

Also provided by the invention is a method for detecting a polypeptide in a biological sample by providing a biological sample containing a first polypeptide, and contacting the sample with a second polypeptide under conditions suitable to form a polypeptide complex.

In another aspect, the invention provides a method for the detection of protein complexes used in assays to detect protein-protein interactions, wherein the interactions include full-length proteins, as well as protein fragments that interact in cell-based and in vitro assays. In another aspect, the identified protein complexes can be used in a diagnostic assay for determining a specific disease or pathological condition or state, as well as for detection of a predisposition to a disease or pathological condition. Included in this aspect is a method for the use of labeled or fusion proteins for detection, and/or the use of antibodies specific for the individual proteins or the protein complex. The method measures the ability of the proteins to form the complex, and includes the identification of mutations or single nucleotide polymorphisms (SNPs), which may affect the ability of the proteins to form the complex or function normally.

In a further aspect, the invention provides a method for determining altered expression of a polypeptide in a subject by providing a biological sample from the subject, measuring the level of polypeptide complex in the sample, and comparing the level of the complex in the sample to the level of complex in a reference sample with a known polypeptide expression level. The present invention includes a method to modulate or regulate a specific phenotype by modulating protein components or complexes, which occur in a related pathway. This can be achieved through modulation with a drug or antibody or antisense oligonucleotides, the activity of a protein or complex, the ability of a protein or complex to interact with its biological partner, or the elimination of a protein from a pathway or a complex. Such changes can be observed through monitoring modulation in gene expression of target genes, or the presence or absence of phenotype specific markers. Included as an embodiment of this aspect are vectors, antibodies, libraries of compounds, gene specific antisense oligonucleotides, and cell lines.

In a still further aspect, the invention provides a method of treating or preventing a disease or disorder involving altered levels of a complex described herein or a polypeptide described herein, by administering, to a subject in need thereof, a therapeutically-effective amount of at least one molecule that modulates the function of the complex or polypeptide. In one embodiment, the agent modulates the function of a polypeptide selected from the polypeptides recited in Tables 1-7, columns 2, 3, 5 or 6.

In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference herein in their entirety

### BRIEF DESCRIPTION OF THE DRAWINGS

FIG. 1 is is a graphical illustration showing the interactions between human host proteins and HPV 16 E5, HPV 16 E6 and HPV 16 E7 and the relevant biological events tied to the activity of proteins involved in host cell proliferation.

### DETAILED DESCRIPTION OF THE INVENTION

Definitions

The term “pre-cancerous cervical lesions” is intended to refer to those abnormalities which clinically may be described as “pre-malignant” conditions and which may, without treatment, proceed to full malignancies.

The present invention makes possible the accurate diagnosis and treatment of infections associated with HPV. The invention further clarifies the mechanism of action or biochemical pathways that lead to phenotypes indicative of a disease or abnormal condition. Preferred protein interactions include interactions described in Tables 1-7, most preferred interactions include those described in Tables 4-7. The invention further provides complexes of interacting polypeptides which have not heretofore been shown to interact directly and methods of using these complexes. In another aspect, certain interactions and complexes reveal utilities for known molecules that have not previously been associated with HPV infection. In one embodiment, interactions between viral proteins and phosphatases that regulate the activity of growth factor receptors are disclosed.

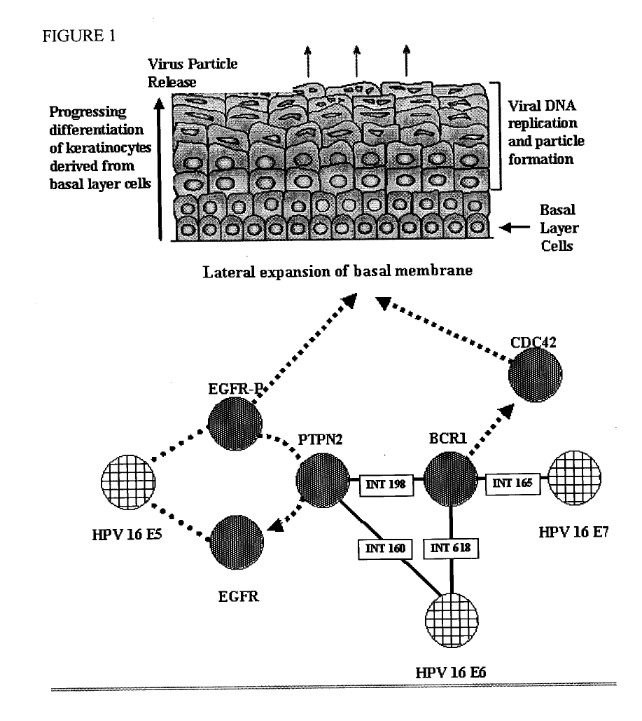

Some interacting polypeptides were identified by determining which of the predicted open-reading frames (ORFs) of the yeast encode polypeptides that interact in a yeast two-hybrid system. The interacting pairs were identified to include (i) interactions that place functionally unclassified proteins in a biological context, (ii) novel interactions between proteins involved in the same biological function, (iii) novel interactions that link together biological functions into larger cellular processes (iv) and identification of potential therapeutic targets and/or diagnostic markers useful in a human clinical setting. In a specific embodiment, protein interactions and pathways in a natural host cell which interact with critical proteins of HPV 1a, HPV 16 or HPV 11 have been identified. The interactions provide a better understanding of the biology of HPV infection as they relate to clinically related HPV strains. More specifically, the present invention provides elucidation of the biochemistry in the strains of virus that are known, respectively, to cause warts, non-cancerous lesions, and tumors. All interactors identified in this invention are potential diagnostic markers for HPV infection and a subset of these have additional value as therapeutic targets. The interactions of the invention are listed in Tables 1 through 7 below. In one embodiment, tabular data depicted in the tables have been exemplified as in FIG. 1.

Prey identified from screens of different tissues were directly tested against all baits from HPV 1, 11, and 16 directly as well as the converse test which is the basis of the one-by-one matrix reaction through Pathcalling (see also U.S. Pat. Nos. 6,057,101 and 6,083,693). A subset of the observed interactions was observed to be specific for each strain tested and others were observed to be conserved interactions for all the strains tested. For example, those interactions that were specific for HPV 16 were analyzed for their potential role in the development of malignancies. Those interactions that were unique to low risk strains are indicative of low-risk infection and the associated consequences, such as common warts (HPV 1a) and benign cervical lesions (HPV 11). Interactions common to both HPV 11 and HPV 16 are relevant to cervical infection, while those common to HPV 1a and HPV 11 are relevant to benign response to infection. All of the above interactions, and the interactions common to any two or more HPV strains are relevant to the general phenomenon of warts.

New insights into novel interactions between proteins involved in the same biological function are also provided. In one embodiment, proteins involved in inactivation of the discs large tumor (DLG) suppressor have been described in Kuhne, et al., Oncogene 30:18(40) pp. 5487-596 (1999). The present invention describes the novel interaction between proteins HPV16 E6 or E7 with DLG that is related to proteosome-mediated degradation. In another embodiment, HPV16 E6 and ubiquitin-specific protease 9 as well as HPV16 E7 and proteosome 26S subunit are novel interactions. These protein interactions may indicate their involvement in proteosome-mediated degradation.

In another aspect, the interactions described by the present invention provide new insights into E6 and E7 mechanisms of action. E7 may enhance the transforming activity of E6 by binding to and inactivating downstream tumor suppressor effects of p53. Because E6 and E7 have several common interactors, concerted action on common biological pathways is indicated. E6 and E7 tranforming activity may also be mediated by interactions with other candidate tumor suppressor or proteins involved in cellular proliferation and apoptosis. Therefore, inhibition or regulation of these interactions are suitable as targets for candidate therapeutics.

In another embodiment, interaction was also identified between complexes of HPV16 E6 and TCP-1, proteosome subunit PMSC1 and syntaxin 4. The interactions have relevance to tumor suppression via protein folding, unfolding and trafficking pathways. Such interactions provide opportunities to develop tools against various pathologic situations in which signaling through these proteins via p53 which are involved in cancer.

The newly identified protein-protein interactions pf the present invention connect biological functions into larger cellular processes. For example, FIG. 1 depicts a diagram of the protein-protein interactions and the relevant biological events tied to the activity of proteins involved in host cell proliferation described in Table A. Viral proteins are depicted as hatched circles, human proteins as closed circles, and the solid lines between the circles represent the physical interactions observed as a consequence of the yeast two-hybrid matrix assay. The broken arrow indicates the predicted direct/indirect activity of PTPN2 as an enzyme that may directly or indirectly affect signal transduction activity of EGFR in the presence of its ligand (Boonstra, Rijken et al. 1995).

TABLE A

| INT | cbe_SEQ CG | COMMON | PT/AT/ | cbe_SEQ CG ID | COMMON | | |
|---|---|---|---|---|---|---|---|
| ID | ID Interactor A | NAME INT A | SMT/NA | Interactor B | NAME INT B | PT/AT/SMT/NA | Screen/Matrix/MS |
| 160 | cbe_251445 | HPV 16 E6 | NA | cbe_2576474 | PTPN2 | SMT | MatrixMating/PathCalling |
| 165 | cbe_251448 | HPV 16 E7 | NA | cbe_422236 | BCR1 | SMT | MatrixMating/PathCalling |
| 198 | cbe_422236 | BCR1 | SMT | cbe_2576474 | PTPN2 | SMT | MatrixMating |
| 618 | cbe_422236 | BCR1 | SMT | cbe_251445 | HPV 16 E6 | NA | MatrixMating |

The viral life cycle of HPV has been well described in both general terms and in terms of HPV's role in cancer (zur Hausen 2002). In short, the basal layer of cells of epithelia depicted in this figure is typically infected by HPV as a result of the exposure of the cells to virus that has penetrated via microlesions. Viral proliferation, formation, and egress from the host is coordinated with the proliferation of the basal layer of cells and their differentiation into keratinocytes. As the keratinocytes mature they release the mature viral particles. Part of the viral life cycle includes the induction of the lateral proliferation of the basal layer. Under normal conditions the proliferation is in a vertical direction as indicated. The co-opting of the proliferative capacity of the basal layer of cells is essential for the successful reproduction of the virus; therefore it is essential that the virus take control of this process.

In some circumstances, co-opting of the proliferation results in a benign lesions. In other cases the results may lead to a cancer in a susceptible tissue such as the cervix. HPV16 is well known to induce cancers of the anogenital tract as well as oral-laryngeal cancers. This highly frequent consequence of chronic HPV 16 infection is the result of the integration of two viral proto-oncogenes, HPV 16 E6 and HPV 16 E7. One or both of these proteins is capable of transforming cells independently of viral infection when transformed into cells (Munger, Phelps et al. 1989; zur Hausen 2002). HPV 16 E5 is known to play a role early in infection by interacting with epidermal growth factor receptor (EGFR) (Hwang, Nottoli et al. 1995; zur Hausen 2002). EGFR is a growth factor receptor whose activity is controlled by its phosphorylation state. Phosphorylated EGFR (EGFR-P) is the consequence of ligand binding and leads to the induction of proliferative intracellular signals (Boonstra, Rijken et al. 1995). PTPN2 is a poorly characterized protein tyrosine phosphatase that can dephosphorylate phospho-tyrosine (Johnson, Cool et al. 1993). The importance of tyrosine dephosphorylation in regulating enzymatic activity has been demonstrated for EGFR (Boonstra, Rijken et al. 1995). Breakpoint cluster region 1 protein (BCR1) is a GTPase-activating protein (GAP) for RAC1 and CDC42 and promotes the exchange of RAC or CDC42-bound GDP by GTP, thereby activating them (Ahmed, Lee et al. 1994). CDC42 is well known to affect cell polarity (Takai, Sasaki et al. 2001).

Some newly disclosed interactions (INT 160, 165, 198, and 618) provide new biological context for these proteins as well as strongly indicating a previously unanticipated functional role for HPV 16 proteins as individual entities as well as components of a complex present in active infections. A diagnostic device that measures the presence of complexes of proteins is the basis to identify active infection as opposed to a late stage infection under which circumstances the transforming consequence of the infection is the result of the integration and aberrant expression of HPV 16 E6 and/or HPV 16 E7. HPV 16 E5 protein's role occurs early in the viral life cycle (zur Hausen 2002).

Novel interactions between HPV 16 and the cell polarity pathway controlled by CDC2 via the direct interactions between HPV 16 viral oncogenes E6 and E7 (INT 165 and INT 618, and indirectly via INT 160 and INT 198) indicates that agents which affect the activity of BCR1 and other GAPs or other effectors of CDC42, such as guanine nucleotide exchange factors (GEFs), or enzymes involved in the post translation modification of CDC42 but not previously associated with HPV infection, are drug targets for the treatment of HPV infection. Any existing agent that affects the activity of enzymes in the CDC42-mediated polarity pathway are suitable as drugs for the treatment of HPV infection. Therefore treatment of HPV infection includes treatment modes which inhibit the ability of HPV to cause basal layer cells proliferation.

In one embodiment, INT 160, INT 198, and INT 165 show direct and indirect interactions between HPV 16 viral oncogenes and PTPN2. Phosphatases play a role in the signal transduction pathways necessary for cellular proliferation (Carr, Wang et al. 2002) which has been indicated as a consequence of HPV infection of the basal layer cells (zur Hausen 2002). Agents that affect the activity of PTPN2 may be used to treat HPV16 infection. Treatment of the infection early will reduce the chance of HPV proto-oncogene insertion into the host genome, thereby reducing the probability of tumor induction. In summary, EGFR and CDC42 play roles in cellular proliferation. HPV co-opts the activity of these pathways via novel interaction with PTPN2 and BCR1 as identified by the present invention. Therefore, clinical intervention in these pathways is to treat HPV infection is encompassed by the present invention. Identification of the interactions described in FIG. 1 leads to diagnosis of an active HPV infection at an early stage possible to intervene in the course of the infection and the course of the disease leading to conditions as mild as warts and as severe as tumors.

The complexes disclosed herein are also useful, inter alia, in identifying agents that modulate cellular processes in which one or more members of the complex have previously been associated. For example, many of the interacting proteins identified by INT ID numbers as shown in Tables 1 through 7, have been previously implicated inter alia, in cell growth, cell division, and/or DNA synthesis, protein synthesis, folding or turnover and vesicle trafficking molecules. Accordingly, new agents which modulate cell growth, cell division, and/or DNA synthesis can be identified by evaluating the ability of a test agent to affect formation or dissolution of a complex having INT ID numbers described herein.

Complexes according to the invention can also be used in methods for identifying desired polypeptides in a biological sample by forming a complex of a first polypeptide and a second polypeptide that interacts with the first polypeptide. The presence of the complex indicates that the sample contains the first polypeptide.

The complexes of the invention are useful treating, e.g., alleviating a symptom of, preventing, diagnosing, or screening for compounds to treat or prevent disorders associated with HPV invention. For example, HPV, the human papilloma virus, has been linked to many cancers (e.g., cervical cancer) and intraepithelial neoplasias (commonly called dysplasias, i.e., abnormal cell growth that is generally considered to be precancerous when it occurs in the female reproductive system). Although most of the research that has been done on these conditions is in the female reproductive tract (especially the cervix, but also the vulva and vagina), the virus is linked to cancers and precancerous conditions in all of the following: eye, lung, skin, anus, penis, prostate, esophagus, and stomach.

Symptoms of HPV infection include for example, genital warts are growths or bumps that appear on the vulva, in or around the vagina or anus, on the cervix, or on the penis, scrotum, groin (where the genital area meets the inner thigh). They may be raised or flat, single or multiple, small or large.

The Pap test is the major front-line test for dysplasia. During this test, cells are taken from the cervical area and examined under a microscope for abnormalities. There are two different systems for classifying dysplasia.

The Bethesda, or SIL (squamous intraepithelial lesion) System looks only at individual cells, generally from a Pap test, and classifies them according to the degree of cell abnormality. These break down into:

ASCUS (atypical squamous cells of undetermined significance) means the cells aren't quite right, but they aren't clearly dysplastic, either. This can be caused by a lot of different factors, including hormonal changes, yeast or other infections, medications, or other sources of inflammation. HPV testing is sometimes done at this point. AGUS or AGCUS (atypical glandular cells of undetermined significance) is a finding of atypical glandular cells. This is less common, but since a different type of cancer (adenocarcinoma) develops from glandular (mainly the columnar) cells, this is usually followed up right away with more testing. Adenocarcinoma often doesn't have the extended precancerous phase that squamous cell carcinoma does.

LSIL—low grade squamous intraepithelial lesion—this is also called “mild dysplasia”, however, the true degree and extent of the dysplasia can only be determined upon further evaluation of the cervix itself. Since most LSIL “regresses”—that is, returns to normal without treatment, a woman with LSIL may be advised to return for another Pap test in a few months.

HSIL—high grade squamous intraepithelial lesion—this type of Pap result will always be evaluated further and treated, as it detects cell changes that have progressed beyond the mild stage.

CIN system—The other major system of classifying dysplasia is called the CIN system, for cervical intraepithelial neoplasia. There are corresponding classifications for vaginal and vulvar dysplasia called VAIN and VIN. This system of classification is based both on the degree of dysplasia in the individual cells (like SIL) and how far below the surface of the cervix the dysplasia goes.

CIN I—corresponds to mild dysplasia or LSIL. Additionally, the abnormal cells are only on the very surface of the cervix. As stated under SIL, most of these will regress back to normal over time. About 11% will progress to CIN 3. Only a very small percentage of CIN I leads to cancer.

CIN 2—corresponds to moderate dysplasia or HSIL. About half of the thickness of the epithelium is abnormal (dysplastic). Left alone, about 43% of CIN 2 will regress back to normal, and 20% will progress to CIN 3.

CIN 3—corresponds to severe dysplasia or HSIL. All or almost all of the epithelium is dysplastic. Although some CIN 3 will spontaneously regress, this is almost always treated since the next step is cancer. This is sometimes also referred to as carcinoma in situ.

These utilities, as well as additional utilities, are discussed in greater detail below.

Purified Polypeptide Complexes

In one aspect, the invention includes a purified complex that includes two or more polypeptides. In one embodiment, the invention provides purified complexes of two or more polypeptides. One of the polypeptides includes a polypeptide selected from the polypeptides recited in Tables 1 through 7, column 2 (referenced as cbe_SEQ ID Interactor A) and another includes a polypeptide selected from the polypeptides recited in Table 1 through 7, column 5 (referenced as cbe_SEQ ID Interactor B). In preferred embodiments the first and second polypeptides of the complex are the polypeptides enumerated in Tables 4-7. In some embodiments a first polypeptide is listed as a “bait” polypeptide and a second polypeptide is denoted as “prey” polypeptide, while in other embodiments the first polypeptide corresponds to a “prey” polypeptide and the second is a “bait” polypeptide.

By “corresponding polypeptide” is meant, with reference to Tables 1-7, the polypeptide recited in the same row, reading across from left to right or right to left, as a specific selected peptide. For example, in Table 3, in the first row, the corresponding polypeptide of cbe_{—}251059 is cbe_{—}2599246 (Prey 1054152) These protein pairs are designated as INT ID 137 as is indicated in Table 3. In the second row, however, the corresponding polypeptide of cbe_{—}251059 is cbe_{—}2789381 (Prey 775). These protein pairs are designated as INT ID 138.

Also as used herein, “protein” and “protein complex” are used synonymously with “polypeptide” and “polypeptide complex.” A “purified” polypeptide, protein or biologically active portion thereof is substantially free of cellular material or other contaminating proteins from the cell or tissue source from which the polypeptide is derived, or substantially free from chemical precursors or other chemicals when chemically synthesized. The language “substantially free of cellular material” includes preparations of protein in which the protein is separated from cellular components of the cells from which it is isolated or recombinantly produced. In one embodiment, the language “substantially free of cellular material” includes preparations of polypeptide complex having less than about 30% (by dry weight) of non-complex proteins (also referred to herein as a “contaminating protein”), more preferably less than about 20% of contaminating protein, still more preferably less than about 10% of contaminating protein, and most preferably less than about 5% non-complex protein. When the polypeptide or complex is recombinantly produced, it is also preferably substantially free of culture medium, i.e., culture medium represents less than about 20%, more preferably less than about 10%, and most preferably less than about 5% of the volume of the protein preparation.

TABLE 1
HPV 1a specific interactions.

| INT | cbe_SEQ ID | COMMON | | cbe_SEQ ID | COMMON | | PathCalling Screen and/or |
|---|---|---|---|---|---|---|---|
| ID | Interactor A | NAME INT A | PT/AT/SMT/NA | Interactor B | NAME INT B | PT/AT/SMT/NA | Matrix Assay |
| 383 | cbe_1710 | QP-C | NA | cbe_251917 | HPV 1a E2 | NA | PathCalling |
| 384 | cbe_22607 | Hs.101882 | NA | cbe_251740 | HPV 1a E1-E4 | NA | PathCalling |
| 385 | cbe_92628 | FTHP1 | NA | cbe_251919 | HPV 1a E6 | NA | PathCalling |
| 393 | cbe_251740 | HPV 1a E1-E4 | NA | cbe_251740 | HPV 1a E1-E4 | NA | MatrixMating |
| 394 | cbe_251740 | HPV 1a E1-E4 | NA | cbe_251777 | Prey2316 | NA | PathCalling |
| 395 | cbe_251740 | HPV 1a E1-E4 | NA | cbe_251900 | Prey2598 | NA | PathCalling |
| 396 | cbe_251740 | HPV 1a E1-E4 | NA | cbe_1380530 | RPL13 | NA | PathCalling |
| 397 | cbe_251740 | HPV 1a E1-E4 | NA | cbe_1796502 | CTNND2 | NA | PathCalling |
| 398 | cbe_251740 | HPV 1a E1-E4 | NA | cbe_1807361 | FLJ22729 | NA | PathCalling |
| 399 | cbe_251740 | HPV 1a E1-E4 | NA | cbe_2685998 | Prey2313 | NA | PathCalling |
| 400 | cbe_251740 | HPV 1a E1-E4 | NA | cbe_2830508 | Prey142356 | NA | PathCalling |
| 401 | cbe_251740 | HPV 1a E1-E4 | NA | cbe_3478832 | gbh_y14735 | NA | PathCalling |
| 402 | cbe_251740 | HPV 1a E1-E4 | NA | cbe_3705944 | TNFAIP1 | NA | PathCalling |
| 403 | cbe_251740 | HPV 1a E1-E4 | NA | cbe_3764052 | CKM | SMT | PathCalling |
| 404 | cbe_251740 | HPV 1a E1-E4 | NA | cbe_3833533 | TRIM29 | NA | PathCalling |
| 405 | cbe_251740 | HPV 1a E1-E4 | NA | cbe_3927174 | SCP2 | SMT | PathCalling |
| 406 | cbe_251740 | HPV 1a E1-E4 | NA | cbe_4138433 | KIFAP3 | NA | PathCalling |
| 407 | cbe_251740 | HPV 1a E1-E4 | NA | cbe_4267981 | TNNC1 | NA | PathCalling |
| 408 | cbe_251740 | HPV 1a E1-E4 | NA | cbe_4383836 | KIAA0905 | NA | PathCalling |
| 409 | cbe_251740 | HPV 1a E1-E4 | NA | cbe_4567083 | Prey1207 | NA | PathCalling |
| 410 | cbe_251917 | HPV 1a E2 | NA | cbe_251976 | Prey2777 | NA | MatrixMating/PathCalling |
| 411 | cbe_251917 | HPV 1a E2 | NA | cbe_1469980 | FLJ20259 | NA | MatrixMating/PathCalling |
| 412 | cbe_251917 | HPV 1a E2 | NA | cbe_2826346 | RPS10 | NA | PathCalling |
| 413 | cbe_251917 | HPV 1a E2 | NA | cbe_2830508 | Prey142356 | NA | PathCalling |
| 414 | cbe_251917 | HPV 1a E2 | NA | cbe_3345510 | COX6C | SMT | PathCalling |
| 415 | cbe_251917 | HPV 1a E2 | NA | cbe_3583511 | MGC3222 | NA | PathCalling |
| 416 | cbe_251917 | HPV 1a E2 | NA | cbe_3625721 | EIF2B2 | NA | PathCalling |
| 417 | cbe_251917 | HPV 1a E2 | NA | cbe_3641816 | ANKH | NA | PathCalling |
| 418 | cbe_251917 | HPV 1a E2 | NA | cbe_3826540 | Hs.98226 | NA | MatrixMating/PathCalling |
| 419 | cbe_251917 | HPV 1a E2 | NA | cbe_4268532 | Prey1053593 | NA | PathCalling |
| 420 | cbe_251917 | HPV 1a E2 | NA | cbe_4269834 | GNB2L1 | NA | MatrixMating/PathCalling |
| 421 | cbe_251917 | HPV 1a E2 | NA | cbe_4270118 | Hs.211143 | NA | PathCalling |
| 422 | cbe_251919 | HPV 1a E6 | NA | cbe_251929 | Prey2672 | NA | PathCalling |
| 423 | cbe_251919 | HPV 1a E6 | NA | cbe_3461331 | MET | AT/PT/SMT | PathCalling |
| 424 | cbe_251919 | HPV 1a E6 | NA | cbe_3621798 | XPO1 | NA | PathCalling |
| 425 | cbe_251919 | HPV 1a E6 | NA | cbe_3665307 | HK1 | NA | PathCalling |
| 426 | cbe_251919 | HPV 1a E6 | NA | cbe_4267867 | ANXA2 | NA | PathCalling |
| 427 | cbe_251925 | HPV 1a E7 | NA | cbe_251953 | Prey2724 | NA | MatrixMating/PathCalling |
| 428 | cbe_251925 | HPV 1a E7 | NA | cbe_3753270 | gbh_ac004503 | NA | MatrixMating/PathCalling |
| 429 | cbe_251925 | HPV 1a E7 | NA | cbe_3760926 | gbh_al359334 | NA | MatrixMating/PathCalling |
| 430 | cbe_251925 | HPV 1a E7 | NA | cbe_3789063 | AP2M1 | NA | MatrixMating |
| 431 | cbe_251925 | HPV 1a E7 | NA | cbe_3810032 | gbh_af142421 | NA | MatrixMating/PathCalling |
| 432 | cbe_251925 | HPV 1a E7 | NA | cbe_3820530 | PGAM1 | SMT | MatrixMating/PathCalling |
| 309 | cbe_251925 | HPV 1a E7 | NA | cbe_3834015 | TFPI | AT/PT | MatrixMating |
| 433 | cbe_251925 | HPV 1a E7 | NA | cbe_4265947 | RBP4 | AT/PT | MatrixMating/PathCalling |
| 434 | cbe_251925 | HPV 1a E7 | NA | cbe_4267526 | HRIHFB2206 | NA | MatrixMating/PathCalling |
| 435 | cbe_252016 | HPV 1a L1 | NA | cbe_252039 | Prey2919 | NA | PathCalling |
| 436 | cbe_252016 | HPV 1a L1 | NA | cbe_2611002 | NR1H4 | SMT | MatrixMating/PathCalling |
| 437 | cbe_252016 | HPV 1a L1 | NA | cbe_2980052 | ARHC | SMT | PathCalling |
| 438 | cbe_252016 | HPV 1a L1 | NA | cbe_3800240 | IVD | SMT | PathCalling |
| 439 | cbe_252016 | HPV 1a L1 | NA | cbe_4268726 | NR1H2 | SMT | PathCalling |
| 440 | cbe_252016 | HPV 1a L1 | NA | cbe_4296923 | SCGB1A1 | AT/PT | PathCalling |
| 146 | cbe_251088 | HPV 16 E2 | NA | cbe_252041 | HPV 1a L2 | NA | MatrixMating |
| 441 | cbe_252041 | HPV 1a L2 | NA | cbe_252090 | Prey2820 | NA | MatrixMating |
| 442 | cbe_252041 | HPV 1a L2 | NA | cbe_2321045 | WNT2B | NA | PathCalling |
| 443 | cbe_252041 | HPV 1a L2 | NA | cbe_2603140 | Prey146 | NA | MatrixMating/PathCalling |
| 444 | cbe_252041 | HPV 1a L2 | NA | cbe_3222359 | LOC51748 | NA | PathCalling |
| 445 | cbe_252041 | HPV 1a L2 | NA | cbe_3456824 | gbh_120086 | AT | PathCalling |
| 446 | cbe_252041 | HPV 1a L2 | NA | cbe_3606990 | Prey1053670 | NA | PathCalling |
| 447 | cbe_252041 | HPV 1a L2 | NA | cbe_3770440 | gbh_af217505 | NA | MatrixMating/PathCalling |
| 448 | cbe_252041 | HPV 1a L2 | NA | cbe_3810791 | EPHB1 | NA | MatrixMating/PathCalling |
| 449 | cbe_252041 | HPV 1a L2 | NA | cbe_4248793 | GSN | AT/PT | PathCalling |
| 450 | cbe_252041 | HPV 1a L2 | NA | cbe_4268470 | HSPE1 | NA | MatrixMating/PathCalling |
| 451 | cbe_252041 | HPV 1a L2 | NA | cbe_4269628 | RIL | NA | MatrixMating |
| 452 | cbe_252041 | HPV 1a L2 | NA | cbe_4540466 | PUM1 | NA | MatrixMating/PathCalling |

TABLE 2
HPV11 specific interactions

| INT | cbe_SEQ ID | COMMON | PT/AT/ | cbe_SEQ ID | COMMON NAME | | PathCalling Screen and/or |
|---|---|---|---|---|---|---|---|
| ID | Interactor A | NAME INT A | SMT/NA | Interactor B | INT B | PT/AT/SMT/NA | Matrix Assay |
| 1 | cbe_6464 | Hs.19954 | NA | cbe_250781 | HPV 11 E2 | NA | MatrixMating/PathCalling |
| 2 | cbe_244811 | DGUOK | SMT | cbe_250777 | HPV 11 E4 | NA | MatrixMating/PathCalling |
| 5 | cbe_250777 | HPV 11 E4 | NA | cbe_862174 | MT2A | NA | MatrixMating/PathCalling |
| 6 | cbe_250777 | HPV 11 E4 | NA | cbe_1699351 | SCO1 | NA | MatrixMating/PathCalling |
| 7 | cbe_250777 | HPV 11 E4 | NA | cbe_4344185 | SRA1 | NA | MatrixMating/PathCalling |
| 8 | cbe_250781 | HPV 11 E2 | NA | cbe_251821 | Prey2419 | NA | PathCalling |
| 9 | cbe_250781 | HPV 11 E2 | NA | cbe_251826 | Prey2429 | NA | PathCalling |
| 10 | cbe_250781 | HPV 11 E2 | NA | cbe_251857 | Prey2492 | NA | PathCalling |
| 11 | cbe_250781 | HPV 11 E2 | NA | cbe_251858 | Prey2494 | NA | PathCalling |
| 12 | cbe_250781 | HPV 11 E2 | NA | cbe_366279 | Prey2439 | NA | PathCalling |
| 13 | cbe_250781 | HPV 11 E2 | NA | cbe_408167 | Prey2412 | NA | PathCalling |
| 14 | cbe_250781 | HPV 11 E2 | NA | cbe_3563252 | Hs.306083 | NA | PathCalling |
| 15 | cbe_250781 | HPV 11 E2 | NA | cbe_4269472 | MRG15 | NA | PathCalling |
| 16 | cbe_250781 | HPV 11 E2 | NA | cbe_4269571 | KRT8 | NA | PathCalling |
| 17 | cbe_250808 | HPV 11 E6 | NA | cbe_250809 | Prey152 | NA | PathCalling |
| 18 | cbe_250808 | HPV 11 E6 | NA | cbe_250895 | Prey343 | NA | PathCalling |
| 19 | cbe_250808 | HPV 11 E6 | NA | cbe_250904 | Prey365 | NA | PathCalling |
| 20 | cbe_250808 | HPV 11 E6 | NA | cbe_250906 | Prey370 | NA | PathCalling |
| 21 | cbe_250808 | HPV 11 E6 | NA | cbe_250907 | Prey372 | NA | PathCalling |
| 22 | cbe_250808 | HPV 11 E6 | NA | cbe_250908 | Prey375 | NA | PathCalling |
| 23 | cbe_250808 | HPV 11 E6 | NA | cbe_250912 | Prey385 | NA | PathCalling |
| 24 | cbe_250808 | HPV 11 E6 | NA | cbe_250922 | Prey409 | NA | PathCalling |
| 25 | cbe_250808 | HPV 11 E6 | NA | cbe_250924 | Prey413 | NA | PathCalling |
| 26 | cbe_250808 | HPV 11 E6 | NA | cbe_250925 | Prey415 | NA | PathCalling |
| 27 | cbe_250808 | HPV 11 E6 | NA | cbe_2541164 | GPRK5 | SMT | PathCalling |
| 28 | cbe_250808 | HPV 11 E6 | NA | cbe_3580897 | HSPA5 | NA | PathCalling |
| 29 | cbe_250808 | HPV 11 E6 | NA | cbe_3590661 | NUP214 | SMT | MatrixMating/PathCalling |
| 30 | cbe_250808 | HPV 11 E6 | NA | cbe_3694420 | ARHGEF2 | NA | PathCalling |
| 31 | cbe_250808 | HPV 11 E6 | NA | cbe_3929669 | CD164 | AT/PT | PathCalling |
| 32 | cbe_250808 | HPV 11 E6 | NA | cbe_4175362 | MAPK9 | SMT | PathCalling |
| 33 | cbe_250808 | HPV 11 E6 | NA | cbe_4268235 | KIAA0410 | NA | MatrixMating/PathCalling |
| 34 | cbe_250834 | HPV 11 E7 | NA | cbe_250878 | Prey302 | NA | MatrixMating/PathCalling |
| 35 | cbe_250834 | HPV 11 E7 | NA | cbe_250883 | Prey244 | NA | MatrixMating/PathCalling |
| 36 | cbe_250834 | HPV 11 E7 | NA | cbe_250888 | Prey228 | NA | MatrixMating/PathCalling |
| 37 | cbe_250834 | HPV 11 E7 | NA | cbe_3056756 | KIAA0440 | NA | MatrixMating/PathCalling |
| 38 | cbe_250834 | HPV 11 E7 | NA | cbe_3454406 | gbh_af252829 | NA | MatrixMating/PathCalling |
| 39 | cbe_250834 | HPV 11 E7 | NA | cbe_3760926 | gbh_al359334 | NA | MatrixMating/PathCalling |
| 40 | cbe_250834 | HPV 11 E7 | NA | cbe_3845697 | Hs.191063 | NA | MatrixMating/PathCalling |
| 41 | cbe_250834 | HPV 11 E7 | NA | cbe_4260642 | PTPN21 | SMT | MatrixMating/PathCalling |
| 42 | cbe_250834 | HPV 11 E7 | NA | cbe_4265947 | RBP4 | AT/PT | MatrixMating/PathCalling |
| 43 | cbe_250834 | HPV 11 E7 | NA | cbe_4268935 | RPL26 | NA | MatrixMating/PathCalling |
| 44 | cbe_250834 | HPV 11 E7 | NA | cbe_4269451 | gbh_af038159 | NA | MatrixMating/PathCalling |
| 45 | cbe_250834 | HPV 11 E7 | NA | cbe_4269681 | Hs.224712 | NA | MatrixMating/PathCalling |
| 46 | cbe_250834 | HPV 11 E7 | NA | cbe_4269818 | Hs.73680 | NA | MatrixMating/PathCalling |
| 47 | cbe_250834 | HPV 11 E7 | NA | cbe_4270015 | RPS26 | NA | MatrixMating/PathCalling |
| 48 | cbe_250834 | HPV 11 E7 | NA | cbe_4270441 | Prey1052970 | NA | PathCalling |
| 49 | cbe_250834 | HPV 11 E7 | NA | cbe_4280774 | ITGB4 | AT/PT | MatrixMating/PathCalling |
| 50 | cbe_251663 | HPV 11 E1 | NA | cbe_3643627 | DKFZP434I1735 | NA | PathCalling |
| 51 | cbe_251663 | HPV 11 E1 | NA | cbe_3842044 | PSMB3 | NA | PathCalling |
| 52 | cbe_251663 | HPV 11 E1 | NA | cbe_4269915 | gbh_u23460 | AT/SMT | PathCalling |
| 53 | cbe_251663 | HPV 11 E1 | NA | cbe_4270423 | Prey1053526 | AT | PathCalling |
| 99 | cbe_251445 | HPV 16 E6 | NA | cbe_4270015 | RPS26 | NA | MatrixMating/PathCalling |

TABLE 3
HPV16 specific interactions

| INT | cbe_SEQ CG ID | COMMON | PT/AT/ | cbe_SEQ CG ID | COMMON | PT/AT/ | |
|---|---|---|---|---|---|---|---|
| ID | Interactor A | NAME INT A | SMT/NA | Interactor B | NAME INT B | SMT/NA | Screen/Matrix/MS |
| 137 | cbe_251059 | HPV 16 E1-E4 | NA | cbe_2599246 | Prey1054152 | NA | PathCalling |
| 138 | cbe_251059 | HPV 16 E1-E4 | NA | cbe_2789381 | Prey775 | NA | MatrixMating |
| 139 | cbe_251059 | HPV 16 E1-E4 | NA | cbe_3472582 | RABIF | NA | PathCalling |
| 140 | cbe_251059 | HPV 16 E1-E4 | NA | cbe_3621899 | pcnp | NA | PathCalling |
| 141 | cbe_251059 | HPV 16 E1-E4 | NA | cbe_3880186 | FLJ20343 | NA | MatrixMating/PathCalling |
| 142 | cbe_251059 | HPV 16 E1-E4 | NA | cbe_4269156 | PCDH1 | AT/PT | PathCalling |
| 143 | cbe_251059 | HPV 16 E1-E4 | NA | cbe_4269329 | SP260 | AT | PathCalling |
| 144 | cbe_251063 | HPV 16 E1 | NA | cbe_251099 | Prey802 | NA | MatrixMating/PathCalling |
| 145 | cbe_251063 | HPV 16 E1 | NA | cbe_4465094 | Prey1053092 | NA | PathCalling |
| 146 | cbe_251088 | HPV 16 E2 | NA | cbe_252041 | HPV 1a L2 | NA | MatrixMating |
| 147 | cbe_251088 | HPV 16 E2 | NA | cbe_2321057 | EFEMP1 | AT/PT | MatrixMating/PathCalling |
| 148 | cbe_251088 | HPV 16 E2 | NA | cbe_3670766 | ACTC | NA | MatrixMating/PathCalling |
| 149 | cbe_251088 | HPV 16 E2 | NA | cbe_3922906 | BSMAP | AT | PathCalling |
| 150 | cbe_251088 | HPV 16 E2 | NA | cbe_4268208 | DAD1 | AT | MatrixMating/PathCalling |
| 151 | cbe_251088 | HPV 16 E2 | NA | cbe_4279962 | DKKL1-pending | AT/PT | PathCalling |
| 159 | cbe_251445 | HPV 16 E6 | NA | cbe_2253980 | gbh_d38112_5 | NA | MatrixMating/PathCalling |
| 160 | cbe_251445 | HPV 16 E6 | NA | cbe_2576474 | PTPN2 | SMT | MatrixMating/PathCalling |
| 161 | cbe_251445 | HPV 16 E6 | NA | cbe_4269405 | gbh_af155653 | NA | MatrixMating/PathCalling |
| 162 | cbe_251445 | HPV 16 E6 | NA | cbe_4269703 | LOC51182 | NA | MatrixMating/PathCalling |
| 99 | cbe_251445 | HPV 16 E6 | NA | cbe_4270015 | RPS26 | NA | MatrixMating/PathCalling |
| 163 | cbe_251448 | HPV 16 E7 | NA | cbe_251468 | Prey1628 | NA | MatrixMating/PathCalling |
| 164 | cbe_251448 | HPV 16 E7 | NA | cbe_251502 | Prey1609 | NA | MatrixMating/PathCalling |
| 165 | cbe_251448 | HPV 16 E7 | NA | cbe_422236 | BCR1 | SMT | MatrixMating/PathCalling |
| 166 | cbe_251448 | HPV 16 E7 | NA | cbe_2693325 | Prey1673 | NA | MatrixMating/PathCalling |
| 167 | cbe_251448 | HPV 16 E7 | NA | cbe_2898065 | Prey1620 | NA | MatrixMating/PathCalling |
| 168 | cbe_251448 | HPV 16 E7 | NA | cbe_3474077 | KRT4 | NA | MatrixMating |
| 169 | cbe_251448 | HPV 16 E7 | NA | cbe_3773649 | EEF1A1L14 | NA | MatrixMating/PathCalling |
| 170 | cbe_251448 | HPV 16 E7 | NA | cbe_3787129 | LOC55828 | NA | MatrixMating/PathCalling |
| 171 | cbe_251448 | HPV 16 E7 | NA | cbe_3789063 | AP2M1 | NA | MatrixMating/PathCalling |
| 172 | cbe_251448 | HPV 16 E7 | NA | cbe_3834015 | TFPI | AT/PT | MatrixMating |
| 173 | cbe_251448 | HPV 16 E7 | NA | cbe_3921620 | MPP3 | AT | MatrixMating/PathCalling |
| 174 | cbe_251448 | HPV 16 E7 | NA | cbe_4260642 | PTPN21 | SMT | MatrixMating/PathCalling |
| 175 | cbe_251448 | HPV 16 E7 | NA | cbe_4265947 | RBP4 | AT/PT | MatrixMating |
| 176 | cbe_251448 | HPV 16 E7 | NA | cbe_4268611 | NDUFS3 | SMT | MatrixMating/PathCalling |
| 177 | cbe_251448 | HPV 16 E7 | NA | cbe_4268684 | MIZF | NA | MatrixMating/PathCalling |
| 178 | cbe_251448 | HPV 16 E7 | NA | cbe_4268740 | ZNF-U69274 | NA | MatrixMating/PathCalling |
| 179 | cbe_251448 | HPV 16 E7 | NA | cbe_4269658 | PSMC1 | NA | MatrixMating/PathCalling |
| 180 | cbe_251448 | HPV 16 E7 | NA | cbe_4269876 | SBP2 | NA | MatrixMating/PathCalling |
| 181 | cbe_251448 | HPV 16 E7 | NA | cbe_4270186 | STX4A | AT | MatrixMating/PathCalling |
| 182 | cbe_251737 | HPV 16 L2 | NA | cbe_422227 | Prey277610 | NA | PathCalling |
| 183 | cbe_251737 | HPV 16 L2 | NA | cbe_422247 | Prey277642 | NA | PathCalling |
| 184 | cbe_251737 | HPV 16 L2 | NA | cbe_3056756 | KIAA0440 | NA | MatrixMating/PathCalling |
| 185 | cbe_251737 | HPV 16 L2 | NA | cbe_3473488 | gbh_al121903 | NA | PathCalling |
| 186 | cbe_251737 | HPV 16 L2 | NA | cbe_3575702 | SUI1 | NA | PathCalling |
| 187 | cbe_251737 | HPV 16 L2 | NA | cbe_3663966 | KIAA1389 | NA | PathCalling |
| 188 | cbe_251737 | HPV 16 L2 | NA | cbe_3849002 | HSPC224 | AT | PathCalling |
| 189 | cbe_251737 | HPV 16 L2 | NA | cbe_3884811 | Hs.30289 | NA | PathCalling |
| 190 | cbe_251737 | HPV 16 L2 | NA | cbe_4268684 | MIZF | NA | MatrixMating |
| 191 | cbe_251737 | HPV 16 L2 | NA | cbe_4270335 | KIAA0276 | NA | PathCalling |
| 192 | cbe_251784 | HPV 16 L1 | NA | cbe_251790 | Prey2347 | NA | PathCalling |
| 193 | cbe_251784 | HPV 16 L1 | NA | cbe_422204 | Prey2335 | NA | PathCalling |
| 194 | cbe_251784 | HPV 16 L1 | NA | cbe_552305 | Prey2354 | NA | PathCalling |
| 195 | cbe_251784 | HPV 16 L1 | NA | cbe_967711 | Hs.201037 | NA | PathCalling |
| 196 | cbe_251784 | HPV 16 L1 | NA | cbe_1505425 | HCDI | NA | PathCalling |
| 197 | cbe_251784 | HPV 16 L1 | NA | cbe_4281271 | CRYAB | NA | PathCalling |
| 316 | cbe_2599246 | Prey1054152 | NA | cbe_3929637 | HPV 16 E5 | AT/PT | PathCalling |
| 618 | HPV 16 | BCR1 | SMT | 251445 | HPV 16 E6 | NA | MatrixMating |
| | E6422236 | | | | | | |
| 317 | cbe_2830508 | Prey142356 | NA | cbe_3929637 | HPV 16 E5 | AT/PT | PathCalling |

TABLE 4
HPV1a and HPV 11 specific interactions

| INT | cbe_SEQ CG ID | COMMON | PT/AT/ | cbe_SEQ CG ID | COMMON | PT/AT/ | |
|---|---|---|---|---|---|---|---|
| ID | Interactor A | NAME INT A | SMT/NA | Interactor B | NAME INT B | SMT/NA | |
| | | | | | | | PathCalling Screen and/or |
| | | | | | | | Matrix Assay |
| 137 | cbe_251059 | HPV 16 E1-E4 | NA | cbe_2599246 | Prey1054152 | NA | PathCalling |
| 138 | cbe_251059 | HPV 16 E1-E4 | NA | cbe_2789381 | Prey775 | NA | MatrixMating |
| 139 | cbe_251059 | HPV 16 E1-E4 | NA | cbe_3472582 | RABIF | NA | PathCalling |
| 140 | cbe_251059 | HPV 16 E1-E4 | NA | cbe_3621899 | pcnp | NA | PathCalling |
| 141 | cbe_251059 | HPV 16 E1-E4 | NA | cbe_3880186 | FLJ20343 | NA | MatrixMating/PathCalling |
| 142 | cbe_251059 | HPV 16 E1-E4 | NA | cbe_4269156 | PCDH1 | AT/PT | PathCalling |
| 143 | cbe_251059 | HPV 16 E1-E4 | NA | cbe_4269329 | SP260 | AT | PathCalling |
| 144 | cbe_251063 | HPV 16 E1 | NA | cbe_251099 | Prey802 | NA | MatrixMating/PathCalling |
| 145 | cbe_251063 | HPV 16 E1 | NA | cbe_4465094 | Prey1053092 | NA | PathCalling |
| 146 | cbe_251088 | HPV 16 E2 | NA | cbe_252041 | HPV 1a L2 | NA | MatrixMating |
| 147 | cbe_251088 | HPV 16 E2 | NA | cbe_2321057 | EFEMP1 | AT/PT | MatrixMating/PathCalling |
| 148 | cbe_251088 | HPV 16 E2 | NA | cbe_3670766 | ACTC | NA | MatrixMating/PathCalling |
| 149 | cbe_251088 | HPV 16 E2 | NA | cbe_3922906 | BSMAP | AT | PathCalling |
| 150 | cbe_251088 | HPV 16 E2 | NA | cbe_4268208 | DAD1 | AT | MatrixMating/PathCalling |
| 151 | cbe_251088 | HPV 16 E2 | NA | cbe_4279962 | DKKL1-pending | AT/PT | PathCalling |
| 159 | cbe_251445 | HPV 16 E6 | NA | cbe_2253980 | gbh_d38112_5 | NA | MatrixMating/PathCalling |
| 160 | | HPV 16 E6 | NA | cbe_2576474 | PTPN2 | AT/SMT | MatrixMating/PathCalling |
| 161 | cbe_251445 | HPV 16 E6 | NA | cbe_4269405 | gbh_af155653 | NA | MatrixMating/PathCalling |
| 162 | cbe_251445 | HPV 16 E6 | NA | cbe_4269703 | LOC51182 | NA | MatrixMating/PathCalling |
| 99 | cbe_251445 | HPV 16 E6 | NA | cbe_4270015 | RPS26 | NA | MatrixMating/PathCalling |
| 163 | cbe_251448 | HPV 16 E7 | NA | cbe_251468 | Prey1628 | NA | MatrixMating/PathCalling |
| 164 | cbe_251448 | HPV 16 E7 | NA | cbe_251502 | Prey1609 | NA | MatrixMating/PathCalling |
| 165 | cbe_251448 | HPV 16 E7 | NA | cbe_422236 | BCR1 | NA | MatrixMating/PathCalling |
| 166 | cbe_251448 | HPV 16 E7 | NA | cbe_2693325 | Prey1673 | NA | MatrixMating/PathCalling |
| 167 | cbe_251448 | HPV 16 E7 | NA | cbe_2898065 | Prey1620 | NA | MatrixMating/PathCalling |
| 168 | cbe_251448 | HPV 16 E7 | NA | cbe_3474077 | KRT4 | NA | MatrixMating |
| 169 | cbe_251448 | HPV 16 E7 | NA | cbe_3773649 | EEF1A1L14 | NA | MatrixMating/PathCalling |
| 170 | cbe_251448 | HPV 16 E7 | NA | cbe_3787129 | LOC55828 | NA | MatrixMating/PathCalling |
| 171 | cbe_251448 | HPV 16 E7 | NA | cbe_3789063 | AP2M1 | NA | MatrixMating/PathCalling |
| 172 | cbe_251448 | HPV 16 E7 | NA | cbe_3834015 | TFPI | AT/PT | MatrixMating |
| 173 | cbe_251448 | HPV 16 E7 | NA | cbe_3921620 | MPP3 | AT | MatrixMating/PathCalling |
| 174 | cbe_251448 | HPV 16 E7 | NA | cbe_4260642 | PTPN21 | SMT | MatrixMating/PathCalling |
| 175 | cbe_251448 | HPV 16 E7 | NA | cbe_4265947 | RBP4 | AT/PT | MatrixMating |
| 176 | cbe_251448 | HPV 16 E7 | NA | cbe_4268611 | NDUFS3 | SMT | MatrixMating/PathCalling |
| 177 | cbe_251448 | HPV 16 E7 | NA | cbe_4268684 | MIZF | NA | MatrixMating/PathCalling |
| 178 | cbe_251448 | HPV 16 E7 | NA | cbe_4268740 | ZNF-U69274 | NA | MatrixMating/PathCalling |
| 179 | cbe_251448 | HPV 16 E7 | NA | cbe_4269658 | PSMC1 | NA | MatrixMating/PathCalling |
| 180 | cbe_251448 | HPV 16 E7 | NA | cbe_4269876 | SBP2 | NA | MatrixMating/PathCalling |
| 181 | cbe_251448 | HPV 16 E7 | NA | cbe_4270186 | STX4A | AT | MatrixMating/PathCalling |
| 182 | cbe_251737 | HPV 16 L2 | NA | cbe_422227 | Prey277610 | NA | PathCalling |
| 183 | cbe_251737 | HPV 16 L2 | NA | cbe_422247 | Prey277642 | NA | PathCalling |
| 184 | cbe_251737 | HPV 16 L2 | NA | cbe_3056756 | KIAA0440 | NA | MatrixMating/PathCalling |
| 185 | cbe_251737 | HPV 16 L2 | NA | cbe_3473488 | gbh_al12 1903 | NA | PathCalling |
| 186 | cbe_251737 | HPV 16 L2 | NA | cbe_3575702 | SUI1 | NA | PathCalling |
| 187 | cbe_251737 | HPV 16 L2 | NA | cbe_3663966 | KIAA1389 | NA | PathCalling |
| 188 | cbe_251737 | HPV 16 L2 | NA | cbe_3849002 | HSPC224 | AT | PathCalling |
| 189 | cbe_251737 | HPV 16 L2 | NA | cbe_3884811 | Hs.30289 | NA | PathCalling |
| 190 | cbe_251737 | HPV 16 L2 | NA | cbe_4268684 | MIZF | NA | MatrixMating |
| 191 | cbe_251737 | HPV 16 L2 | NA | cbe_4270335 | KIAA0276 | NA | PathCalling |
| 192 | cbe_251784 | HPV 16 L1 | NA | cbe_251790 | Prey2347 | NA | PathCalling |
| 193 | cbe_251784 | HPV 16 L1 | NA | cbe_422204 | Prey2335 | NA | PathCalling |
| 194 | cbe_251784 | HPV 16 L1 | NA | cbe_552305 | Prey2354 | NA | PathCalling |
| 195 | cbe_251784 | HPV 16 L1 | NA | cbe_967711 | Hs.201037 | NA | PathCalling |
| 196 | cbe_251784 | HPV 16 L1 | NA | cbe_1505425 | HCDI | NA | PathCalling |
| 197 | cbe_251784 | HPV 16 L1 | NA | cbe_4281271 | CRYAB | NA | PathCalling |
| 316 | cbe_2599246 | Prey1054152 | NA | cbe_3929637 | HPV 16 E5 | AT/PT | PathCalling |
| 317 | cbe_2830508 | Prey142356 | NA | cbe_3929637 | HPV 16 E5 | AT/PT | PathCalling |
| | | | | | | | Screen/Matrix/MS |
| 137 | cbe_251059 | HPV 16 E1-E4 | NA | cbe_2599246 | Prey1054152 | NA | PathCalling |
| 138 | cbe_251059 | HPV 16 E1-E4 | NA | cbe_2789381 | Prey775 | NA | MatrixMating |
| 139 | cbe_251059 | HPV 16 E1-E4 | NA | cbe_3472582 | RABIF | NA | PathCalling |
| 140 | cbe_251059 | HPV 16 E1-E4 | NA | cbe_3621899 | pcnp | NA | PathCalling |
| 141 | cbe_251059 | HPV 16 E1-E4 | NA | cbe_3880186 | FLJ20343 | NA | MatrixMating/PathCalling |
| 142 | cbe_251059 | HPV 16 E1-E4 | NA | cbe_4269156 | PCDH1 | AT/PT | PathCalling |
| 143 | cbe_251059 | HPV 16 E1-E4 | NA | cbe_4269329 | SP260 | AT | PathCalling |

TABLE 5
HPV1a and HPV 16 specific interactions

| INT | cbe_SEQ ID | COMMON | PT/AT/ | cbe_SEQ ID | COMMON | | PathCalling Screen and/or |
|---|---|---|---|---|---|---|---|
| ID | Interactor A | NAME INT A | SMT/NA | Interactor B | NAME INT B | PT/AT/SMT/NA | Matrix Assay |
| 137 | cbe_251059 | HPV 16 E1-E4 | NA | cbe_2599246 | Prey1054152 | NA | PathCalling |
| 138 | cbe_251059 | HPV 16 E1-E4 | NA | cbe_2789381 | Prey775 | NA | MatrixMating |
| 139 | cbe_251059 | HPV 16 E1-E4 | NA | cbe_3472582 | RABIF | NA | PathCalling |
| 140 | cbe_251059 | HPV 16 E1-E4 | NA | cbe_3621899 | pcnp | NA | PathCalling |
| 141 | cbe_251059 | HPV 16 E1-E4 | NA | cbe_3880186 | FLJ20343 | NA | MatrixMating/PathCalling |
| 142 | cbe_251059 | HPV 16 E1-E4 | NA | cbe_4269156 | PCDH1 | AT/PT | PathCalling |
| 143 | cbe_251059 | HPV 16 E1-E4 | NA | cbe_4269329 | SP260 | AT | PathCalling |
| 144 | cbe_251063 | HPV 16 E1 | NA | cbe_251099 | Prey802 | NA | MatrixMating/PathCalling |
| 145 | cbe_251063 | HPV 16 E1 | NA | cbe_4465094 | Prey1053092 | NA | PathCalling |
| 146 | cbe_251088 | HPV 16 E2 | NA | cbe_252041 | HPV 1a L2 | NA | MatrixMating |
| 147 | cbe_251088 | HPV 16 E2 | NA | cbe_2321057 | EFEMP1 | AT/PT | MatrixMating/PathCalling |
| 148 | cbe_251088 | HPV 16 E2 | NA | cbe_3670766 | ACTC | NA | MatrixMating/PathCalling |
| 149 | cbe_251088 | HPV 16 E2 | NA | cbe_3922906 | BSMAP | AT | PathCalling |
| 150 | cbe_251088 | HPV 16 E2 | NA | cbe_4268208 | DAD1 | AT | MatrixMating/PathCalling |
| 151 | cbe_251088 | HPV 16 E2 | NA | cbe_4279962 | DKKL1-pending | AT/PT | PathCalling |
| 159 | cbe_251445 | HPV 16 E6 | NA | cbe_2253980 | gbh_d38112_5 | NA | MatrixMating/PathCalling |
| 160 | cbe_251445 | HPV 16 E6 | NA | cbe_2576474 | PTPN2 | AT/SMT | MatrixMating/PathCalling |
| 161 | cbe_251445 | HPV 16 E6 | NA | cbe_4269405 | gbh_af155653 | NA | MatrixMating/PathCalling |
| 162 | cbe_251445 | HPV 16 E6 | NA | cbe_4269703 | LOC51182 | NA | MatrixMating/PathCalling |
| 99 | cbe_251445 | HPV 16 E6 | NA | cbe_4270015 | RPS26 | NA | MatrixMating/PathCalling |
| 163 | cbe_251448 | HPV 16 E7 | NA | cbe_251468 | Prey1628 | NA | MatrixMating/PathCalling |
| 164 | cbe_251448 | HPV 16 E7 | NA | cbe_251502 | Prey1609 | NA | MatrixMating/PathCalling |
| 165 | cbe_251448 | HPV 16 E7 | NA | cbe_422236 | BCR1 | NA | MatrixMating/PathCalling |
| 166 | cbe_251448 | HPV 16 E7 | NA | cbe_2693325 | Prey1673 | NA | MatrixMating/PathCalling |
| 167 | cbe_251448 | HPV 16 E7 | NA | cbe_2898065 | Prey1620 | NA | MatrixMating/PathCalling |
| 168 | cbe_251448 | HPV 16 E7 | NA | cbe_3474077 | KRT4 | NA | MatrixMating |
| 169 | cbe_251448 | HPV 16 E7 | NA | cbe_3773649 | EEF1A1L14 | NA | MatrixMating/PathCalling |
| 170 | cbe_251448 | HPV 16 E7 | NA | cbe_3787129 | LOC55828 | NA | MatrixMating/PathCalling |
| 171 | cbe_251448 | HPV 16 E7 | NA | cbe_3789063 | AP2M1 | NA | MatrixMating/PathCalling |
| 172 | cbe_251448 | HPV 16 E7 | NA | cbe_3834015 | TFPI | AT/PT | MatrixMating |
| 173 | cbe_251448 | HPV 16 E7 | NA | cbe_3921620 | MPP3 | AT | MatrixMating/PathCalling |
| 174 | cbe_251448 | HPV 16 E7 | NA | cbe_4260642 | PTPN21 | SMT | MatrixMating/PathCalling |
| 175 | cbe_251448 | HPV 16 E7 | NA | cbe_4265947 | RBP4 | AT/PT | MatrixMating |
| 176 | cbe_251448 | HPV 16 E7 | NA | cbe_4268611 | NDUFS3 | SMT | MatrixMating/PathCalling |
| 177 | cbe_251448 | HPV 16 E7 | NA | cbe_4268684 | MIZF | NA | MatrixMating/PathCalling |
| 178 | cbe_251448 | HPV 16 E7 | NA | cbe_4268740 | ZNF-U69274 | NA | MatrixMating/PathCalling |
| 179 | cbe_251448 | HPV 16 E7 | NA | cbe_4269658 | PSMC1 | NA | MatrixMating/PathCalling |
| 180 | cbe_251448 | HPV 16 E7 | NA | cbe_4269876 | SBP2 | NA | MatrixMating/PathCalling |
| 181 | cbe_251448 | HPV 16 E7 | NA | cbe_4270186 | STX4A | AT | MatrixMating/PathCalling |
| 383 | cbe_1710 | QP-C | NA | cbe_251917 | HPV 1a E2 | NA | PathCalling |
| 410 | cbe_251917 | HPV 1a E2 | NA | cbe_251976 | Prey2777 | NA | MatrixMating/PathCalling |
| 411 | cbe_251917 | HPV 1a E2 | NA | cbe_1469980 | FLJ20259 | NA | MatrixMating/PathCalling |
| 412 | cbe_251917 | HPV 1a E2 | NA | cbe_2826346 | RPS10 | NA | PathCalling |
| 413 | cbe_251917 | HPV 1a E2 | NA | cbe_2830508 | Prey142356 | NA | PathCalling |
| 414 | cbe_251917 | HPV 1a E2 | NA | cbe_3345510 | COX6C | SMT | PathCalling |
| 415 | cbe_251917 | HPV 1a E2 | NA | cbe_3583511 | MGC3222 | NA | PathCalling |
| 416 | cbe_251917 | HPV 1a E2 | NA | cbe_3625721 | EIF2B2 | NA | PathCalling |
| 417 | cbe_251917 | HPV 1a E2 | NA | cbe_3641816 | ANKH | NA | PathCalling |
| 418 | cbe_251917 | HPV 1a E2 | NA | cbe_3826540 | Hs.98226 | NA | MatrixMating/PathCalling |
| 419 | cbe_251917 | HPV 1a E2 | NA | cbe_4268532 | Prey1053593 | NA | PathCalling |
| 420 | cbe_251917 | HPV 1a E2 | NA | cbe_4269834 | GNB2L1 | NA | MatrixMating/PathCalling |
| 421 | cbe_251917 | HPV 1a E2 | NA | cbe_4270118 | Hs.211143 | NA | PathCalling |
| 441 | cbe_252041 | HPV 1a L2 | NA | cbe_252090 | Prey2820 | NA | MatrixMating |
| 442 | cbe_252041 | HPV 1a L2 | NA | cbe_2321045 | WNT2B | NA | PathCalling |
| 443 | cbe_252041 | HPV 1a L2 | NA | cbe_2603140 | Prey146 | NA | MatrixMating/PathCalling |
| 444 | cbe_252041 | HPV 1a L2 | NA | cbe_3222359 | LOC51748 | NA | PathCalling |
| 445 | cbe_252041 | HPV 1a L2 | NA | cbe_3456824 | gbh_l20086 | AT | PathCalling |
| 446 | cbe_252041 | HPV 1a L2 | NA | cbe_3606990 | Prey1053670 | NA | PathCalling |
| 447 | cbe_252041 | HPV 1a L2 | NA | cbe_3770440 | gbh_af217505 | NA | MatrixMating/PathCalling |
| 448 | cbe_252041 | HPV 1a L2 | NA | cbe_3810791 | EPHB1 | NA | MatrixMating/PathCalling |
| 449 | cbe_252041 | HPV 1a L2 | NA | cbe_4248793 | GSN | AT/PT | PathCalling |
| 450 | cbe_252041 | HPV 1a L2 | NA | cbe_4268470 | HSPE1 | NA | MatrixMating/PathCalling |
| 451 | cbe_252041 | HPV 1a L2 | NA | cbe_4269628 | RIL | NA | MatrixMating |
| 452 | cbe_252041 | HPV 1a L2 | NA | cbe_4540466 | PUM1 | NA | MatrixMating/PathCalling |
| 576 | cbe_252090 | Prey2820 | NA | cbe_552975 | HPV 1a E1 | NA | MatrixMating |
| 579 | cbe_71919 | FLJ13236 | NA | cbe_552975 | HPV 1a E1 | NA | PathCalling |
| 580 | cbe_251071 | Prey734 | NA | cbe_552975 | HPV 1a E1 | NA | MatrixMating |
| 581 | cbe_552975 | HPV 1a E1 | NA | cbe_3001381 | IRF1 | NA | MatrixMating |
| 582 | cbe_552975 | HPV 1a E1 | NA | cbe_3340492 | TRIP | NA | MatrixMating |
| 583 | cbe_552975 | HPV 1a E1 | NA | cbe_3926508 | TDE1 | AT | PathCalling |
| 584 | cbe_552975 | HPV 1a E1 | NA | cbe_4269167 | TLK2 | SMT | MatrixMating |
| 585 | cbe_552975 | HPV 1a E1 | NA | cbe_4269681 | Hs.224712 | NA | MatrixMating |
| 586 | cbe_552975 | HPV 1a E1 | NA | cbe_4269703 | LOC51182 | NA | MatrixMating |
| 587 | cbe_552975 | HPV 1a E1 | NA | cbe_4269931 | NDUFAB1 | SMT | MatrixMating |
| 280 | cbe_2585091 | SLC5A6 | AT/SMT | cbe_3929637 | HPV 16 E5 | AT/PT | MatrixMating/PathCalling |
| 431 | cbe_251925 | HPV 1a E7 | NA | cbe_3810032 | gbh_af142421 | NA | MatrixMating/PathCalling |
| 316 | cbe_2599246 | Prey1054152 | NA | cbe_3929637 | HPV 16 E5 | AT/PT | PathCalling |
| 317 | cbe_2830508 | Prey142356 | NA | cbe_3929637 | HPV 16 E5 | AT/PT | PathCalling |

TABLE 6
HPV 11, and HPV 16 specific interactions

| | cbe_SEQ ID | COMMON | PT/AT/ | cbe_SEQ ID | COMMON | | PathCalling Screen and/or |
|---|---|---|---|---|---|---|---|
| INT ID | Interactor A | NAME INT A | SMT/NA | Interactor B | NAME INT B | PT/AT/SMT/NA | Matrix Assay |
| 34 | cbe_250834 | HPV 11 E7 | NA | cbe_250878 | Prey302 | NA | MatrixMating/PathCalling |
| 35 | cbe_250834 | HPV 11 E7 | NA | cbe_250883 | Prey244 | NA | MatrixMating/PathCalling |
| 36 | cbe_250834 | HPV 11 E7 | NA | cbe_250888 | Prey228 | NA | MatrixMating/PathCalling |
| 37 | cbe_250834 | HPV 11 E7 | NA | cbe_3056756 | KIAA0440 | NA | MatrixMating/PathCalling |
| 38 | cbe_250834 | HPV 11 E7 | NA | cbe_3454406 | gbh_af252829 | NA | MatrixMating/PathCalling |
| 39 | cbe_250834 | HPV 11 E7 | NA | cbe_3760926 | gbh_al359334 | NA | MatrixMating/PathCalling |
| 40 | cbe_250834 | HPV 11 E7 | NA | cbe_3845697 | Hs.191063 | NA | MatrixMating/PathCalling |
| 41 | cbe_250834 | HPV 11 E7 | NA | cbe_4260642 | PTPN21 | SMT | MatrixMating/PathCalling |
| 42 | cbe_250834 | HPV 11 E7 | NA | cbe_4265947 | RBP4 | AT/PT | MatrixMating/PathCalling |
| 43 | cbe_250834 | HPV 11 E7 | NA | cbe_4268935 | RPL26 | NA | MatrixMating/PathCalling |
| 44 | cbe_250834 | HPV 11 E7 | NA | cbe_4269451 | gbh_af038189 | NA | MatrixMating/PathCalling |
| 45 | cbe_250834 | HPV 11 E7 | NA | cbe_4269681 | Hs.224712 | NA | MatrixMating/PathCalling |
| 46 | cbe_250834 | HPV 11 E7 | NA | cbe_4269818 | Hs.73680 | NA | MatrixMating/PathCalling |
| 47 | cbe_250834 | HPV 11 E7 | NA | cbe_4270015 | RPS26 | NA | MatrixMating/PathCalling |
| 48 | cbe_250834 | HPV 11 E7 | NA | cbe_4270441 | Prey1052970 | NA | PathCalling |
| 49 | cbe_250834 | HPV 11 E7 | NA | cbe_4280774 | ITGB4 | AT/PT | MatrixMating/PathCalling |
| 159 | cbe_251445 | HPV 16 E6 | NA | cbe_2253980 | gbh_d38112_5 | NA | MatrixMating/PathCalling |
| 160 | cbe_251445 | HPV 16 E6 | NA | cbe_2576474 | PTPN2 | AT/SMT | MatrixMating/PathCalling |
| 161 | cbe_251445 | HPV 16 E6 | NA | cbe_4269405 | gbh_af155653 | NA | MatrixMating/PathCalling |
| 162 | cbe_251445 | HPV 16 E6 | NA | cbe_4269703 | LOC51182 | NA | MatrixMating/PathCalling |
| 99 | cbe_251445 | HPV 16 E6 | NA | cbe_4270015 | RPS26 | NA | MatrixMating/PathCalling |
| 163 | cbe_251448 | HPV 16 E7 | NA | cbe_251468 | Prey1628 | NA | MatrixMating/PathCalling |
| 164 | cbe_251448 | HPV 16 E7 | NA | cbe_251502 | Prey1609 | NA | MatrixMating/PathCalling |
| 165 | cbe_251448 | HPV 16 E7 | NA | cbe_422236 | BCR1 | NA | MatrixMating/PathCalling |
| 166 | cbe_251448 | HPV 16 E7 | NA | cbe_2693325 | Prey1673 | NA | MatrixMating/PathCalling |
| 167 | cbe_251448 | HPV 16 E7 | NA | cbe_2898065 | Prey1620 | NA | MatrixMating/PathCalling |
| 168 | cbe_251448 | HPV 16 E7 | NA | cbe_3474077 | KRT4 | NA | MatrixMating |
| 169 | cbe_251448 | HPV 16 E7 | NA | cbe_3773649 | EEF1A1L14 | NA | MatrixMating/PathCalling |
| 170 | cbe_251448 | HPV 16 E7 | NA | cbe_3787129 | LOC55828 | NA | MatrixMating/PathCalling |
| 171 | cbe_251448 | HPV 16 E7 | NA | cbe_3789063 | AP2M1 | NA | MatrixMating/PathCalling |
| 172 | cbe_251448 | HPV 16 E7 | NA | cbe_3834015 | TFPI | AT/PT | MatrixMating |
| 173 | cbe_251448 | HPV 16 E7 | NA | cbe_3921620 | MPP3 | AT | MatrixMating/PathCalling |
| 174 | cbe_251448 | HPV 16 E7 | NA | cbe_4260642 | PTPN21 | SMT | MatrixMating/PathCalling |
| 175 | cbe_251448 | HPV 16 E7 | NA | cbe_4265947 | RBP4 | AT/PT | MatrixMating |
| 176 | cbe_251448 | HPV 16 E7 | NA | cbe_4268611 | NDUFS3 | SMT | MatrixMating/PathCalling |
| 177 | cbe_251448 | HPV 16 E7 | NA | cbe_4268684 | MIZF | NA | MatrixMating/PathCalling |
| 178 | cbe_251448 | HPV 16 E7 | NA | cbe_4268740 | ZNF-U69274 | NA | MatrixMating/PathCalling |
| 179 | cbe_251448 | HPV 16 E7 | NA | cbe_4269658 | PSMC1 | NA | MatrixMating/PathCalling |
| 180 | cbe_251448 | HPV 16 E7 | NA | cbe_4269876 | SBP2 | NA | MatrixMating/PathCalling |
| 181 | cbe_251448 | HPV 16 E7 | NA | cbe_4270186 | STX4A | AT | MatrixMating/PathCalling |
| 182 | cbe_251737 | HPV 16 L2 | NA | cbe_422227 | Prey277610 | NA | PathCalling |
| 183 | cbe_251737 | HPV 16 L2 | NA | cbe_422247 | Prey277642 | NA | PathCalling |
| 184 | cbe_251737 | HPV 16 L2 | NA | cbe_3056756 | KIAA0440 | NA | MatrixMating/PathCalling |
| 185 | cbe_251737 | HPV 16 L2 | NA | cbe_3473488 | gbh_al121903 | NA | PathCalling |
| 186 | cbe_251737 | HPV 16 L2 | NA | cbe_3575702 | SUI1 | NA | PathCalling |
| 187 | cbe_251737 | HPV 16 L2 | NA | cbe_3663966 | KIAA1389 | NA | PathCalling |
| 188 | cbe_251737 | HPV 16 L2 | NA | cbe_3849002 | HSPC224 | AT | PathCalling |
| 189 | cbe_251737 | HPV 16 L2 | NA | cbe_3884811 | Hs.30289 | NA | PathCalling |
| 190 | cbe_251737 | HPV 16 L2 | NA | cbe_4268684 | MIZF | NA | MatrixMating |
| 191 | cbe_251737 | HPV 16 L2 | NA | cbe_4270335 | KIAA0276 | NA | PathCalling |

TABLE 7
HPV1a, HPV 11, and HPV 16 specific interactions

| INT | cbe_SEQ ID | COMMON | | cbe_SEQ ID | COMMON | | PathCalling Screen and/or |
|---|---|---|---|---|---|---|---|
| ID | Interactor A | NAME INT A | PT/AT/SMT/NA | Interactor B | NAME INT B | PT/AT/SMT/NA | Matrix Assay |
| 34 | cbe_250834 | HPV 11 E7 | NA | cbe_250878 | Prey302 | NA | MatrixMating/PathCalling |
| 35 | cbe_250834 | HPV 11 E7 | NA | cbe_250883 | Prey244 | NA | MatrixMating/PathCalling |
| 36 | cbe_250834 | HPV 11 E7 | NA | cbe_250888 | Prey228 | NA | MatrixMating/PathCalling |
| 37 | cbe_250834 | HPV 11 E7 | NA | cbe_3056756 | KIAA0440 | NA | MatrixMating/PathCalling |
| 38 | cbe_250834 | HPV 11 E7 | NA | cbe_3454406 | gbh_af252829 | NA | MatrixMating/PathCalling |
| 39 | cbe_250834 | HPV 11 E7 | NA | cbe_3760926 | gbh_al359334 | NA | MatrixMating/PathCalling |
| 40 | cbe_250834 | HPV 11 E7 | NA | cbe_3845697 | Hs.191063 | NA | MatrixMating/PathCalling |
| 41 | cbe_250834 | HPV 11 E7 | NA | cbe_4260642 | PTPN21 | SMT | MatrixMating/PathCalling |
| 42 | cbe_250834 | HPV 11 E7 | NA | cbe_4265947 | RBP4 | AT/PT | MatrixMating/PathCalling |
| 43 | cbe_250834 | HPV 11 E7 | NA | cbe_4268935 | RPL26 | NA | MatrixMating/PathCalling |
| 44 | cbe_250834 | HPV 11 E7 | NA | cbe_4269451 | gbh_af038189 | NA | MatrixMating/PathCalling |
| 45 | cbe_250834 | HPV 11 E7 | NA | cbe_4269681 | Hs.224712 | NA | MatrixMating/PathCalling |
| 46 | cbe_250834 | HPV 11 E7 | NA | cbe_4269818 | Hs.73680 | NA | MatrixMating/PathCalling |
| 47 | cbe_250834 | HPV 11 E7 | NA | cbe_4270015 | RPS26 | NA | MatrixMating/PathCalling |
| 48 | cbe_250834 | HPV 11 E7 | NA | cbe_4270441 | Prey1052970 | NA | PathCalling |
| 49 | cbe_250834 | HPV 11 E7 | NA | cbe_4280774 | ITGB4 | AT/PT | MatrixMating/PathCalling |
| 163 | cbe_251448 | HPV 16 E7 | NA | cbe_251468 | Prey1628 | NA | MatrixMating/PathCalling |
| 164 | cbe_251448 | HPV 16 E7 | NA | cbe_251502 | Prey1609 | NA | MatrixMating/PathCalling |
| 165 | cbe_251448 | HPV 16 E7 | NA | cbe_422236 | BCR1 | NA | MatrixMating/PathCalling |
| 166 | cbe_251448 | HPV 16 E7 | NA | cbe_2693325 | Prey1673 | NA | MatrixMating/PathCalling |
| 167 | cbe_251448 | HPV 16 E7 | NA | cbe_2898065 | Prey1620 | NA | MatrixMating/PathCalling |
| 168 | cbe_251448 | HPV 16 E7 | NA | cbe_3474077 | KRT4 | NA | MatrixMating |
| 169 | cbe_251448 | HPV 16 E7 | NA | cbe_3773649 | EEF1A1L14 | NA | MatrixMating/PathCalling |
| 170 | cbe_251448 | HPV 16 E7 | NA | cbe_3787129 | LOC55828 | NA | MatrixMating/PathCalling |
| 171 | cbe_251448 | HPV 16 E7 | NA | cbe_3789063 | AP2M1 | NA | MatrixMating/PathCalling |
| 172 | cbe_251448 | HPV 16 E7 | NA | cbe_3834015 | TFPI | AT/PT | MatrixMating |
| 173 | cbe_251448 | HPV 16 E7 | NA | cbe_3921620 | MPP3 | AT | MatrixMating/PathCalling |
| 174 | cbe_251448 | HPV 16 E7 | NA | cbe_4260642 | PTPN21 | SMT | MatrixMating/PathCalling |
| 175 | cbe_251448 | HPV 16 E7 | NA | cbe_4265947 | RBP4 | AT/PT | MatrixMating |
| 176 | cbe_251448 | HPV 16 E7 | NA | cbe_4268611 | NDUFS3 | SMT | MatrixMating/PathCalling |
| 177 | cbe_251448 | HPV 16 E7 | NA | cbe_4268684 | MIZF | NA | MatrixMating/PathCalling |
| 178 | cbe_251448 | HPV 16 E7 | NA | cbe_4268740 | ZNF-U69274 | NA | MatrixMating/PathCalling |
| 179 | cbe_251448 | HPV 16 E7 | NA | cbe_4269658 | PSMC1 | NA | MatrixMating/PathCalling |
| 180 | cbe_251448 | HPV 16 E7 | NA | cbe_4269876 | SBP2 | NA | MatrixMating/PathCalling |
| 181 | cbe_251448 | HPV 16 E7 | NA | cbe_4270186 | STX4A | AT | MatrixMating/PathCalling |
| 427 | cbe_251925 | HPV 1a E7 | NA | cbe_251953 | Prey2724 | NA | MatrixMating/PathCalling |
| 428 | cbe_251925 | HPV 1a E7 | NA | cbe_3753270 | gbh_ac004503 | NA | MatrixMating/PathCalling |
| 429 | cbe_251925 | HPV 1a E7 | NA | cbe_3760926 | gbh_al359334 | NA | MatrixMating/PathCalling |
| 430 | cbe_251925 | HPV 1a E7 | NA | cbe_3789063 | AP2M1 | NA | MatrixMating |
| 431 | cbe_251925 | HPV 1a E7 | NA | cbe_3810032 | gbh_af142421 | NA | MatrixMating/PathCalling |
| 432 | cbe_251925 | HPV 1a E7 | NA | cbe_3820530 | PGAM1 | SMT | MatrixMating/PathCalling |
| 309 | cbe_251925 | HPV 1a E7 | NA | cbe_3834015 | TFPI | AT/PT | MatrixMating |
| 433 | cbe_251925 | HPV 1a E7 | NA | cbe_4265947 | RBP4 | AT/PT | MatrixMating/PathCalling |
| 434 | cbe_251925 | HPV 1a E7 | NA | cbe_4267526 | HRIHFB2206 | NA | MatrixMating/PathCalling |

In certain embodiments, the first polypeptide is labeled. In other embodiments, the second polypeptide is labeled, while in some embodiments, both the first and second polypeptides are labeled. Labeling can be performed using any art-recognized method for labeling polypeptides. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes complexes of two or more polypeptides in which at least one of the polypeptides is present as a fragment of a complex-forming polypeptide according to the invention. For example, one or more polypeptides may include an amino acid sequence sufficient to bind to its corresponding polypeptde. A binding domain of a given first polypeptide can be any number of amino acids sufficient to specifically bind to, and complex with, the corresponding second polypeptide under conditions suitable for complex formation. The binding domain can be the minimal number of amino acids required to retain binding affinity, or may be a larger fragment or derivative of the polypeptides listed in Tables 1 through 7, columns 2 and 5.

Procedures for identifying binding domains can be readily identified by one of ordinary skill in the art including the procedures described herein. For example, nucleic acid sequences containing various portions of a “bait” protein can be tested in a yeast two hybrid screening assay in combination with a nucleic acid encoding the corresponding “prey” protein.

In certain embodiments, the “bait” polypeptide of the complex are HPV derived proteins L1, L2, E2, E4, E5, E6 and E7. In some embodiments the “prey” protein of the complex are identified as cellular proliferation and transformation proteins, proteins involved in protein synthesis, folding or turnover and vesicle trafficking molecules when screened against several human prey libraries.

In other embodiments, the complexes are human ortholog complexes, chimeric complexes, or specific complexes implicated in fungal pathways, as discussed in detail below.

Polypeptides forming the complexes according to the invention can be made using techniques known in the art. For example, one or more of the polypeptides in the complex can be chemically synthesized using art-recognized methods for polypeptide synthesis. These methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989).

Alternatively, polypeptides can be made by expressing one or both polypeptides from a nucleic acid and allowing the complex to form from the expressed polypeptides. Any known nucleic acids that express the polypeptides, whether yeast or human (or chimerics of these polypeptides) can be used, as can vectors and cells expressing these polypeptides. Sequences of yeast ORFs and human polypeptides as referenced in Tables 3 and 7 are publicly available, e.g. at the Saccharomyces Genome Database (SGD) and GenBank (see, e.g. Hudson et al., Genome Res. 7: 1169-1173 (1997). If desired, the complexes can then be recovered and isolated.

Recombinant cells expressing the polypeptide, or a fragment or derivative thereof, may be obtained using methods known in the art, and individual gene product or complex may be isolated and analyzed (See, e.g., e.g., as described in Sambrook et al., eds., MOLECULAR CLONING: A LABORATORY MANUAL, 2nd Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993). This is achieved by assays that are based upon the physical and/or functional properties of the protein or complex. The assays can include, e.g., radioactive labeling of one or more of the polypeptide complex components, followed by analysis by gel electrophoresis, immunoassay, cross-linking to marker-labeled products. Polypeptide complex may be isolated and purified by standard methods known in the art (either from natural sources or recombinant host cells expressing the proteins/protein complex). These methods can include, e.g., column chromatography (e.g., ion exchange, affinity, gel exclusion, reverse-phase, high pressure, fast protein liquid, etc), differential centrifugation, differential solubility, or similar methods used for the purification of proteins.

Combinatorial libraries recognized in the art may be used to provide “prey” proteins. In a preferred embodiment, a keratinocyte library, one of the cell types from tissues normally infected by HPV strains is disclosed.

Complexes Useful for Identifying HPV Infection

The invention further provides complexes of polypeptides useful, inter alia, in identifying agents that inhibit viral proliferation and cell proliferation leading to cancer.

There have been recent breakthroughs in vaccine and immunotherapy for HPV infection (Koutsky, Ault et al. 2002), these therapeutic modes are still under investigation. If these, or other new modalities of therapy prove to be successful in treating HPV infection, it will be necessary to stage and monitor the progression of HPV-induced disease and the course of treatment. The tools described herein are certain to have utility for assessing the stage of disease and the success of treatment.

Protein interactions which are useful for identifying HPV infection are considered below.

Protein interactions that are unique to HPV 1a may be used, for example, in a diagnostic embodiment to determine if an infection by HPV is of a potentially cancer causing nature or not. An interaction that is unique to HPV 1a indicates that the infection is not of a kind that is known to cause cancer or cervical lesions. In one embodiment, an ELISA assay that uses an antibody against one of the non-viral proteins indicated in Tables 1 through 7 as a first antibody and an antibody specific for a virally encoded protein may be used to determine if an infection is actively producing viral protein. Those interactions (indicated “INT ID”) that correspond to the interaction between a protein encoded by the nucleotide sequence indicated by a SEQ ID for Interactor A (“SEQ ID INT A”) with the protein encoded by the nucleic acid represented by a SEQ ID for Interactor B (“SEQ ID INT B”), one or both of which may have a common name (indicated “COMMON NAME INT A”; “COMMON NAME INT B”) one or both of which may have utility as a protein therapeutic (PT), antibody target (AT), or a small molecule target (SMT), and/or may have a therapeutic utility for in indication that may or may not directly involve the interaction of HPV1a, HPV11, HPV16, a combination of the these viruses, or members of the family of viruses they represent (those that cause warts, non-cancerous lesions, or cancerous lesions of the anal-genital tract, and/or head/neck/oral cancers or the non-cancerous versions of the same and the like)(NA). In all cases the interactions have application in the diagnosis of any HPV infections and conditions induced by such infections, active or latent.

Interactions with any one or more PT, AT, SMT, and/or NA for which there exists or may be developed a molecule that therapeutically affects any one or more of the indicated proteins in an interaction with one or more HPV proteins of the strains listed or family members of such viruses may be used as a therapeutic agent for the treatment of HPV infections and/or the conditions caused directly or indirectly by HPV infection.

Chimeric Polypeptides, DNA, Vectors and Recombinant Cells

In a further aspect, the invention provides a chimeric polypeptide that includes sequences of two interacting proteins according to the invention. The interacting proteins can be, e.g., the interacting protein pairs disclosed in Tables 1-7, herein. Also included are chimeric polypeptides including multimers, i.e., sequences from two or more pairs of interacting proteins. An example of such a chimeric polypeptide is a polypeptide that includes amino acid sequences from INT A and INT B, and from INT ID Interactor A and INT ID Interactor B. The chimeric polypeptide includes a region of a first protein covalently linked, e.g. via peptide bond, to a region of a second protein.

In some embodiments, the chimeric polypeptide(s) of the complex include(s) six or more amino acids of a first protein covalently linked to six or more amino acids of a second protein. In other embodiments, the chimeric polypeptide includes at least one binding domain of a first or second protein.

Preferably, the chimeric polypeptide includes a region of amino acids of the first polypeptide able to bind to a second polypeptide. Alternatively, or in addition, the chimeric polypeptide includes a region of amino acids of the second polypeptide able to bind to the first polypeptide.

Nucleic acid encoding the chimeric polypeptide, as well as vectors and cells containing these nucleic acids, are within the scope of the present invention. The chimeric polypeptides can be constructed by expressing nucleic acids encoding chimeric polypeptides using recombinant methods, described above, then recovering the chimeric polypeptides, or by chemically synthesizing the chimeric polypeptides. Host-vector systems that can be used to express chimeric polypeptides include, e.g.: (i) mammalian cell systems which are infected with vaccinia virus, adenovirus; (ii) insect cell systems infected with baculovirus; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

The expression of the specific proteins may be controlled by any promoter/enhancer known in the art including, e.g.: (i) the SV40 early promoter (see e.g., Bernoist & Chambon, Nature 290: 304-310 (1981)); (ii) the promoter contained within the 3′-terminus long terminal repeat of Rous Sarcoma Virus (see e.g., Yamamoto, et al., Cell 22: 787-797 (1980)); (iii) the Herpesvirus thymidine kinase promoter (see e.g., Wagner, et al., Proc. Natl. Acad. Sci. USA 78: 1441-1445 (1981)); (iv) the regulatory sequences of the metallothionein gene (see e.g., Brinster, et al., Nature 296: 39-42 (1982)); (v) prokaryotic expression vectors such as the p-lactamase promoter (see e.g., Villa-Kamaroff, et al., Proc. Natl. Acad. Sci. USA 75: 3727-3731 (1978)); (vi) the tac promoter (see e.g., DeBoer, et al., Proc. Natl. Acad. Sci. USA 80: 21-25 (1983)).

Plant promoter/enhancer sequences within plant expression vectors may also be utilized including, e.g.: (i) the nopaline synthetase promoter (see e.g., Herrar-Estrella, et al., Nature 303: 209-213 (1984)); (ii) the cauliflower mosaic virus 35S RNA promoter (see e.g., Garder, et al., Nuc. Acids Res. 9: 2871 (1981)) and (iii) the promoter of the photosynthetic enzyme ribulose bisphosphate carboxylase (see e.g., Herrera-Estrella, et al., Nature 310: 115-120 (1984)).

Promoter/enhancer elements from yeast and other fungi (e.g., the Gal4 promoter, the alcohol dehydrogenase promoter, the phosphoglycerol kinase promoter, the alkaline phosphatase promoter), as well as the following animal transcriptional control regions, which possess tissue specificity and have been used in transgenic animals, may be utilized in the production of proteins of the present invention.

Other animal transcriptional control sequences derived from animals include, e.g.: (i) the insulin gene control region active within pancreatic β-cells (see e.g., Hanahan, et al., Nature 315: 115-122 (1985)); (ii) the immunoglobulin gene control region active within lymphoid cells (see e.g., Grosschedl, et al., Cell 38: 647-658 (1984)); (iii) the albumin gene control region active within liver (see e.g., Pinckert, et al., Genes and Devel. 1: 268-276 (1987)); (iv) the myelin basic protein gene control region active within brain oligodendrocyte cells (see e.g., Readhead, et al., Cell 48: 703-712 (1987)); and (v) the gonadotrophin-releasing hormone gene control region active within the hypothalamus (see e.g., Mason, et al., Science 234: 1372-1378 (1986)).

The vector may include a promoter operably-linked to nucleic acid sequences which encode a chimeric polypeptide, one or more origins of replication, and optionally, one or more selectable markers (e.g., an antibiotic resistance gene). A host cell strain may be selected which modulates the expression of chimeric sequences, or modifies/processes the expressed proteins in a desired manner. Moreover, different host cells possess characteristic and specific mechanisms for the translational and post-translational processing and modification (e.g., glycosylation, phosphorylation, and the like) of expressed proteins. Appropriate cell lines or host systems may thus be chosen to ensure the desired modification and processing of the foreign protein is achieved. For example, protein expression within a bacterial system can be used to produce an unglycosylated core protein; whereas expression within mammalian cells ensures “native” glycosylation of a heterologous protein.

Antibodies Specific for Polypeptide Complexes

The invention further provides antibodies and antibody fragments (such as Fab or (Fab)₂ fragments) that bind specifically to the complexes described herein. By “specifically binds” is meant an antibody that recognizes and binds to a particular polypeptide complex of the invention, but which does not substantially recognize or bind to other molecules in a sample, or to any of the polypeptides of the complex when those polypeptides are not complexed.

For example, a purified complex, or a portion, variant, or fragment thereof, can be used as an immunogen to generate antibodies that specifically bind the complex using standard techniques for polyclonal and monoclonal antibody preparation.

A full-length polypeptide complex can be used, if desired. Alternatively, the invention provides antigenic fragments of polypeptide complexes for use as immunogens. In some embodiments, the antigenic complex fragment includes at least 6, 8, 10, 15, 20, or 30 or more amino acid residues of a polypeptide. In one embodiment, epitopes encompassed by the antigenic peptide include the binding domains of the polypeptides, or are located on the surface of the protein, e.g., hydrophilic regions.

If desired, peptides containing antigenic regions can be selected using hydropathy plots showing regions of hydrophilicity and hydrophobicity. These plots may be generated by any method well known in the art, including, for example, the Kyte Doolittle or the Hopp Woods methods, either with or without Fourier transformation. See, e.g., Hopp and Woods, Proc. Nat. Acad. Sci. USA 78:3824-3828 (1981); Kyte and Doolittle, J. Mol. Biol. 157:105-142 (1982).

The term “antibody” as used herein refers to immunoglobulin molecules and immunologically active portions of immunoglobulin molecules, i.e., molecules that contain an antigen binding site that specifically binds (immunoreacts with) an antigen, such as a polypeptide complex. Such antibodies include, e.g., polyclonal, monoclonal, chimeric, single chain, Fab and F(ab′)₂ fragments, and an Fab expression library. In specific embodiments, antibodies to human ortholog complexes.

Various procedures known within the art may be used for the production of polyclonal or monoclonal antibodies. For example, for the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed polypeptide complex. Alternatively, the immunogenic polypeptides or complex may be chemically synthesized, as discussed above. The preparation can further include an adjuvant. Various adjuvants used to increase the immunological response include, e.g., Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against complex can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of a polypeptide complex. A monoclonal antibody composition thus typically displays a single binding affinity for a particular protein with which it immunoreacts. For preparation of monoclonal antibodies directed towards a particular complex, or polypeptide, any technique that provides for the production of antibody molecules by continuous cell line culture may be utilized. Such techniques include, e.g., the hybridoma technique (see Kohler & Milstein, Nature 256: 495-497 (1975)); the trioma technique; the human B-cell hybridoma technique (see Kozbor, et al., Immunol Today 4: 72 (1983)); and the EBV hybridoma technique to produce human monoclonal antibodies (see Cole, et al., In: Monoclonal Antibodies and Cancer Therapy, Alan R. Liss, Inc., (1985) pp. 77-96). If desired, human monoclonal antibodies may be prepared by using human hybridomas (see Cote, et al., Proc. Natl. Acad. Sci. USA 80: 2026-2030 (1983)) or by transforming human B-cells with Epstein Barr Virus in vitro (see Cole, et al., In: Monoclonal Antibodies and Cancer Therapy, supra).

Methods can be adapted for the construction of Fab expression libraries (see e.g. Huse, et al., Science 246: 1275-1281 (1989)) to allow rapid and effective identification of monoclonal F_{ab} fragments with the desired specificity for the desired protein or derivatives, fragments, analogs or homologs thereof. Non-human antibodies can be “humanized” by techniques well known in the art. See e.g., U.S. Pat. No. 5,225,539. Antibody fragments that contain the idiotypes to a polypeptide or polypeptide complex may be produced by techniques known in the art including, e.g.: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Chimeric and humanized monoclonal antibodies against the polypeptide complexes, or polypeptides, described herein are also within the scope of the invention, and can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al., Science 240: 1041-1043 (1988); Liu et al., Proc. Nat. Acad. Sci. USA 84: 3439-3443 (1987); Liu et al., J. Immunol. 139: 3521-3526 (1987); Sun et al., Proc. Nat. Acad. Sci. USA 84: 214-218 (1987); Nishimura et al., Cancer Res. 47: 999-1005 (1987); Wood et al., Nature 314: 446-449 (1985); Shaw et al., J. Natl. Cancer Inst. 80: 1553-1559 (1988); Morrison, Science 229: 1202-1207 (1985); Oi et al., BioTechniques 4: 214 (1986); U.S. Pat. No. 5,225,539; Jones et al., Nature 321: 552-525 (1986); Verhoeyan et al., Science 239: 1534 (1988); and Beidler et al., J. Immunol. 141: 4053-4060 (1988).

Methods for the screening of antibodies that possess the desired specificity include, e.g., enzyme-linked immunosorbent assay (ELISA) and other immunologically-mediated techniques known within the art. For example, selection of antibodies that are specific to a particular domain of a polypeptide complex is facilitated by generation of hybridomas that bind to the complex, or fragment thereof, possessing such a domain.

In certain embodiments of the invention, antibodies specific for the polypeptide complexes described herein may be used in various methods, such as detection of complex, and identification of agents which disrupt complexes. These methods are described in more detail, below. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Polypeptide complex-specific, or polypeptide-specific antibodies, can also be used to isolate complexes using standard techniques, such as affinity chromatography or immunoprecipitation. Thus, the antibodies disclosed herein can facilitate the purification of specific polypeptide complexes from cells, as well as recombinantly produced complexes expressed in host cells.

Kits

In a specific embodiment, the invention provides kits containing a reagent, for example, an antibody described above, which can specifically detect a polypeptide complex, or a constituent polypeptide, described herein. Such kits can contain, for example, reaction vessels, reagents for detecting complex in sample, and reagents for development of detected complex, e.g. a secondary antibody coupled to a detectable marker. The label incorporated into the anti-complex, or anti-polypeptide antibody may include, e.g., a chemiluminescent, enzymatic, fluorescent, colorimetric or radioactive moiety. Kits of the present invention may be employed in diagnostic and/or clinical screening assays.

Pharmaceutical Compositions

The invention further provides pharmaceutical compositions of purified complexes suitable for administration to a subject, most preferably, a human, in the treatment of disorders involving altered levels of such complexes. Such preparations include a therapeutically-effective amount of a complex, and a pharmaceutically acceptable carrier. As utilized herein, the term “pharmaceutically acceptable” means approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils.

The therapeutic amount of a complex which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. However, suitable dosage ranges for intravenous administration of the complexes of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

Various delivery systems are known and can be used to administer a pharmaceutical preparation of a complex of the invention including, e.g.: (i) encapsulation in liposomes, microparticles, microcapsules; (ii) recombinant cells capable of expressing the polypeptides of the complex; (iii) receptor-mediated endocytosis (see, e.g., Wu et al., J. Biol. Chem. 262: 4429-4432 (1987)); (iv) construction of a nucleic acid encoding the polypeptides of the complex as part of a retroviral or other vector, and the like.

Methods of administration include, e.g., intradermal, intramuscular, intraperitoneal, intravenous, subcutaneous, intranasal, epidural, and oral routes. The pharmaceutical preparations of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the pharmaceutical preparation into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the pharmaceutical preparation locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Alternatively, pharmaceutical preparations of the invention may be delivered in a vesicle, in particular a liposome, (see, e.g., Langer, Science 249:1527-1533 (1990)) or via a controlled release system including, e.g., a delivery pump (see, e.g., Saudek, et al., New Engl. J. Med. 321: 574 (1989) and a semi-permeable polymeric material (see, e.g., Howard, et al., J. Neurosurg. 71: 105 (1989)). Additionally, the controlled release system can be placed in proximity of the therapeutic target (e.g., the brain), thus requiring only a fraction of the systemic dose. See, e.g., Goodson, In: Medical Applications of Controlled Release, 1984 (CRC Press, Bocca Raton, Fla.).

Screening, Diagnostic, and Therapeutic Methods

The invention further provides methods of identifying an agent which modulate formation or stability a polypeptide complex described herein. By modulate is meant to increase or decrease the rate at which the complex is assembled or dissembled, or to increase or decrease the stability of an assembled complex. Thus, an agent can be tested for its ability to disrupt a complex, or to promote formation or stability of a complex.

In one embodiment, the invention provides a method of identifying an agent that promotes disruption of a complex. The method includes providing a polypeptide complex, contacting the complex with a test agent, and detecting the presence of a polypeptide displaced from the complex. The presence of displaced polypeptide indicates the disruption of the complex by the agent. In some embodiments, the complex is a human ortholog complex, as described above, which includes “bait” and “prey” proteins selected from those recited in Tables 4 through 7. Agents which disrupt complexes of the invention may present novel modulators of cell processes and pathways in which the complexes participate. For example, agents which disrupt complexes involving EGFR proteins, DLG, ubiquitin 9 or may be selected as potential HPV therapeutics. In another embodiment, against which modulate the activity of BCR1, GAP or other effectors of CDC42-pathways are encompassed by the invention.

Any compound or other molecule (or mixture or aggregate thereof) can be used as a test agent. In some embodiments, the agent can be a small peptide, or other small molecule produced by e.g., combinatorial synthetic methods known in the art. Disruption of the complex by the test agent, e.g. binding of the agent to the complex, can be determined using art recognized methods, e.g., detection of polypeptide using polypeptide-specific antibodies, as described above. Bound agents can alternatively be identified by comparing the relative electrophoretic mobility of complexes exposed to the test agent to the mobility of complexes that have not been exposed to the test agent.

Agents identified in the screening assays can be further tested for their ability to alter and/or modulate cellular functions, particularly those functions in which the complex has been implicated. These functions include, e.g., control of cell-cycle progression; regulation of transcription; control of intracellular signal transduction, etc., as described in detail above.

In another embodiment, the invention provides methods for inhibiting the interaction of a polypeptide with a ligand, by contacting a complex of the protein and the ligand with an agent that disrupts the complex, as described above. In certain embodiments, the polypeptides are associated with protein synthesis, folding or turnover and vesicle trafficking molecules. In certain embodiments, the ligand is an interacting polypeptide, and the polypeptide and ligands are selected from those recited in Tables 1-7, preferably Tables 4-7, and most preferably interactions involving HPV 16. Inhibition of complex formation allows for modulation of cellular functions and pathways in which the targeted complexes participate.

In another embodiment, the invention provides a method for identifying a polypeptide complex in a subject. The method includes the steps of providing a biological sample from the subject, detecting, if present, the level of polypeptide complex. In some embodiments, the complex includes a first polypeptide (a “bait” polypeptide) selected from the polypeptides recited in Tables 4-7, column 2 or 3, and a second polypeptide (“prey” polypeptide) selected from the polypeptides recited in Tables 4-7, column 5 or 6. Any suitable biological sample potentially containing the complex may be employed, e.g. blood, urine, cerebral-spinal fluid, plasma, skin, etc. Complexes may be detected by, e.g., using complex-specific antibodies as described above. The method provides for diagnostic screening, including in the clinical setting, using, e.g., the kits described above.

In still another embodiment, the present invention provides methods for detecting a polypeptide in a biological sample, by providing a biological sample containing the polypeptide, contacting the sample with a corresponding polypeptide to form a complex under suitable conditions, and detecting the presence of the complex. A complex will form if the sample does, indeed, contain the first polypeptide. In some embodiments, the polypeptide being detecting is a “prey” protein selected from the polypeptides recited in Tables 1 through 7, columns 2 or 3, and is detected by complexing with the corresponding “bait” protein recited in Tables 1-7, column 5 or 6. Conversely, in other embodiments the polypeptide being detected is the “bait” protein. Alternatively, a yeast “bait” or “prey” ortholog may be employed to form a chimeric complex with the polypeptide in the biological sample.

In still another embodiment, the invention provides methods for removing a first polypeptide from a biological sample by contacting the biological sample with the corresponding second peptide to form a complex under conditions suitable for such formation. The complex is then removed from the sample, effectively removing the first polypeptide. As with the methods of detecting polypeptide described above, the polypeptide being removed may be either a “bait” or “prey” protein, and the second corresponding polypeptide used to remove it may be either a yeast or human ortholog polypeptide.

Methods of determining altered expression of a polypeptide in a subject, e.g. for diagnostic purposes, are also provided by the invention. Altered expression of proteins involved in cell processes and pathways can lead to deleterious effects in the subject. Altered expression of a polypeptide in a given pathway leads to altered formation of complexes which include the polypeptide, hence providing a means for indirect detection of the polypeptide level. The method involves providing a biological sample from a subject, measuring the level of a polypeptide complex of the invention in the sample, and comparing the level to the level of complex in a reference sample having known polypeptide expression. A higher or lower complex level in the sample versus the reference indicates altered expression of either of the polypeptides that forms the complex. The detection of altered expression of a polypeptide can be use to diagnose a given disease state, and or used to identify a subject with a predisposition for a disease state. Any suitable reference sample may be employed, but preferably the test sample and the reference sample are derived from the same medium, e.g. both are urine, etc. The reference sample should be suitably representative of the level polypeptide expressed in a control population.

The invention further provides methods for treating or preventing a disease or disorder involving altered levels of a polypeptide complex, or polypeptide, disclosed herein, by administering to a subject a therapeutically-effective amount of at least one molecule that modulates the function of the complex. As discussed above, altered levels of polypeptide complexes described herein may be implicated in disease states resulting from a deviation in normal function of the pathway in which a complex is implicated. For example, altered levels of the observed complex between E6 or E7 and DLG tumor suppressor or Ubiquitin protease 9 may be implicated in abrogation of p53 interactions. In subjects with a deleteriously high level of complex, modulation may consist, for example, by administering an agent which disrupts the complex, or an agent which does not disrupt, but down-regulates, the functional activity of the complex. Alternatively, modulation in subjects with a deleteriously low level of complex may be achieved by pharmaceutical administration of complex, constituent polypeptide, or an agent which up-regulates the functional activity of complex. Pharmaceutical preparations suitable for administration of complex are described above.

In one embodiment, a disease or disorder involving altered levels of a polypeptide selected from the polypeptides recited in Tables 1 through 7 or the corresponding polypeptides in columns 2 or 5, are treated by administering a molecule that modulates the function of the polypeptide. In certain embodiments, the modulating molecule is the corresponding polypeptide, e.g. administering a “prey” protein corresponding to a “bait” protein modulates the latter by forming a complex with it.

The details of one or more embodiments of the invention are set forth in the description above. Although any methods and materials similar or equivalent to those described herein can be used in the practice of the present invention, the preferred methods and materials are now described. For example, additional interactions can be identified using other two-hybrid systems (i.e. using a LexA binding domain fusion or HIS3 as a reporter gene), including variables such as different protein domains or genomic activation domain libraries. Other features, objects, and advantages of the invention will be apparent from the description and from the claims.

The practice of the present invention generally employs conventional techniques of molecular biology, microbiology, recombinant DNA, and immunology, which are within the skill of the art. Such techniques are explained fully in the literature. See for example J. Sambrook et al., “Molecular Cloning; A Laboratory Manual” (1989); “DNA Cloning”, Vol. I and II (D. N. Glover ed., 1985); “Oligonucleotide Synthesis” (M. J. Gait ed., 1984); “Nucleic Acid Hybridization” (B. D. Hames and S. J. Higgins eds., 1984); “Transcription and Translation” (B. D. Hames & S. J. Higgins eds., 1984); “Animal Cell Culture” (R. I. Freshney ed., 1986); “Immobilized Cells and Enzymes” (IRL Press, 1986); “A Practical Guide to Molecular Cloning” (B. Perbal, 1984); the series, “Methods in Enzymology” (Academic Press, Inc.); “Gene Transfer Vectors for Mammalian Cells” (J. H. Miller and M. P. Calos eds., 1987, Cold Spring Harbor Laboratory); Meth. Enzymol. (1987) 154 and 155 (Wu and Grossman, and Wu eds., respectively); “Immunochemical Methods in Cell and Molecular Biology” (Academic Press, London); “Protein Purification: Principles and Practice”, Third Ed. (Scopes, Springer-Verlag, N.Y., 1994); and “Handbook of Experimental Immunology”, Volumes I-IV (Weir and Blackwell, eds., 1986).

The following examples are presented in order to more fully illustrate the preferred embodiments of the invention. These examples should in no way be construed as limiting the scope of the invention, as defined by the appended claims.

### EXAMPLE 1

### SeqCalling™ Technology

cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly was included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

### EXAMPLE 2

### Identifying Nucleic Acids and Proteins by PathCalling™

The sequences of the HPV proteins and interactors in this application were derived by laboratory cloning of cDNA fragments and by in silico prediction of the sequence as described in Example A. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof.

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids.

Preparation of Yeast Cells

Yeast lysates were produced as follows: 1-1.5 ml samples from a yeast culture were removed, samples were frozen on dry ice. On ice, low-salt lysis Buffer was added to the cell pellets. Glass beads were added, the cells were resuspended by a brief vortexing. The cells were lysed by beating the beads for 90 sec. The lysate was placed on ice for 5 min and the beads beaten again for 90 sec. The sample were put back on ice. Once the lysate was recovered free of beads, the lysate was centrifuge at maximum speed in a microcentrifuge for 3 to 5 min at 4° C. and put back on ice. 25 to 50 μl were removed from the supernatant and mixed with an equal volume of 2× Protein Sample Buffer then saved for Western analysis.

Immunoprecipitation from Yeast

The lysate samples were thawed and the desired volume (based on the protein concentration) were put into a fresh microcentrifuge tube. All the samples were made into the same volume with fresh low-salt lysis Buffer. Antibody was diluted in Low-Salt Lysis Buffer (10 μl per sample) and mixed by vortexing. This was incubated on ice for 30 min.

ProteinA-Sepharose/Antibody Binding

Protein A-Sepharose beads were equilibrated with low-salt lysis Buffer by suspending the beads in low-salt Buffer, centrifuging briefly to sediment the beads and removing the supernatant. This equilibration was repeated then a wash step was peformed for 2 or 3 times. The Buffer-equilibrated beads were aliquoted into fresh 0.5 ml microcentrifuge tubes making sure that all the tubes had an equal amount of beads. The antibody/extract mixture was centrifuged in a microcentrifuge at full speed for 1 min at 4° C. The supernatant was recovered and added to the proteinA-Sepharose. The mixture was mixed in an end-over-end rotator for 1 to 2 hr at 4° C. then centrifuged briefly in a microcentrifuge (bringing the centrifuge up to full speed and then back down). The supernatant was removed. Keeping the samples on ice as much as possible, the beads were washed by adding 400 μl of bead Buffer. The beads were resuspended and centrifuged again. The supernatant was removed. The beads were resuspended in bead Buffer and the mixture transferred to a fresh tube. The old tube was rinsed with more bead-Buffer to recover residual beads to the new tube. The beads were centrifuged, the supernatant removed and the beads washed with Bead Buffer.

If the immunoprecipitate is only for analysis of radio-labeled proteins bound, the beads can be simply resuspended in protein sample Buffer, boiled for 90 sec and electrophoresed. If an enzymatic assay of some sort is involved, the beads should be washed in the reaction Buffer 1 or 2 times.

In cases where interactions cannot be validated in the yeast system, the interacting proteins are tagged with different epitopes at the N or the C-terminus and expressed in appropriate mammalian cell lines by transient transfection. The cells are grown for 48-72 h, lysed, and the substrate protein is immunoprecipitated using antibody specific to the epitope and analyzed by western blotting as described for the yeast system.

### EXAMPLE 3

### Yeast 2 Hybrid Analysis of Interactions

The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corporation proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries.

Physical clone: the cDNA fragment derived by the screening procedure is a recombinant DNA covering the entire open reading frame. The cDNA was cloned into pACT2 plasmid (Clontech) and used to make the cDNA library. The recombinant plasmid was inserted into the host and selected by the yeast hybrid diploid generated during the screening procedure by the mating of both CuraGen Corporation proprietary yeast strains N₁O₆′ and YULH (U.S. Pat. Nos. 6,057,101 and 6,083,693) to provide the clones.

Interaction protein pairs are added to CuraGen's PathCalling™ Protein Interaction Database. This database allows for the discovery of novel pharmaceutical drug targets by virtue of their interactions and/or presence in pathologically related signaling pathways. Protein interactions are subsequently analyzed using bioinformatic tools within GeneScape™, which provides a means of visualization of binary protein interactions, protein complex formation, as well as complete cellular signaling pathways. Specifically, the sequences, which encode proteins identified by INT ID in Tables 1 through 3 were found to interact and resulted in the formation of a protein complex within a series of complexes resulting in identification of a protein interaction. The interaction is specifically relevant to HPV pathology. The specific interactions, which constitute the specific complexes, is useful for therapeutic intervention through the use of recombinant protein or antibody therapies, small molecule drugs, or gene therapy approaches. Protein interactions, which are identified through the mining of the PathCalling™ database, can be screened in vitro and in vivo to provide expression, functional, biochemical, and phenotypic information. Assays for expression, functional, biochemical, phenotypic, diagnostic, prognostic, monitoring of HPV-induced tumors' and/or lesions' response to therapy, immunization, therapeutic immunization, immunotherapy, tumor burden monitoring, ELISA assay to determine if an infection is active or latent, and the like may be used alone or in conjunction and include, but are not limited to the following technologies; RTQ-PCR, transfection of recombinant proteins, co-immunoprecipitation and mass spectrometry, FRET, Affinity Chromatography, Immunohistochemisty or Immunocytochemistry, gene CHIP hybridizations, antisense (i.e. knock-down, knock-up), GeneCalling experiments, and/or biochemical assays (phosphorylation, dephosphorylation, protease, etc . . . ).

Example D Protein-Protein Interactions

The amino acid sequences of the polypeptides involved in the novel protein-protein interactions and the nucleic acid sequences of the polynucleotides which encode them are listed below.

Protein SEQ IDs:

TABLE 10

| | |
|---|---|
| cbe_1380530 RPL13 | |
| ribosomal protein L13 | |
| length = 211 | |
| MAPSRNGMVLKPHFHKDWQRRVATWFNQPARKIRRRKARQAKARRIAPRPASGPIRPIVR | SEQ ID NO: 1 |
| CPTVRYHTKVRAGRGFSLEELRVAGIHKKVARTIGISVDPRRRNKSTESLQTNVQRLKEY | |
| RSKLILFPRKPSAPKKGDSSAEELKLATQLTGPVMPVRNVYKKEKARVITEEEKNFKAFA | |
| SLRMARANARLFGIRAKRAKEAAEQDVEKKK | |

TABLE 11

| | |
|---|---|
| cbe_1469980 FLJ20259 | |
| hypothetical protein FLJ20259 | |
| length = 776 | |
| MNNSLENTISFEEYIRVKARSVPQHRMKEFLDSLASKGPEALQEFQQTATTTMVYQQGGN | SEQ ID NO: 2 |
| CIYTDSTEVAGSLLELACPVTTSVQPQTQQEQQIQVQQPQQVQVQVQVQQSPQQVSAQLS | |
| PQLTVHQPTEQPIQVQVQIQGQAPQSAAPSIQTPSLQSPSPSQLQAAQIQVQHVQAAQQI | |
| QAAEIPEEHIPHQQIQAQLVAGQSLAGGQQIQIQTVGALSPPPSQQGSPREGERRVGTAS | |
| VLQPVKKRKVEMPITVSYAISGQPVATVLAIPQGQQQSYVSLRPDLLTVDSAHLYSATGT | |
| ITSPTGETWTIPVYSAQPRGDPQQQSITHIAIPQEAYNAVHVSGSPTALAAVKLEDDKEK | |
| MVGTTSVVKNSHEEVVQTLANSLFPAQFMNGNIHIPVAVQAVAGTYQNTAQTVHIWDPQQ | |
| QPQQQTPQEQTPPPQQQQQQLQVTCSAQTVQVAEVEPQSQPQPSPELLLPNSLKPEEGLE | |
| VWKNWAQTKNAELEKDAQNRLAPIGRRQLLRFQEDLISSAVAELNYGLCLMTREARNGEG | |
| EPYDPDVLYYIFLCIQKYLFENGRVDDIFSDLYYVRFTEWLHEVLKDVQPRVTPLGYVLP | |
| SHVTEEMLWECKQLGAHSPSTLLTTLMFFNTKYFLLKTVDQHMKLAFSKVLRQTKKNPSN | |
| PKDKSTSIRYLKALGIHQTGQKVTDDMYAEQTENPENPLRCPIKLYDFYLFKCPQSVKGR | |
| NDTFYLTPEPVVAPNSPIWYSVQPISREQMGQMLTRILVIREIQEAIAVANASTMH | |

TABLE 12

| | |
|---|---|
| cbe_1505425 HCDI | |
| HCDI protein | |
| length = 319 | |
| MGWRRKRVPQRGRKAPPPQLHGNINNLYFPIRWRDRLHWDSPNPAAECQRHEVTLVSRKP | SEQ ID NO: 3 |
| GPGRITWDELAASGLPSCDAAVNLAGENILNPLRRWNETFQKEVLGSRLETTQLLAKAIT | |
| KAPQPPKAWVLVTGVAYYQPSLTAEYDEDSPGGDFDFFSNLVTKWEAAARLPGDSTRQVV | |
| VRSGVVLGRGGGAMGHMLLPFRLGLGGPIGSGHQFFPWIHIGDLAGILTHALEANHVHGV | |
| LNGVAPSSATNAEFAQTLGAALGRRAFIPLPSAVVQAVFGRQRAIMLLEGQKVIPQRTLA | |
| TGYQYSFPELGAALKEIVA | |

TABLE 13

| | |
|---|---|
| cbe_1710 QP-C | |
| low molecular mass ubiquinone-binding protein (9.5 kD) | |
| length = 93 | |
| MGREFGNLTRMRHVISYSLSPFEQRAYPHVFTKGIPNVLRRIRESFFRVVPQFVVFYLIY | SEQ ID NO: 4 |
| TWGTEEFERSKRRIQLPMKMTNEQRIRMTVPCL | |

TABLE 14

cbe_1796502 CTNND2
catenin (cadherin-associated protein), delta 2 (neural plakophilin-related
arm-repeat protein)
length = 1225

| | |
|---|---|
| MFARKPPGAAPLGAMPVPDQPSSASEKTSSLSPGLNTSNGDGSETETTSAILASVKEQEL | SEQ ID NO: 5 |
| QFERLTRELEAERQIVASQLERCKLGSETGSMSSMSSAEEQFQWQSQDGQKDIEDELTTG | |
| LELVDSCIRSLQESGILDPQDYSTCERPSLLSQSALQLNSKPEGSFQYPASYHSNQTLAL | |
| GETTPSQLPARGTQARATGQSFSQGTTSPAGHLAGPEPAPPPPPPPREPFAPSLGSAFHL | |
| PDAPPAAAAAALYYSSSTLPAPPRGGSPLAAPQGGSPTKLQRGGSAPEGATYAAPRGSSP | |
| KQSPSRLAKSYSTSSPINIVVSSAGLSPIRVTSPPTVQSTISSSPIHQLSSTIGTYATLS | |
| PTKRLVHASEQYSKHSQELYATATLQRPGSLAAGSRASYSSQHGHLGPELRALQSPEHHI | |
| DPIYEDRVYQKPPMRSLSQSQCDPLPPAHTGTYRTSTAPSSPGVDSVPLQRTGSQHGPQN | |
| AAAATFQRASYAAGPASNYADPYRQLQYCPSVESPYSKSGPALPPEGTLARSPSIDSIQK | |
| DPREFGWRDPELPEVIQMLQHQFPSVQSNAAAYLQHLCFGDNKIKAEIRRQGGIQLLVDL | |
| LDHRMTEVHRSACGALRNLVYGKANDDNKIALKWCGGIPALVRLLRKTTDLEIRELVTGV | |
| LWNLSSCDALKMPIIQDALAVLTNAVIIPHSGWENSPLQDDRKIQLHSSQVLRNATGCLR | |
| NVSSAGEEARRRMRECDGLTDALLYVIQSALGSSEIDSKTVENCVCILRNLSYRLAAETS | |
| QGQHMGTDELDGLLCGEANGKDAESSGCWGKKKKKKKSQDQWDGVGPLPDCAEPPKGIQM | |
| LWHPSIVKPYLTLLSECSNPDTLEGAACALQNLAAGSWKWSVYIRAAVRKEKGLPILVEL | |
| LRIDNDRVVCAVATALRNMALDVRNKELIGKYAMRDLVHRLPGGNNSNNTASKANSDDTV | |
| TAVCCTLHEVITKNMENAKALRDAGGIEKLVGISKSKGDKHSPKVVKAASQVLNSMWQYR | |
| DLRSLYKKDGWSQYHFVASSSTIERDRQRPYSSSRTPSISPVRVSPNNRSASAPASPREM | |
| ISLKERKTDYECTGSNATYHGAKGEHTSRKDANTAQNTGISTLYRNSYGAPAEDIKHNQV | |
| SAQPVPQEPSRKDYETYQPFQNSTRNYDESFFEDQVHHRPPASEYTMHLGLKSTGNYVDF | |
| YSAARPYSELNYETSHYPASPDSWV | |

TABLE 15

| | |
|---|---|
| cbe_1807361 FLJ22729 | |
| hypothetical protein FLJ22729 | |
| length = 158 | |
| MRGIYSSSVYLEEISSIISKMPKADFYVLEKTGLSIQNSSLFPILLHFHIMEANLYALLN | SEQ ID NO: 6 |
| KTFAQDGQHQVLSMNRNAVGKHFELMIGDSRTSGKELVKQFLFDSILKADPRVFFPSDKI | |
| VHYRQMFLSTELQRVEELYDSLLQAIAFYELAVFDSQP | |

TABLE 16

| | |
|---|---|
| cbe_2253980 Prey1135 | |
| Homo sapiens, clone MGC: 14302, mRNA, | |
| complete cds. | |
| length = 24 | |
| MHYSPDASTAFSSIAHITRDVNYG | SEQ ID NO: 7 |

TABLE 17

| | |
|---|---|
| cbe_244811 DGUOK | |
| deoxyguanosine kinase | |
| length = 189 | |
| MAAGRLFLSRLRAPFSSMAKSPLEGVSSSRGLHAGRGPRRLSIEGNIAVGKSTFVKLLTK | SEQ ID NO: 8 |
| TYPEWHVATEPVATWQNIQAAGTQKACTAQSLGNLLDMMYREPARWSYTFQTFSFLSRLK | |
| VQLEPFPEKLLQARKPVQIFERSVYSDRLHFEALMNIPVLVLDVNDDFSEEVTKQEDLMR | |
| EVNTFVKNL | |

TABLE 18

| | |
|---|---|
| cbe_250777 HPV 11 E4 | |
| Human papillomavirus 11 E4 | |
| length = 108 | |
| MVVPIIGKYVMAAQLYVLLHLYLALYEKYPLLNLLHTPPHRPPPLQCPPAPRKTACRRRL | SEQ ID NO: 9 |
| GSEHVDRPLTTPCVWPTSDPWTVQSTTSSLTITTSTKEGTTVTVQLRL | |

TABLE 19

| | |
|---|---|
| cbe_250781 HPV 11 E2 | |
| Human papillomavirus 11 E2 | |
| length = 367 | |
| MEAIAKRLDACQDQLLELYEENSIDIHKHIMHWKCIRLESVLLHKAKQMGLSHIGLQVVP | SEQ ID NO: 10 |
| PLTVSETKGHNAIEMQMHLESLAKTQYGVEPWTLQDTSYEMWLTPPKRCFKKQGNTVEVK | |
| FDGCEDNVMEYVVWTHIYLQDNDSWVKVTSSVDAKGIYYTCGQFKTYYVNFNKEAQKYGS | |
| TNHWEVCYGSTVICSPASVSSTVREVSIAEPTTYTPAQTTAPTVSACTTEDGVSAPPRKR | |
| ARGPSTNNTLCVANIRSVDSTINNIVTDNYNKHQRRNNCHSAATPIVQLQGDSNCLKCFR | |
| YRLNDKYKHLFELASSTWHWASPEAPHKNAIVTLTYSSEEQRQQFLNSVKIPPTIRHKVG | |
| FMSLHLL | |

TABLE 20

| | |
|---|---|
| cbe_250808 HPV 11 E6 | |
| Human papillomavirus 11 E6 | |
| length = 150 | |
| MESKDASTSATSIDQLCKTFNLSLHTLQIQCVFCRNALTTAEIYAYAYKNLKVVWRDNFP | SEQ ID NO: 11 |
| FAACACCLELQGKINQYRHFNYAAYAPTVEEETNEDILKVLIRCYLCHKPLCEIEKLKHI | |
| LGKARFIKLNNQWKGRCLHCWTTCMEDLLP | |

TABLE 21

| | |
|---|---|
| cbe_250834 HPV 11 E7 | |
| Human papillomavirus 11 E7 | |
| length = 98 | |
| MHGRLVTLKDIVLDLQPPDPVGLHCYEQLEDSSEDEVDKVDKQDAQPLTQHYQILTCCCG | SEQ ID NO: 12 |
| CDSNVRLVVECTDGDIRQLQDLLLGTLNIVCPICAPKP | |

TABLE 22

| | |
|---|---|
| cbe_251059 HPV 16 E1-E4 | |
| HPV 16 E1-E4 | |
| length = 92 | |
| MADPAAATKYPLLKLLGSTWPTTPPRPIPKPSPWAPKKHRRLSSDQDQSQTPETPATPLS | SEQ ID NO: 13 |
| CCTETQWTVLQSSLHLTAHTKDGLTVIVTLHP | |

TABLE 23

| | |
|---|---|
| cbe_251063 HPV 16 E1 | |
| Human papillomavirus 16 E1 | |
| length = 649 | |
| MADPAGTNGEEGTGCNGWFYVEAVVEKKTGDAISDDENENDSDTGEDLVDFIVNDNDYLT | SEQ ID NO: 14 |
| QAETETAHALFTAQEAKQHRDAVQVLKRKYLGSPLSDISGCVDNNISPRLKAICIEKQSR | |
| AAKRRLFESEDSGYGNTEVETQQMLQVEGRHETETPCSQYSGGSGGGCSQYSSGSGGEGV | |
| SERHTICQTPLTNILNVLKTSNAKAAMLAKFKELYGVSFSELVRPFKSNKSTCCDWCIAA | |
| FGLTPSIADSIKTLLQQYCLYLHIQSLACSWGMVVLLLVRYKCGKNRETIEKLLSKLLCV | |
| SPMCMMIEPPKLRSTAAALYWYKTGISNISEVYGDTPEWIQRQTVLQHSFNDCTFELSQM | |
| VQWAYDNDIVDDSEIAYKYAQLADTNSNASAFLKSNSQAKIVKDCATMCRHYKRAEKKQM | |
| SMSQWIKYRCDRVDDGGDWKQIVMFLRYQGVEFMSFLTALKRFLQGIPKKNCILLYGAAN | |
| TGKSLFGMSLMKFLQGSVICFVNSKSHFWLQPLADAKIGMLDDATVPCWNYIDDNLRNAL | |
| DGNLVSMDVKHRPLVQLKCPPLLITSNINAGTDSRWPYLHNRLVVFTFPNEFPFDENGNP | |
| VYELNDKNWKSFFSRTWSRLSLHEDEDKENDGDSLPTFKCVSGQNTNTL | |

TABLE 24

| | |
|---|---|
| cbe_251088 HPV 16 E2 | |
| Human papillomavirus 16 E2 | |
| length = 365 | |
| METLCQRLNVCQDKILTHYENDSTDLRDHIDYWKHMRLECAIYYKAREMGFKHINHQVVP | SEQ ID NO: 15 |
| TLAVSKNKALQAIELQLTLETIYNSQYSNEKWTLQDVSLEVYLTAPTGCIKKHGYTVEVQ | |
| FDGDICNTMHYTNWTHIYICEEASVTVVEGQVDYYGLYYVHEGIRTYFVQFKDDAEKYSK | |
| NKVWEVHAGGQVILCPTSVFSSNEVSSPEIIRQHLANHPAATHTKAVALGTEETQTTIQR | |
| PRSEPDTGNPCHTTKLLHRDSVDSAPILTAFNSSHKGRINCNSNTTPIVHLKGDANTLKC | |
| LRYRFKKHCTLYTAVSSTWHWTGHNVKHKSAIVTLTYDSEWQRDQFLSQVKIPKTITVST | |
| GFMSI | |

TABLE 25

| | |
|---|---|
| cbe_251445 HPV 16 E6 | |
| Human papillomavirus 16 E6 | |
| length = 158 | |
| MHQKRTAMFQDPQERPRKLPQLCTELQTTIHDIILECVYCKQQLLRREVYDFAFRDLCIV | SEQ ID NO: 16 |
| YRDGNPYAVCDKCLKFYSKISEYRHYCYSLYGTTLEQQYNKPLCDLLIRCINCQKPLCPE | |
| EKQRHLDKKQRFHNIRGRWTGRCMSCCRSSRTRRETQL | |

TABLE 26

| | |
|---|---|
| cbe_251448 HPV 16 E7 | |
| Human papillomavirus 16 E7 | |
| length = 98 | |
| MHGDTPTLHEYMLDLQPETTDLYCYEQLNDSSEEEDEIDGPAGQAEPDRAHYNIVTFCCK | SEQ ID NO: 17 |
| CDSTLRLCVQSTHVDIRTLEDLLMGTLGIVCPICSQKP | |

TABLE 27

| | |
|---|---|
| cbe_251663 HPV 11 E1 | |
| Human papillomavirus 11 E1 | |
| length = 649 | |
| MADDSGTENEGSGCTGWFMVEAIVEHTTGTQISEDEEEEVEDSGYDMVDFIDDRHITQNS | SEQ ID NO: 18 |
| VEAQALFNRQEADAHYATVQDLKRKYLGSPYVSPISNVANAVESEISPRLDAIKLTTQPK | |
| KVKRRLFETRELTDSGYGYSEVEAATQVEKHGDPENGGDGQERDTGRDIEGEGVEHREAE | |
| AVDDSTREHADTSGILELLKCKDIRSTLHGKFKDCFGLSFVDLIRPFKSDRTTCADWVVA | |
| GFGIHHSIADAFQKLIEPLSLYAHIQWLTNAWGMVLLVLIRFKVNKSRCTVARTLGTLLN | |
| IPENHMLIEPPKIQSGVRALYWFRTGISNASTVIGEAPEWITRQTVIEHSLADSQFKLTE | |
| MVQWAYDNDICEESEIAFEYAQRGDFDSNARAFLNSNMQAKYVKDCAIMCRHYKHAEMKK | |
| MSIKQWIKYRGTKVDSVGNWKPIVQFLRHQNIEFIPFLSKLKLWLHGTPKKNCIAIVGPP | |
| DTGKSCFCMSLIKFLGGTVISYVNSCSHFWLQPLTDAKVALLDDATQPCWTYMDTYMRNL | |
| LDGNPMSIDRKHRALTLIKCPPLLVTSNIDISKEEKYKYLHSRVTTFTFPNPFPFDRNGN | |
| AVYELSDANWKCFFERLSSSLDIEDSEDEEDGSNSQAFRCVPGSVVRTL | |

TABLE 28

| | |
|---|---|
| cbe_251737 HPV 16 L2 | |
| Human papillomavirus 16 L2 | |
| length = 473 | |
| MRHKRSAKRTKRASATQLYKTCKQAGTCPPDIIPKVEGKTIAVQILQYGSMGVFFGGLGI | SEQ ID NO: 19 |
| GTGSGTGGRTGYIPLGTRPPTATDTLAPVRPPLTVDPVGPSDPSIVSLVEETSFIDAGAP | |
| TSVPSIPPDVSGFSITTSTDTTPAILDINNTVTTVTTHNNPTFTDPSVLQPPTPAETGGH | |
| FTLSSSTISTHNYEEIPMDTFIVSTNPNTVTSSTPIPGSRPVARLGLYSRTTQQVKVVDP | |
| AFVTTPTKLITYDNPAYEGIDVDNTLYFSSNDNSINIAPDPDFLDIVALHRPALTSRRTG | |
| IRYSRIGNKQTLRTRSGKSIGAKVHYYYDLSTIDPAEEIELQTITPSTYTTTSHAASPTS | |
| INNGLYDIYADDFITDTSTTPVPSVPSTSLSGYIPANTTIPFGGAYNIPLVSGPDIPINI | |
| TDQAPSLIPIVPGSPQYTIIADAGDFYLHPSYYMLRKRRKRLPYFFSDVSLAA | |

TABLE 29

| | |
|---|---|
| cbe_251740 HPV 1a E1-E4 | |
| Human Papillomavirus 1a E1-E4 | |
| length = 125 | |
| MADNKAPQGLLGLLQYTPTTQPYPRVTPPSNRRPSTTPNSQDRGRPRRSDKDSRKHLYAD | SEQ ID NO: 20 |
| GLTDGEDPEVPEVEDEEKENQRPLGHPDLSLLRETLEVYTQRLKRDILQDLDDFCRKLGI | |
| HPWSV | |

TABLE 30

| | |
|---|---|
| cbe_251784 HPV 16 L1 | |
| Human papillomavirus 16 L1 | |
| length = 531 | |
| MQVTFIYILVITCYENDVNVYHIFFQMSLWLPSEATVYLPPVPVSKVVSTDEYVARTNIY | SEQ ID NO: 21 |
| YHAGTSRLLAVGHPYFPIKKPNNNKILVPKVSGLQYRVFRIHLPDPNKFGFPDTSFYNPD | |
| TQRLVWACVGVEVGRGQPLGVGISGHPLLNKLDDTENASAYAANAGVDNRECISMDYKQT | |
| QLCLIGCKPPIGEHWOKGSPCTNVAVNPGDCPPLELINTVIQDGDMVHTGFGANDFTTLQ | |
| ANKSEVPLDICTSICKYPDYIKMVSEPYGDSLFFYLRREQMFVRHLFNRAGTVGENVPDD | |
| LYIKGSGSTANLASSNYFPTPSGSMVTSDAQIFNKPYWLQRAQCHNNGICWGNQLFVTVV | |
| DTTRSTNMSLCAAISTSETTYKNTNFKEYLRHGEEYDLQFIFQLCKITLTADVMTYIHSM | |
| NSTILEDWNFGLQPPPGGTLEDTYRFVTQAIACQKHTPPAPKEDDPLKKYTFWEVNLKEK | |
| FSADLDQFPLGRKFLLQAGLKAKPKFTLGKRKATPTTSSTSTTAKRKKRIKL | |

TABLE 31

| | |
|---|---|
| cbe_251917 HPV 1a E2 | |
| Human Papillomavirus 1a E2 | |
| length = 401 | |
| MENLSSRLDLLQEQLMNLYEQDSKLIEDQIKQWNLIRQEQVLFHFARKNGVMRIGLQAVP | SEQ ID NO: 22 |
| SLASSQEKAKTAIEMVLHLESLKDSPYGTEDWSLQDTSRELFLAPPAGTFKKSGSTLEVT | |
| YDNNPDNQTRHTIWNHVYYQNGDDVWRKVSSGVDAVGVYYLEHDGYKNYYVLFAEEASKY | |
| STTGQYAVNYRGKRFTNVMSSTSSPRAAGAPAVHSDYPTLSESDTAQQSTSIDYTELPGQ | |
| GETSQVRQRQQKTPVRRRPYGRRRSRSPRGGGRREGESTPSRTPGSVPSARDVGSIHTTP | |
| QKGHSSRLRRLLQEAWDPPVVCVKGGANQLKCLRYRLKASTQVDFDSTSTTWHWTDRKNT | |
| ERIGSARMLVKFIDEAQREKFLERVALPRSVSVFLGQFNGS | |

TABLE 32

| | |
|---|---|
| cbe_251919 HPV 1a E6 | |
| Human Papillomavirus 1a E6 | |
| length = 140 | |
| MATPIRTVRQLSESLCIPYIDVLLPCNFCNYFLSNAEKLLFDHFDLHLVWRDNLVFGCCQ | SEQ ID NO: 23 |
| GCARTVSLLEFVLYYQESYEVPEIEElLDRPLLQIELRCVTCIKKLSVAEKLEVVSNGER | |
| VHRVRNRLKAKCSLCRLYAI | |

TABLE 33

| | |
|---|---|
| cbe_251925 HPV 1a E7 | |
| Human Papillomavirus 1a E7 | |
| length = 93 | |
| MVGEMPALKDLVLQLEPSVLDLDLYCYEEVPPDDIEEELVSPQQPYAVVASCAYCEKLVR | SEQ ID NO: 24 |
| LTVLADHSAIRQLEELLLRSLNIVCPLCTLQRQ | |

TABLE 34

| | |
|---|---|
| cbe_251976 FLJ23584 | |
| hypothetical protein FLJ23584 | |
| length = 234 | |
| MNPVPHWGEVFLLVGGEGEHLASQGTTPARDHRVGTSPASQQAQPESWRRRQRDKGVDPE | SEQ ID NO: 25 |
| KAPSLTRQSQNPPSLTAPLGMPSACSCLPCGPAPEAAIILAGPPTALTVLPKGTGLKKSK | |
| RLLLESLMRRRIAHLKWGLPRRILESYFLFNFLGSCSLTLAGARISGLNTGQELQAQQER | |
| YCEAQGSPPGLKSPERFQRVQRPDRKSSKLPIQARALERNRPHMSEPIKHFHPA | |

TABLE 35

| | |
|---|---|
| cbe_252016 HPV 1a L1 | |
| Human Papillomavirus 1a L1 | |
| length = 508 | |
| MYNVFQMAVWLPAQNKFYLPPQPITRILSTDEYVTRTNLFYHATSERLLLVGHPLFEISS | SEQ ID NO: 26 |
| NQTVTIPKVSPNAFRVFRVRFADPNRFAFGDKAIFNPETERLVWGLRGIEIGRGQPLGIG | |
| ITGHPLLNKLDDAENPTNYINTHANGDSRQNTAFDAKQTQMFLVGCTPASGEHWTSSRCP | |
| GEQVKLGDCPRVQMIESVIEDCDMMDIGFGAMDFAALQQDKSDVPLDVVQATCKYPDYIR | |
| MNHEAYGNSMFFFARREQMYTRHFFTRGGSVGDKEAVPQSLYLTADAEPRTTLATTNYVG | |
| TPSGSMVSSDVQLFNRSYWLQRCQGQNNGICWRNQLFITVGDNTRGTSLSISMKNNASTT | |
| YSNANFNDFLRHTEEFDLSFIVQLCKVKLTPENLAYIHTMDPNILEDWQLSVSQPPTNPL | |
| EDQYRFLGSSLAAKCPEQAPPEPQTDPYSQYKFWEVDLTERMSEQLDQFPLGRKFLYQSG | |
| MTQRTATSSTTKRKTVRVSTSAKRRRKA | |

TABLE 36

| | |
|---|---|
| cbe_252041 HPV 1a L2 | |
| Human Papillomavirus 1a L2 | |
| length = 507 | |
| MYRLRRKRAAPKDIYPSCKISNTCPPDIQNKIEHTTIADKILQYGSLGVFLGGLGIGTAR | SEQ ID NO: 27 |
| GSGGRIGYTPLGEGGGVRVATRPTPVRPTIPVETVGPSEIFPIDVVDPTGPAVIPLQDLG | |
| RDFPIPTVQVIAEIHPISDIPNIVASSTNEGESAILDVLRGNATIRTVSRTQYNNPSFTV | |
| ASTSNISAGEASTSDIVFVSNGSGDRVVGEDIPLVELNLGLETDTSSVVQETAFSSSTPI | |
| AERPSFRPSRFYNRRLYEQVQVQDPRFVEQPQSMVTFDNpAFEPELDEVSIIFQRDLDAL | |
| AQTPVPEFRDXIVYLSKPTFSREPGGRLRVSRLGKSSTRTRLGTAIGARTHFFYDLSSIA | |
| PEDSIELLPLGEHSQTTVISSNLGDTAFIQGETAEDDLEVISLETPQLYSEEELLDTNES | |
| VGENLQLTITNSEGEVSILDLTQSRVRPPFGTEDTSLHVYYPNSSKGTPIINPEESFTPL | |
| VIIALNNSTGDFELHPSLRKRRKRAYV | |

TABLE 37

| | |
|---|---|
| cbe_2685998 Prey2313 | |
| Homo sapiens, clone IMAGE: 3625550, mRNA, partial cds. | |
| length = 361 | |
| AICLDYFTDPVSIGCGHNFCRVCVTQLWGGEDEEDRDELDREEEEEDGEEEEVEAVGAGA | SEQ ID NO: 28 |
| GWDTPMRDEDYEGDMEEEVEEEEEGVFWTSGMSRSSWDNMDYVWEEEDEEEDLDYYLGDM | |
| EEEDLRGEDEEDEEEVLEEVEEEDLDPVTPLPPPPAPRRCFTCPQCRKSFPRRSFRPNLQ | |
| LANMVQVIRQMHPTPGRGSRVTDQGICPKHQEALKLFCEVDEEAICVVCRESRSHKQHSV | |
| VPLEEVVQEYKAKLQGHVEPLRKHLEAVQKMKAKEERRVTELKSQMKSELAAVASEFGRL | |
| TRFLAEEQAGLERRLREMHEAQLGRAGAAASRLAEQAAQLSRLLAEAQERSQQGGLRLLQ | |
| V | |

TABLE 38

| | |
|---|---|
| cbe_2826346 RPS10 | |
| ribosomal protein S10 | |
| length = 165 | |
| MLMPKKNRIAIYELLFKEGVMVAKKDVHMPKHPELADKNVPNLHVMKAMQSLKSRGYVKE | SEQ ID NO: 29 |
| QFAWRHFYWYLTNEGIQYLRDYLHLPPEIVPATLRRSRPETGRPRPKGLEGERPARLTRG | |
| EADRDTYRRSAVPPGADKKAEAGAGSATEFQFRGGFGRGRGQPPQ | |

TABLE 39

| | |
|---|---|
| cbe_2830508 Prey292051 | |
| Homo sapiens, clone MGC: 12344, | |
| mRNA, complete cds. | |
| length = 20 | |
| MQLKTLSLATPPRETAVINL | SEQ ID NO: 30 |

TABLE 40

| | |
|---|---|
| cbe_3001381 IRF1 | |
| interferon regulatory factor 1 | |
| length = 325 | |
| MPITRMRMRPWLEMQINSNQIPGLIWINKEEMIFQIPWKHAAKHGWDINKDACLFRSWAI | SEQ ID NO: 31 |
| HTGRYKAGEKEPDPKTWKANFRCAMNSLPDIEEVKDQSRNKGSSAVRVYRMLPPLTKNQR | |
| KERKSKSSRDAKSKAKRKSCGDSSPDTFSDGLSSSTLPDDHSSYTVPGYMQDLEVEQALT | |
| PALSPCAVSSTLPDWHIPVEVVPDSTSDLYNFQVSPMPSTSEATTDEDEEGKLPEDIMKL | |
| LEQSEWQPTNVDGKGYLLNEPGVQPTSVYGDFSCKEEPEIDSPGGDIGLSLQRVFTDLKN | |
| MDATWLDSLLTPVRLPSIQAIPCAP | |

TABLE 41

| | |
|---|---|
| cbe_3056756 KIAA0440 | |
| signal-induced proliferation-associated 1 like 1 | |
| length = 1804 | |
| MTSLKRSQTERPLATDRASVVGTDGTPKVHTDDFYMRRFRSQNGSLGSSVMAPVGPPRSE | SEQ ID NO: 32 |
| GSHHITSTPGVPKMGVRARIADWPPRKENIKESSRSSQEIETSSCLDSLSSKSSPVSQGS | |
| SVSLNSNDSAMLKSIQNTLKNKTRPSENMDSRFLMPEAYPSSPRKALRRIRQRSNSDITI | |
| SELDVDSFDECISPTYKTGPSLHREYGSTSSIDKQGTSGESFFDLLKGYKDDKSDRGPTP | |
| TKLSDFLITGGGKGSGFSLDVIDGPISQRENLRLFKEREKPLKRRSKSETGDSSIFRKLR | |
| NAKGEELGKSSDLEDNRSEDSVRPWTCPKCFAHYDVQSILFDLNEAIMNRHNVIKRRNTT | |
| TGASAAAVASLVSGPLSHSASFSSPMGSTEDLNSKGSLSMDQGDDKSNELVMSCPYFRNE | |
| IGGEGERKISLSKSNSGSFSGCESASFESTLSSHCTNAGVAVLEVPKENLVLHLDRVKRY | |
| IVEHVDLGAYYYRKFFYQKEHWNYFGADENLGPVAVSIRREKPDEMKENGSPYNYRIIFR | |
| TSELMTLRGSVLEDAIPSTAKHSTARGLPLKEVLEHVVPELNVQCLRLAFNTPKVTEQLM | |
| KLDEQGLNYQQKVGIMYCKAGQSTEEEMYNNESAGPAFEEFLQLLGERVRLKGFEKYRAQ | |
| LDTKTDSTGTHSLYTTYKDYEIMFHVSTMLPYTPNNKQQLLRKRHIGNDIVTIVFQEPGA | |
| QPFSPKNIRSHFQHVFVIVRVHNPCSDSVCYSVAVTRSRDVPSFGPPIPKGVTFPKSNVF | |
| RDFLIAKVINAENAAHKSEKFRAMATRTRQEYLKDLAEKNVTNTPIDPSGKFPFISLASK | |
| KKEKSKPYPGAELSSMGAIVWAVRAEDYNKAMELDCLLGISNEFIVLIEQETKSVVFNCS | |
| CRDVIGWTSTDTSLKIFYERGECVSVGSFINIEEIKEIVKRLQFVSKGCESVEMTLRRNG | |
| LGQLGFHVNYEGIVADVEPYGYAWQAGLRQGSRLVEICKVAVATLSHEQMIDLLRTSVTV | |
| KVVIIPPHDDCTPRRSCSETYRMPVMEYKMNEGVSYEFKFPFRNNNKWQRNASKGPHSPQ | |
| VPSQVQSPMTSRLNAGKGDGKMPPPERAANIPRSISSDGRPLERRLSPGSDIYVTVSSMA | |
| LARSQCRNSPSNLSSSSDTGSVGGTYRQKSMPEGFGVSRRSPASIDRQNTQSDIGGSGKS | |
| TPSWQRSEDSIADQMAYSYRGPQDFNSFVLEQHEYTEPTCHLPAVSKVLPAFRESPSGRL | |
| MRQDPVVHLSPNKQGHSDSHYSSHSSSNTLSSNASSAHSDEKWYDGDRTESELNSYNYLQ | |
| GTSADSGIDTTSYGPSHGSTASLGAATSSPRSGPGKEKVAPLWHSSSEVISMADRTLETE | |
| SHGLDRKTESSLSLDIHSKSQAGSTPLTRENSTFSINDAASHTSTMSSRHSASPVVFTSA | |
| RSSPKEELHPAAPSQLAPSFSSSSSSSSGPRSFYPRQGATSKYLIGWKKPEGTINSVGFM | |
| DTRKRHQSDGNEIAHTRLRASTRDLRASPKPTSKSTIEEDLKKLIDLESPTPESQKSFKF | |
| HALSSPQSPFPSTPTSRRALHRTLSDESIYNSQREHFFTSRASLLDQALPNDVLFSSTYP | |
| SLPKSLPLRRPSYTLGMKSLHGEFSASDSSLTDIQETRRQPMPDPGLMPLPDTAADLDWS | |
| NLVDAAKAYEVQRASFFAASDENHRPLSAASNSDQLEDQALAQMKPYSSSKDSSPTLASK | |
| VDQLEGMLKMLREDLKKEKEDKAHLQAEVQHLREDNLRLQEESQNASDKLKKFTEWVFNT | |
| IDMS | |

TABLE 42

| | |
|---|---|
| cbe_3340492 TRIP | |
| TRAF interacting protein | |
| length = 469 | |
| MPIRALCTICSDFFDHSRDVAAIHCGHTFHLQCLIQSFETAPSRTCPQCRIQVGKRTIIN | SEQ ID NO: 33 |
| KLFFDLAQEEENVLDREFLKNELDNVRAQLSQKDKEKRDSQVIIDTLRDTLEERNATVVS | |
| LQQALGKAEMLCSTLKKQMKYLEQQQDETKQAQEEAGRLRSKMKTMEQIELLLQSQLPEV | |
| EEMIRDMGVGQSAVEQLAVYCVSLKKEYENLKEARKASGEVADKLRKDLFSSRSKLQTVY | |
| SELDQAKLELKSAQKDLQSADKEIMSLKKKLTMLQETLNLPPVASETVDRLVLESPAPVE | |
| VNLKLRRPSFRDDIDLNATFDVDTPPARPSSSQHGYYEKLCLEKSHSPIQDVPKKICKGP | |
| RKESQLSLGGQSCAGEPDEELVGAFPIFVRNAILGQKQPKRPRSESSCSKDVVRTGFDGL | |
| GGRTKFIQPTDTVMIRPLPVKPKTKVKQRVRVKTVPSLFQAKLDTFLWS | |

TABLE 43

| | |
|---|---|
| cbe_3345510 COX6C | |
| cytochrome c oxidase subunit VIc | |
| length = 75 | |
| MAPEVLPKPRMRGLLARRLRNHMAVAFVLSLGVAALYKFRVADQRKKAYADFYRNYDVMK | SEQ ID NO: 34 |
| DFEEMRKAGIFQSVK | |

TABLE 44

| | |
|---|---|
| cbe_3454406 gbh_af252829 | |
| Homo sapiens chromosome 8 clone BAC 495D4 map 8q24, *** SEQUENCING | |
| length = 338 | |
| MGKKQNRKTGNSKTQSTSPPPKERSSSPATDQSWMENDFDELREEGFRRSNYSELREDIQ | SEQ ID NO: 35 |
| TKGKEVENFEKNLEECITRITNTEKCLKELMELKTKARELREECRSLRSQCDQLEERVSA | |
| MEDEMNEMKQEGKFREKRIKRNEQSLQEIWDYVKRPNLRLIGVPESDAENGTKLENTLQD | |
| IIQETFPNLARQANVQIQEIQRTPQRYSSRRATPRHIIVRFTKVEMKEKMLRAAREKGRV | |
| TLKGKPIRLTADLSAETLQARREWCPILNILKEKNFQPRISYPAKLSFISEGEIKYFTDK | |
| QMLTDFVTSRPALKELLKEALNMERNNRYQPLQNHAKM | |

TABLE 45

| | |
|---|---|
| cbe_3461331 MET | |
| met proto-oncogene (hepatocyte growth factor receptor) | |
| length = 1408 | |
| MKAPAVLAPGILVLLFTLVQRSNGECKEALAKSEMNVNNKYQLPNFTAETPIQNVILEEN | SEQ ID NO: 36 |
| HIFLGATNYIYVLNEEDLQKVAEYKTGPVLEHPDCFPCQDCSSKANLSGGVWKDNINMAL | |
| VVDTYYDDQLISCGSVNRGTCQRHVFPHNHTALIQSEVHCIFSPQIEEPSQCPDCVVSAW | |
| GAKVLSSVKDRFINFFVGNTINSSYFPDHPLHSISVRRLKETKDGFNFLTDQSYIDVLPE | |
| FRDSYPIKYVHAFESNNFIYFLTVQRETLDAQTFHTRIIRFCSINSGLHSYMEMPLECIL | |
| TEKRKKRSTKKEVFNILQAAYVSKPGAQLARQIGASLNDDILFGVFAQSKPDSAEFMDRS | |
| ANCAFPIKYVNDFFNKIVNKNNVRCLQHFYGPNHEHCFNRTLLRNSSGCEARRDEYRTEF | |
| TTALQRVDLFMGQFSEVLLTSISTEIKGDLTIANLGTSEGRFMQVVVSRSGPSTPHVNFL | |
| LDSHPVSPEVIVEHTLNQNGYTLVITGKKITKIPLNGLGCRHFQSCSQCLSAPPFVQCGW | |
| CHDKCVRSEECLSGTWTQQICLPAIYKVFPNSAPLEGGTRLTICGWDFGFRRNNKFDLKK | |
| TRVLLGNESCTLTLSESTMNTLKCTVGPAMNKHFNMSIIISNGHGTTQYSTFSYVDPVIT | |
| SISPKYGPNAGGTLLTLTGNYLNSGNSRHISIGGKTCTLKSVSNSILECYTPAQTISTEF | |
| AVKLKIDLANRETSIFSYREDPIVYEIHPTKSFISTWWKEPLNIVSFLFCFASGGSTITG | |
| VGKNLNSVSVPRMVINVHEAGRNFTVACQHRSNSEIICCTTPSLQQLNLQLPLKTKAFFM | |
| LDGILSKYFDLIYVHNPVFKPFEKPVMISMGNENVLEIKGNDIDPEAVKGEVLKVGNKSC | |
| ENIHLHSEAVLCTVPNDLLKLNSELNIEWKQAISSTVLGKVIVQPDQNFTGLIAGWSIS | |
| TALLLLLGFFLWLKKRKQIKDLGSELVRYDARVHTPHLDRLVSARSVSPTTEMVSNESVD | |
| YRATFPEDQFPNSSQNGSCRQVQYPLTDMSPILTSGDSDISSPLLQNTVHIDLSALNPEL | |
| VQAVQHVVICPSSLIVHFNEVICRGHFGCVYHGTLLDNDGKKIHCAVKSLNRITDICEVS | |
| QFLTEGIIMKDPSHPNVLSLLGICLRSEGSPLVVLPYMKHGDLRNFIRNETHNPTVKDLI | |
| GFGLQVAKAMKYLASKKFVHRDLAARNCMLDEKFTVKVADFGLARDMYDKEYYSVHNKTG | |
| AKLPVKWMALESLQTQKFTTKSDVWSFGVVLWELMTRGAPPYPDVNTFDITVYLLQGRRL | |
| LQPEYCPDPLYEVMLKCWHPKAEMRPSFSELVSRISAIFSTFIGEHYVHVNATYVNVKCV | |
| APYPSLLSSEDNADDEVDTRPASFWETS | |

TABLE 46

| | |
|---|---|
| cbe_3473488 gbh_al121903 | |
| Human DNA sequence from clone RP1-155G6 on chromosome 20 Contains | |
| length = 641 | |
| VGCNPNEDVAIFAVDSLRQLSMKFLEKGELANFRFQKDFLRPFEHIMKKNRSPTIRDMAI | SEQ ID NO: 37 |
| RCIAQMVNSQAANIRSGWKNIFAVFHQAASDHDGNIVELAFQTTCHIVTTIFQHHFPAAI | |
| DSFQDAVKCLSEFACNAAFPDTSMEAIRLIRFCGKYVSERPRVLQEYTSDDMNVAPGDRV | |
| WVRGWFPILFELSCIINRCKLDVRTRGLTVMFEIMKSYGHTFEKHWWQDLFRIVFRIFDN | |
| MKLPEQLSEKSEWMTTTCNHALYAICDVFTQFYEALNEVLLSDVFAQLQWCVKQDNEQLA | |
| RSGTNCLENLVISNGEKFSPEVWDETCNCMLDIFKTTIPHVLLTWRPVGMEEDSSEKHLD | |
| VDLDRQSLSSIDKNPSERGQSQLSNPTDDSWKGRPYANQKLFASLLIKCVVQLELIQTID | |
| NIVFYPATSKKEDAEHMVAAQQDTLDADIHIETEDQGMYKYMSSQHLFKLLDCLQESHSF | |
| SKAFNSNYEQRTVLWRAGFKGKSKPNLLKQETSSLACCLRILFRNYVDENRRDSWEEIQQ | |
| RLLTVCSEALAYFITVNSESHREAWTSLLLLLLTKTLKINDEKFKAHASMYYPYLCEIMQ | |
| FDLIPELRAVLRKFFLRIGVVYKIWIPEEPSQVPAALSPVW | |

TABLE 47

| | |
|---|---|
| cbe_3474077 KRT4 | |
| keratin 4 | |
| length = 534 | |
| MIARQQCVRGGPRGFSCGSAIVGGGKRGAFSSVSMSGGAGRCSSGGFGSRSLYNLRGNKS | SEQ ID NO: 38 |
| ISMSVAGSRQGACFGGAGGFGTGGFGAGGFGAGFGTGGFGGGFGGSFSGKGGPGFPVCPA | |
| GGIQEVTINQSLLTPLHVEIDPEIQKVRTEEREQIKLLNNKFASFIDKVQFLEQQNKVLE | |
| TKWNLLQQQTTTTSSKNLEPLFETYLSVLRKQLDTLGNDKGRLQSELKTMQDSVEDFKTK | |
| YEEEINKRTAAENDFVVLKKDVDAAYLNKVELEAKVDSLNDEINFLKVLYDAELSQMQTH | |
| VSDTSVVLSMDNNRNLDLDSIIAEVRAQYEEIAQRSKAEAEALYQTKVQQLQISVDQHGD | |
| NLKNTKSEIAELNRMIQRLRAEIENIKKQCQTLQVSVADAEQRGENALKDAHSKRVELEA | |
| ALQQAKEELARMLREYQELMSVKLALDIEIATYRKLLEGEEYRMSGECQSAVSISVVSGS | |
| TSTGGISGGLGSGSGFGLSSGFGSGSGSGFGFGGSVSGSSSSKIISTTTLNKRR | |

TABLE 48

| | |
|---|---|
| cbe_3563252 LOC91689 | |
| hypothetical gene supported by AL449243 | |
| length = 107 | |
| MASGAARWLVLAPVRSGALRSGPSLRKDGDVSAAWSGSGRSLVPSGSVIVTRSGAILPKP | SEQ ID NO: 39 |
| VKMSFGLLRVFSIVIPFLYVGTLISKNFAALLEEHDIFVPEDDDDDD | |

TABLE 49

| | |
|---|---|
| cbe_3575702 SUI1 | |
| putative translation initiation factor | |
| length = 113 | |
| MSAIQNLHSFDPFADASKGDDLLPAGTEDYIHIRIQQRNGRKTLTTVQGIADDYDKKKLV | SEQ ID NO: 40 |
| KAFKKKFACNGTVIEHPEYGEVIQLQGDQRKNICQFLVEIGLAKDDQLKVHGF | |

TABLE 50

cbe_3580897 HSPA5
heat shock 70 kDa protein 5 (glucose-regulated protein, 78 kDa)
length = 654

| | |
|---|---|
| MKLSLVAAMLLLLSAARAEEEDKKEDVGTVVGIDLGTTYSCVGVFKNGRVEIIANDQGNR | SEQ ID NO:41 |
| ITPSYVAFTPEGERLIGDAAKNQLTSNPENTVFDAKRLIGRTWNDPSVQQDIKFLPFKVV | |
| EKKTKPYIQVDIGGGQTKTFAPEEISAMVLTKMKETAEAYLGKKVTHAVVTVPAYFNDAQ | |
| RQATKDAGTIAGLNVMRIINEPTAAAIAYGLDKREGEKNILVFDLGGGTFDVSLLTIDNG | |
| VFEVVATNGDTHLGGEDFDQRVMEHFIKLYKKKTGKDVRKDNRAVQKLRREVEKAKRALS | |
| SQHQARIEIESFYEGEDFSETLTRAKFEELNMDLFRSTMKPVQKVLEDSDLKKSDIDEIV | |
| LVGGSTRIPKIQQLVKEFFNGKEPSRGINPDEAVAYGAAVQAGVLSGDQDTGDLVLLDVC | |
| PLTLGIETVGGVMTKLIPRNTVVPTKKSQIFSTASDNQPTVTIKVYEGERPLTKDNHLLG | |
| TFDLTGIPPAPRGVPQIEVTFEIDVNGILRVTAEDKGTGNKNKITITNDQNRLTPEEIER | |
| MVNDAEKFAEEDKKLKERIDTRNELESYAYSLKNQIGDKEKLGGKLSSEDKETMEKAVEE | |
| KIEWLESHQDADIEDFKAKKKELEEIVQPIISKLYGSAGPPPTGEEDTAEKDEL | |

TABLE 51

cbe_3583511 MGC3222
hypothetical protein MGC3222
length = 255

| | |
|---|---|
| VVSPDSIHSVAPENEGRLVHIIGALRTSKLLSDPNYGVHLPAVKLRRHVEMYQWVETEES | SEQ ID NO:42 |
| REYTEDGQVKKETRYSYNTEWRSEIINSKNFDREIGHNNPSAMAVESFTATAPFVQIGRF | |
| FLSSGLIDKVDNFKSLSLSKLEDPHVDIIRRGDFFYHSENPKYPEVGDLRVSFSYAGLSG | |
| DDPDLGPAHVVTVIARQRGDQLVPFSTKSGDTLLLLHHGDFSAEVSAVPYSYGGGTSMSF | |
| LPSSGYLIRSHYQGS | |

TABLE 52

cbe_3590661 NUP214
nucleoporin 214 kDa
length = 2127

| | |
|---|---|
| MRAAGAEGRKFAVERPGFRGQGRGRQRWLLRHTEGGAMGDEMDAMIPEREMKDFQFRALK | SEQ ID NO:43 |
| KVRIFDSPEELPKERSSLLAVSNKYGLVFAGGASGLQIFPTKNLLIQNKPGDDPNKIVDK | |
| VQGLLVPMKFPIHHLALSCDNLTLSACMMSSEYGSIIAFFDVRTFSNEAKQQKRPFAYHK | |
| LLKDAGGMVIDMKWNPTVPSMVAVCLADGSIAVLQVTETVKVCATLPSTVAVTSVCWSPK | |
| GKQLAVGKQNGTVVQYLPTLQEKKVIPCPPFYESDHPVRVLDVLWIGTYVFAIVYAAADG | |
| TLETSPDVVMALLPKKEEKHPEIFVNFMEPCYGSCTERQHHYYLSYIEEWDLVLAASAAS | |
| TEVSILARQSDQINWESWLLEDSSRAELPVTDKSDDSLPMGVVVDYTNQVEITISDEKTL | |
| PPAPVLMLLSTDGVLCPFYMINQNPGVKSLIKTPERLSLEGERQPKSPGSTPTTPTSSQA | |
| PQKLDASAAAAPASLPPSSPAAPIATFSLLPAGGAPTVFSFGSSSLKSSATVTGEPPSYS | |
| SGSDSSKAAPGPGPSTFSFVPPSKASLAPTPAASPVAPSAASFSFGSSGFKPTLESTPVP | |
| SVSAPNIAMKPSFPPSTSAVKVNLSEKFTAAATSTPVSSSQSAPPMSPFSSASKPAASGP | |
| LSHPTPLSAPPSSVPLKSSVLPSPSGRSAQGSSSPVPSMVQKSPRITPPAAKPGSPQAKS | |
| LQPAVAEKQGHQWKDSDPVMAGIGEEIAHFQKELEELKARTSKACFQVGTSEEMKMLRTE | |
| SDDLHTFLLEIKETTESLHGDISSLKTTLLEGFAGVEEAREQNERNRDSGYLHLLYKRPL | |
| DPKSEAQLQEIRRLHQYVKFAVQDVNDVLDLEWDQHLEQKKKQRHLLVPERETLFNTLAN | |
| NREIINQQRKRLNHLVDSLQQLRLYKQTSLWSLSSAVPSQSSIHSFDSDLESLCNALLKT | |
| TIESHTKSLPKVPAKLSPMKQAQLRNFLAKRKTPPVRSTAPASLSRSAFLSQRYYEDLDE | |
| VSSTSSVSQSLESEDARTSCKDDEAVVQAPRHAPVVRTPSIQPSLLPHAAPFAKSHLVHG | |
| SSPGVMGTSVATSASKIIPQGADSTMLATKTVKHGAPSPSHPISAPQAAAAAALRRQMAS | |
| QAPAVNTLTESTLKNVPQVVNVQELKNNPATPSTAMGSSVPYSTAKTPHPVLTPVAANQA | |
| KQGSLINSLKPSGPTPASGQLSSGDKASGTAKIETAVTSTPSASGQFSKPFSFSPSGTGF | |
| NFGIITPTPSSNFTAAQGATPSTKESSQPDAFSSGGGSKPSYEAIPESSPPSGITSASNT | |
| TPGEPAASSSRPVAPSGTALSTTSSKLETPPSKLGELLFPSSLAGETLGSFSGLRVGQAD | |
| DSTKPTNKASSTSLTSTQPTKTSGVPSGFNFTAPPVLGKHTEPPVTSSATTTSVAPPAAT | |
| STSSTAVFGSLPVTSAGSSGVISFGGTSLSAGKTSFSFGSQQTNSTVPPSAPPPTTAATP | |
| LPTSFPTLSFGSLLSSATTPSLPMSAGRSTEEATSSALPEKPGDSEVSASAASLLEEQQS | |
| AQLPQAPPQTSDSVKKEPVLAQPAVSNSGTAASSTSLVALSAEATPATTGVPDARTEAVP | |
| PASSFSVPGQTAVTAAAISSAGPVAVETSSTPIASSTTSIVAPGPSAEAAAFGTVTSGSS | |
| VFAQPPAASSSSAFNQLTNNTATAPSATPVFGQVAASTAPSLFGQQTGSTASTAAATPQV | |
| SSSGFSSPAFGTTAPGVFGQTTFGQASVFGQSASSAASVFSFSQPGFSSVPAFGQPASST | |
| PTSTSGSVFGAASSTSSSSSFSFGQSSPNTGGGLFGQSNAPAFGQSPGFCQGGSVFGGTS | |
| AATTTAATSGFSFCQASGFGSSNTGSVFGQAASTGGIVFGQQSSSSSGSVFGSGNTGRGG | |
| GFFSGLGGKPSQDAANKNPFSSASGGFGSTATSNTSNLFGNSGAKTFGGFASSSFGEQKP | |
| TGTFSSGGGSVASQGFGFSSPNKTGGFGAAPVFGSPPTFGGSPGFGGVPAFGSAPAFTSP | |
| LGSTGGKVFGEGTAAASAGGFGFGSSSNTTSFGTLASQNAPTFGSLSQQTSGFGTQSSGF | |
| SGFGSGTGGFSFGSNNSSVQGFGGWRS | |

TABLE 53

cbe_3606990 Prey1053670
Homo sapiens, Similar to CG5604 gene product, clone MGC:11330, mRNA,
complete cds.
length = 81

| | |
|---|---|
| MSSDERKAFLQFTTGCSTLPPGGLANLHPRLTVVRKVDATDASYPSVNTCVHYLKLPEYS | SEQ ID NO:44 |
| SEEIMRERLLAATMEKGFHLN | |

TABLE 54

cbe_3621798 XPO1
exportin 1 (CRM1 homolog, yeast)
length = 1071

| | |
|---|---|
| MPAIMTMLADHAARQLLDFSQKLDINLLDNVVNCLYHGEGAQQRMAQEVLTHLKEHPDAW | SEQ ID NO:45 |
| TRVDTILEFSQNMNTKYYGLQILENVIKTRWKILPRNQCEGIKKYVVGLIIKTSSDPTCV | |
| EKEKVYIGKLNMILVQILKQEWPKHWPTFISDIVGASRTSESLCQNNMVILKLLSEEVFD | |
| FSSGQITQVKSKHLKDSMCNEFSQIFQLCQFVMENSQNAPLVHATLETLLRFLNWIPLGY | |
| IFETKLISTLIYKFLNVPMFRNVSLKCLTEIAGVSVSQYEEQFVTLFTLTMMQLKQMLPL | |
| NTNIRLAYSNGKDDEQNFIQNLSLFLCTFLKEHDQLIEKRLNLRETLMEALHYMLLVSEV | |
| EETEIFKICLEYWNHLAAELYRESPFSTSASPLLSGSQHFDVPPRRQLYLPMLFKVRLLM | |
| VSRMAKPEEVLVVENDQGEVVREFMKDTDSINLYKNMRETLVYLTHLDYVDTERIMTEKL | |
| HNQVNGTEWSWKNLNTLCWAIGSISGAMHEEDEKRFLVTVIKDLLGLCEQKRGKDNKAII | |
| ASNIMYIVGQYPRFLRAHWKFLKTVVNKLFEFMHETHDGVQDMACDTFIKIAQKCRRHFV | |
| QVQVGEVMPFIDEILNNINTIICDLQPQQVHTFYEAVGYMIGAQTDQTVQEHLIEKYMLL | |
| PNQVWDSIIQQATKNVDILKDPETVKQLGSILKTNVRACKAVGHPFVIQLGRIYLDMLNV | |
| YKCLSENISAAIQANGEMVTKQPLIRSMRTVKRETLKLISGWVSRSNDPQMVAENFVPPL | |
| LDAVLIDYQRNVPAAREPEVLSTMAIIVNKLGGHITAEIPQIFDAVFECTLNMINKDFEE | |
| YPEHRTNFFLLLQAVNSHCFPAFLAIPPTQFKLVLDSIIWAFKHTMRNVADTGLQILFTL | |
| LQNVAQEEAAAQSFYQTYFCDILQHIFSVVTDTSHTAGLTMHASILAYMFNLVEEGKIST | |
| SLNPGNPVNNQIFLQEYVANLLKSAFPHLQDAQVKLFVTGLFSLNQDIPAFKEHLRDFLV | |
| QIKEFAGEDTSDLFLEEREIALRQADEEKHKRQMSVPGIFNPHEIPEEMCD | |

TABLE 55

cbe_3621899 PCNP
PEST-containing nuclear protein
length = 178

| | |
|---|---|
| MADGKAGDEKPEKSQRAGAAGGPEEEAEKPVKTKTVSSSNGGESSSRSAEKRSAEEEAAD | SEQ ID NO:46 |
| LPTKPTKISKFGFAIGSQTTKKASAISIKLGSSKPKETVPTLAPKTLSVAAAFNEDEDSE | |
| PEEMPPEAKMRMKNIGRDTPTSAGPNSFNKGKHGFSDNQKLWERNIKSHLGNVHDQDN | |

TABLE 56

cbe_3625721 EIF2B2
eukaryotic translation initiation factor 2B, subunit 2 beta, 39 kDa
length = 351

| | |
|---|---|
| MPGSAAKGSELSERIESFVETLKRGGGPRSSEEMARETLGLLRQIITDHRWSNAGELMEL | SEQ ID NO:47 |
| IRREGRRMTAAQPSETTVGNMVRRVLKIIREEYGRLHGRSDESDQQESLHKLLTSGGLNE | |
| DFSFHYAQLQSNIIEAINELLVELEGTMENIAAQALEHIHSNEVIMTIGFSRTVEAFLKE | |
| AARKRKFHVIVAECAPFCQGHEMAVNLSKAGIETTVMTDAAIFAVMSRVNKVIIGTKTIL | |
| ANGALRAVTGTHTLALAAKHHSTPLIVCAPMFKLSPQFPNEEDSFHKFVAPEEVLPFTEG | |
| DILEKVSVHCPVFDYVPPELITLFISNIGGNAPSYIYRLMSELYHPDDHVL | |

TABLE 57

| | |
|---|---|
| cbe_3641816 ANKH | a |

nkylosis, progressive homolog (mouse)
length = 492

| | |
|---|---|
| MVKFPALTHYWPLIRFLVPLGITNIAIDFGEQALNRGIAAVKEDAVEMLASYGLAYSLMK | SEQ ID NO:48 |
| FFTGPMSDFKNVGLVFVNSKRDRTKAVLCMVVAGAIAAVFHTLIAYSDLGYYIINKLHHV | |
| DESVGSKTRRAFLYLAAFPFMDAMAWTHAGILLKHKYSFLVGCASISDVIAQVVFVAILL | |
| HSHLECREPLLIPILSLYMGALVRCTTLCLGYYKNIHDIIPDRSGPELGGDATIRKMLSF | |
| WWPLALILATQRISRPIVNLFVSRDLGGSSAATEAVAILTATYPVGHMPYGWLTEIRAVY | |
| PAFDKNNPSNKLVSTSNTVTAAHIKKFTFVCMALSLTLCFVMFWTPNVSEKILIDIIGVD | |
| FAFAELCVVPLRIFSFFPVPVTVRAHLTGWLMTLKKTFVLAPSSVLRIIVLIASLVVLPY | |
| LGVHGATLGVGSLLAGFVGESTMVAIAACYVYRKQKKKMENESATEGEDSAMTDMPPTEE | |
| VTDIVEMREENE | |

TABLE 58

cbe_3643627 DKFZP434I1735
DKFZP434I1735 protein
length = 1590

| | |
|---|---|
| VILHPSISVRLAAAWCLHCIAVALPSYLTPLLDRCLERLTGHKSSPEAVTGFSFAVAALL | SEQ ID NO:49 |
| GAVKHCPLGIPHGKGKIIMTLAEDLLCSAAQNSRLSAQRTQAGWLLISALMTLGPAVVSH | |
| HLARVLLLWKCVFPASPKDLETEKSRGDSFTWQVTLEGRAGALCAIKSFVSHCGDLLTEE | |
| VTQRLLPPLPCAVDLLTQLSSILKMYGSPLKTPSVVYRQRLYELLILLPPETYEGNLCAI | |
| LRELAADLTAPDIQVAASTFLLPPLCHQDDLLILSPFLQETDHRFIEEQLLLGNGVACGS | |
| LEYDPYSIYEKDVEGDSVPKPLPPALSVISSASKLFGVVCAHVGETQRLLILEQLLDSIK | |
| HTKGARQQVVQLHVVSSVSSFLKYVAGSKGCLGPEEMKRFALTLVMGALESPNPLLRCAA | |
| AESWARLAQMVDDGAFTAGLAQVSFDKLKSARDVVTRTGHSLALGSLHRYLGGISSSQHL | |
| NSCIGILYTLAQDSTSPDVQTWALHSLSLIIDSAGPLYYVHVEPTLSLIIMLLLNVPPTH | |
| AEVHQSLGRCLNALITTLGPELQGNSTSISTLRTSCLLGCAVMQDNPDCLVQAQAISCLQ | |
| QLHMFAPRHVNLSSLVSCLCVNLCSPYLLLRRAVLACLRQLVQREAAEVSEHAVMLAKDS | |
| REELTPDANIREVGLEGALLILLDKETDERLCHDIKETLNYMLTSMAVEKLSLWLKLCKD | |
| VLAASADFTAVTCVDTMQEEEGDKGDDASVLTTRRDEKSHPFTNPRWATRVFAAECVCRI | |
| INQCENANSAHFDIALAQEMKKRDSRNDFLVLHLADLIRMAFMAATDHSDQLRLSGLEML | |
| LVVIRRFATVPEPEFPGHVILEQYQANVGAALRPAFTSETPPDVTAKACQVCSAWIASGV | |
| VSDLNDLRRVHQLLVSSLTKIQAGKEALSHLYNESASTMEILAVLKAWAEVYIIAVQRHK | |
| NHRQPLKTTTCLEDGIRNGSCSSDGLLDLVYADLGTLSRLWLAALQDFALLTLPSEFASQ | |
| LPAEGGAFYTAETSENAKLHYYNSWALILHATALWLTSTGFVVADPDEGASNLSRPVTPT | |
| SMCQGSSSGATIKSPEDVYTDRFHLILGISVEFLCSLRSDATMESITACLHALQALLDVP | |
| WPRSKIGSDQDLGIELLNVLHRVILTRESPSIQLASLEVVRQIICAAQEHVKEKRRSAEV | |
| DDGAAEKETLPEFGEGKDTGGLVPGKSLVFATLELCVCILVRQLPELNPKLTGSPGVKAT | |
| KPQILLEDGSRLVSAALVILSELPAVCSPEGSISILPTILYLTIGVLRETAVKLPGGQLS | |
| STVAASLQALKGILSSPMARAEKSRTAWTDLLRSALTTILDCWDPVDETHQELDEVSLLT | |
| AITVFILSTSPEVTTIPCLQKRCIDKFKATLEIKDPVVQIKTYQLLHSIFQYPNPAVSYP | |
| YIYSLASCIMEKLQEIDKRKPENTAELEIFQEGIKVLETLVTVAEEHHRAQLVACLLPIL | |
| ISFLLDENSLGSATSIMRNLHDFALQNLMQIGPQYSSVFKSLVASSPALKARLEAAIKGN | |
| QESVKVKIPTSKYTKSPGKNSSIQLKTSFL | |

TABLE 59

cbe_3663966 KIAA1389
KIAA1389 protein
lenqth = 1514

| | |
|---|---|
| FSTRYPLSIANYDHKAMVPFGFPEFFRCDPAISPSLHAAAQISRGEFVRISGLDYVDSAL | SEQ ID NO:50 |
| LMGRDRDKPFKRRLKSESVETSLFRKLRTVKSEHETFKFTSELEESRLERGIRPWNCQRC | |
| FAHYDVQSILFNINEAMATRANVGKRKNITTGASAASQTQMPTGQTGNCESPLGSKEDLN | |
| SKENLDADEGDGKSNDLVLSCPYFRNETGGEGDRRIALSRANSSSFSSGESCSFESSLSS | |
| HCTNAGVSVLEVPRENQPIHREKVKRYIIEHIDLGAYYYRKFFYGKEHQNYFGIDENLGP | |
| VAVSIRREKVEDAKEKEGSQFNYRVAFRTSELTTLRGAILEDAIPSTARHGTARGLPLKE | |
| VLEYVIPELSIQCLRQASNSPKVSEQLLKLDEQGLSFQHKIGILYCKAGQSTEEEMYNNE | |
| TAGPAFEEFLDLLGQRVRLKGFSKYRAQLDNKTDSTGTHSLYTTYKDYELMFHVSTLLPY | |
| MPNNRQQLLRKRHIGNDIVTIVFQEPGALPFTPKSIRSHFQHVFVIVKVHNPCTENVCYS | |
| VGVSRSKDVPPFGPPIPKGVTFPKSAVFRDFLLAKVINAENAAHKSEKFRAMATRTRQEY | |
| LKDLAENFVTTATVDTSVKFSFITLGAKKKEKVKPRKDAHLFSIGAIMWHVIARDFGQSA | |
| DIECLLGISNEFIMLIEKDSKNVVFNCSCRDVIGWTSGLVSIKVFYERGECVLLSSVDNC | |
| AEDIREIVQRLVIVTRGCETVEMTLRRNGLGQLGFHVNFEGIVADVEPFGFAWKAGLRQG | |
| SRLVEICKVAVATLTHEQMIDLLRTSVTVKVVIIQPHDDGSPRRGCSELCRIPMVEYKLD | |
| SEGTPCEYKTPFRRNTTWHRVPTPALQPLSRASPIPGTPDRLPCQQLLQQAQAAIPRSTS | |
| FDRKLPDGTRSSPSNQSSSSDPGPGGSGPWRPQVGYDGCQSPLLLEHQGSGPLECDGARE | |
| REDTMEASRHPETKWHGPPSKVLGSYKERALQKDGSCKDSPNKLSHIGDKSCSSHSSSNT | |
| LSSNTSSNSDDKHFGSGDLMDPELLGLTYIKGASTDSGIDTAPCMPATILGPVHLAGSRS | |
| LIHSRAEQWADAADVSGPDDEPAKLYSVHGYASAISAGSAAEGSMGDLSEISSHSSGSHH | |
| SGSPSAHCSKSSGSLDSSKVYIVSHSSGQQVPGSMSKPYHRQGAVNKYVIGWKKSEGSPP | |
| PEEPEVTECPGMYSEMDVMSTATQHQTVVGDAVAETQHVLSKEDFLKLMLPDSPLVEEGR | |
| RKFSFYGNLSPRRSLYRTLSDESICSNRRGSSFGSSRSSVLDQALPNDILFSTTPPYHST | |
| LPPRAHPAPSMGSLRNEFWFSDGSLSDKSKCADPGLMPLPDTATGLDWTHLVDAARAFEG | |
| LDSDEELGLLCHHTSYLDQRVASFCTLTDMQHGQDLEGAQELPLCVDPGSGKEFMDTTGE | |
| RSPSPLTGKVNQLELILRQLQTDLRKEKQDKAVLQAEVQHLRQDNMRLQEESQTATAQLR | |
| KFTEWFFTTIDKKS | |

TABLE 60

| | |
|---|---|
| cbe_3665307 HK1 | |
| hexokinase 1 | |
| length = 905 | |
| MAKRALRDFIDKYLYAMRLSDETLIDIMTRFRKEMKNGLSRDFNPTATVKMLPTFVRSIP | SEQ ID NO: 51 |
| DGSEKGDFIALDLGGSSFRILRVQVNHEKNQNVHMESEVYDTPENIVHGSGSQLFDHVAE | |
| CLGDFMEKRKIKDKKLPVGFTFSFPCQQSKIDEAILITWTKRFKASGVEGADVVKLLNKA | |
| IKKRGDYDANIVAVVNDTVGTMMTCGYDDQHCEVGLIIGTGTNACYMEELRHIDLVEGDE | |
| GRMCINTEWGAFGDDGSLEDIRTEFDREIDRGSLNPGKQLFEKMVSGMYLGELVRLILVK | |
| MAKEGLLFEGRITPELLTRGKFNTSDVSAIEKNKEGLHNAKEILTRLGVEPSDDDCVSVQ | |
| HVCTIVSFRSANLVAATLGAILNRLRDNKGTPRLRTTVGVDGSLYKTHPQYSRRFHKTLR | |
| RLVPDSDVRFLLSESGSGKGAAMVTAVAYRLAEQHRQIEETLAHFHLTKDMLLEVKKRMR | |
| AEMELGLRKQTHNNAVVKMLPSFVRRTPDGTENGDFLALDLGGTNFRVLLVKIRSGKKRT | |
| VEMHNKIYAIPIEIMQGTGEELFDHIVSCISDFLDYMGIKGPRMPLGFTFSFPCQQTSLD | |
| AGILITWTKGFKATDCVGHDVVTLLRDAIKRREEFDLDVVAVVNDTVGTMMTCAYEEPTC | |
| EVGLIVGTGSNACYNEEMKNVEMVEGDQGQMCINMEWGAFGDNGCLDDIRTHYDRLVDEY | |
| SLNAGKQRYEKNISGMYLGEIVRNILIDFTKKGFLFRGQISETLKTRGIFETKFLSQIES | |
| DRLALLQVRAILQQLGLNSTCDDSILVKTVCGVVSRRAAQLCGAGMAAVVDKIRENRGLD | |
| RLNVTVGVDGTLYKLHPHFSRIMHQTVKELSPKCNVSFLLSEDGSGKGAALITAVGVRLR | |
| TEASS | |

TABLE 61

| | |
|---|---|
| cbe_3670766 ACTC | |
| actin, alpha, cardiac muscle | |
| length = 377 | |
| MCDDEETTALVCDNGSGLVKACFAGDDAPRAVFPSIVGRPRHQGVMVGMGQKDSYVGDEA | SEQ ID NO: 52 |
| QSKRGILTLKYPIEHGIITNWDDMEKIWHHTFYNELRVAPEEHPTLLTEAPLNPKANREK | |
| MTQIMFETFNVPAMYVAIQAVLSLYASGRTTGIVLDSGDGVTHNVPIYEGYALPHAIMRL | |
| DLAGRDLTDYLMKILTERGYSFVTTAEREIVRDIKEKLCYVALDFENEMATAASSSSLEK | |
| SYELPDGQVITIGNERFRCPETLFQPSFIGMESAGIHETTYNSIMKCDIDIRKDLYANNV | |
| LSGGTTMYPGIADRMQKEITALAPSTMKIKIIAPPERKYSVWIGGSILASLSTFQQMWIS | |
| KQEYDEAGPSIVHRKCF | |

TABLE 62

| | |
|---|---|
| cbe_3694420 ARHGEF2 | |
| rho/rac guanine nucleotide exchange factor (GEF) 2 | |
| length = 958 | |
| MKEAKDARYTNGHLFTTISVSGMTMCYACNKSITAKEALICPTCNVTIHNRCKDTLANCT | SEQ ID NO: 53 |
| KVKQKQQKAALLKNNTALQSVSLRSKTTIRERPSSAIYPSDSFRQSLLGSRRGRSSLSLA | |
| KSVSTTNIAGHFNDESPLGLRRILSQSTDSLNNRNRTLSVESLIDEEVIYSELMSDFEMD | |
| EKDFAADSWSLAVDSSFLQQHKKEVMKQQDVIYELIQTELHHVRTLKIMTRLFRTGMLEE | |
| LHLEPGVVQGLFPCVDELSDIHTRFLSQLLERRRQALCPGSTRNFVIHRLGDLLISQFSG | |
| PSAEQMCKTYSEFCSRHSKALKLYKELYARDKRFQQFIRKVTRPAVLKRHGVQECILLVT | |
| QRITKYPLLISRILQHSHGIEEERQDLTTALGLVKELLSNVDEGIYQLEKGARLQEIYNR | |
| MDPRAQTPVPGKGPFGREELLRRKLIHDGCLLWKTATGRFKDVLVLLMTDVLVFLQEKDQ | |
| KYIFPTLDKPSVVSLQNLIVRDIANQEKGMFLISAAPPEMYEVHTASRDDRSTWIRVIQQ | |
| SVRTCPSREDFPLIETEDEAYLRRIKMELQQKDRALVELLREKVGLFAEMTHFQAEEDGG | |
| SGMALPTLPRGLFRSESLESPRGERLLQDAIREVEGLKDLLVGPGVELLLTPREPALPLE | |
| PDSGGNTSPGVTANGEARTFNGSIELCRADSDSSQRDRNGNQLRSPQEEALQRLVNLYGL | |
| LHGLQAAVAQQDTLMEARFPEGPERREKLCRANSRDGEAGRAGAAPVAPEKQATELALLQ | |
| RQHALLQEELRRCRRLGEERATEAGSLEARLRESEQARALLEREAEEARRQLAALGQTEP | |
| LPAEAPWARRPVDPRRRSLPAGDALYLSFNPPQPSRGTDRLDLPVTTRSVHRNFEDRERQ | |
| ELGSPEERLQDSSDPDTGSEEEGSSRLSPPHSPRDFTRMQDIPEETESRDGEAVASES | |

TABLE 63

| | |
|---|---|
| cbe_3705944 TNFAIP1 | |
| tumor necrosis factor, alpha-induced protein 1 (endothelial) | |
| length = 316 | |
| MSGDTCLCPASGAKPKLSGFKGGGLGNKYVQLNVGGSLYYTTVRALTRHDTMLKAMFSGR | SEQ ID NO: 54 |
| MEVLTDKEGWILIDRCGKHFGTILNYLRDDTITLPQNRQEIKELMAEAKYYLIQGLVNMC | |
| QSALQDKKDSYQPVCNIPIITSLKEEERLIESSTKPVVKLLYNRSNNKYSYTSNSDDHLL | |
| KNIELFDKLSLRFNGRVLFIKDVIGDEICCWSFYGQGRKLAEVCCTSIVYATEKKQTKVE | |
| FPEARIYEETLNVLLYETPRVPDNSLLEATSRSRSQASPSEDEETFELRDRVRRIHVKRY | |
| STYDDRQLGHQSTHRD | |

TABLE 64

| | |
|---|---|
| cbe_3753270 gbh_ac004503 | |
| Homo sapiens chromosome 5, P1 clone 1354A7 (LBNL H47), complete | |
| length = 515 | |
| MALFVRLLALALALALGPAATLAGPAKSPYQLVLQHSRLRGRQHGPNVCAVQKVIGTNRK | SEQ ID NO: 55 |
| YFTNCKQWYQRKICGKSTVISYECCPGYEKVPGEKGCPAALPLSNLYETLGVVGSTTTQL | |
| YTDRTEKLRPEMEGPGSFTIFAPSNEAWASLPAEVLDSLVSNVNIELLNALRYHMVGRRV | |
| LTDELKHGMTLTSMYQNSNIQIHHYPNGIVTVNCARLLKADHHATNGVVHLIDKVISTIT | |
| NNIQQIIEIEDTFETLRAAVAASGLNTMLEGNGQYTLLAPTNEAFEKIPSETLNRILGDP | |
| EALRDLLNNHILKSAMCAEAIVAGLSVETLEGTTLEVCCSGDMLTINGKAIISNKDILAT | |
| NGVIHYIDELLIPDSAKTLFELAAESDVSTAIDLFRQAGLGNHLSGSERLTLLAPLNSVF | |
| KDGTPPIDAHTRNLLRNHIIKDQLASKYLYHGQTLETLGGKKLRVFVYRNSLCIENSCIA | |
| AHDKRGRYGTLFTMDRVLTPPMGTVMDVLKGDNRF | |

TABLE 65

| | |
|---|---|
| cbe_3764052 MARK4 | |
| MAP/microtubule affinity-regulating kinase 4 | |
| length = 381 | |
| MPFGNTHNKFKLNYKPEEEYPDLSKHNNHMAKVLTLELYKKLRDKETPSGFTVDDVIQTG | SEQ ID NO: 56 |
| VDNPGHPFIMTVGCVAGDEESYEVFKELFDPIISDRHGGYKPTDKHKTDLNHENLKGGDD | |
| LDPNYVLSSRVRTGRSIKGYTLPPHCSRGERRAVEKLSVEALNSLTGEFKGKYYPLKSMT | |
| EKEQQQLIDDHFLFDKPVSPLLLASGMARDWPDARGIWHNDNKSFLVWVNEEDHLRVISM | |
| EKGGNMKEVFRRFCVGLQKIEEIFKKAGHPFMWNQHLGYVLTCPSNLGTGLRGGVHVKLA | |
| HLSKHPKFEEILTRLRLQKRGTGGVDTAAVGSVFDVSNADRLGSSEVEQVQLVVDGVKLM | |
| VEMEKKLEKGQSIDDMIPAQK | |

TABLE 66

| | |
|---|---|
| cbe_3770440 gbh_af217505 | |
| Homo sapiens uncharacterized bone marrow protein BM028 mRNA, | |
| length = 116 | |
| MWTKKDAGKKVVPCRHDWHQTGGEVTISVYAKNSLPELSRVEANSTLLNVHIVFEGEKEF | SEQ ID NO: 57 |
| DQNVKLWGVIDVKRSYVTMTATKIEITMRKAEPMQWASLELPAAKKQEKQKDDTTD | |

TABLE 67

| | |
|---|---|
| cbe_3772244 PIGQ | |
| phosphatidylinositol glycan, class Q | |
| length = 760 | |
| MVLKAFFPTCCVSTDSGLLVGRWVPEQSSAVVLAVLHFPFIPIQVKQLLAQVRQASQVGV | SEQ ID NO: 58 |
| AVLGTWCHCRQEPEESLGRFLESLGAVFPHEPWLRLCRERGGTFWSCEATHRQAPTAPGA | |
| PGEDQVMLIFYDQRQVLLSQLHLPTVLPDRQAGATTASTGGLAAVFDTVARSEVLFRSDR | |
| FDEGPVRLSHWQSEGVEASILAELARRASGPICLLLASLLSLVSAVSACRVFKLWPLSFL | |
| GSKLSTCEQLRHRLEHLTLIFSTRKAENPAQLMRKANTVASVLLDVALGLMLLSWLHGRS | |
| RIGHLADALVPVADHVAEELQHLLQWLMGAPAGLKMNRALDQVLGRFFLYHIHLWISYIH | |
| LMSPFVEHILWHVGLSACLGLTVALSLLSDIIALLTFHIYCFYVYGARLYCLKIHGLSSL | |
| WRLFRGKKWNVLRQRVDSCSYDLDQLFIGTLLFTILLFLLPTTALYYLVFTLLRLLVVAV | |
| QGLIHLLVDLINSLPLYSLGLRLCRPYRLADKPTALQPRGAHLPPPQLWLPPQALLGRPV | |
| PQAVPWGAHLPLEAERGQAGLRELLARLAPPHGHSQPSALPGWHQLSWRMSCALWTLLCA | |
| PEHGRPCYHTLGLEVIGSEQMWGWPARLAALHHWHCLPWDPLPTCCGHHGGEHSNPRCPE | |
| HCPMPTLCTQVQRVRPPQQPQVECWSPWGLPSGSALAVGVEGPCQDEPPSPRHPLAPSAE | |
| QHPASGGLKQSLTPVPSGPGPSLPEPHGVYLRNFPGEVAL | |

TABLE 68

| | |
|---|---|
| cbe_3773649 EEF1A1L14 | |
| eukaryotic translation elongation factor 1 alpha 1-like 14 | |
| length = 398 | |
| MQSERGITIDISLWKFETSKYYVTIIDAPGHRDFIQNMITGTSQADCAVLIVAAGVGEFE | SEQ ID NO: 59 |
| AGISKNGQTREHALLAYTLGVKQLIVGVNKMDSTEPPYSQKRYEEIVKEVSTYIKKIGYN | |
| PDTVAFVPISGWNGDNMLEPSANMPWFKGWKVTRKDGNASGTTLLEALDCILPPTRPTDK | |
| PLGLPLQDVYKICGIGTVPVGRVETGVLKPGMVVTFGPVNVTTEVKSVEMHHEALGEALP | |
| GDNVGFNVKNVSVKDVRRCNVAGDSKNDPPMEAAGFPAQVIILNHPGQISAGYAPVLDCH | |
| TAHIACKFAELKEKIDRRSGKKLEDGPKFLKSGDAAIVDMVPGKPMCVESFSDYPPLGCF | |
| AVRDMRQTVAVGVIKAVDKKAACAGKVTKSAQKAQKAK | |

TABLE 69

| | |
|---|---|
| cbe_3787129 LOC55828 | |
| zinc finger protein ZNF140-like protein | |
| length = 478 | |
| MSQVTFSDVAIDFSHEEWACLDSAQRDLYKDVMVQNYENLVSVGLSVTKPYVIMLLEDGK | SEQ ID NO: 60 |
| EPWMMEKKLSKAYPFPLSHSVPASVNFGFSALFEHCSEVTEIFELSELCVFWVLHFLSNS | |
| PNSTVEAFSRSKKKKKKKKKRQCFAFLIYFRLGIKMGKQGIINKEGYLYEDSPQPVTMEK | |
| VVKQSYEFSNSNKNLEYTECDTFRSTFHSKSTLSEPQNNSAEGNSHKYDILKKNLSKKSV | |
| IKSERINGGKKLLNSNKSGAAFNQSKSLTLPQTCNREKIYTCSECGKAFGKQSILSRHWR | |
| IHTGEKPYECRECGKTFSHGSSLTRHQISHSGEKPYKCIECGKAFSHGSSLTNHQSTHTG | |
| EKPYECMNCGKSFSRVSLLIQHLRIHTQEKRYECRICGKAFIHSSSLIHHQKSHTGEKPY | |
| ECRECGKAFCCSSHLTQHQRIHSMKYKYECNKCLKVFSSFSFLVQHQSIHTEEKPFEV | |

TABLE 70

| | |
|---|---|
| cbe_3789063 AP2M1 | |
| adaptor-related protein complex 2, mu 1 subunit | |
| length = 435 | |
| MIGGLFIYNHKGEVLISRVYRDDIGRNAVDAFRVNVIHARQQVRSPVTNIARTSFFHVKR | SEQ ID NO: 61 |
| SNIWLAAVTKQNVNAAMVFEFLYKMCDVMAAYFGKISEENIKNNFVLIYELLDEILDFGY | |
| PQNSETGALKTFITQQGIKSQHQTKEEQSQITSQVTGQIGWRREGIKYRRNELFLDVLES | |
| VNLLMSPQGQVLSAHVSGRVVMKSYLSGMPECKFGMNDKIVIEKQGKGTADETSKSGKQS | |
| IAIDDCTFHQCVRLSKFDSERSISFIPPDGEFELMRYRTTDDIILPFRVIPLVREVGRTK | |
| LEVKVVIKSNFKPSLLAQKIEVRIPTPLNTSGVQVICMKCKAKYKASENAIVWKIKRMAG | |
| MKESQISAEIELLPTNDKKKWARPPISMNFEVPFAPSGLKVRYLKVFEPKLNYSDHDVIK | |
| WVRYIGRSGIYETRC | |

TABLE 71

| | |
|---|---|
| cbe_3800240 IVD | |
| isovaleryl Coenzyme A dehydrogenase | |
| length = 423 | |
| MATATRLLGWRVASWRLRPPLAGFVSQRAYSLLPVDDAINGLSEEQRQLRQTMAKFLQEH | SEQ ID NO: 62 |
| LAPKAQEIDRSNEFKNLREFWKQLGNLGVLGITAPVQYCGSGLGYLEHVLVMEEISRASG | |
| AVGLSYGAHSNLCINQLVRNGNEAQKEKYLPKLISGEYIGALAMSEPNAGSDVVSMKLKA | |
| EKKGNHYILNGNKFWITNGPDADVLIVYAKTDLAAVPASRGITAFIVEKGMPGFSTSKKL | |
| DKLGMRGSNTCELIFEDCKIPAANILGHENKGVYVLMSGLDLERLVLAGGPLGLMQAVLD | |
| HTIPYLHVREAFGQKIGHFQLMQGKMADMYTRLMACRQYVYNVAKACDEGHCTAKDCAGV | |
| ILYSAECATQVALDGIQCFGGNGYINDFPMGRFLRDAKLYEIGAGTSEVRRLVIGRAFNA | |
| DFH | |

TABLE 72

| | |
|---|---|
| be_3810032 gbh_af142421 | |
| Homo sapiens QUAKING isoform 5 (QUAKING) mRNA, complete cds. | |
| length = 337 | |
| METKEKPKPTPDYLMQLMNDKKLMSSLPNFCGIFNHLERLLDEEISRVRKDMYNDTLNGS | SEQ ID NO: 63 |
| TEKRSAELPDAVGPIVQLQEKLYVPVKEYPDFNFVGRILGPRGLTAKQLEAETGCKIMVR | |
| GKGSMRDKKKEEQNRGKPNWEHLNEDLHVLITVEDAQNRAEIKLKRAVEEVKKLLVPAAE | |
| GEDSLKKMQLMELAILNGTYRDANIKSPALAFSLAATAQAAPRIITGPAPVLPPAALRTP | |
| TPAGPTIMPLIRQIQTAVMPNGTPHPTAAIVPPGPEAGLIYTPYEYPYTLAPATSILEYP | |
| IEPSGVLGAVATKVRRHDMRVHPYQRIVTADRAATGN | |

TABLE 73

| | |
|---|---|
| cbe_3810791 EPHB1 | |
| EphB1 | |
| length = 984 | |
| MALDYLLLLLLASAVAANEETLMDTRTATAELGWTANPASGWEEVSGYDENLNTIRTYQV | SEQ ID NO: 64 |
| CNVFEPNQNNWLLTTFINRRGAHRIYTEMRFTVRDCSSLPNVPGSCKETFNLYYYETDSV | |
| IATKKSAFWSEAPYLKVDTIAADESFSQVDFGGRLMKVNTEVRSFGPLTRNGFYLAFQDY | |
| GACMSLLSVRVFFKKCPSIVQNFAVFPETMTGAESTSLVIARGTCIPNAEEVDVPIKLYC | |
| NGDGEWMVPIGRCTCKPGYEPENSVACKACPAGTFKASQEAEGCSHCPSNSRSPAEASPI | |
| CTCRTGYYRADFDPPEVACTSVPSGPRNVISIVNETSIILEWHPPRETGGRDDVTYNIIC | |
| KKCRADRRSCSRCDDNVEFVPRQLGLTECRVSISSLWAHTPYTFDIQAINGVSSKSPFPP | |
| QHVSVNITTNQAAPSTVPIMHQVSATMRSITLSWPQPEQPNGIILDYEIRYYEKEHNEFN | |
| SSMARSQTNTARIDGLRPGMVYVVQVRARTVAGYGKFSGKMCFQTLTDDDYKSELREQLP | |
| LIAGSAAAGVVFVVSLVAISIVCSRKRAYSKEAVYSDKLQHYSTGRGSPGMKIYIDPFTY | |
| EDPNEAVREFAKEIDVSFVKIEEVIGAGEFGEVYKGRLKLPGKREIYVAIKTLKAGYSEK | |
| QRRDFLSEASIMGQFDHPNIIRLEGVVTKSRPVMIITEFMENGALDSFLRQNDGQFTVIQ | |
| LVGMLRGIAAGMKYLAEMNYVHRDLAARNILVNSNLVCKVSDFGLSRYLQDDTSDPTYTS | |
| SLGGKIPVRWTAPEAIAYRKFTSASDVWSYGIVMWEVMSFGERPYWDMSNQDVINAIEQD | |
| YRLPPPMDCPAALHQLMLDCWQKDRNSRPRFAEIVNTLDKMIRNPASLKTVATITAVPSQ | |
| PLLDRSIPDFTAFTTVDDWLSAIKMVQYRDSFLTAGFTSLQLVTQMTSEDLLRIGITLAG | |
| HQKKILNSIHSMRVQISQSPTAMA | |

TABLE 74

| | |
|---|---|
| cbe_3820530 PGAM1 | |
| phosphoglycerate mutase 1 (brain) | |
| length = 254 | |
| MAAYKLVLIRHGESAWNLENRFSGWYDADLSPAGHEEAKRGCQALRDAGYEFDICFTSVQ | SEQ ID NO: 65 |
| KRAIRTLWTVLDAIDQMWLPVVRTWRLNERHYGCLTGLNKAETAAKHGEAQVKIWRRSYD | |
| VPPPPMEPDHPFYSNISKDRRYADLTEDQLPSCESLKDTIARALPFWNEEIVPQIKEGKR | |
| VLIAAHGNSLRGIVKHLEGLSEEAIMELNLPTGIPIVYELDKNLKPIKPMQFLGDEETVR | |
| KANEAVAAQCKAKK | |

TABLE 75

| | |
|---|---|
| cbe_3825897 POLR2L | |
| polymerase (RNA) II (DNA directed) polypeptide L, 7.6 kDa | |
| length = 67 | |
| MIIPVRCFTCGKIVGNKWEAYLGLLQAEYTEGDALDALGLKRYCCRRMLLAHVDLIEKLL | SEQ ID NO: 66 |
| NYAPLEK | |

TABLE 76

| | |
|---|---|
| cbe_3826540 NYD-TSP1 | |
| testis-specific protein NYD-TSP1 | |
| length = 430 | |
| MASSAKSAEMPTISKTLNPTPDPHQEYLDPRITIALFEIGSHSPSSWGSLPFLKNSSHQV | SEQ ID NO: 67 |
| TEQQTAQKFNNLLKEIKDILKNMAGFEEKITEAKELFEETNITEDVSAHKENIRGLDKIN | |
| EMLSTNLPVSLAPEKEDNEKKQEMILETNITEDVSAHKENIRGLDKINEMLSTNLPVSLA | |
| PEKEDNEKKQQMIMENQNSENTAQVFARDLVNRLEEKKVLNETQQSQEKAKNRLNVQEET | |
| MKIRNNMEQLLQEAEHWSKQHTELSKLIKSYQKSQKDISETLGNNGVGFQTQPNNEVSAK | |
| HELEEQVKKLSHDTYSLQLMAALLENECQILQQRVEILKELHHQKQGTLQEKPIQINYKQ | |
| DKKNQKPSEAKKVEMYKQNKQAMKGTFWKKDRSCRSLDVCLNKKACNTQFNIHVARKALR | |
| GKMRSASSLR | |

TABLE 77

| | |
|---|---|
| cbe_3833533 TRIM29 | |
| tripartite motif-containing 29 | |
| length = 588 | |
| MEAADASRSNGSSPEARDARSPSGPSGSLENGTKADGKDAKTTNGHGGEAAEGKSLGSAL | SEQ ID NO: 68 |
| KPGEGRSALFAGNEWRRPIIQFVESGDDKNSNYFSMDSMEGKRSPYAGLQLGAAKKPPVT | |
| FAEKGELRKSIFSESRKPTVSIMEPGETRRNSYPRADTGLFSRSKSGSEEVLCDSCIGNK | |
| QKAVKSCLVCQASFCELHLKPHLEGAAFRDHQLLEPIRDFEARKCPVHGKTMELFCQTDQ | |
| TCICYLCMFQEHKNHSTVTVEEAKAEKETELSLQKEQLQLKIIEIEDEAEKWQKEKDRIK | |
| SFTTNEKAILEQNFRDLVRDLEKQKEEVRAALEQREQDAVDQVKVIMDALDERAKVLHED | |
| KQTREQLHSISDSVLFLQEFGALMSNYSLPPPLPTYHVLLEGEGLGQSLGNFKDDLLNVC | |
| MRHVEKMCKADLSRNFIERNEMENGGDHRYVNNYTNSFGGEWSAPDTMKRYSMYLTPKGG | |
| VRTSYQPSSPGRFTKETTQKNFNNLYGTKGNYTSRVWEYSSSIQNSDNDLPVVQGSSSFS | |
| LKGYPSLMRSQSPKAQPQTWKSGKQTMLSHYRPFYVNKGNGIGSNEAP | |

TABLE 78

| | |
|---|---|
| cbe_3834015 TFPI | |
| tissue factor pathway inhibitor (lipoprotein-associated coagulation inhibitor) | |
| length = 304 | |
| MIYTMKKVHALWASVCLLLNLAPAPLNADSEEDEEHTIITDTELPPLKLMHSFCAFKADD | SEQ ID NO: 69 |
| GPCKAIMKRFFFNIFTRQCEEFIYGGCEGNQNRFESLEECKKMCTRDNANRIIKTTLQQE | |
| KPDFCFLEEDPGICROYITRYFYNNQTKQCERFKYGGCLGNMNNFETLEECKNICEDGPN | |
| GFQVDNYGTQLNAVNNSLTPQSTKVPSLFEFHGPSWCLTPADRGLCRANENRFYYNSVIG | |
| KCRPFKYSGCGGNENNFTSKQECLRACKKGFIQRISKCGLIKTKRKRKKQRVKIAYEEIF | |
| VKNM | |

TABLE 79

| | |
|---|---|
| cbe_3842044 PSMB3 | |
| proteosome (prosome, macropain) subunit, beta type, 3 | |
| length = 205 | |
| MSIMSYNGGAVMAMKGKNCVAIAADRRFGIQAQLVTTDFQKIFPMGDRLYIGLACLATDV | SEQ ID NO: 70 |
| QTVAQRLKFRLNLYELKEGRQIKPYTLMSMVANLLYEKRFGPYYTEPVIAGLDPKTFKPF | |
| ICSLDLIGCPMVTDDFVVSGTCAEQMYGMCESLWEPNMDPDHLFETISQAMLNAVDRDAV | |
| SGMGVIVHIIEKDKITTRTLKARMD | |

TABLE 80

| | |
|---|---|
| cbe_3845697 Hs. 191063 | |
| Human DNA sequence from clone RP1-63M2 on chromosome 20 Contains the gene for | |
| CBFA2T2 (core-binding factor, runt domain, alpha subunit 2; translocated to, | |
| 2), part of a gene for a protein similar to ACTIN, the E2F1 (E2F transcription | |
| factor 1) gene, a nov | |
| length = 412 | |
| DRGREVSGRRSRVARRSGAFSPAAGPSPKVASVSGSRRVHRPSSLGRIAVVVDQGSGFTK | SEQ ID NO: 71 |
| AGFAGENQPRIVLKSSSLVPSWDRPVLPGAPGCELAGGVAPAHPIKHGVVADWEALEGLW | |
| ERLLVGGLRVCPEQWPVLVSDSPLAPPAGRERVAELLFETLAVPACHMASTALLALCSTG | |
| AFSGLAVEAGAGVCHATPIYAGHSWHQATFRLNVAGSTLSRYLRDLLVAANPDLLQQALP | |
| RKAITHLKKRSCYVSLDFEGDLRDPARHHPASFSVGNGCCVCLSSERFRCPEPIFQPGLL | |
| GQAEQGLPALAFRALQKMPKTLRTRLADTVVLAGGSTLFPGFAERLDKELEAQCRRHGYA | |
| ALRPHLVAKHGRGMAVWTGGSMVASLHSFQRRWITRAMYQECGSRLLYDVFN | |

TABLE 81

| | |
|---|---|
| cbe_3849002 CKLF1 | |
| chemokine-like factor 1 | |
| length = 152 | |
| MDNVQPKIKHRPFCFSVKGHVKMLRLALTVTSMTFFIIAQAPEPYIVITGFEVTVILFFI | SEQ ID NO: 72 |
| LLYVLRLDRLMKWLFWPLLDIINSLVTTVFMLIVSVLALIPETTTLTVGGGVFALVTAVC | |
| CLADGALIYRKLLFNPSGPYQKKPVHEKKEVL | |

TABLE 82

| | |
|---|---|
| cbe_3880186 FLJ20343 | |
| hypothetical protein FLJ20343 | |
| length = 501 | |
| MAQPHTTSVPYFARSPAPPPPSRSGAPPQPPATLRPSRRRTRPPRPADRRDAPADCAYLW | SEQ ID NO: 73 |
| RILTPRRGRARRSDVGARHRACGRRDVLLSRQGPANPEGARRVVGGQERVWPAVRRGRCG | |
| SMFRLLSWSLGRGFLRAAGRRCRGCSARLLPGLAGGPGPEVQVPPSRVAPHGRGPGLLPL | |
| LAALAWFSRPAAAEEEEQQGADGAAAEDGADEAEAEIIQLLKRAKLSIMKDEPEEAELIL | |
| HDALRLAYQTDNKKAITYTYDLMANLAFIRGQLENAEQLFKATMSYLLGGGMKQEDNAII | |
| ElSLKLASIYAAQNRQEFAVAGYEFCISTLEEKIEREKELAEDIMSVEEKANTHLLLGMC | |
| LDACARYLLFSKQPSQAQRMYEKALQISEEIQGERHPQTIVLMSDLATTLDAQGRFDEAY | |
| IYMQRASDLARQINHPELHMVLSNLAAVLMHRERYTQAKEIYQEALKQAKLKKDEISVQH | |
| IREELAELSKKSRPLTNSVKL | |

TABLE 83

| | |
|---|---|
| cbe_3884811 RCP | |
| Rab coupling protein | |
| length = 649 | |
| MSLMVSAGRGLGAVWSPTHVQVTVLQARGLRAKGPGGTSDAYAVIQVGKEKYATSVSERS | SEQ ID NO: 74 |
| LGAPVWREEATFELPSLLSSGPAAAATLQLTVLHRALLGLDKFLGRAEVDLRDLHRDQCR | |
| RKTQWYKLKSKPGKKDKERGEIEVDIQFMRNNMTASMFDLSMKDKSRNPFGKLKDKIKGK | |
| NKDSGSDTASAIIPSTTPSVDSDDESVVKDKKKKSKIKTLLSKSNLQKTPLSQSMSVLPT | |
| SKPEKVLLRPGDFQSQWDEDDNEDESSSASDVMSHKRTASTDLKQLNQVNFTLPKKEGLS | |
| FLGGLRSKNDVLSRSNVCINGNHVYLEQPQPTGEIKDSSPSSSPSPKGFRKKTLFSSTEN | |
| LAAGSWKEPAEGGGLSTDRDVSESSTKDSLKSMTLPTYRPAPLISGDLREKMAPANSEAT | |
| KEAKESKKPESRRSSLLSLMTGKKDVAKGSECENLLTVPGREKEGMLMGVKPGEDASGPA | |
| EDLVRRSEKDTAAVVSRQGSSLNLFEDVQITEPEAEPESKSEPRPPISSPRAPQTRAVKP | |
| RLHPVKPMNATATKVANCSLGTATIISENLNNEVMMKKYSPSDPAFAYAQLTHDELIQLV | |
| LKQKETISKKEFQVRELEDYIDNLLVRVMEETPNILRIPTQVGKKAGKM | |

TABLE 84

| | |
|---|---|
| cbe_4138433 KIFAP3 | |
| kinesin-associated protein 3 | |
| length = 792 | |
| MQGEDARYLKRKVKGGNIDVHPSEKALIVHYEVEATILGEMGDPMLGERKECQKIIRLKS | SEQ ID NO: 75 |
| LNANTDITSLARKVVEECKLIHPSKLNEVEQLLYYLQNRRDSLSGKEKKEKSSKPKDPPP | |
| FEGMEIDEVANINDMDEYIELLYEDTPDKVRGSALILQLARNPDNLEELLLNETALGALA | |
| RVLREDWKQSVELATNIIYIFFCFSSFSQFHGLITHYKIGALCMNIIDHELKRHELWQEE | |
| LSKKKKAVDEDPENQTLRKDYEKTFKKYQGLVVKQEQLLRVALYLLLNLAEDTRTELKMR | |
| NKNIVHMLVKALDRDNFELLILVVSFLKKLSIFMENKNDMVEMDIVEKLVKMIPCEHEDL | |
| LNITLRLLLNLSFDTGLRNKMVQVGLLPKLTALLGNDNYKQIANCVLYHISMDDRFKSMF | |
| AYTDCIPQLMKMLFECSDERIDLELISFCINLAANKRNVQLICEGNGLKMLMKRALKFKD | |
| PLLMKMIRNISQHDGPTKNLFIDYVGDLAAQISNDEEEEFVIECLGTLANLTIPDLDWEL | |
| VLKEYKLVPYLKDKLKPGAAEDDLVLEVVIMIGTVSMDDSCAALLAKSGIIPALIELLNA | |
| QQEDDEFVCQIIYVFYQMVFHQATRDVIIKETQAPAYLIDLMHDKNNEIRKVCDNTLDII | |
| AEYDEEWAKKIQSEKFRWHNSQWLEMVESRQMDESEQYLYGDDRIEPYIHEGDILERPDL | |
| FYNSDGLIASEGAISPDFFNDYHLQNGDVVGQHSFPGSLGMDGFGQPVGILGRPATAYGF | |
| RPDEPYYYGYGS | |

TABLE 85

| | |
|---|---|
| cbe_4279962 DKKL1-pending | |
| soggy-1 gene | |
| length = 242 | |
| MGEASPPAPARRHLLVLLLLLSTLVIPSAAAPIHDADAQESSLGLTGLQSLLQGFSRLFL | SEQ ID NO: 76 |
| KGNLLRGIDSLFSAPMDFRGLPGNYHKEENQEHQLGNNTLSSHLQIDKMTDNKTGEVLIS | |
| ENVVASIQPAEGSFEGDLKVPRMEEKEALVPIQKATDSFHTELHPRVAFWIIKLPRRRSH | |
| QDALEGGHWLSEKRHRLQAIRDGLRKGTHKDVLEEGTESSSHSRLSPRKTHLLYILRPSR | |
| QL | |

TABLE 86

| | |
|---|---|
| cbe_4281271 CRYAB | |
| crystallin, alpha B | |
| length = 175 | |
| MDIAIHHPWIRRPFFPFHSPSRLFDQFFGEHLLESDLFPTSTSLSPFYLRPPSFLRAPSW | SEQ ID NO: 77 |
| FDTGLSEMRLEKDRFSVNLDVKHFSPEELKVKVLGDVIEVHGKHEERQDEHGFISREFHR | |
| KYRIPADVDPLTITSSLSSDGVLTVNGPRKQVSGPERTIPITREEKPAVTAAPKK | |

TABLE 87

| | |
|---|---|
| cbe_4344185 SRA1 | |
| steroid receptor RNA activator 1 | |
| length = 237 | |
| MTRCPAGQAEVEMAELYVKPCNKERGWNDPPQFSYGLQTQAGGPRRSLLTKRVAAPQDGS | SEQ ID NO: 78 |
| PRVPASETSPGPPPMGPPPPSSKAPRSPPVGSGPASGVEPTSFPVESEARLMEDVLRPLE | |
| QALEDCRGHTRKQVCDDISRRLALLQEQWAGCKLSIPVKKRMALLVQELSSHRWDAADDI | |
| HRSLMVDHVTEVSQWMVGVKRLIAEKRSLFSEEAANEEKSAATAEKNHTIPGFQQAS | |

TABLE 88

| | |
|---|---|
| cbe_4383836 KIAA0905 | |
| yeast Sec31p homolog | |
| length = 1220 | |
| MKLKEVDRTAMQAWSPAQNHPIYLATGTSAQQLDATFSTNASLEIFELDLSDPSLDMKSC | SEQ ID NO: 79 |
| ATFSSSHRYHKLIWGPYKMDSKGDVSGVLIAGGENGNIILYDPSKIIAGDKEVVIAQNDK | |
| HTGPVRALDVNIFQTNLVASGANESEIYIWDLNNFATPMTPGAKTQPPEDISCIAWNRQV | |
| QHILASASPSGRATVWDLRKNEPIIKVSDHSNRMHCSGLAWHPDVATQMVLASEDDRLPV | |
| IQMWDLRFASSPLRVLENHARGILAIAWSMADPELLLSCGKDAKILCSNPNTGEVLYELP | |
| TNTQWCFDIQWCPRNPAVLSAASFDGRISVYSINGGSTDGLRQKQVDKLSSSFGNLDPFG | |
| TGQPLPPLQIPQQTAQHSIVLPLKKPPKWIRRPVGASFSFGGKLVTFENVRMPSHQGAEQ | |
| QQQQHHVFISQVVTEKEFLSRSDQLQQAVQSQGFINYCQKKIDASQTEFEKNVWSFLKVN | |
| FEDDSRGKYLELLGYRKEDLGKKIALALNKVDGANVALKDSDQVAQSDGEESPAAEEQLL | |
| GEHIKEEKEESEFLPSSGGTFNISVSGDIDGLITQALLTGNFESAVDLCLHDNRMADAII | |
| LAIAGGQELLARTQKKYFAKSQSKITRLITAVVMKNWKEIVESCDLKNWREALAAVLTYA | |
| KPDEFSALCDLLGTRLENEGDSLLQTQACLCYICAGNVEKLVACWTKAQDGSHPLSLQDL | |
| IEKVVILRKAVQLTQAMDTSTVGVLLAAKMSQYANLLAAQGSIAAALAFLPDNTNQPNIM | |
| QLRDRLCRAQGEPVAGHESPKIPYEKQQLPKGRPGPVAGHHQMPRVQTQQYYPHGENPPP | |
| PGFIMHGNVNPNASGQLPTSPGHMHTQVPPYPQPQPYQPAQPYPFGTGGSAMYRPQQPVA | |
| PPTSNAYPNTPYISSASSYTGQSQLYAAQHQASSPTSSPATSFPPPPSSGASFQHGGPGA | |
| PPSSSAYALPPGTTGTLPAASELPASQRTGPQNGWNDPPALNRVPKKKKMPENFMPPVPI | |
| TSPIMNPLGDPQSQMLQQQPSAPVPLSSQSSFPQPHLPGGQPFHGVQQPLGQTGMPPSFS | |
| KPNIEGAPGAPIGNTFQHVQSLPTKKITKKPIPDEHLILKTTFEDLIQRCLSSATDPQTK | |
| RKLDDASKRLEFLYDKLREQTLSPTITSGLHNIARSIETRNYSEGLTMHTHIVSTSNFSE | |
| TSAFMPVLKVVLTQANKLGV | |

TABLE 89

| | |
|---|---|
| cbe_4540466 PUM1 | |
| pumilio homolog 1 (Drosophila) | |
| length = 1186 | |
| MSVACVLKRKAVLWQDSFSPHLKHHPQEPANPNNPVVLTSGTGSQAQPQPAANQALAAGT | SEQ ID NO: 80 |
| HSSPVPGSIGVAGRSQDDANVDYFFQRQHGEQLGGGGSGGGGYNNSKHRWPTGDNIHAEH | |
| QVRSMDELNHDFQALALEGPANGEQLLPGKKFWETDESSKDGPKGIFLGDQWRDSAWGTS | |
| DHSVSQPIMVQRRPGQSFHVNSEVNSVLSPRSESGGLGVSMVEYVLSSSPGDSCLRKGGF | |
| GPRDADSDENDKGEKKNKGTFDGDKLGDLKEEGDVMDKTNGLPVQNGIDADVKDFSRTPG | |
| NCQNSANEVDLLGPNQNGSEGLAQLTSTNGAKPVEDFSNMESQSVPLDPMEHVGMEPLQF | |
| DYSGTQVPVDSAAATVGLFDYNSQQQLFQRPNALAVQQLTAAQQQQYALAAAHQPHIGLA | |
| PAAFVPNPYIISAAPPGTDPYTAGLAAAATLGPAVVPHQYYGVTPWGVYPASLFQQQAAA | |
| AAAATNSANQQTTPQAQQGQQQVLRGGASQRPLTPNQNQQGQQTDPLVAAAAVNSALAFG | |
| QGLAAGMPGYPVLAPAAYYDQTGALVVNAGARNGLGAPVRLVAPAPVIISSSAAQAAVAA | |
| AAASANGAAGGLAGTTNGPFRPLGTQQPQPQPQQQPNNNLASSSFYGNNSLNSNSQSSSL | |
| FSQGSAQPANTSLGFGSSSSLGATLGSALGGFGTAVANSNTGSGSRRDSLTGSSDLYKRT | |
| SSSLTPIGHSFYNGLSFSSSPGPVGMPLPSQGPGHSQTPPPSLSSHGSSSSLNLGGLTNG | |
| SGRYISAAPGAEAKYRSASSASSLFSPSSTLFSSSRLRYGMSDVMPSGRSRLLEDFRNNR | |
| YPNLQLREIAGHIMEFSQDQHGSRFIQLKLERATPAERQLVFNEILQAAYQLMVDVFGNY | |
| VIQKFFEFGSLEQKLALAERIRGHVLSLALQMYGCRVIQKALEFIPSDQQNEMVRELDGH | |
| VLKCVKDQNGNHVVQKCIECVQPQSLQFIIDAFKGQVFALSTHPYGCRVIQRILEHCLPD | |
| QTLPILEELHQHTEQLVQDQYGNYVIQHVLEHGRPEDKSKIVAEIRGNVLVLSQHKFASN | |
| VVEKCVTHASRTERAVLIDEVCTMNDGPHSALYTMMKDQYANYVVQKMIDVAEPGQRKIV | |
| MHKIRPHIATLRKYTYGKHILAKLEKYYMKNGVDLGPICGPPNGII | |

TABLE 90

| | |
|---|---|
| cbe_4567083 LOC157378 | |
| hypothetical protein BC017881 | |
| length = 178 | |
| MEALNTAQGARDFIYSLHSTERSCLLKELHRFESIAIAQEKLEAPPPTPGQLRYVFIHNA | SEQ ID NO: 81 |
| IPFIGFGFLDNAIMIVAGTHIEMSIGIILGISTMAAAALGNLVSDLAGLGLAGYVEALAS | |
| RLGLSIPDLTPKQVDMWQTRLSTHLGKAVGVTIGCILGMFPLIFFGGGEEDEKLETKS | |

TABLE 91

| | |
|---|---|
| cbe_552975 HPV 1a E1 | |
| Human Papillomavirus 1a E1 | |
| length = 612 | |
| MADNKGTENDWFLVEATDCEETLEETSLGDLDNVSCVSDLSDLLDEAPQSQGNSLELFHK | SEQ ID NO: 82 |
| QESLESEQELNALKRKLLYSPQARSADETDIASISPRLETISITKQDKKRYRRQLFSQDD | |
| SGLELSLLQDETENIDESTQVDQQQKEHTGEVGAAGVNILKASNIRAALLSRFKDTAGVS | |
| FTDLTRSYKSNKTCCGDWVLAVWGVRENLIDSVKELLQTHCVYIQLEHAVTEKNRFLFLL | |
| VRFKAQKSRETVIKLITTILPVDASYILSEPPKSRSVAAALFWYKRSMSSTVFTWGTTLE | |
| WIAQQTLINHQLDSESPFELCKMVQWAYDNGHTEECKIAYYYAVLADEDENARAFLSSNS | |
| QAKYVKDCAQMVRHYLRAEMAQMSMSEWIFRKLDNVEGSGNWKEIVRFLRFQEVEFISFM | |
| IAFKDLLCGKPKKNCLLIFGPPNTGKSMFCTSLLKLLGGKVISYCNSKSQFWLQPLADAK | |
| IGLLDDATKPCWDYMDIYMRNALDGNTICIDLKHRAPQQIKCPPLLITSNIDVKSDTCWM | |
| YLHSRISAFKFAHEFPFKDNGDPGFSLTDENWKSFFERFWQQLELSDQEDEGNDGKPQQS | |
| LRLTARAANEPI | |

TABLE 92

| | |
|---|---|
| cbe_552976 HPV 11 L2 | |
| Human papillomavirus 11 L2 | |
| length = 455 | |
| MKPRARRRKRASATQLYQTCKATGTCPPDVIPKVEHTTIADQILKWGSLGVFFGGLGIGT | SEQ ID NO: 83 |
| GAGSGGRAGYIPLGSSPKPAITGGPAARPPVLVEPVAPSDPSIVSLIEESAIINAGAPEV | |
| VPPTQGGFTITSSESTTPAILDVSVTNHTTTSVFQNPLFTEPSVIQPQPPVEASGHILIS | |
| APTITSQHVEDIPLDTFVVSSSDSGPTSSTPLPRAFPRPRVGLYSRALQQVQVTDPAFLS | |
| TPQRLVTYDNPVYEGEDVSLQFTHESIHNAPDEAFMDIIRLHRPAITSRRGLVRFSRIGQ | |
| RGSMYTRSGQHIGARIHYFQDISPVTQAAEEIELHPLVAAENDTFDIYAEPFDPIPDPVQ | |
| HSVTQSYLTSTPNTLSQSWGNTTVPLSIPSDWFVQSGPDITFPTASMGTPFSPVTPALPT | |
| GPVFITGSDFYLHPTWYFARRRRKRIPLFFTDVAA | |

TABLE 93

| | |
|---|---|
| cbe_71919 FLJ13236 | |
| hypothetical protein FLJ13236 | |
| length = 341 | |
| MAKGLLVTYALWAVGGPACLHHLYLGRDSHALLWMLTLGGGGLGWLWEFWKLPSFVAQAN | SEQ ID NO: 84 |
| RAQGQRQSPRGVTPPLSPIRFAAQVIVGIYFGLVALISLSSMVNFYIVALPLAVGLGVLL | |
| VAAVGNQTSDFKNTLGSAFLTSPIFYGRPIAILPISVAASITAQRHRRYKALVASEPLSV | |
| RLYRLGLAYLAFTGPLAYSALCNTAATLSYVAETFGSFLNWFSFFPLLGRLMEFVLLLPY | |
| RIWRLLMGETGFNSSCFQEWAKLYEFVHSFQDEKRQLAYQVLGLSEGATNEEIHRSYQEL | |
| VKVWHPDHNLDQTEEAQRHFLEIQAAYEVLSQPRKPWGSRR | |

TABLE 94

| | |
|---|---|
| cbe_1380530 RPL13 | |
| ribosomal protein L13 | |
| length = 1110 | |
| ctttccgctcggctgttttcctgcgcaggagccgcagggccgtaggcagccatggcgccc | SEQ ID NO: 85 |
| agccggaatggcatggtcttgaagccccacttccacaaggactggcagcggcgcgtggcc | |
| acgtggttcaaccagccggcccgtaagatccgcagacgtaaggcccggcaagccaaggcg | |
| cgccgcatcgccccgcgccccgcgtcgggtcccatccggcccatcgtgcgctgccccacg | |
| gttcggtaccacacgaaggtgcgcgccggccgcggcttcagcctggaggagctcagggtg | |
| gccggcattcacaagaaggtggcccggaccatcggcatttctgtggatccgaggaggcgg | |
| aacaagtccacggagtccctgcaggccaacgtgcagcggctgaaggagtaccgctccaaa | |
| ctcatcctcttccccaggaagccctcggcccccaagaagggagacagttctgctgaagaa | |
| ctgaaactggccacccagctgaccggaccggtcatgcccgtccggaacgtctataagaag | |
| gagaaagctcgagtcatcactgaggaagagaagaatttcaaagccttcgctagtctccgt | |
| atggcccgtgccaacgcccggctcttcggcatacgggcaaaaagagccaaggaagccgca | |
| gaacaggatgttgaaaagaaaaaataaagccctcctggggacttggaatcagtcggcagt | |
| catgctgggtctccacgtggtgtgtttcgtgggaacaactgggcctgggatggggcttca | |
| ctgctgtgacttcctcctgccaggggatttggggctttcttgaaagacagtccaagccct | |
| ggataatgctttactttctgtgttgaagcactgttggttgtttggttagtgactgatgta | |
| aaacggttttcttgtggggaggttacagaggctgacttcagagtggacttgtgttttttc | |
| tttttaaagaggcaaggttgggctggtgctcacagctgtaatcccagcactttgaggttg | |
| gctgggagttcaagaccagcctggccaacatgtcagaactactaaaaataaagaaatcag | |
| ccatgaaaaaaaaaaaaaaaaaaaaaaaaa | |

TABLE 95

| | |
|---|---|
| cbe_1469980 FLJ20259 | |
| hypothetical protein FLJ20259 | |
| length = 3101 | |
| gagggttgaatgtaagatggcgcccagggagctgtgaggagaaaaccctgtcggtcttgg | SEQ ID NO: 86 |
| agcgacgacggcagaaccagggtccctggcggtgcggcggggccggcgggtgcagcggag | |
| gcggcggcggcggcggcagtgacgtcgccggaatattagaagtcttaagaactcaggaca | |
| agcagcagaaatacatgcaacatggtgactggaaccctaaggactctgcaatatgaataa | |
| ttccctagagaacaccatctcctttgaagagtacatccgagtaaaggcacggtctgtccc | |
| gcaacacaggatgaaggaatttctggactcactggcctctaaggggccagaagcccttca | |
| ggagttccagcagacagccaccactaccatggtgtaccaacagggtgggaactgcatata | |
| cacagacagcactgaagtggctgggtctttgcttgaacttgcctgtccagtcaccaccag | |
| tgttcagccacaaacccagcaagaacagcagatccaggttcagcagccgcagcaggttca | |
| ggtccaggtgcaggtacagcagtctccgcaacaggtctcggctcagctctccccacaact | |
| caccgttcaccagcctactgagcaacccatccaggtccaggtgcagatccaaggccaggc | |
| accacagtcagcagccccctccattcagaccccgtctctgcagagtcccagtccctcgca | |
| gctgcaagcagctcagatccaggtgcagcacgtgcaagcagcccagcagatccaggctgc | |
| agaaatcccggaggagcacatcccacatcagcaaatccaggctcagctggtggctggcca | |
| gtctcttgctggtggtcagcagatccaaatccagaccgtgggtgccctttccccaccacc | |
| atcccagcagggctcaccccgggaaggggagcggcgggttggcacggccagtgtcctcca | |
| accagtgaagaagcgcaaagtggagatgcccatcactgtgtcctacgccatctcagggca | |
| gccggtggccaccgtgctggccattccacagggccagcagcagagttatgtgtctttgag | |
| gccagacttactgacagtagacagtgcccacctgtacagtgccactgggaccattactag | |
| ccctacaggagaaacctggaccatccctgtttattctgcccagccccggggggaccctca | |
| gcagcagagcattacccacattgccattccccaggaagcctacaacgcagttcacgtcag | |
| tggctcacccacagccctggcagctgttaagctggaggatgacaaggagaagatggtggg | |
| caccacatctgtagtgaaaaactcccatgaagaggtagtgcagacccttgcaaactctct | |
| ctttccagcacagttcatgaatggcaacatccacattccagtggctgtgcaggctgtggc | |
| aggcacgtaccagaatacggctcaaactgtccatatatgggacccccaacagcagccgca | |
| gcagcaaactccccaggaacagacaccaccaccacagcagcagcagcagcaactccaagt | |
| tacttgttcagctcaaactgtccaggttgctgaagttgaaccacagtcacagccacagcc | |
| ttccccagaacttctgcttccaaattctttgaagccagaagaagggcttgaagtatggaa | |
| aaactgggcccagaccaagaatgctgaactagagaaggatgctcagaacagattggcacc | |
| cattgggaggcgccaactgctgcgattccaggaagatctcatctcctctgctgtggcaga | |
| gttgaattatgggctctgtctaatgacacgggaagctcgaaatggagaaggtgaacccta | |
| tgacccagatgtgctctactatattttcctgtgtattcaaaagtatctttttgaaaatgg | |
| aagggtagatgacattttctccgatctttattatgttcggttcacggagtggctacatga | |
| agttctgaaggatgttcagccccgggtcactccacttggctatgtcttgcccagccacgt | |
| gactgaggagatgctatgggagtgcaagcagcttggggctcactccccctccaccttgct | |
| gaccaccctcatgttctttaataccaagtacttcctattgaagacagtggaccagcacat | |
| gaagctggccttctccaaggtcttgcgacagacaaagaagaacccctctaatcccaagga | |
| taaaagcacgagtatccggtacttgaaggcccttggaatacaccagactggccagaaagt | |
| tacagatgacatgtatgcagaacagacggaaaatccagagaatccattgagatgtcccat | |
| caagctctatgatttctacctcttcaaatgcccccagagtgtgaaaggccggaatgacac | |
| cttttacctgacacctgagccagtggtggcccccaacagcccaatctggtactcagtcca | |
| gcctatcagcagagagcagatgggacaaatgctgacgcggatcctggtgataagagaaat | |
| tcaggaggccatcgcagtggccaatgcaagcactatgcactgagatgccttggccatggc | |
| acaagagaaaccagccaggaaaaaccagacagactttcacactaaagaagaggcctccat | |
| ttttttttttcttttttttattggtgtagttacgaagcctttcaggctgcttctgtttaa | |
| aatataaaagaaaactttgccccctttgcatcttcataaacctgctgcggcagactcctc | |
| agccgatggtggctctgggtttccttgagtgtcatatgtcccagaaagttgctggctgac | |
| tcttttttgtctggggcctggggaaagggcttggactgtgaaaagaaatgtggccccttt | |
| ccatcttcaagagagatggaattaatgatggatggaccctggagggaatctccccagccg | |
| acttccactgggctgacagactttgctgaccacaggggaacgatgttcttttctttcttc | |
| atgatcagacataaacttagcatcttaatggaagaaaaatgaggggaacttcaattatga | |
| tttattaaagacaatttctattaaaaaaaaaaaaaaaaaaa | |

TABLE 96

| | |
|---|---|
| cbe_1505425 HCDI | |
| HCDI protein | |
| length = 1522 | |
| agtcgctatgcgtgtcttgtgggtgagggagggcagaaagggagagtgctgggcgggctt | SEQ ID NO: 87 |
| agtcggagattgaggactgggaatccgcttccgggagggcactgtctagtgcacaggcaa | |
| cctggccttcgcctcctagcccgagaagccgaatctccctaatccctgtgacctgtgtca | |
| cctctgcatcgcgaggagggggataagtggggagaagtctggtgtcagatgggatggcgc | |
| cggaagagggtgccacagcggggacggaaggcgcccccaccccaactccacgggaatata | |
| aacaatttgtattttccgatcaggtggcgggacaggcttcattgggacagccctaaccca | |
| gctgctgaatgccagaggcacgaagtgacgttggtctcccgaaagcccgggcccggccgg | |
| atcacgtgggatgagctcgctgcatcggggctgccgagctgcgatgccgccgtcaacctg | |
| gccggagagaacatcctcaaccctctccgaagatggaatgaaaccttccaaaaagaggtt | |
| ctcggcagccgcctagagaccacccaattgctggctaaagccatcaccaaagccccacaa | |
| ccccccaaggcctgggtcttagtcacaggtgtagcttactaccagcccagtctgactgcg | |
| gagtatgatgaagacagcccaggaggggactttgactttttctccaacctcgtaaccaaa | |
| tgggaagctgcagccaggcttcctggagattctacacgccaggtggtggtgcgctcaggg | |
| gttgtgctgggccgtgggggtggtgccatgggccacatgctgctgccctttcgcctgggc | |
| ctggggggccccatcggctcaggccaccaattcttcccctggatacacatcggggacctg | |
| gcaggaatcctgacccatgcccttgaagcaaaccacgtgcacggggtcctgaatggagtg | |
| gctccatcctccgccactaatgctgagtttgcccagaccttgggtgctgccctgggccgc | |
| cgagccttcatccctctccccagcgctgtggtgcaagctgtctttgggcgacagcgtgcc | |
| atcatgctgctggagggccagaaggtgatcccacagcgaacactggccactggctaccag | |
| tattccttcccagagctaggggctgccttaaaggaaattgtagcctaagtaggtcgtggc | |
| aagggcctgaggcctgttcctcacaggcttccaggttaggcactgtgaataggctcagct | |
| cctctagagagctgaagccatctggttcttagattcctctcccagtcctctttcccattg | |
| ttctgttgctccaccttattgtctcaaggccgtaatctcatcaggttgggacattaatct | |
| tttcaactccttgtaagatttcccagtttggtttctctacatgtcctgcagctgccccac | |
| ttctcctttacgctgtgtagagaatgctctgcagtttaggcaataaaaataaattgtctc | |
| actaaaaaaaaaaaaaaaaaaa | |

TABLE 97

| | |
|---|---|
| cbe_1710 QP-C | |
| low molecular mass ubiquinone-binding protein (9.5 kD) | |
| length = 428 | |
| aaaaataagtaggaagtgctcaattttaatttggttcagttttccggagggcgagctgag | SEQ ID NO: 88 |
| ccctggccgccgccacaatgggccgcgagtttgggaatctgacgcggatgcggcatgtga | |
| tcagctacagcttgtcaccgttcgagcagcgcgcctatccgcacgtcttcactaaaggaa | |
| tccccaatgttctgcgccgcattcgggagtctttctttcgcgtggtgccgcagtttgtag | |
| tgttttatcttatctacacatgggggactgaagagttcgagagatccaagaggaggatcc | |
| agctgcctatgaaaatgacaaatgagcaacgcatccggatgacggttccctgtctctgaa | |
| agacctttctctggaagaggagtctgcattgtagtgtctcaaagacacaataaacttcct | |
| atggtctg | |

TABLE 98

cbe_1796502 CTNND2
catenin (cadherin-associated protein), delta 2 (neural plakophilin-related
arm-repeat protein)
length = 4746

| | |
|---|---|
| gccagcatcccttgtcccgcggccggctcagacaacaaaagcggaagatgctgcagttgg | SEQ ID NO: 89 |
| gcaaggtcaggaccttgccttgaaagccgggcggcgccgcgcaacgcctcttcccggact | |
| gaggagctgtcgccggcggagggtgcatgtttgcgaggaagccgccgggcgccgcgcctt | |
| tgggagctatgcctgttccagaccagccttcatcagcctcagagaagacgagttccctga | |
| gccccggcttaaacacctccaacggggatggctctgaaacagaaaccacctctgccatcc | |
| tcgcctcagtcaaagaacaggaattacagtttgaaaggctgacccgagagctggaggctg | |
| aacggcagatcgtagccagccagctggagcgatgcaagctcggatccgagactggcagca | |
| tgagcagcatgagttcagcagaagagcagtttcagtggcagtcacaagatggtcaaaaag | |
| atatcgaagatgagcttacaacaggtctcgagctggtggactcctgtattaggtcactac | |
| aggaatcaggaatacttgacccacaggattattctacaggtgaaaggcccagcctgctct | |
| cccagagtgcacttcagctcaattccaaacctgaagggtctttccagtatccggccagct | |
| accatagcaaccagaccctggccctgggggaaaccaccccttcacagctcccggcccgag | |
| gcacacaagcccgagctacgggccagagcttcagccagggcacgaccagccgcgccggcc | |
| acctggcggggcccgagcccgcgccgccgccgccgccgccgccgcgggagccgttcgcgc | |
| ccagcctgggcagcgccttccacctgcccgacgcgccgcccgccgccgccgccgccgcgc | |
| tctactactccagctccacgctgcccgcgccgccgcgcgggggctccccgctggccgcgc | |
| cccagggcggttcgcccaccaagctgcagcgcggcggctcggcccccgagggcgccacct | |
| acgccgcgccgcgcggctcctcgcccaagcagtcgcccagccgcctggccaagtcctaca | |
| gcaccagctcgcccatcaacatcgtcgtgtcctcggccggcctgtccccgatccgcgtga | |
| cctcgccccccaccgtgcagtccaccatctcctcctcgcccatccaccagctgagctcca | |
| ccatcggcacgtacgccaccctgtcgcccaccaagcgcctggtccacgcgtccgagcagt | |
| acagcaagcactcgcaggagctgtatgccacggccaccctccagaggccgggcagcctgg | |
| cagctggttcccgagcctcatacagcagccagcatgggcacctgggcccagagttgcggg | |
| ccctgcagtccccagaacaccacatagatcccatctatgaagaccgcgtctatcagaagc | |
| cccctatgaggagtctcagccagagccagggggaccctctgccgccagcacacaccggca | |
| cctaccgcacgagcacagccccatcttcccctggtgtcgactccgtccccttgcagcgca | |
| caggcagccagcacggcccacagaatgccgccgcggccaccttccagagggccagctatg | |
| ccgccggcccagcctccaattacgcggacccctaccgacagctgcagtattgtccctctg | |
| ttgagtctccatacagcaaatccggccctgctctcccgcctgaaggcaccttggccaggt | |
| ccccgtccattgatagcattcagaaagatcccagagaatttggatggagagacccggaac | |
| tgccggaagtgattcagatgttgcagcaccagtttccctcggtccagtctaacgcggcag | |
| cctacttgcaacacctctgttttggagacaacaaaattaaagccgagataaggagacaag | |
| gaggcatccagctcctggtggacctgttggatcatcggatgaccgaagtccaccgtagtg | |
| cctgtggagctctgagaaacctggtgtatgggaaggccaacgatgataacaaaattgccc | |
| tgaaaaactgtggtggcatcccagcactggtgaggttactccgcaagacgactgacctgg | |
| agatccgggagctggtcacaggagtcctttggaacctctcctcatgcgatgcactcaaaa | |
| tgccaatcatccaggatgccctagcagtactgaccaacgcggtgattatcccccactcag | |
| gctgggaaaattcgcctcttcaggatgatcggaaaatacagctgcattcatcacaggtgc | |
| tgcgtaacgccaccgggtgcctaaggaatgttagttcggccggagaggaggcccgcagaa | |
| ggatgagagagtgtgatgggcttacggatgccttgctgtacgtgatccagtctgcgctgg | |
| ggagcagtgagatcgatagcaagaccgttgaaaactgtgtgtgcattttaaggaacctct | |
| cgtaccggctggcggcagaaacgtctcagggacagcacatgggcacggacgagctggacg | |
| ggctactctgtggcgaggccaatggcaaggatgctgagagctctgggtgctggggcaaga | |
| agaagaagaaaaagaaatcccaagatcagtgggatggagtaggacctcttccagactgtg | |
| ctgaaccaccaaaagggatccagatgctgtggcacccatcaatagtcaaaccctacctca | |
| cactgctctctgagtgctcaaatccagacacgctggaaggggcggcaggcgccctgcaga | |
| acttggctgcagggagctggaagtggtcagtatatatccgagccgctgtccgaaaagaga | |
| aaggcctgcccatcctcgtggagctgctccgaatagacaatgaccgtgtggtgtgcgcgg | |
| tggccactgcgctgcggaacatggccttggacgtcagaaataaggagctcatcggcaaat | |
| acgccatgcgagacctagtccacaggcttccaggagggaacaacagcaacaacactgcaa | |
| gcaaggccatgtcggatgacacagtgacagctgtctgctgcacactgcacgaagtgatta | |
| ccaagaacatggagaacgccaaggccttacgggatgccggtggcatcgagaagttggtcg | |
| gcatctccaaaagcaaaggagataaacactctccaaaagtggtcaaggctgcatctcagg | |
| tcctcaacagcatgtggcagtaccgagatctgaggagtctctacaaaaaggatggatggt | |
| cacaataccactttgtagcctcgtcttcaaccatcgagagggaccggcaaaggccctact | |
| cctcctcccgcacgccctccatctcccctgtgcgcgtgtctcccaacaaccgctcagcaa | |
| gtgccccagcttcacctcgggaaatgatcagcctcaaagaaaggaaaacagactacgagt | |
| gcaccggcagcaacgccacctaccacggagctaaaggcgaacacacttccaggaaagatg | |
| ccatgacagctcaaaacactggaatttcaactttgtataggaattcttatggtgcgcccg | |
| ctgaagacatcaaacacaaccaggtttcagcacagccagtcccacaggagcccagcagaa | |
| aagattacgagacctaccagccatttcagaattccacaagaaattacgatgagtccttct | |
| tcgaggaccaggtccaccatcgccctcccgccagcgagtacaccatgcacctgggtctca | |
| agtccaccggcaactacgttgacttctactcagctgcccgtccctacagtgaactgaact | |
| atgaaacgagccactacccggcctcccccgactcctgggtgtgaggagcagggcacaggc | |
| gctccgggaacagtgcatgtgcatgcataccacaagacatttctttctgttttgtttttt | |
| tctcctgcaaatttagtttgttaaagcctgttccataggaaggctgtgataaccagtaag | |
| gaaatattaagagctattttagaaagctaaatgaatcgcaagttaacttggaaatcagta | |
| gaaagctaaagtgatcctaaatatgacagtgggcagcacctttctagcgtgagctgtaaa | |
| gtaacgagaagtgctttatactgaacgtggttgatgggaggagagacgaggcattcgggc | |
| cggtggggcgtaagggttatcgttaagcacaagacacagaatagtttacacactgtgtgg | |
| gggacggcttctcacgctttgtttactctcttcatccgttgtgactctaggcttcaggtt | |
| gcattggggttcctctgtacagcaagatgtttcttgccttttgttaatgcattgttgtaa | |
| agtatttgatgtacattacagattaaagaagaaaagcgcgttgtgtatattacaccaatg | |
| ccgccgtgtttcctcatctatggttctaaatattgcttcaatttcaaacttttgaaagat | |
| gtatggatttccagtttttctttactttctcccagtatgttttaacaaaaaaaaaaaaaa | |
| gcaggaaaaaaggaatatttagcagtattgttcgttctgatatgtgaatttgtctgtgac | |
| aactaaacaaggcattcagcagtttctgacaattaacatacatcattccacactccttgt | |
| caacaaagtgctttttcactgcctaaaattttagatgtagatatttgaaatagatttttt | |
| catttataccagttttctttatgatgatacagtgttaaaagaaaataaattacaattgat | |
| ctgtca | |

TABLE 99

| | |
|---|---|
| cbe_1807361 FLJ22729 | |
| hypothetical protein FLJ22729 | |
| length = 1278 | |
| aatggaggaagactggacctactgattcaacttggagatgagcgggtctgtcctcttcac | SEQ ID NO: 90 |
| ggcgggagagaggtggagatgctttctgaccccgtcgaggtcatccctgtactgggcctt | |
| acataatttctgctgtcggaaaaaatccactacacctaagaaaattactcccaatgttac | |
| tttttgtgatgaaaatgcaaaggagcccgaaaatgcacttgacaagctcttctcttcaga | |
| acagcaggcttccatcttgcatgtgttgaatacagcatctactaaagaacttgaagcttt | |
| ccgattgcttcgtggaagaaggtccatcaatatcgtagagcacagagaaaactttgggcc | |
| atttcagaatttagagagtttaatgaatgtgcccttgtttaagtataaaagtacagttca | |
| agtttgtaactccatactttgtccaaagactggacgggaaaaaagaaagtcaccggaaaa | |
| ccggttcctgagaaagctcctcaaaccagacatagaaagagaaagacttaagaattgcct | |
| gggctcaccttgatcgtaagttgacagtgctggactggcagcaaagtgaccgttggagtt | |
| taatgagaggaatatactcatcatcagtctatttagaagagatttcctcgatcatttcaa | |
| agatgcctaaagcagatttctatgttctggaaaaaacaggactatccattcagaactcat | |
| ctctgtttccaatactgttacattttcatatcatggaagccatgctgtatgccttattaa | |
| ataaaacttttgcccaggatgggcagcatcaggtgctgagcatgaatcgaaatgcagtgg | |
| ggaagcattttgaactgatgattggtgactcccggactagtggaaaagagctagtgaagc | |
| agtttctcttcgattctatactgaaggcggatcctcgggtgttcttcccatcagataaaa | |
| tagttcactacagacagatgtttttatctactgaactacaaagagtagaagagctttatg | |
| attcattattacaagctattgccttctatgaattagcagtgtttgactctcagccttaga | |
| attctgaggttaacgtgctaaagtataattattagctctaacgtaacaccaactgttgtg | |
| aacatccatgttattggaaaagaacacattttcagtgtattttagatgtttaaattctga | |
| cttttggctattaaatggtttacacaataagccaagaccaaatcaataaacattttatga | |
| gaaaaaaaaaaaaaaaaa | |

TABLE 100

| | |
|---|---|
| cbe_2253980 Prey1135 | |
| Homo sapiens, clone MGC: 14302, mRNA, complete cds. | |
| length = 1863 | |
| ggcacgaggcaggggctctgctgcgacctttacggtagtgaggttagggacgatgggaga | SEQ ID NO: 91 |
| gcagctttgttcctgaagtgccgtcgaggattaggatcgagtttcttcgatcgtgagggc | |
| aattattagtggcctctagccccccagatggcatcttgaggagagagaatccacgctacg | |
| agaactaacatagtggacatcgcagttgtccgtagccgatttgcgggttacggagttgtt | |
| gcaagtaaagaaggggaaatccgagcgttctcgcgttggatttcctccacgtgtgaagtg | |
| ggaatggtagtgatatctcgtgctatctctgtcaagggcaagaaggagaaggaatttatt | |
| tgaggccctacgacgtggtgcggtggctcacgcctgtaatcccaacactttgggaggccg | |
| aggcaggcggatcatctgagatcgagagttcgagaccagcttgaccaacatggagaaacc | |
| ccgtctctactaaaagtacaaaattagccaggcatggtggcacatgcccgtaatcccagc | |
| tactcgggaggctgaggcaggagagtagcttgagcccaggaggcggaggttgtggtgagc | |
| cgagatcgcgccattgcacagcctgggcaacgagcgaaactccatctcaaaaaaataaat | |
| aaaaatttttttaaaaaaaaaaaaaaaaaactcgaggcaaaactaaccccctaataaaat | |
| taattaaccactcattcatcgacctccccaccccatccaacatctccgcatgatgaaact | |
| tcggctcactccttggcgcctgcctgatcctccaaatcaccacaggactattcctagcca | |
| tgcactactcaccagacgcctcaaccgccttttcatcaatcgcccacatcactcgagacg | |
| taaattatggctgaatcatccgctaccttcacgccaatggcgcctcaatattctttatct | |
| gcctcttcctacacatcgggcgaggcctatattacggatcatttctctactcagaaacct | |
| gaaacatcggcattatcctcctgcttgcaactatagcaacagccttcataggctatgtcc | |
| tcccgtgaggccaaatatcattctgaggggccacagtaattacaaacttactatccgcca | |
| tcccatacattgggacagacctagttcaatgaatctgaggaggctactcagtagacagtc | |
| ccaccctcacacgattctttacctttcacttcatcttgcccttcattattgcagccctag | |
| cagcactccacctcctattcttgcacgaaacgggatcaaacaaccccctaggaatcacct | |
| cccattccgataaaatcaccttccacccttactacacaatcaaagacgccctcggcttac | |
| ttctcttccttctctccttaatgacattaacactattctcaccagacctcctaggcgacc | |
| cagacaattataccctagccaaccccttaaacacccctccccacatcaagcccgaatgat | |
| atttcctattcgcctacacaattctccgatccgtccctaacaaactaggaggcgtccttg | |
| ccctattactatccatcctcatcctagcaataatccccatcctccatatatccaaacaac | |
| aaagcataatatttcgcccactaagccaatcactttattgactcctagccgcagacctcc | |
| tcattctaacctgaatcggaggacaaccagtaagctacccttttaccatcattggacaag | |
| tagcatccgtactatacttcacaacaatcctaatcctaataccaactatctccctaattg | |
| aaaacaaaatactcaaatgggcctaaaaaaaaaaaaaaaaaaaaaaaaaaaaaaaaaaaa | |
| aaa | |

TABLE 101

| | |
|---|---|
| cbe_22607 Hs. 101882 | |
| 601276943F1 Homo sapiens cDNA, 5′ end | |
| length = 796 | |
| ccgcggccgctcgcagcttgctggcctctcccgcgcctcacgtcggactccgtctccgcg | SEQ ID NO: 92 |
| gcaggaagcagcatggaagctgaggacatccaggaggagttgacctgccccatctgcctg | |
| gactatttccaggacccggtgtccatcgagtgcggacacaacttctgccgcggctgcctg | |
| caccgcaacatgggcgccgggcggcaggaccgtatcccctgccccgaatgtcggcaccca | |
| tcggcgcccgccgcgctgcagacccaactgggccctggccaggctgactgagaagacgca | |
| gcgccggcgcctgggccccgtgcccccgggcctgtgcggccgccactgggagccgctgcg | |
| gctcttctgcgaggacgaccagcggccagtgtgcctggtgtgcagggagtcccaggagca | |
| ccagactcacgccatggcacccatcgacgaggcttcgagagctaccgggagaaacttctt | |
| aagtctcagcgtactctcgtggccaacgatgacgcacacgtcatgcatttacaggatgtc | |
| gacagtgacgaaccgccacacacgtggcaggataacgcatacggagtcacgcgaaatgag | |
| acatcacgcacggcgtttttaacagcgtggcaccacttcctggttgaagaacgaggacct | |
| gtctcttccagcagcttgcacaacgaagaagcacagaccagcacgacgctgaatgacacc | |
| acttcacactccgtccacttcgctcctgagaccctcttaaggggggacaacccgcccccc | |
| tgggcgtcacacccca | |

TABLE 102

| | |
|---|---|
| cbe_244811 DGUOK | |
| deoxyguanosine kinase | |
| length = 880 | |
| aacggtgcgctggagcgagtgagcagcgatacctagggcggaagtgctctcggcggaagt | SEQ ID NO: 93 |
| gatcgctgtgtgaatcgtgggtgggatggccgcgggccgcctctttctaagtcggcttcg | |
| agcacccttcagttccatggccaagagcccactcgagggcgtttcctcctccagaggcct | |
| gcacgcggggcgcgggccccgaaggctctccatcgaaggcaacattgctgtgggaaagtc | |
| cacgtttgtgaagttactcacgaaaacttacccagaatggcacgtagctacagaacctgt | |
| agcaacatggcagaatatccaggctgctggcacccaaaaagcctgcactgcccaaagtct | |
| tggaaacttgctggatatgatgtaccgggagccagcacgatggtcctacacattccagac | |
| attttcctttttgagccgcctgaaagtacagctggagcccttccctgagaaactcttaca | |
| ggccaggaagccagtacagatctttgagaggtctgtgtacagtgacaggctccactttga | |
| ggctctgatgaacattccagtgctggtgttggatgtcaatgatgatttttctgaggaagt | |
| aaccaaacaagaagacctcatgagagaggtaaacacctttgtaaagaatctgtaaccaat | |
| accatgaagttcaggctgtgatctgggctccctgactttctgaagctagaaaaatgttgt | |
| gtctcccaaccacctttccatccccagcccctctcatccctggagcactctgccgctcaa | |
| gagctggtttgttaattattgttagactttgccattgttttcttttgtacctgaagcatt | |
| ttgaaaataaagtttacttaagttatgcttgtttttctaa | |

TABLE 103

| | |
|---|---|
| cbe_250777 HPV 11 E4 | |
| Human papillomavirus 11 E4 | |
| length = 324 | |
| atggtagtaccaatcattgggaagtatgttatggcagcacagttatatgttctcctgcat | SEQ ID NO: 94 |
| ctgtatctagcactgtacgagaagtatccattgctgaacctactacatacacccccgcac | |
| agaccaccgcccctacagtgtccgcctgcaccacggaagacggcgtgtcggcgccgccta | |
| ggaagcgagcacgtggaccgtccactaacaacaccctgtgtgtggccaacatcagatccg | |
| tggacagtacaatcaacaacatcgtcactgacaattacaacaagcaccaaagaaggaaca | |
| actgtcacagtgcagctacgccta | |

TABLE 104

| | |
|---|---|
| cbe_250781 HPV 11 E2 | |
| Human papillomavirus 11 E2 | |
| length = 1104 | |
| atggaagcaatagccaagcgtttagatgcgtgccaggatcagttgttagaactttatgaa | SEQ ID NO: 95 |
| gaaaacagtattgatatacacaaacacattatgcattggaaatgcatacgattggaaagt | |
| gtattactacacaaagcaaaacaaatgggcctgagccacatcgggttacaagtagtacca | |
| ccattaactgtgtcagagactaaaggacataatgctattgaaatgcaaatgcatttagaa | |
| tccttagcaaaaactcagtatggtgtggaaccttggacattacaggacaccagttatgaa | |
| atgtggctaacaccacccaaacggtgctttaaaaaacagggaaatactgtggaggtaaaa | |
| tttgatggctgtgaagacaatgtaatggagtatgtggtatggacacatatatacctgcag | |
| gacaacgactcatgggtaaaagtaactagttccgtagatgccaagggcatatattataca | |
| tgtggacaatttaaaacatattatgtaaattttaataaagaggcacaaaagtatggtagt | |
| accaatcattgggaagtatgttatggcagcacagttatatgttctcctgcatctgtatct | |
| agcactgtacgagaagtatccattgctgaacctactacatacacccccgcacagaccacc | |
| gcccctacagtgtccgcctgcaccacggaagacggcgtgtcggcgccgcctaggaagcga | |
| gcacgtggaccgtccactaacaacaccctgtgtgtggccaacatcagatccgtggacagt | |
| acaatcaacaacatcgtcactgacaattacaacaagcaccaaagaaggaacaactgtcac | |
| agtgcagctacgcctatagtgcaactgcaaggtgattccaattgtttaaaatgttttaga | |
| tatagactgaatgacaaatataaacatttgtttgaattagcatcttcaacgtggcattgg | |
| gcctcacctgaggcaccacataaaaatgcaattgtaacattaacatatagcagtgaggaa | |
| caacgtcagcaatttttaaacagtgtaaaaataccacccaccattaggcataaggtgggg | |
| tttatgtcattacatttattgtaa | |

TABLE 105

| | |
|---|---|
| cbe_250808 HPV 11 E6 | |
| Human papillomavirus 11 E6 | |
| length = 453 | |
| atggaaagtaaagatgcctccacgtctgcaacatctatagaccagttgtgcaagacgttt | SEQ ID NO: 96 |
| aatctttctttgcacactctgcaaattcagtgcgtgttttgcaggaatgcactgaccacc | |
| gcagagatatatgcatatgcctataagaacctaaaggttgtgtggcgagacaactttccc | |
| tttgcagcgtgtgcctgttgcttagaactgcaagggaaaattaaccaatatagacacttt | |
| aattatgctgcatatgcacctacagtagaagaagaaaccaatgaagatattttaaaagtg | |
| ttaattcgttgttacctgtgtcacaagccgttgtgtgaaatagaaaaactaaagcacata | |
| ttgggaaaggcacgcttcataaaactaaataaccagtggaagggtcgttgcttacactgc | |
| tggacaacatgcatggaagacttgttaccctaa | |

TABLE 106

| | |
|---|---|
| cbe_250809 Prey152 | |
| PathCalling Prey Sequence 152 | |
| length = 515 | |
| cgcggccgcgtcgacgagagaactagtctcgagttttttttcaagtcttttccttttttt | SEQ ID NO: 97 |
| ttctattttcctggttgagctatttactatttatttagaagagttctttatataatcaga | |
| aacctttgtcagctgtatgtgctgccaatgtcttctcccacaacgtgggttgccttttca | |
| cattcttactgataacttttgataaaaagatgttcttaccattagtaaaacttattgtct | |
| ctcctttgagtactgattttgaatatcttttaagaaatctctgccttctcccaaattatg | |
| aagatattcttatatgttaccttataaactttattattttactttccatatgtaaattta | |
| taaatcatctgaaattgtttttgtcaaaggaatgagaaaacaaaattcttacttaaaaaa | |
| atagcccatccacaaagtcttactgattgataatacattcgttcattcttttcccattgc | |
| actgcagtattgaatttctgtgtttctctgagaat | |

TABLE 107

| | |
|---|---|
| cbe_250834 HPV 11 E7 | |
| Human papillomavirus 11 E7 | |
| length = 297 | |
| atgcatggaagacttgttaccctaaaggatatagtactagacctgcagcctcctgaccct | SEQ ID NO: 98 |
| gtagggttacattgctatgagcaattagaagacagctcagaagatgaggtggacaaggtg | |
| gacaaacaagacgcacaacctttaacacaacattaccaaatactgacctgttgctgtgga | |
| tgtgacagcaacgtccgactggttgtggagtgcacagacggagacatcagacaactacaa | |
| gaccttttgctgggcacactaaatattgtgtgtcccatctgcgcaccaaaaccataa | |

TABLE 108

| | |
|---|---|
| cbe_250883 Prey244 | |
| PathCalling Prey Sequence 244 | |
| length = 447 | |
| aaaaaaagaaaaagaaaattctgccttatatttccattgaataaaactgacattccagaa | SEQ ID NO: 99 |
| agctgcattgtgttttatcctgtggattcatcacttctgtgtaccaccagaggtctgcat | |
| agtctggtctgaaaactgctgatttaaggccgggcgcagtggctcatgcctgtaatccca | |
| gcactttggaaggtcgaggcgggcggatcacctgaggttcaggaattcgagaccagcctg | |
| gccgacatggcgaggccccaactctactaaaaatatggaaattaggcatggtggtgggcg | |
| cctgtaattccagctgctcgggagactgagacaggagaatcgcctgaacccggaaggcgg | |
| aggttgcagtgagccgagatcgtgccattgcactccagcctgggcaacaagagcaaaagt | |
| ccatctcaaaaaaaaaaaaaaaaaccc | |

TABLE 109

| | |
|---|---|
| cbe_250888 Prey228 | |
| PathCalling Prey Sequence 228 | |
| length = 507 | |
| aacagcttttcagataagaacaaggagcatagctttttcatcacagactctgatgcttct | SEQ ID NO: 100 |
| ggaggagatttttggagagaaagatcaggtgaacatacacaagaaaccaactcacctcat | |
| tcactgaaaaaggatgtagaaaatatggggaaagaagaacttcagaaggttttatttgaa | |
| caaatagatttacggagacgactggaacaagaattccaggtgttaaaaggaaacacatct | |
| ttcccagtattcaataattttcaggatcagatgaaaagggagctagcctaccgagaagaa | |
| atggtgcaacagttacaaattatcccctatgcagcaagtttgatcaggaaagaaaagctt | |
| ggcgcccatctcagcaaaagctaaaaggtgcacagaactacttactcttgttctcttgta | |
| agatacagcctgccatggagcacttctgacccgtgttaaagactgcagatcgaaagggct | |
| tgggaattaaatacaaaatcaggttca | |

TABLE 110

| | |
|---|---|
| cbe_250907 Prey372 | |
| PathCalling Prey Sequence 372 | |
| length = 183 | |
| gtactttgtagtttttcatccctactctctttaactggaaatatagaaattagcacattc | SEQ ID NO: 101 |
| atgtcaaacactgtccctttctgtcatgttctggatgagcaattaatcttgtttatactg | |
| aataaaattttatttgagttgtgaacataaaaaaaaaaaaaaaaaaaaaaaaaaaaccca | |
| aaa | |

TABLE 111

| | |
|---|---|
| cbe_250925 Prey415 | |
| PathCalling Prey Sequence 415 | |
| length = 448 | |
| cttagaggtagcagattgtcatatttgaatggaggatttgagtctgagaagtggctgaga | SEQ ID NO: 102 |
| gttattctgatacaaatagggtgtgcaatttttggtgagtcccattcatcagcttctaaa | |
| aaggagtagcagaaacacttgcacagcagcaggagttacttacccttcctgaatggctgc | |
| atcttcttaaggctgtttttttccctcctaaacagaaaatcatacattctgcaacaaaag | |
| ctggaaacatcctaaatgcctggaagcagtttagttttaagggttgaggccaggtgcggt | |
| ggcttggctgggcacagtggctcacacctgtaatcccagctactcaggaggctgaggcag | |
| gagaatcacttggaccagggaggtggaggttgcagtgagccaggattgtgccactgcact | |
| ccagcctgggcggcagagtgagactctc | |

TABLE 112

| | |
|---|---|
| cbe_251059 HPV 16 E1-E4 | |
| HPV 16 E1-E4 | |
| length = 276 | |
| atggctgatcctgcagcagcaacgaagtatcctctcctgaaattattaggcagcacttgg | SEQ ID NO: 103 |
| ccaaccaccccgccgcgacccataccaaagccgtcgccttgggcaccgaagaaacacaga | |
| cgactatccagcgaccaagatcagagccagacaccggaaacccctgccacaccactaagt | |
| tgttgcacagagactcagtggacagtgctccaatcctcactgcatttaacagctcacaca | |
| aaggacggattaactgtaatagtaacactacaccca | |

TABLE 113

| | |
|---|---|
| cbe_251063 HPV 16 E1 | |
| Human papillomavirus 16 E1 | |
| length = 1947 | |
| atggctgatcctgcaggtaccaatggggaagagggtacgggatgtaatggatggttttat | SEQ ID NO: 104 |
| gtagaggctgtagtggaaaaaaaaacaggggatgctatatcagatgacgagaacgaaaat | |
| gacagtgatacaggtgaagatttggtagattttatagtaaatgataatgattatttaaca | |
| caggcagaaacagagacagcacatgcgttgtttactgcacaggaagcaaaacaacataga | |
| gatgcagtacaggttctaaaacgaaagtatttgggtagtccacttagtgatattagtgga | |
| tgtgtagacaataatattagtcctagattaaaagctatatgtatagaaaaacaaagtaga | |
| gctgcaaaaaggagattatttgaaagcgaagacagcgggtatggcaatactgaagtggaa | |
| actcagcagatgttacaggtagaagggcgccatgagactgaaacaccatgtagtcagtat | |
| agtggtggaagtgggggtggttgcagtcagtacagtagtggaagtgggggagagggtgtt | |
| agtgaaagacacactatatgccaaacaccacttacaaatattttaaatgtactaaaaact | |
| agtaatgcaaaggcagcaatgttagcaaaatttaaagagttatacggggtgagtttttca | |
| gaattagtaagaccatttaaaagtaataaatcaacgtgttgcgattggtgtattgctgca | |
| tttggacttacacccagtatagctgacagtataaaaacactattacaacaatattgttta | |
| tatttacacattcaaagtttagcatgttcatggggaatggttgtgttactattagtaaga | |
| tataaatgtggaaaaaatagagaaacaattgaaaaattgctgtctaaactattatgtgtg | |
| tctccaatgtgtatgatgatagagcctccaaaattgcgtagtacagcagcagcattatat | |
| tggtataaaacaggtatatcaaatattagtgaagtgtatggagacacgccagaatggata | |
| caaagacaaacagtattacaacatagttttaatgattgtacatttgaattatcacagatg | |
| gtacaatgggcctacgataatgacatagtagacgatagtgaaattgcatataaatatgca | |
| caattggcagacactaatagtaatgcaagtgcctttctaaaaagtaattcacaggcaaaa | |
| attgtaaaggattgtgcaacaatgtgtagacattataaacgagcagaaaaaaaacaaatg | |
| agtatgagtcaatggataaaatatagatgtgatagggtagatgatggaggtgattggaag | |
| caaattgttatgtttttaaggtatcaaggtgtagagtttatgtcatttttaactgcatta | |
| aaaagatttttgcaaggcatacctaaaaaaaattgcatattactatatggtgcagctaac | |
| acaggtaaatcattatttggtatgagtttaatgaaatttctgcaagggtctgtaatatgt | |
| tttgtaaattctaaaagccatttttggttacaaccattagcagatgccaaaataggtatg | |
| ttagatgatgctacagtgccctgttggaactacatagatgacaatttaagaaatgcattg | |
| gatggaaatttagtttctatggatgtaaagcatagaccattggtacaactaaaatgccct | |
| ccattattaattacatctaacattaatgctggtacagattctaggtggccttatttacat | |
| aatagattggtggtgtttacatttcctaatgagtttccatttgacgaaaacggaaatcca | |
| gtgtatgagcttaatgataagaactggaaatcctttttctcaaggacgtggtccagatta | |
| agtttgcacgaggacgaggacaaggaaaacgatggagactctttgccaacgtttaaatgt | |
| gtgtcaggacaaaatactaacacatta | |

TABLE 114

| | |
|---|---|
| cbe_251071 Prey734 | |
| PathCalling Prey Sequence 734 | |
| length = 473 | |
| cgcggccgcgtcgacccggttcccgggaggatgaagttcgtgtacaaagaagagcatccg | SEQ ID NO: 105 |
| ttcgagaagcgccgctctgagggcgagaagatccgaaagaaatacccggaccgggtgccg | |
| gtgatagtagaaaaggctcccaaagctcggataggagacctggacaaaaagaaatacctg | |
| gtgccttctgatctcacagttggtcagttctacttcttgatccggaagcgaattcatctc | |
| cgagctgaggatgccttgtttttctttgtcaacaatgtcattccacccaccagtgccaca | |
| atgggtcagctgtaccaggaacaccatgaagaagacttctttctctacattgcctacagt | |
| gacgaaagtgtctacggtctgtgaagctgctgcccctgagctggaggggggtctcattct | |
| acaaagagagaggtggccccctttctgacctctcctccttcagctcaacacac | |

TABLE 115

| | |
|---|---|
| cbe_251088 HPV 16 E2 | |
| Human papillomavirus 16 E2 | |
| length = 1098 | |
| atggagactctttgccaacgtttaaatgtgtgtcaggacaaaatactaacacattatgaa | SEQ ID NO: 106 |
| aatgatagtacagacctacgtgaccatatagactattggaaacacatgcgcctagaatgt | |
| gctatttattacaaggccagagaaatgggatttaaacatattaaccaccaagtggtgcca | |
| acactggctgtatcaaagaataaagcattacaagcaattgaactgcaactaacgttagaa | |
| acaatatataactcacaatatagtaatgaaaagtggacattacaagacgttagccttgaa | |
| gtgtatttaactgcaccaacaggatgtataaaaaaacatggatatacagtggaagtgcag | |
| tttgatggagacatatgcaatacaatgcattatacaaactggacacatatatatatttgt | |
| gaagaagcatcagtaactgtggtagagggtcaagttgactattatggtttatattatgtt | |
| catgaaggaatacgaacatattttgtgcagtttaaagatgatgcagaaaaatatagtaaa | |
| aataaagtatgggaagttcatgcgggtggtcaggtaatattatgtcctacatctgtgttt | |
| agcagcaacgaagtatcctctcctgaaattattaggcagcacttggccaaccaccccgcc | |
| gcgacccataccaaagccgtcgccttgggcaccgaagaaacacagacgactatccagcga | |
| ccaagatcagagccagacaccggaaacccctgccacaccactaagttgttgcacagagac | |
| tcagtggacagtgctccaatcctcactgcatttaacagctcacacaaaggacggattaac | |
| tgtaatagtaacactacacccatagtacatttaaaaggtgatgctaatactttaaaatgt | |
| ttaagatatagatttaaaaagcattgtacattgtatactgcagtgtcgtctacatggcat | |
| tggacaggacataatgtaaaacataaaagtgcaattgttacacttacatatgatagtgaa | |
| tggcaacgtgaccaatttttgtctcaagttaaaataccaaaaactattacagtgtctact | |
| ggatttatgtctatatga | |

TABLE 116

| | |
|---|---|
| cbe_251128 Prey870 | |
| PathCalling Prey Sequence 870 | |
| length = 490 | |
| gaggagcaatgccctgatgccctcctccttcgctctctatgggctggcttcagctaccat | SEQ ID NO: 107 |
| ctgtacagctgcagctccaaaatctcttccacagtccacacctcccactcagctccagat | |
| cccatcactgctgccagctggtcatccccccgaaggaggctcagagtctacctcccaggc | |
| ccttggccactgagctcttggagagtcctctctccctgtgccctttgcagctccaccaac | |
| ccaaccaccagctccctcctccaagcctcacattctccacctccaggctttgatcaggca | |
| cagaacgcccttccatggaatgcccctccatgcctgtgtntccaacctactgaatctggt | |
| ggactgactgcccgaatctgagacttaggtcagaaggcacctctgtgaagcctgccatga | |
| cacattataacatgcgtcacattatagtttaccatttacacattcaaaaaaaaaaaaaaa | |
| aaaaaaaacc | |

TABLE 117

| | |
|---|---|
| cbe_251445 HPV 16 E6 | |
| Human papillomavirus 16 E6 | |
| length = 477 | |
| atgcaccaaaagagaactgcaatgtttcaggacccacaggagcgacccagaaagttacca | SEQ ID NO: 108 |
| cagttatgcacagagctgcaaacaactatacatgatataatattagaatgtgtgtactgc | |
| aagcaacagttactgcgacgtgaggtatatgactttgcttttcgggatttatgcatagta | |
| tatagagatgggaatccatatgctgtatgtgataaatgtttaaagttttattctaaaatt | |
| agtgagtatagacattattgttatagtttgtatggaacaacattagaacagcaatacaac | |
| aaaccgttgtgtgatttgttaattaggtgtattaactgtcaaaagccactgtgtcctgaa | |
| gaaaagcaaagacatctggacaaaaagcaaagattccataatataaggggtcggtggacc | |
| ggtcgatgtatgtcttgttgcagatcatcaagaacacgtagagaaacccagctgtaa | |

TABLE 118

| | |
|---|---|
| cbe_251448 HPV 16 E7 | |
| Human papillomavirus 16 E7 | |
| length = 297 | |
| atgcatggagatacacctacattgcatgaatatatgttagatttgcaaccagagacaact | SEQ ID NO: 109 |
| gatctctactgttatgagcaattaaatgacagctcagaggaggaggatgaaatagatggt | |
| ccagctggacaagcagaaccggacagagcccattacaatattgtaaccttttgttgcaag | |
| tgtgactctacgcttcggttgtgcgtacaaagcacacacgtagacattcgtactttggaa | |
| gacctgttaatgggcacactaggaattgtgtgccccatctgttctcagaaaccataa | |

TABLE 119

| | |
|---|---|
| cbe_251468 Prey1628 | |
| PathCalling Prey Sequence 1628 | |
| length = 486 | |
| gtcgacgcggccgcgggagctgatgggcagcctgcgccaccctggatcaccgtcactcgg | SEQ ID NO: 110 |
| cagaagcggagggggaccttggaccagccacccaaccaggaagacaagcctggggcactg | |
| accctgaagtctgaaccaggaaagcaagccaaggtgcccgagagaggccaggagcctgtg | |
| aagcaagctgactttgttcgcagcaagtctttcctgataacccctgtgaagcccgctgtg | |
| gaccggaagcagggggcaaagctcaacttcaaggaggggctgcaaagaggaatctcattg | |
| tcccatcagaacttggctcagtctgcagtgatgatggagaaggaactgcatcagctgaag | |
| agagccagttatgccagtacagatcagccatcctggatggaacttgccagaaagaaatct | |
| caagcttggagtgacatgccccagattataaaataggtgggcagtgtgctgataaagcat | |
| gtccac | |

TABLE 120

| | |
|---|---|
| cbe_251502 Prey1609 | |
| PathCalling Prey Sequence 1609 | |
| length = 506 | |
| ttccccgcctctcggctagctccggcctcccggggcttctgggtttggagagtttgggga | SEQ ID NO: 111 |
| gcggagacacctcccgaggctcctccgcgttggaccctatttgcgagggcgggagcgggg | |
| tgggccgacggcgaagctccagaggtgggaccctctcccttcagcagctcttcctgactt | |
| catgattctgcccctcttctcattgcaggtcacaccgacaaatctgaagcgggtttgcca | |
| ggggagtgtacccgtctccccagcctgggccacccggtccacagggattccatttccggc | |
| cttacactccgctccacgatctcatccaggcgctgcagctgtgcagctgctctacagact | |
| gcagggacctgctgagacgaatccccggacgaggtgatggctggaccggcggaggaggcg | |
| ggagcccattgtcccgagagcctgtggcctctgcctccgcaggtgtcaccaagagtgacc | |
| tacacacgagtgagcccagggcaggc | |

TABLE 121

| | |
|---|---|
| cbe_251663 HPV 11 E1 | |
| Human papillomavirus 11 E1 | |
| length = 1947 | |
| atggcggacgattcaggtacagaaaatgaggggtcggggtgtacaggatggtttatggta | SEQ ID NO: 112 |
| gaagccatagtagagcacactacaggtacacaaatatcagaagatgaggaagaggaggtg | |
| gaggacagtgggtatgacatggtggactttattgatgacaggcatattacacaaaattct | |
| gtggaagcacaggcattgtttaataggcaggaggcggatgctcattatgcgactgtgcag | |
| gacctaaaacgaaagtatttaggcagtccatatgtaagtcctataagcaatgtagctaat | |
| gcagtagaaagtgagataagtccacggttagacgccattaaacttacaacacagccaaaa | |
| aaggtaaagcgacggctgtttgaaacacgggaattaacggacagtggatatggctattct | |
| gaagtggaagctgcaacgcaggtagagaaacatggcgacccggaaaatgggggagatggt | |
| caggaaagggacacagggagggacatagagggtgagggggtggaacatagagaggcggaa | |
| gcagtagacgacagcacccgagagcatgcagacacatcaggaatattagaattactaaaa | |
| tgtaaggatatacgatctacattacatggtaagtttaaagactgctttgggctgtcattt | |
| gttgatttaattaggccatttaaaagtgatagaaccacatgtgccgattgggtggttgca | |
| ggatttggtatacatcatagcatagcagatgcatttcaaaagttaattgagccattaagt | |
| ttatatgcacatatacaatggcttacaaatgcatggggaatggtactattagtattaata | |
| aggtttaaagtaaataagagcagatgtaccgtggcacgtacattaggtacgttattaaat | |
| atacctgaaaatcacatgttaattgagcctcctaaaatacaaagtggcgtacgagccctg | |
| tattggtttaggacaggcatttcaaatgcaagtacagttataggggaggcgccggaatgg | |
| ataacgcgccagaccgttattgaacatagtttggctgacagtcaatttaaattaactgaa | |
| atggtgcagtgggcatatgataatgatatttgtgaagaaagtgagatagcatttgaatat | |
| gcacagcgtggagactttgactccaatgcaagggcctttttaaatagtaatatgcaggct | |
| aaatatgtaaaagattgtgcaattatgtgcagacattataaacatgcagaaatgaaaaag | |
| atgtctattaaacaatggattaagtataggggtactaaagttgacagtgtaggtaactgg | |
| aagccaattgtgcagtttctaagacatcaaaacatagaatttattccatttttaagcaaa | |
| ctaaaattatggctgcacggaacgcccaaaaaaaattgtatagccattgtagggccacct | |
| gacactgggaagtcgtgcttttgcatgagtttaattaagtttttggggggaacagttatt | |
| agttatgttaattcctgcagccatttctggctacagccactaacggatgcaaaagtggca | |
| ttattggatgatgccacacaaccatgttggacatatatggatacatatatgagaaaccta | |
| ttagatggtaatcctatgagcatagatagaaaacatagagcattaacattaattaagtgt | |
| ccaccgctactggttacatcaaatatagacattagcaaagaggagaaatacaaatattta | |
| catagtagagttaccacatttacatttccaaatccattcccctttgacagaaatgggaat | |
| gcagtatatgaactatcagatgcaaactggaaatgtttctttgaaagactgtcgtccagc | |
| ctagacattgaggattcagaggacgaggaagatggaagcaatagccaagcgtttagatgc | |
| gtgccaggatcagttgttagaacttta | |

TABLE 122

| | |
|---|---|
| cbe_251737 HPV 16 L2 | |
| Human papillomavirus 16 L2 | |
| length = 1422 | |
| atgcgacacaaacgttctgcaaaacgcacaaaacgtgcatcggctacccaactttataaa | SEQ ID NO: 113 |
| acatgcaaacaggcaggtacatgtccacctgacattatacctaaggttgaaggcaaaact | |
| attgctgtacaaatattacaatatggaagtatgggtgtattttttggtgggttaggaatt | |
| ggaacagggtcgggtacaggcggacgcactgggtatattccattgggaacaaggcctccc | |
| acagctacagatacacttgctcctgtaagaccccctttaacagtagatcctgtgggccct | |
| tctgatccttctatagtttctttagtggaagaaactagttttattgatgctggtgcacca | |
| acatctgtaccttccattcccccagatgtatcaggatttagtattactacttcaactgat | |
| accacacctgctatattagatattaataatactgttactactgttactacacataataat | |
| cccactttcactgacccatctgtattgcagcctccaacacctgcagaaactggagggcat | |
| tttacactttcatcatccactattagtacacataattatgaagaaattcctatggataca | |
| tttattgttagcacaaaccctaacacagtaactagtagcacacccataccagggtctcgc | |
| ccagtggcacgcctaggattatatagtcgcacaacacaacaggttaaagttgtagaccct | |
| gcttttgtaaccactcccactaaacttattacatatgataatcctgcatatgaaggtata | |
| gatgtggataatacattatatttttctagtaatgataatagtattaatatagctccagat | |
| cctgactttttggatatagttgctttacataggccagcattaacctctaggcgtactggc | |
| attaggtacagtagaattggtaataaacaaacactacgtactcgtagtggaaaatctata | |
| ggtgctaaggtacattattattatgatttaagtactattgatcctgcagaagaaatagaa | |
| ttacaaactataacaccttctacatatactaccacttcacatgcagcctcacctacttct | |
| attaataatggattatatgatatttatgcagatgactttattacagatacttctacaacc | |
| ccggtaccatctgtaccctctacatctttatcaggttatattcctgcaaatacaacaatt | |
| ccttttggtggtgcatacaatattcctttagtatcaggtcctgatatacccattaatata | |
| actgaccaagctccttcattaattcctatagttccagggtctccacaatatacaattatt | |
| gctgatgcaggtgacttttatttacatcctagttattacatgttacgaaaacgacgtaaa | |
| cgtttaccatattttttttcagatgtctctttggctgcctag | |

TABLE 123

| | |
|---|---|
| cbe_251740 HPV 1a E1-E4 | |
| Human Papillomavirus 1a E1-E4 | |
| length = 375 | |
| atggcagataataaagctccccaagggctgctggggctcctgcagtacactccgactacc | SEQ ID NO: 114 |
| caaccctatccgagagtgacaccgcccagcaatcgacgtccatcgactacaccgaactcc | |
| caggacagggggagacctcgcaggtccgacaaagacagcagaaaacacctgtacgcagac | |
| ggccttacggacggcgaagatccagaagtcccagaggtggaggacgaagagaaggagaat | |
| caacgccctctaggacacccggatctgtcccttctgcgcgagacgttggaagtatacaca | |
| caacgcctcaaaagggacattcttcaagacttagacgacttctgcaggaagcttgggatc | |
| cacccgtggtctgtg | |

TABLE 124

| | |
|---|---|
| cbe_251784 HPV 16 L1 | |
| Human papillomavirus 16 L1 | |
| length = 1596 | |
| atgcaggtgacttttatttacatcctagttattacatgttacgaaaacgacgtaaacgtt | SEQ ID NO: 115 |
| taccatattttttttcagatgtctctttggctgcctagtgaggccactgtctacttgcct | |
| cctgtcccagtatctaaggttgtaagcacggatgaatatgttgcacgcacaaacatatat | |
| tatcatgcaggaacatccagactacttgcagttggacatccctattttcctattaaaaaa | |
| cctaacaataacaaaatattagttcctaaagtatcaggattacaatacagggtatttaga | |
| atacatttacctgaccccaataagtttggttttcctgacacctcattttataatccagat | |
| acacagcggctggtttgggcctgtgtaggtgttgaggtaggtcgtggtcagccattaggt | |
| gtgggcattagtggccatcctttattaaataaattggatgacacagaaaatgctagtgct | |
| tatgcagcaaatgcaggtgtggataatagagaatgtatatctatggattacaaacaaaca | |
| caattgtgtttaattggttgcaaaccacctataggggaacactggggcaaaggatcccca | |
| tgtaccaatgttgcagtaaatccaggtgattgtccaccattagagttaataaacacagtt | |
| attcaggatggtgatatggttcatactggctttggtgctatggactttactacattacag | |
| gctaacaaaagtgaagttccactggatatttgtacatctatttgcaaatatccagattat | |
| attaaaatggtgtcagaaccatatggcgacagcttatttttttatttacgaagggaacaa | |
| atgtttgttagacatttatttaatagggctggtactgttggtgaaaatgtaccagacgat | |
| ttatacattaaaggctctgggtctactgcaaatttagccagttcaaattattttcctaca | |
| cctagtggttctatggttacctctgatgcccaaatattcaataaaccttattggttacaa | |
| cgagcacagggccacaataatggcatttgttggggtaaccaactatttgttactgttgtt | |
| gatactacacgcagtacaaatatgtcattatgtgctgccatatctacttcagaaactaca | |
| tataaaaatactaactttaaggagtacctacgacatggggaggaatatgatttacagttt | |
| atttttcaactgtgcaaaataaccttaactgcagacgttatgacatacatacattctatg | |
| aattccactattttggaggactggaattttggtctacaacctcccccaggaggcacacta | |
| gaagatacttataggtttgtaacccaggcaattgcttgtcaaaaacatacacctccagca | |
| cctaaagaagatgatccccttaaaaaatacactttttgggaagtaaatttaaaggaaaag | |
| ttttctgcagacctagatcagtttcctttaggacgcaaatttttactacaagcaggattg | |
| aaggccaaaccaaaatttacattaggaaaacgaaaagctacacccaccacctcatctacc | |
| tctacaactgctaaacgcaaaaaacgtaagctgtaa | |

TABLE 125

| | |
|---|---|
| cbe_251790 Prey2347 | |
| PathCalling Prey Sequence 2347 | |
| length = 313 | |
| gagacaccagcactagccagcaggtgccccttcctcaggcttctgagtttcagccccatg | SEQ ID NO: 116 |
| cgatcactcctccaagcttctaggttttaataattccaacttttccccctttgtccctca | |
| gaggtggtagctgcttccagaaattgctaccttcatgataccacagagttctcttttttg | |
| ctttttgagttcttcgataactagttaatgattccttatacttagctaaccattctctat | |
| attaaattctctctgttaaaacaaaaaaaaaaaaaaaaaaaaaaaaaaaaaaaaaaaaaa | |
| aaaaccaaaaaaa | |

TABLE 126

| | |
|---|---|
| cbe_251821 Prey2419 | |
| PathCalling Prey Sequence 2419 | |
| length = 439 | |
| ctgaaatgcacatacatgctttagtgtaatgcagaatgcatttattggagaactcataaa | SEQ ID NO: 117 |
| catcctataaaattttcttccctgagatgcaactataaaacttggccttattctgagaat | |
| gcttaacatagatttcatccatactgtaacactgattttgttgttgttgtccttaaagca | |
| gctcagcttcctgaggtagtgttatgtctctgtggcaacaaggtgaaaatgtctagctta | |
| ttttgtcaaagtcaacaataatccacagactccagacctcaatatctgtcccaatttgcc | |
| attttactttagtgctccaaaaatatggcttatagaaaaaacaataggtgttttaaagag | |
| atttacctgaatgatatagagaatgtctagatattttctggctatcaggtaaaacctacc | |
| cttcaagatggtagaatat | |

TABLE 127

| | |
|---|---|
| cbe_251826 Prey2429 | |
| PathCalling Prey Sequence 2429 | |
| length = 553 | |
| ctttcagcagcagcggtagagcagtgaccaggcgtctccgcgatgggccccggggacttc | SEQ ID NO: 118 |
| cgccgctgcagagagagaatttcccaggggctccagggactcccaggtagagcggagctt | |
| tggttcccacctcgtcccgcgtgcgacttcttcggggacggcaggagcacggacatccag | |
| gaggaggccctcgccgccagcccgctgctggaggacctcagacgacggctgacgcgcgcc | |
| ttccagtgggcggtgcagcgcgcgatctcgaggcgcgtgcaggaggcggcggcggcggcg | |
| gcggcgcgggaggagcagagctggacgggcgttgaggccaccctggccaggctgcgggcg | |
| gagctggtggaaatgcatttccaaaaccaccagctggctagaactttactggacctaaac | |
| atgaaagtgcagcaattgaaaaaggagtatgaactggaaattacatcagactcccaaagc | |
| ccaaaagatgatgctgcgaatccggaatanagaaatgcacacgcaagggctgggcgcggt | |
| ggctcacgcctgg | |

TABLE 128

| | |
|---|---|
| cbe_251857 Prey2492 | |
| PathCalling Prey Sequence 2492 | |
| length = 54 | |
| ggatcagacattcgcgggacgagatcgacccaacgagggagcgcaacctgcctc | SEQ ID NO: 119 |

TABLE 129

| | |
|---|---|
| cbe_251900 Prey2598 | |
| PathCalling Prey Sequence 2598 | |
| length = 410 | |
| tggcgtgcggctttcgccgcgctattgcttgccaggtatggatcccggagcgcgcagagg | SEQ ID NO: 120 |
| agtccctgaggagctagggaccccaaaggcctttcaccgctgcgggccagtcctcagtca | |
| gctggagggtgggtcccacggaatcctgttagtttctaccacctcctcccccttctctgg | |
| agctggtggagcttcagaagtcctgtggagcgtcggctgggatcagtttgtctcggtgaa | |
| aagtaaactttgaaagctttccagagtgttgaatcttccaccagaaaacttgatcacatc | |
| aatatctgcagttccaatttcccaaaaagaagaagtagctgattttcagctttctgtgga | |
| ttctttattggaaaaagacaatgaccattcagaccagatatcaagtcaag | |

TABLE 130

| | |
|---|---|
| cbe_251917 HPV 1a E2 | |
| Human Papillomavirus 1a E2 | |
| length = 1206 | |
| atggaaaacctcagcagtcgcttagacttactgcaagagcagctaatgaacctatatgaa | SEQ ID NO: 121 |
| caggacagtaaattgatagaagatcaaattaagcagtggaatctaattagacaagaacaa | |
| gttcttttccatttcgccagaaaaaatggggtaatgagaattggattgcaggcagttcca | |
| tctttagcgtcctcacaggagaaggcaaagacagctattgaaatggtgttacatttagag | |
| tctttaaaggactcaccttatggcacagaggattggtcacttcaagacactagcagagag | |
| ctgtttttggcacccccagctggcaccttcaagaagagtggcagcacacttgaggttacc | |
| tatgacaataaccctgataatcagacaaggcacacaatttggaatcatgtgtattatcaa | |
| aatggggacgatgtatggagaaaagtatccagtggtgttgatgctgtaggagtgtactat | |
| ttagaacacgatggctataaaaattattatgtgttatttgctgaggaggcctctaagtac | |
| agcacaacaggacaatatgctgtaaattacaggggtaaaaggtttacaaatgttatgtct | |
| tccactagctccccaagggctgctggggctcctgcagtacactccgactacccaacccta | |
| tccgagagtgacaccgcccagcaatcgacgtccatcgactacaccgaactcccaggacag | |
| ggggagacctcgcaggtccgacaaagacagcagaaaacacctgtacgcagacggccttac | |
| ggacggcgaagatccagaagtcccagaggtggaggacgaagagaaggagaatcaacgccc | |
| tctaggacacccggatctgtcccttctgcgcgagacgttggaagtatacacacaacgcct | |
| caaaagggacattcttcaagacttagacgacttctgcaggaagcttgggatccacccgtg | |
| gtctgtgtaaaagggggtgccaatcagcttaagtgtctcaggtacagacttaaagcatct | |
| actcaagttgactttgacagcataagcaccacatggcattggacagatagaaaaaacacc | |
| gagaggataggtagtgctagaatgttagtaaagtttattgatgaggctcaacgagagaag | |
| tttcttgagagagttgctttgcccagatcagtgtctgtgtttttgggacagtttaatggg | |
| tcttaa | |

TABLE 131

| | |
|---|---|
| cbe_251919 HPV 1a E6 | |
| Human Papillomavirus 1a E6 | |
| length = 423 | |
| atggcgacaccaatccggaccgtcagacagctttccgaaagcctctgtatcccatatatt | SEQ ID NO: 122 |
| gatgttttattgccttgtaatttttgtaattattttttgtctaatgctgagaagctgctt | |
| tttgatcattttgatttgcatcttgtctggagagacaatttggtgtttggatgctgtcaa | |
| gggtgtgctagaactgttagcctattggagtttgttttatattatcaggagtcttatgag | |
| gtaccggaaatagaagaaattttggacagacctttattgcaaattgaactccgttgtgtt | |
| acatgcataaaaaaactgagtgttgctgaaaaattggaggttgtgtcaaacggagaaaga | |
| gtgcatagagttagaaacagacttaaagcaaagtgtagtttgtgtcgcttgtatgctata | |
| taa | |

TABLE 132

| | |
|---|---|
| cbe_251925 HPV 1a E7 | |
| Human Papillomavirus 1a E7 | |
| length = 282 | |
| atggtgggcgaaatgccagcactaaaggacctggttcttcaacttgaaccaagcgtccta | SEQ ID NO: 123 |
| gatttagatctttattgttacgaggaggtgcctcctgatgacatagaggaggagttagtg | |
| tcgcctcagcaaccttatgctgtcgttgcttcctgtgcctattgcgagaaactggttcga | |
| ttgaccgtcctcgcggatcacagcgccattagacagctggaggaactccttctgcgatct | |
| ttgaacatcgtgtgcccactgtgcaccctacagcgacagtaa | |

TABLE 133

| | |
|---|---|
| cbe_251929 Prey2672 | |
| PathCalling Prey Sequence 2672 | |
| length = 408 | |
| gtctctacaaataataaaaaattgtctgggcatggtggcctgtgcccgtagtcccagcta | SEQ ID NO: 124 |
| ctcaggagggtgaggtgggaggatcgcttgggcctaggaggtcaagcctgcagtgagttg | |
| taatcacgccactgcactccagcctgggcaacacagcgagatcctatttctcctatttct | |
| ctctgtctctctcacacacacacacacacacaaacacacacataatgtggaatgattaaa | |
| caaagctaattaacatatcggtcacctcctttaatttttccagcaagacatgtgaaattt | |
| attcttttagcctattttgaaatacatgtcatatatcattattaactatagtcaccctgg | |
| tgtgcgatggatctcaaaaacttattttttctgtctaactcaaacttt | |

TABLE 134

| | |
|---|---|
| cbe_251953 Prey2724 | |
| PathCalling Prey Sequence 2724 | |
| length = 617 | |
| ggccgactcccaggatccgttgcggcgcggcggagcagccaatggcgagccccacagtct | SEQ ID NO: 125 |
| cgcgagagtgctcaggcgctcttcgtggctgccctcttagctgctagcggagctcctcag | |
| ggggcggccgggagcctacaattcctagaaagaaaatacgccattccggaaacagaactg | |
| cagttaagaccctcgaaaagatctaagatagtgtgcatcctataacacctgacgaatttc | |
| agaatgtgacaaagcgcagaggatgcattatttcaaaacaaaacagaaggctaaaatttg | |
| cagganaaagaaaatcagtaaaccgggaatcctcggactggattgtaagcaagatttgaa | |
| tgaattagaagctgaaggtatttaggctgtgatataganggtacatatttcatcccacag | |
| agaaaacataatactcgaaatttcgtgaaaaacgcaaactgtcggaaatctcctcagtcc | |
| gactaatgcgcgcttgacatgcgatgtgaatgccctctgtcggcgctgtcgctgacgcac | |
| atctgtccgtggtcgtctatcgatatgtgttgtgttatgctgtgtctatcgcgtcgctgt | |
| cactgtccggttgcctt | |

TABLE 135

| | |
|---|---|
| cbe_251976 FLJ23584 | |
| hypothetical protein FLJ23584 | |
| length = 4992 | |
| ggctatcctactgcagctggctgcatcagatgtgccaggcctgccctctagctcccaaca | SEQ ID NO: 126 |
| ccatctgcctgatctaagctttgccatgttgttgtcactgctgggtgcctgtgctgtggt | |
| ggggccattccatggccctgagtgggagccagtgcagggcctgctctcccagaatcacag | |
| ctgcagggaccctcagtgctgtggcaacctgcttgtcctctgcctctttctggtctggca | |
| ggtccggcactgttggcaccaggtcaccaggacccgcttcagcacaaggaatgtcatcaa | |
| ggtgccactgcagaagcgggcagtgccctccatgaggtgcgaaactgtcttcaagctgac | |
| tcctgaattcttcagtcctggaaagtccaggggcctagattctcaacaatgcgcacaaag | |
| gcagagatggggataccggaggagcctccaggaatcatgggcccagaacctgctctctcc | |
| acagcacccatgtccaggcccaccttcgggtgtccacacccactctgagcccatcttttg | |
| taccacctccatttcaaacacctgtttactgcctcagaacagttcctggaaagcatggca | |
| ggtgccttggtgtctccatgatggtcagactcgccctgccttggacatgtgtcaagagat | |
| ggagcagctgctgcttcactcacaggaaaggttggtgtcactggagcctgtcatcagtgt | |
| gaggtctcgccccacctccatgaccttaaccacctctcttccaaacttactttcagctga | |
| gaggctgcagttctgcccccagagagctcctgcctgatccttcccaccaaacactgagaa | |
| tgtgcacttggaagtcttggcactgtcgaccagaggcctgggaaccagggggtaaaaacc | |
| agaaagcaggcagagaggatagtagagagacccaggctccaaggtgggtgaaccagacag | |
| ggagcagacgggaggatgcttcagaaatccaggcatctggggagtagttcccagtagact | |
| ttggaatggagggtgatgcagagactaacgtgttggagtgtgcaaaccagagactagtaa | |
| taagtgaaactgatggcgagatcttgacaccagggtgggacacccaggaccggatgggag | |
| ttgagagtagaaccaacattcaggaactagggaatagaaaccagagggaggctggaggtg | |
| agaatctccctgaaacccaggcacatatgggagagaaccaagaacagttaagatgtaaaa | |
| ttgatgcagagacccaaacacctgagtgggagaaccaggataagaatggaagtgaggatg | |
| ctgtggagacccagacatttgagaagaaggacaagaaagaggctggagaggaggatgggg | |
| aagagatccaggctcaaggattggggaagcaaggccagactggagatgagaatggtgagg | |
| agacccagacaccacagtgggagaaacaagatcagatgaaaggtgatgcggatgtggaaa | |
| ttcagatggaagaggggagaaacaaggatcaggttggaggtcaggatgctgcacaaaccc | |
| aatcatgtgggagggagaacgtgggagaagtaaaaaaagagaatagtgtagagacccagg | |
| ccttggattggggaaaacaggaatgtgttggaaatgggaatgttacagagatccagacac | |
| caaggtgggagaagcatgatcaaggtggaagtaagaaagctaagaagacccaagcatctg | |
| ggggagagaaccagaaacaattaagtcatgaaattcaagtggggtggggaaataagggcc | |
| tgagaagagatgaagatgctaaggaaacccagatagctaccaagaagaagctcagggaga | |
| taagagaaggattgggtggtgatccaggcactatggtggggaaaccggagacaagtagca | |
| agtgaaatttatagagaatttgagatactatgttgggagaatcagaactggattggaggt | |
| gaacatagagcagaaattcaggcatcagagaagagagaccaaagaaaggatggatgtgag | |
| gatggcacaaatatcctggcacccgaggctgagatccaggaacaattaaaaggtgaaact | |
| gatgtggagactcagagcaatgagccacttagagaagaggatggtacagacattcagtca | |
| ctagggaggagagaggttaaaggtgaggatgataaagacacccaggaacttgggaggaaa | |
| aatcagggtcagttaggaaatgaatttagtggaaagattcacataccaaaggggaagaat | |
| caggaacatattagaggcgaagatggtgcacatacccagatatctgagtcagggaactgg | |
| ggcaaattaacaagtcaaattgatggagaaatgcattcagcagaatggaagaaagatcag | |
| cagattggaggtgagaatggggcagaaattcagatacaagggaagagaaacctgagagaa | |
| gttggaggtgaggacggtgtaaagacctgggcacctgggaaagaaacccagagtcagttt | |
| agaagtgatcttggtagaaagatccttttatcagagtggaagagccagaagcagatggga | |
| agtgagaatggaacagaaattcaggctccagtggagagaaaccagagagaacctggaggt | |
| gaggacggtgtaaagactcagagacctaagagagagaacgaggaccagttagatagtgaa | |
| attggagggagccattcaccagggaggaggaactgggagctgattggaaaggatgttgca | |
| gaaaatcaggcatcagagaagagaaaccagagagaggttggaaacgaggacggtagaatg | |
| atctggaggcttaggggaaaaaactggaggcttagagcgaagaaacagactgttaaaaag | |
| taaagataatggaaagacccgtttatcagagtggaagaaccaggaacagggtggaggtgg | |
| gaatgatgaagaaattcaaatacaggggaagagaaacctgagagggaccacagctgatga | |
| tggtacagagacccaggctcctgcaggagatgaccagggacagttaagagttgaaattgc | |
| tgaagagatccaggtacaagggcaaggaaataagaatgacggtggagttgaggatgttgc | |
| agaactccaggatataggaagccagagaaagtgcacagatgaggatgttggagagcctcg | |
| agcaccaaggggaggaaacaaggatctggtcagaggagaggatgctgtgagggacagtct | |
| ccaagtcgactgttctgggagtgagaggcccacaggcaggaagcacagcctaccatggcc | |
| tccagccttcactggctatggatgtgggacccgggaacaggaacaggcagtggctgtgaa | |
| tggtttcatctctgccccctgtcctgagatgaatcctgtcccccactggggtgaagtctt | |
| cctgctggtgggtggggagggagagcatctggccagccagggcacaacccctgccaggga | |
| tcatagagtggggatcagtccagcctcccagcaggcccaacctgaatcctggagaagacg | |
| acaaagggacaaaggggtggatccagagaaggcccccagcctgactcggcagtcccaaaa | |
| ccctccgtctctgacagctcccttgggcatgccctctgcctgctcctgtctcccgtgtgg | |
| cccagccccggaagctgccatcattctagcgggtcctcccaccgccctcactgtcctgcc | |
| caaggggacaggcctcaagaagagcaaacgactgctcctggagtccctcatgcggaggag | |
| gattgcacacctgaagtggggtcttccccggcggatcctggagtcctatttcctgtttaa | |
| cttcttaggatcttgctcattgacccttgctggggcgaggctctctggactgaacacagg | |
| ccaggagctccaagcccagcaggaaaggtattgtgaggcccaaggctccccaccaggcct | |
| taagtccccagagaggttccagagggttcagcgcccagacagaaaaagctcgaaacttcc | |
| tatacaagccagagctctggagaggaacagaccgcacatgtcagagcccattaagcattt | |
| ccatccagcctgaaaaggccaggagagtcaggccaccagggggcgccagagaaccacagg | |
| agatccaggaagcgcttgctaggaccaagctcccagctcccaggacccccaggccggcag | |
| cggagtccaggagctggtgtggcccacaaagggtcggagagcctcccagtgagaacagta | |
| ggggcaggaaaatgatcaggtcaagggtctcccagctggcagagagggctcccagcagag | |
| tgaggacttcattctctagggcagaccacgcccactggaggaaggaatgtacatcctggg | |
| aggcctctaagctccccagactcaaatgccagcagctcacatactggagaagaggaagcc | |
| tggaatctacagggtgcagaggggctgggcagcagccttcctgccattctgcagaacctg | |
| tcagcttcaaagggaggctccactctgcggtggcaaagctgggcctgacccttctggcca | |
| agatgtcctggtccccacagctcgccaagcgcaagcacttggcccctaacctgagcctga | |
| gggaacctgattctactctgcctcccaaagtgggtgatcctcgtgcaggggaggacagca | |
| tcggagatcacactgcttcacagagggatcttcagctgcaaggtcactgctgtactgggg | |
| ccacccttcctaagacagagagtccccagggccaggaggcacctgggaacccaaatgggg | |
| ctccacagaatacacgagcctccaaaaagtttagtattatgaagcatctgaatttttttt | |
| ctcttccagaatggctttaaaaagtagactcaggcccagagtcctcaggacatatcagaa | |
| aagctttgagagtgtcctgatccctgcaaagcctctaataaattggtcatttgggcaaaa | |
| aaaaaaaaaaaa | |

TABLE 136

| | |
|---|---|
| cbe_252016 HPV 1a L1 | |
| Human Papillomavirus 1a L1 | |
| length = 1527 | |
| atgtataatgtttttcagatggctgtctggttaccagcgcagaataagttctatcttcct | SEQ ID NO: 127 |
| ccccagcccatcactagaatcctgtccactgatgaatatgtaaccagaaccaatctcttc | |
| taccatgcaacatctgaacgtctactgctggtcggacatcctttgtttgagatctccagt | |
| aatcaaactgtaactataccaaaagtgtcaccaaatgcatttagagtttttagggtgcgt | |
| tttgctgatccaaatagatttgcatttggggataaggcaatttttaatccagaaacagaa | |
| agattagtttggggcctaagagggatagagataggtagaggccagcctttaggtatagga | |
| ataacgggccaccctcttttaaataagttagatgatgcagaaaatccaacaaattatatt | |
| aatactcatgcaaatggagattctagacaaaatactgcttttgatgcaaaacagacacaa | |
| atgttcctcgtcggctgtactcctgcttcaggtgaacactggacaagtagtcgttgccca | |
| ggggaacaagtgaaacttggggactgccccagggtgcaaatgatagagtctgtcatagaa | |
| gatggtgacatgatggatattggttttggggctatggattttgctgctttacagcaagac | |
| aagtctgatgtccctttagatgttgttcaagcaacatgcaaatatcctgattatatcaga | |
| atgaaccatgaagcctatggcaactctatgtttttttttgcacgtcgcgagcaaatgtat | |
| accaggcacttttttactcgcgggggttcggtgggtgataaggaggcagtcccacaaagc | |
| ctgtatttaacagcagatgctgaaccaagaacaactttagcaacaacaaattatgtaggc | |
| acaccaagtggctctatggtttcatctgatgtccaattgtttaatagatcttactggctt | |
| cagcgatgtcaaggccagaataatggcatttgctggagaaaccagttatttattacagtt | |
| ggagataataccagaggaacaagtttatctatcagtatgaaaaacaatgcaagtactaca | |
| tattccaatgctaattttaatgattttctaagacatactgaagaatttgatctttctttt | |
| atagttcagctttgtaaagtaaagttaactcccgaaaatctagcctacattcatacaatg | |
| gaccctaatattttagaggattggcaactatctgtatctcaaccacctaccaatcctcta | |
| gaagatcaatataggtttttagggtcttccttggcagcaaaatgtccagaacaggcgcct | |
| cctgagccccagactgatccttatagtcaatataaattctgggaagtcgatctcacagaa | |
| aggatgtccgaacaattagaccaatttccactaggaaggaaatttctatatcaaagtggc | |
| atgacacaacgtactgctactagttccaccacaaagcgcaaaacagtgcgtgtatctacg | |
| tcagccaagcgcaggcgtaaggcttag | |

TABLE 137

| | |
|---|---|
| cbe_252041 HPV 1a L2 | |
| Human Papillomavirus 1a L2 | |
| length = 1524 | |
| atgtatcgcctacgtagaaaacgcgctgcccccaaagatatatacccctcatgcaaaata | SEQ ID NO: 128 |
| tcaaacacctgcccacctgacattcaaaataaaattgagcatacaacaattgctgataaa | |
| atattgcaatatggcagtctgggagtttttttgggaggtttgggcattggaacagccaga | |
| ggctctggaggaagaattggttatactcccctcggtgagggtggtggggttagagttgct | |
| actcgtccaactccagtaaggcctacaatacctgtggaaacagtaggccccagtgaaatt | |
| ttccccatagatgttgtagatcctacaggccctgctgttattcccctacaagatttaggt | |
| agagacttcccaataccaactgtgcaggttattgcagaaattcaccctatttctgacata | |
| ccaaacattgttgcatcttcaacaaatgaaggagaatctgccatattagatgtgttacga | |
| gggaatgcaaccatacgcactgtttcaagaacacaatacaataacccctctttcactgtt | |
| gcatctacatctaatataagtgctggagaagcatcaacatcagatattgtatttgttagc | |
| aatggttcaggtgacagggtggtgggcgaggatatccccttggtagaattaaacttaggc | |
| cttgaaacagacacatcttctgttgtacaagaaacagcattttccagcagcacaccaatt | |
| gctgaaagaccctcttttaggccctcaagattctataataggcgtctatatgaacaggtg | |
| caagtacaagaccctaggttcgttgagcagccacagtcaatggtcacttttgataatcca | |
| gcatttgagccagagcttgatgaggtgtctattatcttccaaagagacttagatgctctt | |
| gctcagacaccagtgcctgaatttagagatgtagtttatctgagcaagcccacattttcg | |
| cgggaaccagggggacggttaagggttagccgccttggcaaaagttcaactattcgtaca | |
| cgcctgggcacagcaattggcgccagaacccactttttctatgatttaagttctattgct | |
| ccagaagactcaattgaattattgcctttaggtgagcatagtcaaacaacagtcattagt | |
| tccaacttaggtgacacagcatttatacaaggtgagacagcagaggatgacttagaagtt | |
| atctctttagaaacaccacaattatattcagaagaagagcttttagacacaaacgaaagt | |
| gtgggcgaaaatttgcaacttactattactaactcagagggtgaggtttctatactagat | |
| ttaacacaaagcagagtcaggccaccttttggcactgaagatactagcttgcatgtatat | |
| tacccaaattcttctaaagggactccaataattaatcctgaagaatcatttacacctttg | |
| gttattatagctcttaacaactcaacaggggattttgagttacatcctagtcttagaaag | |
| cgtcgtaaaagagcttatgtataa | |

TABLE 138

| | |
|---|---|
| cbe_252090 Prey2820 | |
| PathCalling Prey Sequence 2820 | |
| length = 535 | |
| gagcagcacattccacccatgctgagagccactggttgctcccagcttggtctgtatcct | SEQ ID NO: 129 |
| cctgagcagctcccaccccctgaaatgctttggagaagaaagaagaggaggccatgtttg | |
| gaaggaatgcagcagcagggccttgggggagtccccgcccgggtgagggctgtcacttac | |
| cacctggaggacctaagaaggcgtcagagcatcatcaacgaactgaagaaggcccagtgg | |
| ggcagctctggggctgcatctgagccagtggtgcttggcgaagagggctgtggattcccc | |
| agcaccaatgaataccctgatctggaagaggagagagcaacctatccacaggaagaggac | |
| cgttttctcactcctggcagggcccagctgctttggtctccctggagccccctggatcag | |
| gaggaggcttgtgcctccaggcagctgcactctctggcctcgttcagcactgtcacagcc | |
| agaaggaacccccttcacaatccctgggggatggagttggcagcgtctgaagagt | |

TABLE 139

| | |
|---|---|
| cbe_2685998 Prey2313 | |
| Homo sapiens, clone IMAGE: 3625550, mRNA, partial cds. | |
| length = 1922 | |
| gcgccatctgcctcgattacttcacggaccccgtgtccatcggctgcgggcacaacttct | SEQ ID NO: 130 |
| gccgagtttgtgtaacccagttgtggggtggggaggatgaggaggacagagatgagttag | |
| atcgggaggaggaggaggaggacggagaggaggaggaagtggaggctgtgggggctggcg | |
| cggggtgggacacccccatgcgggatgaagactacgagggtgacatggaggaggaggtcg | |
| aggaggaagaagagggtgtgttctggaccagtggcatgagcaggtccagctgggacaaca | |
| tggactatgtgtgggaggaggaggacgaggaggaagacctggactactacttgggggaca | |
| tggaggaggaggacctgaggggggaggatgaggaggacgaggaggaagtgctggaggagg | |
| ttgaggaagaggatctagaccccgtcaccccactgcccccgcctccagcccctcggaggt | |
| gcttcacatgccctcagtgccgaaagagctttcctcggcggagcttccgccccaacctgc | |
| agctggccaatatggtccaggtgattcggcagatgcacccaacccctggtcgagggagcc | |
| gcgtgaccgatcagggcatctgtcccaaacaccaagaagccctgaagctcttctgcgagg | |
| tagacgaagaggccatctgtgtggtgtgccgagaatccaggagccacaaacagcacagcg | |
| tggtgccattggaggaggtggtgcaggagtacaaggccaaactgcaggggcacgtggaac | |
| cactgaggaagcacctggaggcagtgcagaagatgaaagccaaggaggagaggcgagtga | |
| cagaactgaagagccagatgaagtcagagctggcagcggtggcctcggagtttgggcgac | |
| tgacacggtttctggctgaagagcaggcagggctggaacggcgtctcagagagatgcatg | |
| aagcccagctggggcgtgcgggagccgcggctagtcgccttgcagaacaggccgcccagc | |
| tcagccgcctgctggcagaggcccaggagcggagccagcaggggggtctccggctgctcc | |
| aggtgtgaagaggtacagctgcagcccccagaggtctggtcccctgacccgtgccaaccc | |
| catagccatgacttcctgacagatgccatcgtgaggaaaatgagccggatgttctgtcag | |
| gctgcgagagtggacctgacgctggaccctgacacggctcacccggccctgatgctgtcc | |
| cctgaccgccggggggtccgcctggcagagcggcggcaggaggttgctgaccatcccaag | |
| cgcttctcggccgactgctgcgtactgggggcccagggcttccgctccggccggcactac | |
| tgggaggagcctaaagaaccctcctggcctccagctcagccttctctcacctactatgtc | |
| tgtccaacagaccggccagaatttagcttcacttgagagagatctggaatggtcgccatg | |
| attgaaaccacgcaccattacatcatcattacattaattacatcaacataaattatttct | |
| tcccccttcccttttccagcactcaaccaaggagcaaagctcatcccaccccacacccct | |
| cccaggtctgctcactgccaggctcctctcccctttgttcagtggagctggcttttctcc | |
| cagcccctttccatgcctttcactccatttggcaagctctgagggggagcctggggacgg | |
| gtttgggtccccaggaggagagccttgggtataatctatttttctaggagcctcttgcct | |
| tgtcacttgcagctttcgccctctgctttgatggctgaggtgaactcatgttctttggga | |
| aaagggaaggcgtgctgtggaaataaaatgtttatttgcttctcaaaaaaaaaaaaaaaa | |
| aa | |

TABLE 140

| | |
|---|---|
| cbe_2693325 Prey1673 | |
| Prey1673, 194 bp. | |
| length = 194 | |
| tgtacactgccgcaatgtgggtgcagagctgagggactgagctggggcaccagggtgggc | SEQ ID NO: 131 |
| agattggcatgagggagagaggaggacctcaagttgggaagagaggcaaagtcagcactg | |
| gagacagggagtgaaggaagagctgaaggattcagctcctggccagatagttgggatgat | |
| gctcttttagatct | |

TABLE 141

| | |
|---|---|
| cbe_2789381 Prey775 | |
| AL523176 LTI_NFL003_NBC3 Homo sapiens cDNA clone CS0DC001YA04 3 prime, mRNA | |
| sequence (AL523176.1: 98%/1043, p = 0.000000), 1498 bp. | |
| length = 1498 | |
| tactggagggccgggtcccagcctggatgttacccaaccccatgacggaccctttcattg | SEQ ID NO: 132 |
| cagcgatctgcacacgtgtgggtgtttccataggacattgtcaggcacagatggctggtc | |
| cgaagcacgtgcatttcagttttgaggctcctgtcctcaggcagtgtggactcaggccct | |
| gggagtcatgtggatgccagtagcctgggcccacagagctgctgggatgtggggggcata | |
| tcaagttgaagatggcaactctgatctgacgcctctgtgctcagggcccctggaggaggg | |
| gctactgccccttggcccttagacaacaggacagagaccagagggactgggcggcttgtc | |
| ccaggctactgggctgtggtctctgaccccctggaggcctccccacgcctcacagaacac | |
| agtgcagacggtgggcggggagagctcgaggggaggcccagccctgcaggtgggactggc | |
| tctccctccccagccacgagtatgccacagggggctttcaaaaccaggcagggccaagcc | |
| ttggccccactggaggtagagcaccttctgtggctcccgggtgcctttaggcccgtccag | |
| gagttggagcagaggagaagacccagactgtgcatagttccctgaggagggctagcctca | |
| ggtgccacagcctggttcggcctggcggccccaggacgcagcagcaaccttgggcttcct | |
| gcggggtgactaccttgccctccggccctgcccgttgactaactatgctctgtggttttg | |
| tttccagagaactcagattgcacagagctagactcgggcagccagagcggcgagctgagt | |
| aacaggggtaacacctccctgctgcccctccctctctctccctctctctccctctctctt | |
| taccctccccagggctccatctccgcctcaggggcttctccaccccaagtctggctccat | |
| tcctggccttctgttggtgacagacccccccctaaggtgctcgtttgggggctcttcagg | |
| cagcacctcagcctggcacccccactcccctgcacagcccccaggcctcaggaccccacc | |
| cctctgaggcccaggggagccctgttcacgctggtttctccccaggacccatgagcttcc | |
| tggctggcctgggccttgctgtgggactggccctgctcctgtactgctatccgccagacc | |
| ccaagggcctgccagggacccggcgcgtcctcggtttctcgcctgtcatcatcgacagac | |
| atgtcagccgctacctgctggccttcctggcagatgacctaggggggctctgacagaccc | |
| tggacccagggcctcacctgccactcaaccaaagagtcctcgagccggcccgccaagggg | |
| actgctgcttctttttctaaatgcatatttttcattatttataatttgtgtaaaaaacac | |
| accttcaccttacaaggtgctgaccatattaaatgttcaggttctctcaaaaaaaaaa | |

TABLE 142

| | |
|---|---|
| cbe_2826346 RPS10 | |
| ribosomal protein S10 | |
| length = 598 | |
| ctccttcttttccagccccggtaccggaccctgcagccgcagagatgttgatgcctaaga | SEQ ID NO: 133 |
| agaaccggattgccatttatgaactcctttttaaggagggagtcatggtggccaagaagg | |
| atgtccacatgcctaagcacccggagctggcagacaagaatgtgcccaaccttcatgtca | |
| tgaaggccatgcagtctctcaagtcccgaggctacgtgaaggaacagtttgcctggagac | |
| atttctactggtaccttaccaatgagggtatccagtatctccgtgattaccttcatctgc | |
| ccccggagattgtgcctgccaccctacgccgtagccgtccagagactggcaggcctcggc | |
| ctaaaggtctggagggtgagcgacctgcgagactcacaagaggggaagctgacagagata | |
| cctacagacggagtgctgtgccacctggtgccgacaagaaagccgaggctggggctgggt | |
| cagcaaccgaattccagtttagaggcggatttggtcgtggacgtggtcagccacctcagt | |
| aaaattggagaggattcttttgcattgaataaacttacagccaaaaaaccttaaaaaa | |

TABLE 143

| | |
|---|---|
| cbe_2830508 Prey292051 | |
| Homo sapiens, clone MGC: 12344, mRNA, complete cds. | |
| length = 769 | |
| catcaccccataaacaaataggtttggtcctagcctttctattagctcttagtaagatta | SEQ ID NO: 134 |
| cacatgcaagcatccccgttccagtgagttcaccctctaaatcaccacgatcaaaaggga | |
| caagcatcaagcacgcagcaatgcagctcaaaacgcttagcctagccacacccccacggg | |
| aaacagcagtgattaacctttagcaataaacgaaagtttaactaagctatactaacccca | |
| gggttggtcaatttcgtgccagccaccgcggtcacacgattaacccaagtcaatagaagc | |
| cggcgtaaagagtgttttagatcaccccctccccaataaagctaaaactcacctgagttg | |
| taaaaaactccagttgacacaaaatagactacgaaagtggctttaacatatctgaacaca | |
| caatagctaagacccaaactgggattagataccccactatgcttagccctaaacctcaac | |
| agttaaatcaacaaaactgctcgccagaacactacgagccacagcttaaaactcaaagga | |
| cctggcggtgcttcatatccctctagaggagcctgttctgtaatcgataaaccccgatca | |
| acctcaccacctcttgctcagcctatataccgccatcttcagcaaaccctgatgaaggct | |
| acaaagtaagcgcaagtacccacgtaaagacgttaggtcaaggtgtagcccatgaggtgg | |
| caagaaatgggctacattttctaccccagcaacaatatactctgccggc | |

TABLE 144

| | |
|---|---|
| cbe_2898065 Prey1620 | |
| Homo sapiens, DKFZP434F162 protein, clone MGC: 21493 IMAGE: 3871231, mRNA, | |
| complete cds. | |
| length = 2156 | |
| gagctgaagagacctggagccgacagacgggtgaaggccatgccgccttctgggaaagtt | SEQ ID NO: 135 |
| ccccgaaaggagaatctgtggctacagtgtgagtgggggtcctgctcctttgtgtgctca | |
| accatggaaaagttctttgagcatgtcactcagcacctgcagcagcacctgcatggctct | |
| ggggaggaggaggaagaggaagaggaggatgacccacttgaggaagaattctcctgcttg | |
| tggcaggaatgtggcttttgttctctggactgttctgctgacctcatccgccatgtctac | |
| ttccactgctaccacaccaagctgaaacagtgggggctgcaggccttgcaaagccaggct | |
| gaccttggcccctgcatcctggacttccagagccggaacgtcatccctgatatccctgac | |
| cacttcctgtgtctgtgggagcactgtgagaattccttcgacaatcctgagtggttttat | |
| cggcatgtggaagcacacagtctgtgctgtgaatacgaagcagtcggcaaggacaacccg | |
| gtggtgctgtgtggctggaaaggctgtacctgcaccttcaaggaccgcagtaaacttcga | |
| gagcacctccgcagccatacccaggagaaagtggtagcctgccccacctgtgggggcatg | |
| tttgccaacaataccaagttcttagatcacatccgtcgccagacctcattggatcagcag | |
| cacttccagtgttctcactgttccaagagatttgccacagagcggctattgcgggaccac | |
| atgcgcaaccatgtgaatcactataagtgccctctgtgtgacatgacctgcccgctgcct | |
| tcctccctccgcaaccacatgcgctttcgtcacagtgaggaccggccctttaaatgtgac | |
| tgttgtgactacagctgcaagaatcttattgacctccagaagcacctggatacccacagc | |
| gaggagccagcctacaggtgtgattttgagaactgcaccttcagtgcccgatccctctgc | |
| tctatcaagtcccattaccgcaaagtacatgaaggagactctgagccaaggtacaaatgt | |
| catgtgtgtgacaaatgcttcacacggggcaacaacctcaccgtgcaccttcgcaagaag | |
| caccagttcaagtggccctcagggcatccccgttttcggtacaaggaacatgaagatggc | |
| tatatgcggctgcagctggttcgctacgagagtgtagagctgacacagcaactgctgcgg | |
| caaccacaagagggatcgggcctgggaacgtcgctgaacgagagcagcctgcagggcatt | |
| attctagaaacagtgccaggggagccaggacgtaaggaagaggaagaggagggcaagggt | |
| agcgaagggacagccctctcagcctctcaggacaaccccagttctgtcatccacgtggtg | |
| aatcagaccaatgcccaaggccagcaagagattgtctactatgtgctgtctgaagcccca | |
| ggggagcctcccccagtccctgagccaccttcagggggcatcatggaaaagcttcaagga | |
| atagctgaggagccagagatccagatggtttgaaggccgcagagccagaccatttcttcc | |
| ccaggtcctgaagtttgagccaggcaagtggcagtgcccctagtgggcagccgttgccaa | |
| tggatgcctttaggagtggtgccgagagcagtgtggtccactctggcctgggtttgcatc | |
| attctgcagactctaaagacttcccttttctgccagactacattttgtggggagcctgag | |
| gactctggattctttgaggggatcctggatgtgtgtgttcttgttaaagaggctgttatc | |
| aggcttaactataaccctcaagatctgcttgacagtgattaaatccttagctcacatcca | |
| ttcccatctttcgggctccttaggcccaaggatggcatgtgactggtccctgcaagggtc | |
| ctttctttgtcaccagccaaggcattgataaccaagtagccattttcctcttaaggtttc | |
| ctctacaaccccaaggactttcatgattatcctcagggacaggattggaggcattgagcg | |
| tgtttattaacaagatgtttttggtaataaaaaaaaaaaaaaaaaaaaaaaaaaaa | |

TABLE 145

| | |
|---|---|
| cbe_3001381 IRF1 | |
| interferon regulatory factor 1 | |
| length = 2035 | |
| cgagccccgccgaaccgaggccacccggagccgtgcccagtccacgccggccgtgcccgg | SEQ ID NO: 136 |
| cggccttaagaaccaggcaacctctgccttcttccctcttccactcggagtcgcgctccg | |
| cgcgccctcactgcagcccctgcgtcgccgggaccctcgcgcgcgaccagccgaatcgct | |
| cctgcagcagagccaacatgcccatcactcggatgcgcatgagaccctggctagagatgc | |
| agattaattccaaccaaatcccggggctcatctggattaataaagaggagatgatcttcc | |
| agatcccatggaagcatgctgccaagcatggctgggacatcaacaaggatgcctgtttgt | |
| tccggagctgggccattcacacaggccgatacaaagcaggggaaaaggagccagatccca | |
| agacgtggaaggccaactttcgctgtgccatgaactccctgccagatatcgaggaggtga | |
| aagaccagagcaggaacaagggcagctcagctgtgcgagtgtaccggatgcttccacctc | |
| tcaccaagaaccagagaaaagaaagaaagtcgaagtccagccgagatgctaagagcaagg | |
| ccaagaggaagtcatgtggggattccagccctgataccttctctgatggactcagcagct | |
| ccactctgcctgatgaccacagcagctacacagttccaggctacatgcaggacttggagg | |
| tggagcaggccctgactccagcactgtcgccatgtgctgtcagcagcactctccccgact | |
| ggcacatcccagtggaagttgtgccggacagcaccagtgatctgtacaacttccaggtgt | |
| cacccatgccctccacctctgaagctacaacagatgaggatgaggaagggaaattacctg | |
| aggacatcatgaagctcttggagcagtcggagtggcagccaacaaacgtggatgggaagg | |
| ggtacctactcaatgaacctggagtccagcccacctctgtctatggagactttagctgta | |
| aggaggagccagaaattgacagcccagggggggatattgggctgagtctacagcgtgtct | |
| tcacagatctgaagaacatggatgccacctggctggacagcctgctgaccccagtccggt | |
| tgccctccatccaggccattccctgtgcaccgtagcagggcccctgggcccctcttattc | |
| ctctaggcaagcaggacctggcatcatggtggatatggtgcagagaagctggacttctgt | |
| gggcccctcaacagccaagtgtgaccccactgccaagtggggatgggcctccctccttgg | |
| gtcattgacctctcagggcctggcaggccagtgtctgggtttttcttgtggtgtaaagct | |
| ggccctgcctcctgggaagatgaggttctgagaccagtgtatcaggtcagggacttggac | |
| aggagtcagtgtctggctttttcctctgagcccagctgcctggagagggtctcgctgtca | |
| ctggctggctcctaggggaacagaccagtgaccccagaaaagcataacaccaatcccagg | |
| gctggctctgcactaagcgaaaattgcactaaatgaatctcgttccaaagaactacccct | |
| tttcagctgagccctggggactgttccaaagccagtgaatgtgaaggaaactcccctcct | |
| tcggggcaatgctccctcagcctcagaggagctctaccctgctccctgctttggctgagg | |
| ggcttgggaaaaaaacttggcactttttcgtgtggatcttgccacatttctgatcagagg | |
| tgtacactaacatttcccccgagctcttggcctttgcatttatttatacagtgccttgct | |
| cggggcccaccaccccctcaagccccagcagccctcaacaggcccagggagggaagtgtg | |
| agcgccttggtatgacttaaaattggaaatgtcatctaaccattaagtcatgtgtgaaca | |
| cataaggacgtgtgtaaatatgtacatttgtctttttataaaaagtaaaattgtt | |

TABLE 146

| | |
|---|---|
| cbe_3056756 KIAA0440 | |
| signal-induced proliferation-associated 1 like 1 | |
| length = 6028 | |
| ggtgtggacgttgtctaaatttcggtagccatggcacaagaatataagaaagcatgggat | SEQ ID NO: 137 |
| tatggcaaccacagaatctcagtagtacaagttccattcagttttttctgaaagaaagcc | |
| ctctgttaaagtgaagcaaagaaactgttgtggattataacgtttagaagttccaatttt | |
| tcagtgctttacaaataaagcatcatttaaccttttaaatgaaaaagattaagatctcat | |
| gcaactgttgtattttctggaagccattctccaaaagggaagtgcacatttaaaacacag | |
| atatgatggtccttgctgcagggatttaagtctacttgcttttacatcatgaccagcttg | |
| aaacggtcacagacagaaaggcctcttgccactgacagggcctctgttgttggcacagac | |
| ggcacccccaaagtccacactgatgatttctacatgcggcgcttccggtcccaaaatggc | |
| agcttaggatcatcagttatggctcctgtaggacccccccgaagtgaaggttctcaccat | |
| ataacctcaacccccggagtcccaaaaatgggggtaagggcaaggattgcagattggccc | |
| ccaagaaaggaaaacataaaagaatctagccgttcaagccaggaaatagaaacctcaagt | |
| tgccttgatagcctgtcctccaaaagcagtcctgtgagtcagggaagttctgttagcctc | |
| aattccaatgactcagccatgctgaaaagcatacagaacacgctgaaaaacaagacaaga | |
| ccgtcggagaacatggactccagatttctcatgcctgaagcctaccccagctcccccaga | |
| aaagctcttcgcagaatacgccagcgaagcaacagtgatatcaccataagtgaacttgat | |
| gtggatagctttgatgaatgtatctcacctacatacaagactggaccatcactgcacagg | |
| gaatatggtagcacatcttcaattgataaacagggaacatctggagaaagcttttttgat | |
| ttgttaaagggctacaaagatgacaaatctgatcgaggtccaactccaaccaagctcagt | |
| gactttctcattactggtggtggcaagggttctggtttctctttggatgtaatagacggg | |
| cctatctcacagagagagaacctcaggctttttaaggaaagggaaaaaccactcaagcga | |
| cgttcaaaatctgaaactggagactcctctatttttcgtaaattgcgcaatgccaaaggt | |
| gaagaacttgggaagtcatcagatcttgaagataaccgatcagaagactctgtcaggccc | |
| tggacatgtccaaagtgctttgcccactatgatgtccagagtatattatttgatttgaat | |
| gaggcaattatgaacaggcacaatgttattaagaggagaaacaccaccactggagcttcc | |
| gcagctgccgtggcatccttggtctctggacctctgtctcattcagccagttttagctcc | |
| ccaatgggcagcacagaggacctgaattccaaaggaagcctcagcatggaccagggagat | |
| gataaaagcaatgagcttgtaatgagctgtccatattttcggaatgagataggtggagaa | |
| ggggagaggaaaatcagcctttcaaaatcaaattctggctcctttagtggatgtgaaagt | |
| gcctcctttgagtctacccttagttcccattgcacaaatgcaggagtggcagtacttgaa | |
| gtgcccaaggagaacttggtgttgcacctagatagagtgaaaagatacatcgtggaacac | |
| gtagatctgggtgcatactattatagaaaatttttctaccagaaggaacactggaactat | |
| tttggggctgatgagaatcttggtccagtggctgtgagcattcgaagggaaaaaccagat | |
| gaaatgaaagaaaatggatctccgtacaactaccgaataatttttagaactagtgagctc | |
| atgacactgagaggttcggtcctggaggacgccattccgtcgacagccaagcactcgaca | |
| gccagaggcctgcctctcaaagaagtgctggagcacgtggttcctgagctcaatgtccag | |
| tgcctgcggttggccttcaacacacccaaggtcacagagcagctcatgaaactggatgaa | |
| caagggctgaactaccagcagaaagtaggcatcatgtactgcaaagctggacagagcact | |
| gaagaagagatgtacaacaatgagtcagctggcccagcctttgaagaattccttcaacta | |
| ttgggagagcgagttcggctcaaaggatttgagaagtatcgagcacagcttgataccaaa | |
| actgactccactggaacccattctctgtacacaacatacaaagattatgaaattatgttc | |
| catgtttctaccatgctgccatacacacccaacaacaaacaacagctcctgaggaagcgg | |
| cacattggaaatgatatcgtaacaattgttttccaagagcctggagcacagccattcagc | |
| ccaaaaaacatccgatcccacttccagcacgttttcgtcatcgtcagggtgcacaatccg | |
| tgctctgacagtgtctgttatagtgtggctgttaccaggtccagagatgtgccttccttt | |
| gggcctcccattcctaaaggggtcactttccctaagtcaaatgtgttcagggacttcctt | |
| ttggcgaaagtgattaatgcagaaaatgctgctcataaatcggagaagtttcgggccatg | |
| gcaactcggacccgccaggaatacctgaaagatctggcagaaaagaatgtcaccaacacc | |
| cctatcgacccttctggcaagtttccgttcatctctctggcttccaagaagaaggaaaag | |
| tctaagccatatccaggagccgagctcagcagcatgggggccattgtatgggcagtccgg | |
| gctgaagactacaacaaggccatggaactagactgccttttagggatctccaatgagttc | |
| attgtgctcattgaacaggaaacaaagagcgtggtcttcaattgttcctgtagagatgtg | |
| atagggtggacttcaactgacaccagcctcaaaatcttctatgaacgaggagaatgtgtt | |
| tcagtgggtagttttattaacattgaggagatcaaagagattgtcaaaaggttgcagttt | |
| gtttcaaaaggctgtgaatcggtggagatgactctgcgaagaaatgggctaggacagctt | |
| ggcttccatgtcaactatgagggcattgtggcggatgtggagccctacggttatgcctgg | |
| caggcagggctgaggcagggcagtcgcctggtggagatctgcaaggtggcggtagccact | |
| ctgagccatgagcagatgatcgacctcctgagaacatctgtcacggtgaaggttgtcatc | |
| attcccccgcatgatgactgcaccccgcggaggagttgctctgaaacctaccgcatgcca | |
| gtgatggagtacaaaatgaatgaaggtgtttcatacgaattcaagtttcccttccgaaat | |
| aataacaagtggcagaggaacgccagcaaggggcctcattcacctcaagtcccgtcccag | |
| gtgcagagtcccatgacctcgcggctgaatgctggaaaaggagatgggaagatgcctcct | |
| ccagaaagagccgccaacatccctcgaagcatctccagtgacgggcgcccactagagagg | |
| cggctgtctcctggttcggacatctatgtgacggtctcatccatggctttagcaagatcc | |
| cagtgtcggaactctcctagcaacttgtcttcatccagtgatactggttctgtggggggc | |
| acttacaggcagaagtccatgcccgaagggtttggagtgagccgtagatccccagcctcc | |
| attgacaggcagaacacccagtcagatattggtggcagcggaaaatccacgcctagctgg | |
| caaagaagtgaggatagcattgctgaccagatggcttacagttatagaggacctcaggat | |
| ttcaattcttttgtcctcgagcagcatgaatatacagagccaacatgccatctcccagca | |
| gtatcaaaggtactgccagctttccgagagagccccagtgggagattaatgcggcaggat | |
| ccagtggttcatttgtctccaaacaaacaagggcattctgatagccactactcgagccac | |
| tccagtagcaatactctctccagcaatgcgtcaagtgcccatagtgatgagaagtggtac | |
| gatggggaccgcacagaatccgaactcaacagctataactatctgcaaggcacctctgct | |
| gacagtggcattgacaccacctcttatggccccagccacggcagcacagcctcgctgggg | |
| gctgccacatcgtcacctcgctcagggccaggcaaggagaaagtggcacccctatggcac | |
| agctccagtgaagtaatctccatggcagatcggactttggagacagagagccacggcctg | |
| gaccggaaaacagagtcttccctgagcttagacatacacagcaagagccaagccggctcg | |
| acccctctgacaagggagaacagcaccttcagtataaacgatgctgcttcccacacaagt | |
| accatgagctcccgacactctgccagcccagtggttttcaccagtgcccggagttcacct | |
| aaagaagagcttcatccagctgccccctcacagctcgcaccatccttctcctcctcttcc | |
| tcctcctcctctggtcctaggagtttttaccctcgccagggcgctactagcaagtacctg | |
| attggatggaaaaaacccgaaggaaccataaactccgtgggatttatggacacgagaaag | |
| cgtcatcagagcgatggcaatgaaatagcccacaccaggctgcgtgcctcaaccagagac | |
| ctccgggcatctcctaagccaacctccaagtccaccattgaagaagatctaaagaaacta | |
| attgatcttgaaagcccaactcctgaatcacagaagagttttaagttccacgcactctcc | |
| tctcctcagtctcctttccccagcacccccacctcacggcgggccttgcacagaacactg | |
| tcggacgagagcatttacaatagccagagggagcactttttcacctccagggcgtcactt | |
| ctggaccaagccctgcccaacgacgtcctcttcagtagcacgtacccttctctccccaag | |
| tcgctcccgttgaggaggccttcttacaccttaggaatgaaatcgctgcatggagagttc | |
| tcagcctcggacagctccctcactgacatccaggagacccgcaggcagcctatgcccgac | |
| cctggcctgatgcccctgcctgacactgctgcagacttggattggtccaacctggtagat | |
| gctgccaaagcctatgaggtccagagagcctcattttttgctgctagtgatgaaaaccat | |
| cgccccttgagtgctgcatccaacagtgatcagctggaggaccaggctctggcccagatg | |
| aagccttacagcagcagtaaagactcctctcccactctggcttctaaagtggaccagctg | |
| gaaggtatgctgaagatgcttcgggaagatttgaagaaggaaaaagaagacaaagctcac | |
| cttcaggcggaggtgcagcacctgcgagaggacaacctgaggctacaggaggagtcccag | |
| aacgcctcggacaagctgaagaagttcacagaatgggtcttcaacaccatagacatgagc | |
| tagggaaggctgaggaggacaggagaagggcccagacactccctccagtgagtgtcctgc | |
| agcccttattccctccatagaaagcatcctcagagcaccttccctggcttcctactctgc | |
| cccctttcggggagtgcacaacacaatagttgcagatcaacaatcatcacctgccttttg | |
| tagaaaagaaaaacaaaaaaagtaaataaaaattttaaacagtaaaataaaagtttaact | |
| gctaaaaaaaaaaaaaaaaaaaaaaaaa | |

TABLE 147

| | |
|---|---|
| cbe_3340492 TRIP | |
| TRAF interacting protein | |
| length = 2007 | |
| gtgcggtggagcgaaatttgaagcaagcggaggcggggcgctctacgaagccggacctgt | SEQ ID NO: 138 |
| agcagtttctttggctgcctgggccccttgagtccagccatcatgcctatccgtgctctg | |
| tgcactatctgctccgacttcttcgatcactcccgcgacgtggccgccatccactgcggc | |
| cacaccttccacttgcagtgcctaattcagtcctttgagacagcaccaagtcggacctgc | |
| ccacagtgccgaatccaggttggcaaaagaaccattatcaataagctcttctttgatctt | |
| gcccaggaggaggagaatgtcttggatcgagaattcttaaagaatgaactggacaatgtc | |
| agagcccagctttcccagaaagacaaggagaaacgagacagccaggtcatcatcgacact | |
| ctgcgggatacgctggaagaacgcaatgctactgtggtatctctgcagcaggccttgggc | |
| aaggccgagatgctgtgctccacactgaaaaagcagatgaagtacttagagcagcagcag | |
| gatgagaccaaacaagcacaagaggaggcgggccggctcaggagcaagatgaagaccatg | |
| gagcagattgagcttctactccagagccagctccctgaggtggaggagatgatccgagac | |
| atgggtgtgggacagtcagcggtggaacagctggctgtgtactgtgtgtctctcaagaaa | |
| gagtacgagaatctaaaagaggcacggaaggcctcaggggaggtggctgacaagctgagg | |
| aaggatttgttttcctccagaagcaagttgcagacagtctactctgaattggatcaggcc | |
| aagttagaactgaagtcagcccagaaggacttacagagtgctgacaaggaaatcatgagc | |
| ctgaaaaagaagctaacgatgctgcaggaaaccttgaacctgccaccagtggccagtgag | |
| actgtcgaccgcctggttttagagagcccagcccctgtggaggtgaatctgaagctccgc | |
| cggccatccttccgtgatgatattgatctcaatgctacctttgatgtggatactccccca | |
| gcccggccctccagctcccagcatggttactacgaaaaactttgcctagagaagtcacac | |
| tccccaattcaggatgtccccaagaagatatgcaaaggccccaggaaggagtcccagctc | |
| tcactgggtggccagagctgtgcaggagagccagatgaggaactggttggtgccttccct | |
| atttttgtccggaatgccatcctaggccagaaacagcccaaaaggcccaggtcagagtcc | |
| tcttgcagcaaagatgtggtaaggacaggcttcgatgggctcggtggccggacaaaattc | |
| atccagcctactgacacagtcatgatccgcccattgcctgttaagcccaagaccaaggtt | |
| aagcagagggtgagggtgaagaccgtgccttctctcttccaggccaagctggacaccttc | |
| ctgtggtcgtgagaacagtgagtctgaccaatggccagacacatgcctgcaacttgtagg | |
| tcaaggactgtccaggcagggtttgtggacagagccctactttcgggaccagcctgaggt | |
| gtaagggcagacaaacaggtgagggtgagtgtgacacccagagactgctcttcctgccct | |
| caccctgccccactcctacgactgggagctgacatgaccagcccactgatcctgtcagca | |
| ggtcctgctctgttgccaggctcttgtttatagccatgatcagatgtggtcagactcttt | |
| ctgggcctggagaccacggtcacttgttgactgtctctgtggaccagagtgcttgaggca | |
| tctcaggcagcctcagcccaagcttctacctgcctttgacttgcttctagcatagcctgg | |
| gccaagcagggtggggaatggaggatagacatgggatgtatggagaggatggaagatttt | |
| cccgaaaaaaaaaaaaaaaaaaaaaaa | |

TABLE 148

| | |
|---|---|
| che_3345510 COX6C | |
| cytochrome c oxidase subunit VIc | |
| length = 444 | |
| ctttagtcaggaaggacgttggtgttgaggttagcatacgtatcaaggacagtaactacc | SEQ ID NO: 139 |
| atggctcccgaagttttgccaaaacctcggatgcgtggccttctggccaggcgtctgcga | |
| aatcatatggctgtagcattcgtgctatccctgggggttgcagctttgtataagtttcgt | |
| gtggctgatcaaagaaagaaggcatacgcagatttctacagaaactacgatgtcatgaaa | |
| gattttgaggagatgaggaaggctggtatctttcagagtgtaaagtaatcttggaatata | |
| aagaatttcttcaggttgaattacctagaagtttgtcactgacttgtgttcctgaactat | |
| gacacatgaatatgtgggctaagaaatagttcctcttgataaataaacaattaacaaata | |
| ctttgaaaaaaaaaaaaaaaaaaa | |

TABLE 149

| | |
|---|---|
| che_3454406 gbh_af252829 | |
| Homo sapiens chromosome 8 clone BAC 495D4 map 8g24, *** SEQUENCING | |
| length = 488 | |
| ccgagcagcctaactgggaggcaccccccagcagaggcacactgacacctcacacggcag | SEQ ID NO: 140 |
| ggtattccaacagacctgcagctgagggtcctgtctgttagaaggaaaactaacaaccag | |
| aaaggacatctacaccgaaaacccatctgtacatcaccatcatcaaagaccaaaagtaga | |
| taaaaccacaaagatggggaaaaaacagaacagaaaaactggaaactctaaaacgcagag | |
| cgcctctcctcctccaaaggaacgcagttcctcaccagcaatggaacaaagctggatgga | |
| gaatgattttgacgagctgagagaagaaggcttcagacgatcaaattactctgagctacg | |
| ggaggacattcaaaccaaaggcaaagaagttggaaactttgaaaaaaatttagaagaatg | |
| tataactagaataaccaatacagagaagtgcttaaaggagctgatggagctgaaaaccaa | |
| ggctcgag | |

TABLE 150

| | |
|---|---|
| cbe_3461331 MET | |
| met proto-oncogene (hepatocyte growth factor receptor) | |
| length = 4620 | |
| cgccctcgccgcccgcggcgccccgagcgctttgtgagcagatgcggagccgagtggagg | SEQ ID NO: 141 |
| gcgcgagccagatgcggggcgacagctgacttgctgagaggaggcggggaggcgcggagc | |
| gcgcgtgtggtccttgcgccgctgacttctccactggttcctgggcaccgaaagataaac | |
| ctctcataatgaaggcccccgctgtgcttgcacctggcatcctcgtgctcctgtttacct | |
| tggtgcagaggagcaatggggagtgtaaagaggcactagcaaagtccgagatgaatgtga | |
| atatgaagtatcagcttcccaacttcaccgcggaaacacccatccagaatgtcattctac | |
| atgagcatcacattttccttggtgccactaactacatttatgttttaaatgaggaagacc | |
| ttcagaaggttgctgagtacaagactgggcctgtgctggaacacccagattgtttcccat | |
| gtcaggactgcagcagcaaagccaatttatcaggaggtgtttggaaagataacatcaaca | |
| tggctctagttgtcgacacctactatgatgatcaactcattagctgtggcagcgtcaaca | |
| gagggacctgccagcgacatgtctttccccacaatcatactgctgacatacagtcggagg | |
| ttcactgcatattctccccacagatagaagagcccagccagtgtcctgactgtgtggtga | |
| gcgccctgggagccaaagtcctttcatctgtaaaggaccggttcatcaacttctttgtag | |
| gcaataccataaattcttcttatttcccagatcatccattgcattcgatatcagtgagaa | |
| ggctaaaggaaacgaaagatggttttatgtttttgacggaccagtcctacattgatgttt | |
| tacctgagttcagagattcttaccccattaagtatgtccatgcctttgaaagcaacaatt | |
| ttatttacttcttgacggtccaaagggaaactctagatgctcagacttttcacacaagaa | |
| taatcaggttctgttccataaactctggattgcattcctacatggaaatgcctctggagt | |
| gtattctcacagaaaagagaaaaaagagatccacaaagaaggaagtgtttaatatacttc | |
| aggctgcgtatgtcagcaagcctggggcccagcttgctagacaaataggagccagcctga | |
| atgatgacattcttttcggggtgttcgcacaaagcaagccagattctgccgaaccaatgg | |
| atcgatctgccatgtgtgcattccctatcaaatatgtcaacgacttcttcaacaagatcg | |
| tcaacaaaaacaatgtgagatgtctccagcatttttacggacccaatcatgagcactgct | |
| ttaataggacacttctgagaaattcatcaggctgtgaagcgcgccgtgatgaatatcgaa | |
| cagagtttaccacagctttgcagcgcgttgacttattcatgggtcaattcagcgaagtcc | |
| tcttaacatctatatccaccttcattaaaggagacctcaccatagctaatcttgggacat | |
| cagagggtcgcttcatgcaggttgtggtttctcgatcaggaccatcaacccctcatgtga | |
| attttctcctggactcccatccagtgtctccagaagtgattgtggagcatacattaaacc | |
| aaaatggctacacactggttatcactgggaagaagatcacgaagatcccattgaatggct | |
| tgggctgcagacatttccagtcctgcagtcaatgcctctctgccccaccctttgttcagt | |
| gtggctggtgccacgacaaatgtgtgcgatcggaggaatgcctgagcgggacatggactc | |
| aacagatctgtctgcctgcaatctacaaggttttcccaaatagtgcaccccttgaaggag | |
| ggacaaggctgaccatatgtggctgggactttggatttcggaggaataataaatttgatt | |
| taaagaaaactagagttctccttggaaatgagagctgcaccttgactttaagtgagagca | |
| cgatgaatacattgaaatgcacagttggtcctgccatgaataagcatttcaatatgtcca | |
| taattatttcaaatggccacgggacaacacaatacagtacattctcctatgtggatcctg | |
| taataacaagtatttcgccgaaatacggtcctatggctggtggcactttacttactttaa | |
| ctggaaattacctaaacagtgggaattctagacacatttcaattggtggaaaaacatgta | |
| ctttaaaaagtgtgtcaaacagtattcttgaatgttataccccagcccaaaccatttcaa | |
| ctgagtttgctgttaaattgaaaattgacttagccaaccgagagacaagcatcttcagtt | |
| accgtgaagatcccattgtctatgaaattcatccaaccaaatcttttattagtacttggt | |
| ggaaagaacctctcaacattgtcagttttctattttgctttgccagtggtgggagcacaa | |
| taacaggtgttgggaaaaacctgaattcagttagtgtcccgagaatggtcataaatgtgc | |
| atgaagcaggaaggaactttacagtggcatgtcaacatcgctctaattcagagataatct | |
| gttgtaccactccttccctgcaacagctgaatctgcaactccccctgaaaaccaaagcct | |
| ttttcatgttagatgggatcctttccaaatactttgatctcatttatgtacataatcctg | |
| tgtttaagccttttgaaaagccagtgatgatctcaatgggcaatgaaaatgtactggaaa | |
| ttaagggaaatgatattgaccctgaagcagttaaaggtgaagtgttaaaagttggaaata | |
| agagctgtgagaatatacacttacattctgaagccgttttatgcacggtccccaatgacc | |
| tgctgaaattgaacagcgagctaaatatagagtggaagcaagcaatttcttcaaccgtcc | |
| ttggaaaagtaatagttcaaccagatcagaatttcacaggattgattgctggtgttgtct | |
| caatatcaacagcactgttattactacttgggtttttcctgtggctgaaaaagagaaagc | |
| aaattaaagatctgggcagtgaattagttcgctacgatgcaagagtacacactcctcatt | |
| tggataggcttgtaagtgcccgaagtgtaagcccaactacagaaatggtttcaaatgaat | |
| ctgtagactaccgagctacttttccagaagatcagtttcctaattcatctcagaacggtt | |
| catgccgacaagtgcagtatcctctgacagacatgtcccccatcctaactagtggggact | |
| ctgatatatccagtccattactgcaaaatactgtccacattgacctcagtgctctaaatc | |
| cagagctggtccaggcagtgcagcatgtagtgattgggcccagtagcctgattgtgcatt | |
| tcaatgaagtcataggaagagggcattttggttgtgtatatcatgggactttgttggaca | |
| atgatggcaagaaaattcactgtgctgtgaaatccttgaacagaatcactgacataggag | |
| aagtttcccaatttctgaccgagggaatcatcatgaaagattttagtcatcccaatgtcc | |
| tctcgctcctgggaatctgcctgcgaagtgaagggtctccgctggtggtcctaccataca | |
| tgaaacatggagatcttcgaaatttcattcgaaatgagactcataatccaactgtaaaag | |
| atcttattggctttggtcttcaagtagccaaagcgatgaaatatcttgcaagcaaaaagt | |
| ttgtccacagagacttggctgcaagaaactgtatgctggatgaaaaattcacagtcaagg | |
| ttgctgattttggtcttgccagagacatgtatgataaagaatactatagtgtacacaaca | |
| aaacaggtgcaaagctgccagtgaagtggatggctttggaaagtctgcaaactcaaaagt | |
| ttaccaccaagtcagatgtgtggtcctttggcgtcgtcctctgggagctgatgacaagag | |
| gagccccaccttatcctgacgtaaacacctttgatataactgtttacttgttgcaaggga | |
| gaagactcctacaacccgaatactgcccagaccccttatatgaagtaatgctaaaatgct | |
| ggcaccctaaagccgaaatgcgcccatccttttctgaactggtgtcccggatatcagcga | |
| tcttctctactttcattggggagcactatgtccatgtgaacgctacttatgtgaacgtaa | |
| aatgtgtcgctccgtatccttctctgttgtcatcagaagataacgctgatgatgaggtgg | |
| acacacgaccagcctccttctgggagacatcatagtgctagtactatgtcaaagcaacag | |
| tccacactttgtccaatggttttttcactgcctgacctttaaaaggccatcgatattctt | |
| tgctccttgccataggacttgtattgttatttaaattactggattctaaggaatttctta | |
| tctgacagagcatcagaaccagaggcttggtcccacaggccagggaccaatgcgctgcag | |

TABLE 151

| | |
|---|---|
| cbe_3473488 gbh_al121903 | |
| Human DNA sequence from clone RP1-155G6 on chromosome 20 Contains | |
| length = 5694 | |
| aagaaaattgcaatgaaagaaacaaaagagctaacgattgcaaccaaatctactaagcag | SEQ ID NO: 142 |
| aatgtagctagtgaaaagcagcggcggctgctgtacaacttagagatggagcaaatggct | |
| aaaacagccaaagctctgatggaggctgtgagccatgccaaagccccgtttaccagtgcc | |
| actcacctggaccatgtccggccaatgttcaaactggtgtggacgccactattggcagcc | |
| tacagcatcggactccagaactgtgatgacactgaagtggcctccttgtgtttggaaggc | |
| atccgatgtgcaatccgaatcgcctgcatctttggaatgcagctggaacgagatgcctat | |
| gttcaggctcttgctcgcttctccctactcacagccagctccagcatcacagaaatgaag | |
| cagaaaaacatcgacaccattaagacgcttatcacagtggctcacaccgatggcaactac | |
| cttgggaattcctggcatgagatcttgaaatgcatcagccagctggagctcgctcagctg | |
| ataggaaccggtgtgaagacgcgctacctgtctggatctgggcgtgaaagagaagggagc | |
| ctgaagggccacacattggcaggagaagagttcatgggccttggcctcggtaatttggtg | |
| agtggcggagtggataaaagacagatggccagcttccaagaatcggttggtgagaccagc | |
| tcgcagagtgtggttgtagctgtggacaggatttttactgggtctaccagactggatgga | |
| aatgcaatagttgactttgtccgctggctgtgtgctgtgtccatggatgaactggcttcc | |
| ccccaccatcctcgcatgttcagcttgcagaagattgtggagatatcatactacaacatg | |
| aatcggatccgactacagtggtctcgaatatggcatgtgattggagatcacttcaataag | |
| gttggctgcaaccctaatgaagatgtggctatctttgctgttgactcattaaggcaactc | |
| tccatgaagtttcttgagaagggtgaattagccaacttccgtttccagaaagattttctg | |
| aggccctttgagcatattatgaagaaaaacaggtctcccaccatccgggacatggcgatc | |
| cgctgcattgcccagatggtgaactcccaggcggccaacatccgctcaggctggaagaac | |
| atctttgccgtgttccaccaggcagcctctgatcatgatgggaacattgtggagctggcc | |
| ttccagaccacttgccacattgtcacaactattttccagcaccattttcctgcagccatc | |
| gattcctttcaggatgctgtgaagtgcttatcagagttcgcctgcaacgccgctttccct | |
| gacacgagcatggaagcgattcggctcatccgcttctgtggcaaatacgtctctgagagg | |
| cctcgggtgctacaagaatacacaagtgatgacatgaatgtagctcctggtgacagagtc | |
| tgggtccgaggctggttccccatcttattcgaactctcctgcatcattaatagatgcaag | |
| ttagatgtacgaacaaggggactcacagtcatgtttgagatcatgaagagctatggccac | |
| acctttgagaagcactggtggcaggacctgttcagaatcgtgtttcggatttttgacaat | |
| atgaaactccctgagcaactgtcagagaaatctgagtggatgacaacaacctgcaatcac | |
| gcactttatgctatttgtgatgtttttacccagttttatgaagctttgaatgaagttctt | |
| ctttctgatgtatttgcacaattgcagtggtgtgtcaaacaagataatgaacagttggcg | |
| cgatcaggtacaaattgcttagaaaacttagtaatatccaatggagagaaattcagtcct | |
| gaagtctgggatgaaacctgcaactgtatgttggatattttcaaaacaaccatcccacat | |
| gttttgctgacatggagacctgtaggaatggaggaagattcatcagaaaagcatttggat | |
| gtggatctggaccgccagtctttaagcagcatagataaaaatccctctgagaggggacag | |
| agccagctctctaacccaacagatgacagctggaagggtagaccatacgcaaatcagaaa | |
| ctgtttgccagcctcctcatcaagtgtgtggtccagttggaattgatacagaccattgac | |
| aacattgtgttctaccctgcgacgagcaaaaaggaggatgcagagcacatggttgccgcc | |
| cagcaagacacgctggatgcagatatccacatagagacggaggatcagggcatgtataag | |
| tacatgtcttcccagcacctcttcaagctgttggactgtttgcaggaatcccattcattc | |
| tcaaaggccttcaactccaattacgagcagcggactgtcctgtggcgagcaggttttaag | |
| ggcaagtctaaacccaatcttctaaaacaagaaaccagcagcctggcctgttgtttgagg | |
| atcctgtttcgaatgtatgttgatgagaaccgcagggattcctgggaagaaatacagcag | |
| agacttttaactgtttgcagtgaagctcttgcctatttcatcactgtgaattctgagagc | |
| catcgggaggcctggacaagtctcttgttgttacttctaactaaaaccctcaaaataaat | |
| gatgaaaagttcaaagcacatgcttcaatgtactacccctacttgtgtgaaattatgcag | |
| tttgacctgatccctgagctccgagcagttctgcggaagttcttcctacggataggtgtt | |
| gtgtataagatatggataccagaagagccatcacaggtaccagcagcactgtcaccagtg | |
| tggtagccctggctgcccaggccagtgctgcagctctgcagaatgttcagcatgccattt | |
| ctgactggcacatctcgtgaagtttcatagaaacaaggagttggcatcttggatctcaga | |
| atggcctggaaacggatggcctctacgctgttccatcacagtctccaactaaggcttatg | |
| gtatttcattaaactgttgcatacccagttagcacagtaggtggggagtctgcttcattt | |
| ctatcattccatttttctgattaaactgtcaaatctgtcattgcatatgccatcgttttc | |
| tagcaaaatcccatgattggctataaacgttttgtaagaagtcactctccttgaaaatac | |
| tgaacatagctgtataggtttgtgattattagagaatatgttaataaaacttcttgtaac | |
| ggctaactgccacctaaaatatgctgggttttctgttgtttgagtgtgttagagaaattt | |
| gaatgtttttgtcagttacgagtcagccgtaattagattagtttaaggacaggtcaggat | |
| tagagaagagttgtttttgtgttttgttaatgtctgagtgatttttaaagtattttacaa | |
| aaagatattgaaaatttggttgaaggcagagtttagtaattaagttagaattaagagttt | |
| tgcgaggttaaaaaaatgtgcctcgtggatctccctgttttagtaacatggagagaaaaa | |
| gtctacacgaaaaagtgaacaatttaatgaagatgattagccttccttgaaataagtatt | |
| tgtggatgggtgttaaattaaaatttccagaatacactgtccatctcacacactgtgaaa | |
| tctaatatatgaagtagtaatgaaaatgaagtagtaatttaaccagagttcatttatcct | |
| tgaataaacctttttattttcacctcagaaaagtgagtgtactggcagttagtgtcactg | |
| ctttgcacagtccccattaaaggaccctccagagagggacagtaactgtgcatgagaagc | |
| cgctcccataagcctcctcagccagatgtcatgggtggaactggagctgtgtcggggcca | |
| gcacagctgaactgtgacaatggcaggaggtggcatgtgcccagcacttccattaatctg | |
| agcctaggagttgaattctttggcaaggttggattctgaggtccttattatgttaatgat | |
| ggtgcaatactctcacctgcagtagaactgagttctgctgcagcttgtgtaaaagtgggc | |
| agtacacaagtacgtcccaaagcctgtgagcagtatacgtggatgctcacccatgagaag | |
| gagcacacacgcctcattctctgccctcacccactgctcacctagagcatcgctgagcgt | |
| tagacaaagtgttacacagaatgattaaaactttcagacttctacctatgctctttagtc | |
| ctgtaaattgggttgtattgatgtcaactctggtgccttagaagttagtagtttgggaac | |
| ctatctgtaaaatcagatgttttttctttgtagagaaggatttctggtgcttttgcttac | |
| taagagaccgatattcttaagttgttttcttgttttaacagccttgagaaatgtttggtt | |
| ttggccagcagaattcttgtctacttttttctttcccaaaaagtgttttttaatttctct | |
| accaaagaaaaaatgagcaggtttaggtttttacatgacttatatacattagataaaagg | |
| agctgtataatttagcaggaaaggactagggagaatactttactgtgagtggaaaatgtt | |
| agcacatttgactggtttgccctggaatccactgcacctttacactgcaccatgaaacct | |
| acactccctggtatcatagcgcgtcatcacctcaacaagtcagtcgtctccattgatatt | |
| tgtacaaaaggtatacatggggaacacgtgttcattcattaagtccatcttgcgtgcagc | |
| tatatccctgattggttatttttcctttccttctgaggttctcatgtcattttcttcatc | |
| ggatgtgactaaaatttttctggtgtcttctgccctctctttaattttgcctcttgaggg | |
| gtagcagatgtgtcagtgcattttattacttgctgaaacattcaggcttacatttcttat | |
| tagtttagtattttaaaagatttaattttctgaatgaggcatttgaattgtaccagcaat | |
| ggacttttaaaaaattggatgtaaaaccattcagggtgatttttcttgtcagtggacagt | |
| gacgaacagagatttgaaatcccttacctccaataataagccattcagcctaaattcatt | |
| tttatgaataaatctcttctttctcatggtaaatgtggcttgtgccactcaaacactagt | |
| gaaagggtatgtacaaccgcaacatcaggccaggacaccatttatttaaccaagtaatgg | |
| aggagagtgaaacattttccaaggccttatttctttttcagaatgctttaagtgttgatt | |
| atgtgtgctgggtctctagagaagtttttatttgttacaatactgctgctttgagcaatt | |
| ttgtttctcttcattgtctgttaggggaatcaccagctttggcatcttaacaacaaacag | |
| tggatgggtaatttttatttctgatacgcatctttagagtcaaatatatcttttccctgt | |
| actcctcatgtacaaccaaagaacatacattatgaaaattgtattataatatctggaaac | |
| acaacattttcctctggcagttacttttgtacgaaatgaaaaaaaaaaaaaaaa | |

TABLE 152

| | |
|---|---|
| cbe_3474077 KRT4 | |
| keratin 4 | |
| length = 2617 | |
| gacttgctccggtttgcagagctaggaggtggcaggctgtgcgctcaaactcaggctgtc | SEQ ID NO: 143 |
| taactccacattctgtggggtgagaggatgggtgatggggtgtcttttctggaggaggga | |
| ggtgctgtgagcctagcgagatggaggtacagtgggtgtgggcctggagcgctgggccca | |
| ggcaggggcttctgattaggaagccctggggcaccagttcaggttctcccagagagtagt | |
| gtgatgggatccagtaacctgtgccctccagatgacttctgtaggtgtgtttagtgacat | |
| gctcaacgggtgcgggaaggatgggcttgtgccaagggccaagcccagagatgtttcaga | |
| tttttccctttatgcccctgcaaccaagccctgctgctccaggacatataagagacgaag | |
| gctgagggctccagcactcaccggcctgggccctgtcacttctctgatagctcccagctc | |
| gctctctgcagccatgattgccagacagcagtgtgtccgaggcgggccccggggcttcag | |
| ctgtggctcggccattgtaggcggtggcaagagaggtgccttcagctcagtctccatgtc | |
| tggaggtgctggccgatgctcttctgggggatttggcagcagaagcctctacaacctcag | |
| ggggaacaaaagcatctccatgagtgtggctgggtcacgacaaggtgcctgctttggggg | |
| tgctggaggctttggcactggtggctttggtgccggcggcttcggagctggtttcggcac | |
| tggtggctttggtggtggatttgggggctccttcagtggtaagggtggccctggcttccc | |
| cgtctgccccgctgggggaattcaggaggtcaccatcaaccagagcttgctcacccccct | |
| ccacgtggagattgaccctgagatccagaaagtccggacggaagagcgcgaacagatcaa | |
| gctcctcaacaacaagtttgcctccttcatcgacaaggtgcagttcttagagcaacagaa | |
| taaggtcctggagaccaaatggaacctgctccagcagcagacgaccaccacctccagcaa | |
| aaaccttgagcccctctttgagacctacctcagtgtcctgaggaagcagctagatacctt | |
| gggcaatgacaaagggcgcctgcagtctgagctgaagaccatgcaggacagcgtggagga | |
| cttcaagactaagtatgaagaggagatcaacaaacgcacagcagccgagaatgactttgt | |
| ggtcctaaagaaggacgtggatgctgcctacctgaacaaggtggagttggaggccaaggt | |
| ggacagtcttaatgacgagatcaacttcctgaaggtcctctatgatgcggagctgtccca | |
| gatgcagacccatgtcagcgacacgtccgtggtcctttccatggacaacaaccgcaacct | |
| ggacctggacagcattattgccgaggtccgtgcccagtacgaggagattgcccagaggag | |
| caaggctgaggctgaagccctgtaccagaccaaggtccagcagctccagatctcggttga | |
| ccaacatggtgacaacctgaagaacaccaagagtgaaattgcagagctcaacaggatgat | |
| ccagaggctgcgggcagagatcgagaacatcaagaagcagtgccagactcttcaggtatc | |
| cgtggctgatgcagagcagcgaggtgagaatgcccttaaagatgcccacagcaagcgcgt | |
| agagctggaggctgccctgcagcaggccaaggaggagctggcacgaatgctgcgtgagta | |
| ccaggagctcatgagtgtgaagctggccttggacatcgagatcgccacctaccgcaaact | |
| gctggagggcgaggagtacagaatgtctggagaatgccagagtgccgtgagcatctctgt | |
| ggtcagcggtagcaccagcactggaggcatcagcggaggattaggaagtggctccgggtt | |
| tggcctgagtagtggctttggctccggctctggaagtggctttgggtttggtggcagtgt | |
| ctctggcagttccagcagcaagatcatctctaccaccaccctgaacaagagacgatagag | |
| gagacgaggtccctgcagctcactgtgtccagctgggcccagcactggtgtctctgtgct | |
| tccttcacttcacctccatcctctgtctctggggctcatcttactagtatcccctccact | |
| atcccatgggctctctctgccccaggatgatcttctgtgctgggacagggactctgcctc | |
| ttggagtttggtagctacttcttgatttgggcctggtgacccacctggaatgggaaggat | |
| gtcagctgacctctcacctcccatgggcagagaagaaaatgaccaggagtgtcatctcca | |
| gaattattggggtcacatatgtcccttcccagtccaatgccatctcccactagatcctgt | |
| attatccatctacatcagaaccaaactacttctccaacacccggcagcacttggccctgc | |
| aagcttaggatgagaaccacttagtgtcccattctactcctctcattccctcttatccat | |
| ctgcaggtgaatcttcaataaaatgcttttgtcattc | |

TABLE 153

| | |
|---|---|
| cbe_3563252 LOC91689 | |
| hypothetical gene supported by AL449243 | |
| length = 1586 | |
| agcgggctttcttcccgagggcggcacgagggctgggcggtggggtgcgggtgcccgggt | SEQ ID NO: 144 |
| gaggggcggagctgggggcatggcgtccggagcggctcgctggctagtattggcacccgt | |
| caggtccggggctctccggagcgggcctagcttgaggaaagatggcgatgtctccgccgc | |
| atggagcggctcaggccggagcctggtaccgtcggggtcagtcatcgttacccgcagcgg | |
| cgccattttgcccaaaccggtgaaaatgtccttcggccttctgcgtgtgttctccattgt | |
| gatcccctttctctatgtcgggacactcattagcaagaactttgctgctctacttgagga | |
| acatgacatttttgttccagaggatgatgatgatgatgactaacaggaattacagaaagg | |
| agaaagcactaactgaagaaatggtgatgctctcagtttctctgccttccctatcagcag | |
| aaaggctcggggaaggccctcagcctcccagtctggtgaagcttcctgtatggtccatga | |
| ccgtattccaccccaggctctgggaggctccctgagatgtgctgtccactaagcactgca | |
| caaacaagcaatcaaattatgaataaacataataaatatcagccgtgcgtgactgagtga | |
| tggctgcagtttctcagtatccctaggttctagttggtgcagttgtctctgctgtccttt | |
| atttatgggagaaacataggcccaggctatccaggctgcagtggagcctggtgaactatt | |
| ctgggggccctgggaactattttcattgtttacaaaagcccaacagaaactgtgcatttt | |
| cccttaagaaagcttcatgggctaactaaagcctcatgccattctgtgttcagtgccagt | |
| catgacagctctgcttgttagcatactacttaaatataactagaatgattcaaaactcgg | |
| gttctgtgatatgaggatatagataggttttcatctatttcctggcttataactcccaaa | |
| acccttgttttaggcttttgttataatgttgggcacttcgggcctcagaaaacagcaggc | |
| tgtttctcagatcttctcctgacctcctttcacctgctgctttttctccccaaggcaggc | |
| catagaaactaaaagtataatcttcctttgcccatcttccagttggccataaaaagaatc | |
| ctctgacctaccttgtctgattttaggtcatgagacccccatttcagaagggattctgcc | |
| ccatacctgagaggaagaaatgtagacaggccttgttggacttccccactccatctgtat | |
| tagattatgcctcttttgtccaatcccatttctccagtgttgtccatgcttcaatcatcc | |
| ctatccaatgaggtctccataaaaggcccaagaagacaggtttagagagctttcggagaa | |
| cagaacacttggctttgcaaagtggcacgcctggagagaacttggaagctccacgcccct | |
| tctatacctcaccctatgcatctcttcagctgtatcttttgtgatatcctttataataaa | |
| ccagtaaacggaaaaaaaaaaaaaaa | |

TABLE 154

| | |
|---|---|
| cbe_3575702 SUI1 | |
| putative translation initiation factor | |
| length = 1324 | |
| cggcacgagcgccgccgaggattcagcagcctcccccttgagccccctcgcttcccgacg | SEQ ID NO: 145 |
| ttccgttcccccctgcccgccttctcccgccaccgccgccgccgccttccgcaggccgtt | |
| tccaccgaggaaaaggaatcgtatcgtatgtccgctatccagaacctccactctttcgac | |
| ccctttgctgatgcaagtaagggtgatgacctgcttcctgctggcactgaggattatatc | |
| catataagaattcaacagagaaacggcaggaagacccttactactgtccaagggatcgct | |
| gatgattacgataaaaagaaactagtgaaggcgtttaagaaaaagtttgcctgcaatggt | |
| actgtaattgagcatccggaatatggagaagtaattcagctacagggtgaccaacgcaag | |
| aacatatgccagttcctcgtagagattggactggctaaggacgatcagctgaaggttcat | |
| gggttttaagtgcttgtggctcactgaagcttaagtgaggatttccttgcaatgagtaga | |
| atttcccttctctcccttgtcacaggtttaaaaacctcacagcttgtataatgtaaccat | |
| ttggggtccgcttttaacttggactagtgtaactccttcatgcaataaactgaaaagagc | |
| catgctgtctagtcttgaagtccctcatttaaacagaggtcaagcaataggcgcctggca | |
| gtgtcaagcctgaaaccaagcaataccgtcatgtttcagccaagcccagagccctaagat | |
| tacaaacaactatggccggaacctcctcagctctccctctgcagagttccctaccctaag | |
| agaatgttaccacctgaacagtcctcggtgaatctgagaggagaggatggggtaaggcag | |
| aagcaccagctgtactactagaagggagcttttggtggtagatcccctggtgtctccaac | |
| ctgactaggtggacagagctcaaagaggccctcttaccgctagcgaggtgataggacatc | |
| tggcttgccacaaaggtctgttcgaccagacatatcctagctaagggatgtccaaacatc | |
| agaatgtgaggccaaccttctatcagagttaaacttttgacaagggaacaaatctcaaac | |
| tgatccatcagtcatgtagctagctgtagagcttgcaacttaatagcagcagctgcccaa | |
| tgccatgtgaagtaacaaactggtttttggtttttttttccccttcagttttaatgttat | |
| gtgtaatgtatttaaacccttatttaaataaaacttgttttcagaaaaaaaaaaaaaaaa | |
| aaaa | |

TABLE 155

| | |
|---|---|
| cbe_3580897 HSPA5 | |
| heat shock 70 kDa protein 5 (glucose-regulated protein, 78 kDa) | |
| length = 3925 | |
| acagcacagacagattgacctattggggtgtttcgcgagtgtgagagggaagcgccgcgg | SEQ ID NO: 146 |
| cctgtatttctagacctgcccttcgcctggttcgtggcgccttgtgaccccgggcccctg | |
| ccgcctgcaagtcggaaattgcgctgtgctcctgtgctacggcctgtggctggactgcct | |
| gctgctgcccaactggctggcaagatgaagctctccctggtggccgcgatgctgctgctg | |
| ctcagcgcggcgcgggccgaggaggaggacaagaaggaggacgtgggcacggtggtcggc | |
| atcgacctggggaccacctactcctgcgtcggcgtgttcaagaacggccgcgtggagatc | |
| atcgccaacgatcagggcaaccgcatcacgccgtcctatgtcgccttcactcctgaaggg | |
| gaacgtctgattggcgatgccgccaagaaccagctcacctccaaccccgagaacacggtc | |
| tttgacgccaagcggctcatcggccgcacgtggaatgacccgtctgtgcagcaggacatc | |
| aagttcttgccgttcaaggtggttgaaaagaaaactaaaccatacattcaagttgatatt | |
| ggaggtgggcaaacaaagacatttgctcctgaagaaatttctgccatggttctcactaaa | |
| atgaaagaaaccgctgaggcttatttgggaaagaaggttacccatgcagttgttactgta | |
| ccagcctattttaatgatgcccaacgccaagcaaccaaagacgctggaactattgctggc | |
| ctaaatgttatgaggatcatcaacgagcctacggcagctgctattgcttatggcctggat | |
| aagagggagggggagaagaacatcctggtgtttgacctgggtggcggaaccttcgatgtg | |
| tctcttctcaccattgacaatggtgtcttcgaagttgtggccactaatggagatactcat | |
| ctgggtggagaagactttgaccagcgtgtcatggaacacttcatcaaactgtacaaaaag | |
| aagacgggcaaagatgtcaggaaagacaatagagctgtgcagaaactccggcgcgaggta | |
| gaaaaggccaaacgggccctgtcttctcagcatcaagcaagaattgaaattgagtccttc | |
| tatgaaggagaagacttttctgagaccctgactcgggccaaatttgaagagctcaacatg | |
| gatctgttccggtctactatgaagcccgtccagaaagtgttggaagattctgatttgaag | |
| aagtctgatattgatgaaattgttcttgttggtggctcgactcgaattccaaagattcag | |
| caactggttaaagagttcttcaatggcaaggaaccatcccgtggcataaacccagatgaa | |
| gctgtagcgtatggtgctgctgtccaggctggtgtgctctctggtgatcaagatacaggt | |
| gacctggtactgcttgatgtatgtccccttacacttggtattgaaactgtgggaggtgtc | |
| atgaccaaactgattccaaggaacacagtggtgcctaccaagaagtctcagatcttttct | |
| acagcttctgataatcaaccaactgttacaatcaaggtctatgaaggtgaaagacccctg | |
| acaaaagacaatcatcttctgggtacatttgatctgactggaattcctcctgctcctcgt | |
| ggggtcccacagattgaagtcacctttgagatagatgtgaatggtattcttcgagtgaca | |
| gctgaagacaagggtacagggaacaaaaataagatcacaatcaccaatgaccagaatcgc | |
| ctgacacctgaagaaatcgaaaggatggttaatgatgctgagaagtttgctgaggaagac | |
| aaaaagctcaaggagcgcattgatactagaaatgagttggaaagctatgcctattctcta | |
| aagaatcagattggagataaagaaaagctgggaggtaaactttcctctgaagataaggag | |
| accatggaaaaagctgtagaagaaaagattgaatggctggaaagccaccaagatgctgac | |
| attgaagacttcaaagctaagaagaaggaactggaagaaattgttcaaccaattatcagc | |
| aaactctatggaagtgcaggccctcccccaactggtgaagaggatacagcagaaaaagat | |
| gagttgtagacactgatctgctagtgctgtaatattgtaaatactggactcaggaacttt | |
| tgttaggaaaaaattgaaagaacttaagtctcgaatgtaattggaatcttcacctcagag | |
| tggagttgaaactgctatagcctaagcggctgtttactgcttttcattagcagttgctca | |
| catgtctttgggtgggggggagaagaagaattggccatcttaaaaagcaggtaaaaaacc | |
| tgggttagggtgtgtgttcaccttcaaaatgttctatttaacaactgggtcatgtgcatc | |
| tggtgtaggaagttttttctaccataagtgacaccaataaatgtttgttatttacactgg | |
| tctaatgtttgtgagaagcttctaattagatcaattacttattttaggaaatttaagact | |
| agatactcgtgtgtggggtgaggggagggagtatttggtatgttgggataaggaaacact | |
| tctatttaatgcttccagggattttttttttttttttttaaccctcctgggcccaagtga | |
| tccttccacctcagtctcccagctaattgagaccacaggcttgttaccaccatgctcggc | |
| ttttgcattaatctaagaaaaggggagagaagttaatccacatctttactcaggcaaggg | |
| gcatttcacagtgcccaagagtggggttttcttgaacatacttggtttcctatttcccct | |
| tatctttctaaaactgcctttctggtggctttttttaaaattattactaatgatgctttt | |
| atagctgcttggattctctgagaaatgatggggagtgagtgatcactggtattaacttta | |
| tacacttggatttcatttgtaactttaggatgtaaaggtatattgtgaaccctagctgtg | |
| tcagaatctccatccctgaaatttctcattagtggtactggggtgggatcttggatggtg | |
| acattgaaactacactaaatcccctcactatgaatgggttgttaaaggcaatggtttgtg | |
| tcaaaactggtttaggattacttagattgtgttcctgaagaaaagagtccaggtaaatgg | |
| tatgatcaataaaggacaggctggtgctaacataaaatccaatattgtaatcctagcact | |
| ttgggaggccaaggcgggtggatcacaaggtcaagagatagagaccatctttgccaacat | |
| ggtgaaactccatctctactgaaaatacaaaaattagctgggcgtggtagtgcaagctga | |
| aggctgaggcaggagaatcactcgaacccgggaggcagaggttgcagtgagccgagatca | |
| caccactgtactccagcccggcactccagcctggcgacaagagtgagactccacctcaaa | |
| aaaaaaaaaaagaatccaatactgcccaaggataggtattttatagatgggcaactggct | |
| gaaaggttaattctctagggctagtagaactggatcccaacaccaaactcttaattagac | |
| ctaggcctcagctgcactgcccgaaaagcatttgggcagaccctgagcagaatactggtc | |
| tcaggccaagcccaatacagccattaaagatgacctacagtgctgtgtaccctggggcaa | |
| tagggttaaatggtagttagcaactagggctagtcttcccttacctcaaaggctctcact | |
| accgtggaccacctagtctgtaactctttctgaggagctgttactgaatattaaaaagat | |
| agacttcaaaaaaaaaaaaaaaaaa | |

TABLE 156

| | |
|---|---|
| cbe_3583511 MGC3222 | |
| hypothetical protein MGC3222 | |
| length = 5765 | |
| gtggtgtctcccgacagcatccacagtgtggctccggagaatgaaggaaggctggtgcac | SEQ ID NO: 147 |
| atcattggcgccttacggacatccaagcttttgtctgatccaaactatggggtccatctt | |
| ccggctgtgaaactgcggaggcacgtggagatgtaccaatgggtagaaactgaggagtcc | |
| agggagtacaccgaggatgggcaggtgaagaaggagacgaggtattcctacaacactgaa | |
| tggaggtcagaaatcatcaacagcaaaaacttcgaccgagagattggccacaataacccc | |
| agtgccatggcagtggagtcattcacggcaacagccccctttgtccaaattggcaggttt | |
| ttcctctcgtcaggcctcatcgacaaagtcgacaacttcaagtccctgagcctatccaag | |
| ctggaggaccctcatgtggacatcattcgccgtggagactttttctaccacagcgaaaat | |
| cccaagtatccagaggtgggagacttgcgtgtctccttttcctatgctggactgagcggc | |
| gatgaccctgacctgggcccagctcacgtggtcactgtgattgcccggcagcggggtgac | |
| cagctagtcccattctccaccaagtctggggataccttactgctcctgcaccacggggac | |
| ttctcagcagaggtgagtgctgtgccctactcgtacggtggaggaacaagcatgtccttc | |
| cttccttccagtggttatttaataagatcacactaccaggggtcatagccagtgaatgag | |
| gcaagaagaagaaatagcggaatgttgagtatgcctcatcaaagtgtttgaccaggagtg | |
| tttcagattccagatttttttggatttgcaggtatttgtatatacataatgagatctctt | |
| ggggatgagaaccaagtctaaacacagaatctatttatgtttcatgtatactttgtacac | |
| atcgcctgaaggtgattttatacaatgcaacccgtcacatggggtcaagtgtgggatttc | |
| cactagtggcgtcatgttggcattcagaaagttttggattttggagcacttcagcatttg | |
| ggtttttggattgtggatgctgagtctgtcatgaaccctcagtgtgcaggactgtgctag | |
| gcactaaactcactgtgcctcacagactgtccggtagaggagtgagagtgacagaaactc | |
| gtaagtactgatgcatcgtgccgagtgttggaaagaggagaacaggtgctctgataccag | |
| tggacgtcagagtagaacatccgtttctgcagtgatcaagcagggcctctgacaagtgag | |
| ttttgctgacgagaaggggttaaccctagaagaatcagtgatcagtcagttccagcaaga | |
| acagcccagtctgggcacgaaagggttcaccttctaagggggcacccggagtgctgtggc | |
| cacagcccagagcggggagagggagtgggtgggggggctccaccaaagtctgtgtccttc | |
| cagaacattaggagggcggggaagggttttgggcctcagctacctgagtatccctctccc | |
| attgtaaatagggagggctatgcctaccccgggtcccaaggagaaggggtgccaagatgt | |
| gctgattagttcagttctgtgccttaaaagttagcctgtcttctctaggcaggcaatcct | |
| tctctgcagcagaagaattgagtctttagtttttctgttgttgatgaacacaggtatccc | |
| tggtggggcactctatacctgccctaaccaggactgctggggttttcattccagagtggc | |
| cagaaagggtcaaggcagcagacaggcatcaggggttagctgtgaccttttgttcctatt | |
| attagatcctcagcagccatctgtcccattagcctccagcttgtggtggtgcttgggcct | |
| caccaactcctggtacctggccagggctagggtgtggtcaggctgttgcccaggagccca | |
| gttaccaacctcagggtagggctgccacctcccagcctagaaggccatgagtcacccttc | |
| caccagcagctgagggtccccagcgagtttctaaaattaggaggcccagtccaggccttg | |
| ctctctctcccagggctgaggcccagctcacgcagccgcccctccagagaggagtggatg | |
| gggcccatggcttaggtaatgccccagaaaggaaggccctgcagtgcagatgctcaggcg | |
| gcctccttaccccctgggtctcctgctgcttagcagccccaaagccctgtcttcccccag | |
| cccagcccccacccccacacaaggtctggggtctctgccaagtagaaaaaaggcccagag | |
| ccaggcaggcagctcagggcaaaagtcaggatgccccgattccagtcccaggggcaggag | |
| agtacagctgcccaagcaggcacatcaaataaatggtgagtgataaatgtgacctgtttt | |
| tgttgttacggactctgagagagggcccgattcagatcagagggtcagggaaagccctct | |
| gaagaaatggggttcaagcaaagctcaaaagaatatgtaggaagctggccaggcatgact | |
| gcaggcaatcgctgtaggcagagcaaacccacgcgcagatacacaggcgagaggacactt | |
| gacctggactggggacagcagccagcagggtcagggcactggcagcagggtcggtgtccg | |
| gggggtgtggcagggctctggcaggtgactgtgtggagaacacagagtgggtgatagaag | |
| cagggccagtgaggctcctgcgggcagacagtggtaagagactcgggaatatgtggcctg | |
| gactggatggggtggtaggggaggagcgagccatgtctttcccatagatgtgagtgggtg | |
| ggtgggtgtgccatgtgctgagatggagaaggcgccagtgtcagcactagacaggctgag | |
| atgtcaagtggaaatgtcaaggaggtggtggaggtggtgggagctgcagggctgtgaggc | |
| ccgggagagggccagagctgcagctttaaattggagggtcacggcatgtggaggccaagg | |
| aagcagatgggatgtccggggagagagtatggactgaagcagctaaagaattgcagcatc | |
| taatggacaggaagaggggcagcgacagcgatggagcctgagaaggagggtgcagtgtcc | |
| ccaagccatgtggagaggttctgagattaaacagatcacacacatagcgcagagcatgct | |
| gcccagcagacatcctccaacctgctcaggggtggtgggcagggtcctgctcattccagg | |
| tgagaccgtgggctgagggctccaggtgctggcaggctctgagctgaggaaggcccgtac | |
| gttccacagagatctcatgccttgccctgtgctctgccagatgggcagaaggacttagcc | |
| ctataggaggcttgcccccgctccgtctggcctgttcagaaatggccaacagctcccgag | |
| ttggtacagccccctcaggacctgccctgccgactgggtacgccactggccctcagcatc | |
| ctgacctgcccccaccttgtcctgcaggaggtgtttcatagagaactaaggagcaactcc | |
| atgaagacctggggcctgcgggcagctggctggatggccatgttcatgggcctcaacctt | |
| atgacacggatcctctacaccttggtggactggtttcctgttttccgagacctggtcaac | |
| attggcctgaaagcctttgccttctgtgtggccacctcgctgaccctgctgaccgtggcg | |
| gctggctggctcttctaccgacccctgtgggccctcctcattgccggcctggcccttgtg | |
| cccatccttgttgctcggacacgggtgccagccaaaaagttggagtgaaaagaccctggc | |
| acccgcccgacacctgcgtgagccctaggatccaggtcctctctcacctctgacccagct | |
| ccatgccagagcaggagccccggtcaattttggactctgcaccccctctcctcttcaggg | |
| gccagacttggcagcatgtgcaccaggttggtgttcaccagctcatgtcttccccacatc | |
| tcttcttgccagtaagcagctttggtgggcagcagcagctcatgaatggcaagctgacag | |
| cttctcctgctgtttccttcctctcttggactgagtgggtacggccagccactcagccca | |
| ttggcagctgacaacgcagacacgctctacggaggcctgctgataaagggctcagccttg | |
| ccgtgtgctgcttctcatcactgcacacaagtgccatgctttgccaccaccaccaagcac | |
| atctgtgatcctgaagggcggccgttagtcattactgctgagtcctgggtcaccagcaga | |
| cacactgggcatggacccctcaaagcaggcacacccaaaacacaagtctgtggctagaac | |
| ctgatgtggtgtttaaaagagaagaaacactgaagatgtcctgaggagaaaagctggaca | |
| tatactgggcttcacacttatcttatggcttggcagaatctttgtagtgtgtgggatctc | |
| tgaaggccctatttaagtttttcttcgttactttgctgcttcatgtgtactttcctaccc | |
| caagaggaagttttctgaaataagatttaaaaacaaaacaaaaaaaacacttaatatttc | |
| agactgttacaggaaacaccctttagtctgtcagttgaattcagagcactgaaaggtgtt | |
| aaattggggtatgtggtttgattgataaaaagttacctctcagtattttgtgtcactgag | |
| aagctttacaatggatgcttttgaaacaagtatcagcaaaaggatttgttttcactctgg | |
| gaggagagggtggagaaagcacttgctttcatcctctggcatcggaaactcccctatgca | |
| cttgaagatggtttaaaagattaaagaaacgattaagagaaaaggttggaagctttatac | |
| taaatgggctccttcatggtgacgccccgtcaaccacaatcaagaactgaggcctgaggc | |
| tggttgtacaatgcccacgcctgcctggctgctttcacctgggagtgctttcgatgtggg | |
| cacctgggcttcctagggctgcttctgagtggttctttcacgtgttgtgtccatagcttt | |
| agtcttcctaaataagatccacccacacctaagtcacagaatttctaagttccccaacta | |
| ctctcacacccttttaaagataaagtatgttgtaaccaggatgtcttaaatgattctttg | |
| tgtaccttttctgtcatattcagaaaccgttttgtgcctgctgggagtaattcctttagc | |
| aattaagtatttggtagctgaataaggggtcagaacttctgaaaccagagatctgtaatc | |
| atctctattggcctggggtgcctgtgctataaatgagtttcttcacatgaaaaacacagc | |
| cagcccaagatgacttatctgggtttaggattcaatagtattcactaactgcttattaca | |
| tgagcaatttcatcaaatctccaaactcttaaaggatgctttcggaaaacacgctgtata | |
| cctagatgatgactaaatgcaaaatccttgggctttggtttttttctagtaaggatttta | |
| aataactgccgacttcaaaagtgttcttaaaacgaaagataatgttaagaaaaatttgaa | |
| agctttggaaaaccaaatttgtaatatcattgtattttttattaaaagttttgtaataaa | |
| tttct | |

TABLE 157

| | |
|---|---|
| cbe_3590661 NUP214 | |
| nucleoporin 214 kDa | |
| length = 6614 | |
| ctgcgcgccgctggcgctgaggggaggaagtttgctgtcgagcggcctgggttccgtggg | SEQ ID NO: 148 |
| caaggccgtgggaggcagcgttggctgcttcgacacactgagggcggcgcgatgggagac | |
| gagatggatgccatgattcccgagcgggagatgaaggattttcagtttagagcgctaaag | |
| aaggtgagaatctttgactcccctgaggaattgcccaaggaacgctcgagtctgcttgct | |
| gtgtccaacaaatatggtctggtcttcgctggtggagccagtggcttgcagatttttcct | |
| actaaaaatcttcttattcaaaataaacccggagatgatcccaacaaaatagttgataaa | |
| gtccaaggcttgctagttcctatgaaattcccaatccatcacctggccttgagctgtgat | |
| aacctcacactctctgcgtgcatgatgtccagtgaatatggttccattattgcttttttt | |
| gatgttcgcacattctcaaatgaggctaaacagcaaaaacgcccatttgcctatcataag | |
| cttttgaaagatgcaggaggcatggtgattgatatgaagtggaaccccactgtcccctcc | |
| atggtggcagtttgtctggctgatggtagtattgctgtcctgcaagtcacggaaacagtg | |
| aaagtatgtgcaactcttccttccacggtagcagtaacctctgtgtgctggagccccaaa | |
| ggaaagcagctggcagtgggaaaacagaatggaactgtggtccagtatcttcctactttg | |
| caggaaaaaaaagtcattccttgtcctccgttttatgagtcagatcatcctgtcagagtt | |
| ctggatgtgctgtggattggtacctacgtcttcgccatagtgtatgctgctgcagatggg | |
| accctggaaacgtctccagatgtggtgatggctctactaccgaaaaaagaagaaaagcac | |
| ccagagatatttgtgaactttatggagccctgttatggcagctgcacggagagacagcat | |
| cattactacctcagttacattgaggaatgggatttagtgctggcagcatctgcggcttca | |
| acagaagttagtatccttgctcgacaaagtgatcagattaattgggaatcttggctactg | |
| gaggattctagtcgagctgaattgcctgtgacagacaagagtgatgactccttgcccatg | |
| ggagttgtcgtagactatacaaaccaagtggaaatcaccatcagtgatgaaaagactctt | |
| cctcctgctccagttctcatgttactttcaacagatggtgtgctttgtccattttatatg | |
| attaatcaaaatcctggggttaagtctctcatcaaaacaccagagcgactttcattagaa | |
| ggagagcgacagcccaagtcaccaggaagtactcccactaccccaacctcctctcaagcc | |
| ccacagaaactggatgcttctgcagctgcagcccctgcctctctgccaccttcatcacct | |
| gctgctcccattgccactttttctttgcttcctgctggtggagcccccactgtgttctcc | |
| tttggttcttcatctttgaagtcatctgctacggtcactggggagcccccttcatattcc | |
| agtggctccgacagctccaaagcagccccaggccctggcccatcaaccttctcttttgtt | |
| cccccttctaaagcctccctagcccccacccctgcagcgtctcctgtggctccatcagct | |
| gcttcattctcctttggatcatctggttttaagcctaccctggaaagcacaccagtgcca | |
| agtgtgtctgctccaaatatagcaatgaagccctccttcccaccctcaacctctgctgtc | |
| aaagtcaaccttagtgaaaagtttactgctgcagctacctctactcctgttagtagctcc | |
| cagagcgcacccccgatgtcgccattctcttctgcctccaagccagctgcttctggacca | |
| ctcagccaccccacacctctctcagcaccacctagttccgtgccattgaagtcctcagtc | |
| ttgccctcaccatcaggacgatctgctcagggcagttcaagcccagtgccctcaatggta | |
| cagaaatcacccaggataacccctccagcggcaaagccaggctctccccaggcaaagtca | |
| cttcagcctgctgttgcagaaaagcagggacatcagtggaaagattcagatcctgtaatg | |
| gctggaattggggaggagattgcacactttcagaaggagttggaagagttaaaagcccga | |
| acttccaaagcctgtttccaagtgggcacttctgaggagatgaagatgctgcgaacagaa | |
| tcagatgacttgcatacctttcttttggagattaaagagaccacagagtcgcttcatgga | |
| gatataagtagcctgaaaacaactttacttgagggctttgctggtgttgaggaagccaga | |
| gaacaaaatgaaagaaatcgtgactctggttatctgcatttgctttataaaagaccactg | |
| gatcccaagagtgaagctcagcttcaggaaattcggcgccttcatcagtatgtgaaattt | |
| gctgtccaagatgtgaatgatgttctagacttggagtgggatcagcatctggaacaaaag | |
| aaaaaacaaaggcacctgcttgtgccagagcgagagacactgtttaacaccctagccaac | |
| aatcgggaaatcatcaaccaacagaggaagaggctgaatcacctggtggatagtcttcag | |
| cagctccgcctttacaaacagacttccctgtggagcctgtcctcggctgttccttcccag | |
| agcagcattcacagttttgacagtgacctggaaagcctgtgcaatgctttgttgaaaacc | |
| accatagaatctcacaccaaatccttgcccaaagtaccagccaaactgtcccccatgaaa | |
| caggcacaactgagaaacttcttggccaagaggaagaccccaccagtgagatccactgct | |
| ccagccagcctgtctcgatcagcctttctgtctcagagatattatgaagacttggatgaa | |
| gtcagctcaacgtcatctgtctcccagtctctggagagtgaagatgcacggacgtcctgt | |
| aaagatgacgaggcagtggttcaggcccctcggcacgcccccgtggttcgcactccttcc | |
| atccagcccagtctcttgccccatgcagcaccttttgctaaatctcacctggttcatggt | |
| tcttcacctggtgtgatgggaacttcagtggctacatctgctagcaaaattattcctcaa | |
| ggggccgatagcacaatgcttgccacgaaaaccgtgaaacatggtgcacctagtccttcc | |
| caccccatctcagccccgcaggcagctgccgcagcagcactcaggcggcagatggccagt | |
| caggcaccagctgtaaacactttgactgaatcaacgttgaagaatgtccctcaagtggta | |
| aatgtgcaggaattgaagaataaccctgcaaccccttctacagccatgggttcttcagtg | |
| ccctactccacagccaaaacacctcacccagtgttgaccccagtggctgctaaccaagcc | |
| aagcaggggtctctaataaattcccttaagccatctgggcctacaccagcatccggtcag | |
| ttatcatctggtgacaaagcttcagggacagccaagatagaaacagctgtgacttcaacc | |
| ccatctgcttctgggcagttcagcaagcctttctcattttctccatcagggactggcttt | |
| aattttgggataatcacaccaacaccgtcttctaatttcactgctgcacaaggggcaaca | |
| ccctccactaaagagtcaagccagccggacgcattctcatctggtgggggaagcaaacct | |
| tcttatgaggccattcctgaaagctcacctccctcaggaatcacatccgcatcaaacacc | |
| accccaggagaacctgccgcatctagcagcagacctgtggcaccttctggaactgctctt | |
| tccaccacctctagtaagctggaaaccccaccgtccaagctgggagagcttctgtttcca | |
| agttctttggctggagagactctgggaagtttttcaggactgcgggttggccaagcagat | |
| gattctacaaaaccaaccaataaggcttcatccacaagcctaactagtacccagccaacc | |
| aagacgtcaggcgtgccctcagggtttaattttactgcccccccggtgttagggaagcac | |
| acggagccccctgtgacatcctctgcaaccaccacctcagtagcaccaccagcagccacc | |
| agcacttcctcaactgccgtttttggcagtctgccagtcaccagtgcaggatcctctggg | |
| gtcatcagttttggtgggacatctctaagtgctggcaagactagtttttcatttggaagc | |
| caacagaccaatagcacagtgcccccatctgccccaccaccaactacagctgccactccc | |
| cttccaacatcattccccacattgtcatttggtagcctcctgagttcagcaactaccccc | |
| tccctgcctatgtccgctggcagaagcacagaagaggccacttcatcagctttgcctgag | |
| aagccaggtgacagtgaggtctcagcatcagcagcctcacttctagaggagcaacagtca | |
| gcccagcttccccaggctcctccgcaaacttctgactctgttaaaaaagaacctgttctt | |
| gcccagcctgcagtcagcaactctggcactgcagcatctagtactagtcttgtagcactt | |
| tctgcagaggctaccccagccaccacgggggtccctgatgccaggacggaggcagtacca | |
| cctgcttcctccttttctgtgcctgggcagactgctgtcacagcagctgctatctcaagt | |
| gcaggccctgtggccgtcgaaacatcaagtacccccatagcctccagcaccacgtccatt | |
| gttgctcccggcccatctgcagaggcagcagcatttggtaccgtcacttctggctcatcc | |
| gtctttgctcagcctcctgctgccagttctagctcagctttcaaccagctcaccaacaac | |
| acagccactgccccctctgccacgcccgtgtttgggcaagtggcagccagcaccgcacca | |
| agtctgtttgggcagcagactggtagcacagccagcacagcagctgccacaccacaggtc | |
| agcagctcagggtttagcagcccagcttttggtaccacagccccaggggtctttggacag | |
| acaaccttcgggcaggcctcagtctttgggcagtcggcgagcagtgctgcaagtgtcttt | |
| tccttcagtcagcctgggttcagttccgtgcctgccttcggtcagcctgcttcctccact | |
| cccacatccaccagtggaagtgtctttggtgccgcctcaagtaccagtagctccagttcc | |
| ttctcatttggacagtcttctcccaacacaggaggggggctgtttggccaaagcaacgct | |
| cctgcttttgggcagagtcctggctttggacagggaggctctgtctttggtggtacctca | |
| gctgccaccacaacagcagcaacctctgggttcagcttttgccaagcttcaggttttggg | |
| tctagtaatactggttctgtgtttggtcaagcagccagtactggtggaatagtctttggc | |
| cagcaatcatcctcttccagtggtagcgtgtttgggtctggaaacactggaagaggggga | |
| ggtttcttcagtggccttggaggaaaacccagtcaggatgcagccaacaaaaacccattc | |
| agctcggccagtgggggctttggatccacagctacctcaaatacctctaacctatttgga | |
| aacagtggggccaagacatttggtggatttgccagctcgtcgtttggagagcagaaaccc | |
| actggcactttcagctctggaggaggaagtgtggcatcccaaggctttgggttttcctct | |
| ccaaacaaaacaggtggcttcggtgctgctccagtgtttggcagccctcctacttttggg | |
| ggatcccctgggtttggaggggtgccagcattcggttcagccccagcctttacaagccct | |
| ctgggctcgacgggaggcaaagtgttcggagagggcactgcagctgccagcgcaggagga | |
| ttcgggtttgggagcagcagcaacaccacatccttcggcacgctcgcgagtcagaatgcc | |
| cccactttcggatcactgtcccaacagacttctggttttgggacccagagtagcggattc | |
| tctggttttggatcaggcacaggagggttcagctttgggtcaaataactcgtctgtccag | |
| ggttttggtggctggcgaagctgagggcgtgtcagcaggcctttcgatccctgggaccaa | |
| ccgcatcctcagcttcttccccgagaaatgctggagcaggctgttcagaccgacgttgcc | |
| atcaaaacacatacacccagaaagaaacaacagaaaccaaaactcacaaggcgcatgatt | |
| acttgttttatatttcatgttgggttttccctcccactattaaacagtctgtttccgtac | |
| aaaaaaaaaaaaaa | |

TABLE 158

| | |
|---|---|
| cbe_3606990 Prey1053670 | |
| Homo sapiens, Similar to CG5604 gene product, clone MGC: 11330, mRNA, complete | |
| cds. | |
| length = 1206 | |
| ggcacgaggctcaggaaaccagggctgtcacgtgtataacctcaggtttgtcacgtgtat | SEQ ID NO: 149 |
| aacctcaggaaaccagggctgtcacgtgtataacccaggtttcctgaggtttgtgagggt | |
| tttatgtggcatgtcttctgatgaaaggaaagcattcttgcagtttaccactggttgttc | |
| aactctacccccaggtggactggctaacctgcatcccaggctcacggttgtacgcaaggt | |
| tgatgctactgatgcaagctatccatcagtcaatacatgtgtgcattaccttaagttgcc | |
| tgaatattcttccgaggagatcatgagagagcgcctgctagctgctacaatggagaaagg | |
| ctttcatttcaattgagctttgaagtgcaatgggagacatcagagactttaaaaatacta | |
| gtgaagcctcttgtgtttgtgtgcagagaagtatatgatccaccatgctaatgacacttg | |
| cctttttttccaccattaaggctttaagaacatgtggaataagttttttagctgctaatg | |
| acaaaacaaatcctgtaactacccagccagcaagtatatagcacagaacactgtgttact | |
| ttacaagggcttatgtgactggaataaggtggtcccacttgactgttccaaagagcagct | |
| tctcagatcttcagtgttcactggtaaatttctaacagtgtatttgtgtaaagtttgtca | |
| tttcatactccatacactacagttgctgtcactgatccctgttttgctggcttttaagct | |
| acttggtcaaaaatcctgcttccttaaaacatagagaattaatgagcatctcaagctttt | |
| tcttttcctttttaatgatgcctgcactatcaagagtattctagtgttctctctttgttt | |
| ggcatataatcatgcaccaaactttttatttctttaaggtgggagtatatttttatttcc | |
| taaatgccatactatgaagatcaaagtcttaagtgtgtttgcagctcaaaaataaagatg | |
| tattaaggggggaaaacctggtctaagtgcaaggcacacttacagcgagttttactttcg | |
| gttgtattttctttgtatattataaacatttatttaacttgttgccgtttgaagtaaaaa | |
| atttccaaaatgtatgctcaacaataatcattaaaatgtttgcagcgtaaaaaaaaaaaa | |
| aaaaaa | |

TABLE 159

| | |
|---|---|
| cbe_3621798 XPO1 | |
| exportin 1 (CRM1 homolog, yeast) | |
| length = 4148 | |
| aggaaggaaggagcagttggttcaatctctggtaatctatgccagcaattatgacaatgt | SEQ ID NO: 150 |
| tagcagaccatgcagctcgtcagctgcttgatttcagccaaaaactggatatcaacttat | |
| tagataatgtggtgaattgcttataccatggagaaggagcccagcaaagaatggctcaag | |
| aagtactgacacatttaaaggagcatcctgatgcttggacaagagtcgacacaattttgg | |
| aattttctcagaatatgaatacgaaatactatggactacaaattttggaaaatgtgataa | |
| aaacaaggtggaagattcttccaaggaaccagtgcgaaggaataaaaaaatacgttgttg | |
| gcctcattatcaagacgtcatctgacccaacttgtgtagagaaagaaaaggtgtatatcg | |
| gaaaattaaatatgatccttgttcagatactgaaacaagaatggcccaaacattggccaa | |
| cttttatcagtgatattgttggagcaagtaggaccagcgaaagtctctgtcaaaataata | |
| tggtgattcttaaactcttgagtgaagaagtatttgatttctctagtggacagataaccc | |
| aagtcaaatctaagcatttaaaagacagcatgtgcaatgaattctcacagatatttcaac | |
| tgtgtcagtttgtaatggaaaattctcaaaatgctccacttgtacatgcaaccttggaaa | |
| cattgctcagatttctgaactggattcccctgggatatatttttgagaccaaattaatca | |
| gcacattgatttataagttcctgaatgttccaatgtttcgaaatgtctctctgaagtgcc | |
| tcactgagattgctggtgtgagtgtaagccaatatgaagaacaatttgtaacactattta | |
| ctctgacaatgatgcaactaaagcagatgcttcctttaaataccaatattcgacttgcgt | |
| actcaaatggaaaagatgatgaacagaacttcattcaaaatctcagtttgtttctctgca | |
| cctttcttaaggaacatgatcaacttatagaaaaaagattaaatctcagggaaactctta | |
| tggaggcccttcattatatgttgttggtatctgaagtagaagaaactgaaatctttaaaa | |
| tttgtcttgaatactggaatcatttggctgctgaactctatagagagagtccattctcta | |
| catctgcctctccgttgctttctggaagtcaacattttgatgttcctcccaggagacagc | |
| tatatttgcccatgttattcaaggtccgtttattaatggttagtcgaatggctaaaccag | |
| aggaagtattggttgtagagaatgatcaaggagaagttgtgagagaattcatgaaggata | |
| cagattccataaatttgtataagaatatgagggaaacattggtttatcttactcatctgg | |
| attatgtagatacagaaagaataatgacagagaagcttcacaatcaagtgaatggtacag | |
| agtggtcatggaaaaatttgaatacattgtgttgggcaataggctccattagtggagcaa | |
| tgcatgaagaggacgaaaaacgatttcttgttactgttataaaggatctattaggattat | |
| gtgaacagaaaagaggcaaagataataaagctattattgcatcaaatatcatgtacatag | |
| taggtcaatacccacgttttttgagagctcactggaaatttctgaagactgtagttaaca | |
| agctgttcgaattcatgcatgagacccatgatggagtccaggatatggcttgtgatactt | |
| tcattaaaatagcccaaaaatgccgcaggcatttcgttcaggttcaggttggagaagtga | |
| tgccatttattgatgaaattttgaacaacattaacactattatttgtgatcttcagcctc | |
| aacaggttcatacgttttatgaagctgtggggtacatgattggtgcacaaacagatcaaa | |
| cagtacaagagcacttgatagaaaagtacatgttactccctaatcaagtgtgggatagta | |
| taatccagcaggcaaccaaaaatgtggatatactgaaagatcctgaaacagtcaagcagc | |
| ttggtagcattttgaaaacaaatgtgagagcctgcaaagctgttggacacccctttgtaa | |
| ttcagcttggaagaatttatttagatatgcttaatgtatacaagtgcctcagtgaaaata | |
| tttctgcagctatccaagctaatggtgaaatggttacaaagcaaccattgattagaagta | |
| tgcgaactgtaaaaagggaaactttaaagttaatatctggttgggtgagccgatccaatg | |
| atccacagatggtcgctgaaaattttgttccccctctgttggatgcagttctcattgatt | |
| atcagagaaatgtcccagctgctagagaaccagaagtgcttagtactatggccataattg | |
| tcaacaagttagggggacatataacagctgaaatacctcaaatatttgatgctgtttttg | |
| aatgcacattgaatatgataaataaggactttgaagaatatcctgaacatagaacgaact | |
| ttttcttactacttcaggctgtcaattctcattgtttcccagcattccttgctattccac | |
| ctacacagtttaaacttgttttggattccatcatttgggctttcaaacatactatgagga | |
| atgtcgcagatacgggcttacagatactttttacactcttacaaaatgttgcacaagaag | |
| aagctgcagctcagagtttttatcaaacttatttttgtgatattctccagcatatctttt | |
| ctgttgtgacagacacttcacatactgctggtttaacaatgcatgcatcaattcttgcat | |
| atatgtttaatttggttgaagaaggaaaaataagtacatcattaaatcctggaaatccag | |
| ttaacaaccaaatctttcttcaggaatatgtggctaatctccttaagtcggccttccctc | |
| acctacaagatgctcaagtaaagctctttgtgacagggcttttcagcttaaatcaagata | |
| ttcctgctttcaaggaacatttaagagatttcctagttcaaataaaggaatttgcaggtg | |
| aagacacttctgatttgtttttggaagagagagaaatagccctacggcaggctgatgaag | |
| agaaacataaacgtcaaatgtctgtccctggcatctttaatccacatgagattccagaag | |
| aaatgtgtgattaaaatccaaattcatgctgttttttttctctgcaactccgttagcaga | |
| ggaaaacagcatgtgggtatttgtcgaccaaaatgatgccaatttgtaaattaaaatgtc | |
| acctagtggccctttttcttatgtgtttttttgtataagaaattttctgtgaaatatcct | |
| tccattgtttaagcttttgttttggtcatctttatttagtttgcatgaagttgaaaatta | |
| aggcatttttaaaaattttacttcatgcccatttttgtggctgggctggggggaggaggc | |
| aaattcaatttgaacatatacttgtaattctaatgcaaaattatacaatttttcctgtaa | |
| acaataccaatttttaattagggagcattttccttctagtctatttcagcctagaagaaa | |
| agataatgagtaaaacaaattgcgttgtttaaaggattatagtgctgcattgtctgaagt | |
| tagcacctcttggactgaatcgtttgtctagactacatgtattacaaagtctctttggca | |
| agattgcagcaagatcatgtgcatatcatcccattgtaaagcgacttcaaaaatatggga | |
| acacagttagttatttttacacagttctttttgtttttgtgtgtgtgtgctgtcgcttgt | |
| cgacaacagctttttgttttcctcaatgaggagtgttgctcatttgtgagccttcattaa | |
| ctcgaagtgaaatggttaaaaatatttatcctgttagaataggctgcatctttttaacaa | |
| ctcattaaaaaacaaaacaactctggcttttgagatgacttatactaatttacattgttt | |
| accaagctgtagtgctttaagaacactacttaaaaagcaaaataaacttggtttacattt | |
| aaaaaaaa | |

TABLE 160

| | |
|---|---|
| cbe_3621899 PCNP | |
| PEST-containing nuclear protein | |
| length = 602 | |
| ttttttttttttgagtttgacaaatttattggtactttagtaaagacattcatctcagtc | SEQ ID NO: 151 |
| atttctctctcccagcttgaccttaggttaatatttcatttgggtcaagaaaataatacg | |
| taggagaggtatgttattttaacaaacaggaaaatggacaaaaaattgatagtttgccta | |
| cattaaagtaagttaaattcatgtatcgatataattaaatcatgtaagaactaagagttc | |
| tatatacatttccattgtcttacttgggcttattctaaacttaaatgctagtgaacaaag | |
| tgttaggaatatacacaggctgcttctctggagttattaccaactaaaagacctcagcaa | |
| gcagctaccaccaataaaacagtctgaagctgcctccaaatataatattgtaggagtttt | |
| caaggaaatttttatactgtatgcntttgtcngtgactatgcntttaaagatgtgtttaa | |
| cggcnaatttaaaaaaagttcntataccatccaaaattcnaaaccacnccctaccatacc | |
| caatctaccaangtatatgtggtgngatcccaaaatggttggaagatgcatttccttccc | |
| cc | |

TABLE 161

| | |
|---|---|
| cbe_3625721 EIF2B2 | |
| eukaryotic translation initiation factor 2B, subunit 2 beta, 39 kDa | |
| length = 1522 | |
| caggtgtggattccgccggtgaaggctgaaggcagctaccttaaagatgccgggatccgc | SEQ ID NO: 152 |
| agcgaagggctcggagttgtcagagaggatcgagagcttcgtggagaccctgaagcgggg | |
| tggtgggccgcgcagctccgaggaaatggctcgggagaccctagggttgctgcgccagat | |
| catcacggaccaccgctggagcaacgcgggggagctgatggagctgatccgcagagaggg | |
| caggaggatgacggccgctcagccctccgagaccaccgtgggcaacatggtgcggagagt | |
| gctcaagattatccgggaggagtatggcagactccatggacgcagcgacgagagtgatca | |
| gcaggagtccctgcacaaactgttgacatccggaggcctaaacgaggatttcagcttcca | |
| ttatgcccaactccagtccaacatcattgaggcgattaatgagctgctagtggagctgga | |
| agggacaatggagaacattgcagcccaggctctggagcacattcactccaatgaggtgat | |
| catgaccattggcttctcccgaacagtagaggccttcctcaaagaggctgcccgaaagag | |
| gaaattccatgtcattgtagcagagtgtgctcctttctgccagggtcatgaaatggctgt | |
| gaatttgtccaaagcaggtattgagacaactgtcatgactgatgctgccatttttgccgt | |
| tatgtcaagagtcaacaaggtgatcattggcacgaagaccatcctggccaatggggccct | |
| gagagctgtgacaggaactcacactctggcactggcagcaaaacaccattccaccccact | |
| catcgtctgtgcacctatgttcaaactttctccacagttccccaatgaagaagactcatt | |
| tcataagtttgtggctcctgaagaagtcctgccattcacagaaggggacattctggagaa | |
| ggtcagcgtgcattgccctgtgtttgactacgttcccccagagctcattaccctctttat | |
| ctccaacattggtgggaatgcaccttcctacatctaccgcctgatgagtgaactctacca | |
| tcctgatgatcatgttttatgaccgaccacacgtgtcctaagcagattgcttaggcagat | |
| acagaatgaagaggagacttgagtgttgctgctgaagcacatccttgcaatgtgggagtg | |
| cacaggagtccacctaaaaaaaaaatccttgatactgttgcctgcctttttagtcacccc | |
| gtaacaagggcacacatccagcactgtgtcttgcctttcagatcttaacagagcagcagg | |
| gcttaacttgttgattttggagcctcttagtgacctggttgcgtctgtgtcaggaactta | |
| aactttctggttcagtagtgtgttaaacataacactgaataccttactgggatacagatt | |
| tttgctcagaaatggctatgacactttttctaggctctaccaataaaagccacttgaagg | |
| ttcaaaaaaaaaaaaaaaaaaa | |

TABLE 162

| | |
|---|---|
| cbe_3641816 ANKH | |
| ankylosis, progressive homolog (mouse) | |
| length = 4031 | |
| ctcttttttttcccggcagatctttgttgtgtgggagggcagcagggatggacttgagct | SEQ ID NO: 153 |
| tgcggatcccctgctagagcagccgcgctcggagaaggcgccgcagccgcgaggaggagc | |
| cgccgccgccgcgcccgaggccccgccgcccgcggcctctgtcggcccgcgccccgctcg | |
| cccgtcgcccccgtcgcccctcgcctccccgcagagtcccctcgcggcagcagatgtgtg | |
| tggggtcagcccacggcggggactatggtgaaattcccggcgctcacgcactactggccc | |
| ctgatccggttcttggtgcccctgggcatcaccaacatagccatcgacttcggggagcag | |
| gccttgaaccggggcattgctgctgtcaaggaggatgcagtcgagatgctggccagctac | |
| gggctggcgtactccctcatgaagttcttcacgggtcccatgagtgacttcaaaaatgtg | |
| ggcctggtgtttgtgaacagcaagagagacaggaccaaagccgtcctgtgtatggtggtg | |
| gcaggggccatcgctgccgtctttcacacactgatagcttatagtgatttaggatactac | |
| attatcaataaactgcaccatgtggacgagtcggtggggagcaagacgagaagggccttc | |
| ctgtacctcgccgcctttcctttcatggacgcaatggcatggacccatgctggcattctc | |
| ttaaaacacaaatacagtttcctggtgggatgtgcctcaatctcagatgtcatagctcag | |
| gttgtttttgtagccattttgcttcacagtcacctggaatgccgggagcccctgctcatc | |
| ccgatcctctccttgtacatgggcgcacttgtgcgctgcaccaccctgtgcctgggctac | |
| tacaagaacattcacgacatcatccctgacagaagtggcccggagctggggggagatgca | |
| acaataagaaagatgctgagcttctggtggcctttggctctaattctggccacacagaga | |
| atcagtcggcctattgtcaacctctttgtttcccgggaccttggtggcagttctgcagcc | |
| acagaggcagtggcgattttgacagccacataccctgtgggtcacatgccatacggctgg | |
| ttgacggaaatccgtgctgtgtatcctgctttcgacaagaataaccccagcaacaaactg | |
| gtgagcacgagcaacacagtcacggcagcccacatcaagaagttcaccttcgtctgcatg | |
| gctctgtcactcacgctctgtttcgtgatgttttggacacccaacgtgtctgagaaaatc | |
| ttgatagacatcatcggagtggactttgcctttgcagaactctgtgttgttcctttgcgg | |
| atcttctccttcttcccagttccagtcacagtgagggcgcatctcaccgggtggctgatg | |
| acactgaagaaaaccttcgtccttgcccccagctctgtgctgcggatcatcgtcctcatc | |
| gccagcctcgtggtcctaccctacctgggggtgcacggtgcgaccctgggcgtgggctcc | |
| ctcctggcgggctttgtgggagaatccaccatggtcgccatcgctgcgtgctatgtctac | |
| cggaagcagaaaaagaagatggagaatgagtcggccacggagggggaagactctgccatg | |
| acagacatgcctccgacagaggaggtgacagacatcgtggaaatgagagaggagaatgaa | |
| taaggcacgggacgccatgggcactgcagggacagtcagtcaggatgacacttcggcatc | |
| atctcttccctctcccatcgtattttgttcccttttttttgttttgttttggtaatgaaa | |
| gaggccttgatttaaaggtttcgtgtcaattctctagcatactgggtatgctcacactga | |
| cggggggacctagtgaatggtctttactgttgctatgtaaaaacaaacgaaacaactgac | |
| ttcatacccctgcctcacgaaaacccaaaagacacagctgcctcacggttgacgttgtgt | |
| cctcctcccctggacaatctcctcttggaaccaaaggactgcagctgtgccatcgcgcct | |
| cggtcaccctgcacagcaggccacagactctcctgtcccccttcatcgctcttaagaatc | |
| aacaggttaaaactcggcttcctttgatttgcttcccagtcacatggccgtacaaagaga | |
| tggagccccggtggcctcttaaatttcccttccgccacggagttcgaaaccatctactcc | |
| acacatgcaggaggcgggtggcacgctgcagcccggagtccccgttcacactgaggaacg | |
| gagacctgtgaccacagcaggctgacagatggacagaatctcccgtagaaaggtttggtt | |
| tgaaatgccccgggggcagcaaactgacatggttgaatgatagcatttcactctgcgttc | |
| tcctagatctgagcaagctgtcagttctcacccccaccgtgtatatacatgagctaactt | |
| ttttaaattgtcacaaaagcgcatctccagattccagaccctgccgcatgacttttcctg | |
| aaggcttgcttttccctcgcctttcctgaaggtcgcattagagcgagtcacatggagcat | |
| cctaactttgcattttagtttttacagtgaactgaagctttaagtctcatccagcattct | |
| aatgccaggttgctgtagggtaacttttgaagtagatatattacctggttctgctatcct | |
| tagtcataactctgcggtacaggtaattgagaatgtactacggtacttccctcccacacc | |
| atacgataaagcaagacattttataacgataccagagtcactatgtggtcctccctgaaa | |
| taacgcattcgaaatccatgcagtgcagtatatttttctaagttttggaaagcaggtttt | |
| ttcctttaaaaaaattatagacacggttcactaaattgatttagtcagaattcctagact | |
| gaaagaacctaaacaaaaaaatattttaaagatataaatatatgctgtatatgttatgta | |
| atttattttaggctataatacatttcctattttcgcattttcaataaaatgtctctaata | |
| caatacggtgattgcttgtgtgctcaacatacctgcagttgaaacgtattgtatcaatga | |
| acattgtaccttattggcagcagttttataaagtccgtcatttgcatttgaatgtaaggc | |
| tcagtaaatgacagaactatttttcattatgggtaactggggaataaatgggtcactgga | |
| gtaggaatagaagtgcaagctggaaaggcaaaaatgagaaagaaaaaggcaggccctttg | |
| tgtctaccgttttcagtgctgtgtgatcatattgttcctcacagcaaaaaagaatgcaag | |
| ggcataatgttagctgtgaacatgccagggttgcattcacattcctgggtacccagtgct | |
| gatggggtgtgcccacgtggggacatgtccttggcgtgcttcctcagagtggcttttcct | |
| ccattaatacatatatgagtactgaaaaattaagttgcatagctgctttgcagtggtttc | |
| agaggcagatctgagaagattaaaaaaaaatctcaaatgtatcagctttttttaaaggac | |
| attactagaaaattaaacagtattttttaacatgtgtgactttcatgcttctggggttgg | |
| agcttaaagatccaaactgagaaagcaggccgggcatggtggctcatgcctgtaatccca | |
| acactttgggaggccaaggagggtggatcacttaaggtcaggagtttgagaccagcctgg | |
| ccaacatggcgaaaccctgtctctactaaaaacataaaaattagctgggggtggtagcac | |
| atacctgtaatcccagctactcaggaggctgaggcaggagaatttgcttgatcctgggag | |
| gcagaggttgtagtgagccgagatcgcgccatcgcactccagcctgggtgacaagagcaa | |
| aactccatctc | |

TABLE 163

| | |
|---|---|
| cbe_3643627 DKFZP434I1735 | |
| DKFZP434I1735 protein | |
| length = 5477 | |
| agttattcttcatcctagcatttctgttcgactagcagcagcttggtgtttacactgcat | SEQ ID NO: 154 |
| tgccgtggcattaccctcctacctaacaccactcttggatcgttgccttgaacggcttac | |
| tggacataagtcttcacctgaagcagtgactggcttcagttttgctgtagcagctttgtt | |
| gggagcagtaaaacattgtcctttaggaattcctcatggaaaaggcaagattattatgac | |
| attagcagaggatttgctgtgttctgctgctcaaaacagtcgcctttcagctcagcgcac | |
| acaagctggatggttgctgatttctgctctgatgacattaggtcctgcagttgttagcca | |
| tcaccttgctcgagttctgctgttgtggaagtgtgtctttccagcatctcctaaagatct | |
| agaaacagaaaagagccgaggagattcgtttacatggcaggtgaccctggaaggacgagc | |
| tggtgcactgtgtgctatcaagagctttgtttcccactgtggtgatcttcttactgagga | |
| agtaactcagcgtcttcttccaccacttccttgtgctgtggatttgctaactcagctttc | |
| ttcaatactaaaaatgtatggaagtcctttgaaaacaccgtcagtggtttatagacaaag | |
| actttatgaactgttgattttattacctcctgagacctatgaaggaaacctctgtgctat | |
| cctcagagagctggctgctgacttgactgcccctgatattcaggtggcagcatctacatt | |
| tttacttccacccctctgtcatcaggatgatcttttgatactaagtcctttcctacaaga | |
| gactgaccatagatttattgaagaacagctcctgcttggtaatggtgttgcttgcggaag | |
| tcttgaatatgacccttattcgatttatgagaaagatgtagagggagattcagtgcctaa | |
| gcccttacctccagcactatcagttattagttctgcatccaagctctttggggttgtatg | |
| cgctcatgtgggagaaactcaaaggcttcttatactggaacagcttttggacagtataaa | |
| gcacacaaaaggagctcgtcagcaagtggttcagttacatgttgtttcttcagtttctag | |
| tttcttgaagtacgtggctggctccaagggatgtttaggtccagaagaaatgaaaagatt | |
| tgccttaacattagttatgggagccctagaaagccccaaccccttgctgagatgtgcagc | |
| tgcagagtcatgggctagattagcccaaatggtagatgatggagcttttactgctggatt | |
| agctcaagttagctttgacaaattgaaatcagcaagggatgtggttaccagaacaggaca | |
| ctcattggccttggggtccctacataggtatttaggaggaataagttcttctcaacacct | |
| aaattcttgtattggaatcctttatactttggcgcaggacagcacttctcctgatgtgca | |
| gacctgggcattacattctctatcattgatcattgattctgctggcccactctattatgt | |
| gcatgtggaacctaccctttctcttattataatgttgttgttaaatgtgcctcctactca | |
| tgctgaagttcaccaaagccttggtcgctgtttgaatgcccttattaccacgttaggtcc | |
| agagctacaaggtaacagtacttcaatttctaccttaaggacttcctgtctactgggttg | |
| tgcagtaatgcaagataacccagactgccttgttcaagctcaggccatctcttgccttca | |
| gcagcttcatatgtttgctccacgacatgtcaacttgtctagcctggttagctgcctctg | |
| tgtgaatctttgtagcccctacttgttactgagaagagcagtactggcttgcttacgtca | |
| gcttgtacaaagagaagcagctgaagtttcagaacatgctgttatgcttgctaaggatag | |
| cagagaagagttgactccagatgctaacatcagagaagttggccttgagggggcattgtt | |
| gatcttactagacaaggagacagatgagagattatgccatgatatcaaagagactttaaa | |
| ttatatgcttacatctatggcagtggaaaaactctccctgtggttaaagctttgtaaaga | |
| tgtacttgctgcatcagctgattttacagctgtaacttgtgtggatacaatgcaagaaga | |
| agaaggagataaaggggatgatgcctcagtcctgaccaccagacgtgatgaaaaatccca | |
| tccttttaccaatccccgatgggctactagagtctttgctgctgaatgtgtctgtaggat | |
| aattaaccaatgtgagaatgctaacagtgcacattttgacattgctttagcacaagaaat | |
| gaaaaaaagagattcaagaaatgactttttggtactgcatcttgctgacttaattcgcat | |
| ggcttttatggctgccacagatcacagtgaccagctccgtctttctggccttgaaatgct | |
| gttagttgttattcggcgatttgcaactgttccagaaccagagtttccaggtcatgtgat | |
| tctggaacagtatcaagccaatgtaggagcagcgcttagaccagccttcacttcagagac | |
| accacctgatgtcactgccaaagcatgtcaggtttgcagtgcctggatagcaagtggagt | |
| tgtaagtgaccttaatgatctccgaagagttcatcagttgcttgtttcatcgttaactaa | |
| aatacaggctggaaaagaagctctaagtcacttatataatgaaagtgcttctaccatgga | |
| gatcttagctgtgcttaaagcctgggcagaggtctacataattgctgtgcaaagacataa | |
| aaaccacagacaacctttgaagactaccacctgtttagaagacggtatcagaaatggatc | |
| atgttcatcagatggactgcttgacttagtctatgcagatcttggcacactaagcagact | |
| ctggcttgctgcacttcaggattttgctcttttaactttgccttcagaatttgcctccca | |
| acttcctgctgaaggtggtgctttctacacagcagagactagtgaaaatgcaaaattgca | |
| ttattacaactcctgggcacttatcctccatgctacagcattgtggcttacaagcacggg | |
| ttttgttgttgctgacccagatgaaggagcatctaatctctccaggcctgtaacaccaac | |
| ttccatgtgtcagggttcatcatctggggctaccataaagtcccctgaggatgtctacac | |
| tgatagattccatcttattttaggaatcagcgtggaatttctatgttccttacgttcaga | |
| tgcaaccatggaaagcataactgcttgtttacatgcattgcaagcacttctagatgtacc | |
| ttggcccagatcaaaaattggcagtgatcaggacttgggtatagaattgctgaatgttct | |
| acatcgagtaattttaaccagagaatcaccttccattcagttggcttcacttgaagtggt | |
| aaggcagattatctgtgctgctcaagaacatgtgaaggaaaaaagacgaagtgcagaagt | |
| tgatgatggggctgctgaaaaggaaactctgccagagtttggagagggaaaggacaccgg | |
| aggacttgtgcctggaaagtctttggtctttgcaacactggaattgtgtgtatgcattct | |
| agttagacagctcccagaattaaaccctaaattgacaggtagcccaggagtaaaagctac | |
| gaagccacagatactattagaagatggaagtagattggtttcagctgcattggttatcct | |
| ttccgaacttcctgcagtgtgctctcctgaaggaagcatctcaattctccctactatatt | |
| gtacctcacaatcggggtcctcagagagactgctgtgaagttacctgggggccagttatc | |
| ttcgacagttgcagcttccctacaggctttaaaaggaatattatcttctcccatggcccg | |
| ggcagaaaagagccgtactgcttggactgaccttctccgaagtgccttaacaacaattct | |
| tgactgttgggatccagttgatgaaacacaccaagaacttgatgaagtcagtctacttac | |
| tgctatcacagtgtttattttgtctaccagtccagaagtaactaccatcccatgccttca | |
| gaagcgctgtattgataaatttaaagctactcttgagataaaagatcctgtggtacaaat | |
| caagacctaccagctcctacattccatctttcagtatccaaatccagctgtttcctaccc | |
| atacatttactctttagcatcctgtatcatggaaaaactgcaggaaatagacaagagaaa | |
| acctgaaaacactgctgagcttgagatcttccaagaaggcataaaggtcttagaaacact | |
| ggttactgttgctgaagaacaccatcgcgctcagctggtggcctgtcttttgcccatcct | |
| catttccttccttttggatgaaaattctctgggatcagcaacttccataatgagaaattt | |
| acatgactttgctctacaaaatctcatgcaaattggacctcagtattcatctgtttttaa | |
| aagtttagtggcttcttctccagccctaaaagcccgccttgaggctgctataaagggcaa | |
| tcaggaaagtgtcaaagtcaagataccaacatctaaatatactaagagtcctggaaaaaa | |
| ctcaagcatccaattaaagaccagtttcctctgattttttttttggaatagtaagcacct | |
| taatataataaatacttgatcattgcctttggtgacaaaagggacattgtagacacaagt | |
| gcccttagaggtctagttgattgttttaccttaaatagctgtgactcttcaaagtaacag | |
| catacttaacatttttgtgtgtgtgcgtggggtttgtgttttgttttgttttgtttttct | |
| atttaaaaagacaatggagctaacttgccaggaattttaagaaagtgctaaagaactaaa | |
| cttactaaacaatccactgctttaaaaatgaaatgaatttcttctaacagtccatacata | |
| tgtggtatacttaatactgtgacagatataaatatgactctagtgtaaattatctgcact | |
| aatattgtatcccaattgctgatttaaaattctgctttggcccggcacagtggctcatcc | |
| ctgtaattccagcactttggaaggccaaggcgggcggatcacgaggtcgggagtttgaga | |
| ccagcctgaccaacatggtgaaaccccgtctctactaaaaatacaaaaattagccgggtg | |
| tggtggcacacacctgtaatcccagctactcaggaggccgaggcaggaaaatcgcttgaa | |
| cctggcaggcagaggttgcagtgagccaagattgtgccactgcactccagcctgggcgac | |
| agagtgagactccatct | |

TABLE 164

| | |
|---|---|
| cbe_3663966 KIAA1389 | |
| KIAA1389 protein | |
| length = 5801 | |
| caggcaaggcttatctggtgaaaacttttttgctatgctcagagggtaccgagtagaaaa | SEQ ID NO: 155 |
| ttatgacccaaaagggaccattgctttgtggtcataattttctactcggtaccctctgag | |
| catagcaaattatgaccacaaagcaatggtcccttttgggttccctgaatttttccgctg | |
| tgaccctgcaatctctccgagccttcatgcagcagcacagatttctaggggagaatttgt | |
| ccgcatctcaggattagattatgtggacagtgccctcctgatggggagagacagggacaa | |
| gcctttcaaacggaggttgaaatcagagtcggtggaaacatctctcttccgaaagcttcg | |
| aactgttaaaagtgagcacgaaactttcaagttcacgtctgagctggaggagagccgact | |
| ggagaggggcattcgcccttggaattgtcagcgatgttttgcacattatgatgtccagag | |
| cattttgtttaatatcaacgaagccatggctacgagggctaatgtggggaaaaggaaaaa | |
| cataaccactggggcatctgcagcatcccagactcagatgcctacgggccagacaggcaa | |
| ctgtgagtcccctttagggagcaaggaggacctcaactccaaagagaacctggatgccga | |
| tgagggtgatgggaaaagtaacgacctcgtccttagttgtccttactttagaaatgagac | |
| tggaggggaaggcgacaggcggattgcgctctctcgagccaactcatcctctttcagttc | |
| tggggaaagctgctctttcgaatcgtcactcagctctcactgcacaaatgcaggtgtctc | |
| cgtcttggaagtgcccagagaaaaccagcctattcacagggagaaagtgaagcgctacat | |
| catagaacacattgaccttggggcctattattaccgcaaattcttctatgggaaagagca | |
| ccaaaactactttggaatagatgaaaaccttggtccagtagcagtcagcatccggagaga | |
| gaaggtggaagatgccaaggagaaagaaggatcccagttcaactacagggtggctttcag | |
| gacaagtgagcttacaacactgagaggagcaattttagaagatgctataccctctactgc | |
| taggcatggtaccgcacgaggactacctctcaaagaagttttggaatacgtcattccaga | |
| gctgagcattcagtgtttgcgacaggcttccaactcacccaaggtctcagagcagctgct | |
| caagcttgatgaacaagggctgagctttcagcacaagatcgggatcctttattgcaaagc | |
| aggccagagcacagaggaagagatgtataacaatgagacggcgggaccagcttttgaaga | |
| attccttgatcttctgggccagagagtccgactgaaaggatttagtaaatatcgagctca | |
| gctagacaataagactgattccacgggcacgcactctctctataccacatacaaagacta | |
| cgaactcatgttccacgtgtcaaccctgcttccctacatgcccaacaacagacaacagct | |
| actgaggaaaaggcacataggaaatgacatcgtcaccatcgtcttccaggagcctggggc | |
| acttccttttactccaaaaagcatccggtctcactttcagcatgtctttgtcatagtcaa | |
| agtgcataatccatgtaccgaaaatgtgtgttatagtgttggagtttccagatcaaaaga | |
| tgtgccaccatttggcccaccgattcccaaaggtgtaacttttccaaagtcagccgtgtt | |
| ccgggacttccttttagccaaagtaatcaatgcagaaaatgcagcccataaatcagaaaa | |
| gtttcgagcaatggccactcgaacgaggcaggagtacttgaaagatctggcggagaactt | |
| tgtcacaaccgccaccgtggatacctctgtgaagttcagcttcattacgctgggtgcgaa | |
| gaaaaaggagaaggtaaagccaaggaaggatgcccacttgtttagcattggggccatcat | |
| gtggcacgtgatagcccgggacttcggccagtctgctgacattgaatgtcttctcgggat | |
| ctccaatgagttcatcatgttgattgaaaaggattccaagaatgttgtattcaactgttc | |
| ctgcagggatgtgattgggtggacatctggattagtgagtatcaaagtgttttacgaaag | |
| aggagaatgtgtcctcctgtcctcggtagacaactgtgctgaagacatcagggaaattgt | |
| tcagcgattagtaatagtgacgagaggctgcgagactgtggaaatgaccctgaggaggaa | |
| cgggctgggccagcttggcttccatgtgaattttgaaggaattgtcgcagatgtggaacc | |
| ttttggctttgcctggaaggctggccttcgccaagggagccgcctcgtggagatctgcaa | |
| agtagccgtggccactctgacccacgagcagatgatcgacctgctccgtacttctgtgac | |
| tgtgaaggtggtcatcatccagccccatgatgacggctcgccccgaagagggtgttcaga | |
| gctctgccggatccctatggtggaatataaactcgacagcgagggcaccccctgcgagta | |
| taaaacccccttcaggaggaacactacgtggcaccgggtgcccactcctgccctgcagcc | |
| cctctctagagcttcccccatccccggcacgcccgaccggctgccgtgccaacagctgct | |
| ccagcaggcccaggctgccattcctcgaagcacctccttcgaccggaagctgcccgatgg | |
| cacgagaagctcacccagcaaccagtcatcctccagcgaccctggacccggcgggagcgg | |
| accctggagaccacaagtgggctacgacgggtgccagtcccctctactgctcgaacacca | |
| gggctcaggccctttggaatgtgacggagccagggagagggaagacaccatggaagcaag | |
| caggcacccggaaaccaaatggcatggcccaccttccaaagtcctgggttcctataaaga | |
| aagagctctgcagaaagatggaagttgcaaagattcccccaataagctttctcacattgg | |
| ggataaaagttgctccagtcactccagcagcaacacgctctccagcaacacctccagcaa | |
| cagtgacgacaagcactttgggtctggcgacctgatggaccccgaattactggggctgac | |
| ctacatcaaaggggcctccaccgacagtggcatcgacacggccccctgcatgcctgccac | |
| catcctcggccctgtgcacctggcaggcagcaggtccctgatccacagccgggccgagca | |
| gtgggctgatgctgccgacgtctctgggcctgacgacgagccagccaagttatattctgt | |
| gcatggctacgcgtccgccatctccgccggcagtgctgcggaaggcagcatgggcgatct | |
| cagtgagatatcctctcattccagtggttctcaccattcaggaagcccttcagctcactg | |
| ttcaaaaagtagtgggtctctggattcatccaaagtctacatcgtgtctcacagcagcgg | |
| acaacaggttcccgggtccatgtccaagccctaccacagacaaggggcagtgaacaaata | |
| tgtcatcggctggaagaaatcggagggcagcccaccgcccgaggagcctgaagtgactga | |
| atgtcccgggatgtatagtgagatggatgtcatgtccacagcaactcagcatcagacagt | |
| ggtgggagatgctgttgcagagactcaacatgttctgtctaaagaagattttctgaaatt | |
| gatgcttcctgacagccccttagtggaggaggggcgaagaaagttttcgttctatgggaa | |
| cctgtctccaaggaggtcgctttaccgcacgctgtctgacgagagcatctgcagcaacag | |
| gagggggtcctcctttggcagttcccggagttccgtgcttgaccaggccctgcccaacga | |
| cattctgttcagcaccaccccaccctaccacagcacgctgcctccgcgggcccaccccgc | |
| acccagcatggggagcctgagaaatgagttctggttctccgatgggtccttatcagataa | |
| gtccaagtgcgcagatcctggcctgatgcccctcccggacacagccacagggttagattg | |
| gacccacctcgtggatgctgcacgggcatttgaaggtcttgactcagatgaagaactggg | |
| gctgctctgtcaccacacgtcctatctagaccagagggtggcatccttctgcaccctgac | |
| agatatgcagcatgggcaggacctggaaggggcccaagagctgcccttatgtgtagatcc | |
| aggcagtggcaaagagttcatggacacaactggggagcgttctccatcaccactgaccgg | |
| gaaagtcaatcagctggaattaattcttcgacaactccagaccgaccttcggaaggaaaa | |
| acaagacaaggccgttctccaagcagaagtgcagcacctgagacaggacaacatgagact | |
| gcaggaggagtcccagaccgcgacagctcagctgcggaaattcacagaatggtttttcac | |
| caccatcgacaaaaaatcttagccaatccgcacctcatcaagggaccactgggaaatgcc | |
| ccttgtccctttgaagtcacaaacatgtggtttttctgtgtgctttcaaccaatcgtaga | |
| tgtttttgctgttccattctgtgtagcaccattcaccacagcaggatagggagcctcgac | |
| tctttctcggtaaccacggcagagagcagcgccgatgtgaagatgaatgaatgtaactcc | |
| tggtgtgaagatgaatgtaaaccctggcgacagttgagacctttttcttttagactctgc | |
| taaaacagtgctctggcttgggcttacctcaagagggaagatagttgagttttatttcct | |
| gtatatcaggtgacctggtagagatgtaaagcaatttaccatagtttgggctttagtatt | |
| gtaaaataaacatgagaacaaataatcagacatatactttaatgttaaaggtgctctatt | |
| tttttggatgtacagtagttttatttccacagccacattaccatagcaataagaaaggag | |
| gcatagtacatagttggaaaagctttgtggggggattaaaaaaaaaaaaaagcactgttg | |
| tgtttaacactagttcagatgcagttaccttagagacttatttatttgcaggaacaaatg | |
| gtgcctgaatattaacagtgttctgattaaaaacaaaaaaaagatacatatgccttgtaa | |
| atggctcaccgagtggtcagtagtcacttcaactcttagttcacttttgtatagttgctc | |
| tgctggaaagaaatgagagtgaatctgcttactcactagaacttccctgtgtgctgtgag | |
| ccagcggaaccacttgtacaatgccagatttgtttatctttgtacagaagctttgatgaa | |
| gtgtcttgtatttaacacccttatttaagcttatttaaccttaaattgttaattttataa | |
| aatttggtttggcctgcactacgatgagggatggaggtagctgcaggctcagaagagact | |
| gagcttgcacagatcagaccgagaagcagggtgagagattctaacgactggatgctgcta | |
| gtaacacattgtttgtattgctttaccatttttaactgttagatttgagatgaacataca | |
| ttttgcttttttaataaatgtttaaaagaagtccacataag | |

TABLE 165

| | |
|---|---|
| cbe_3665307 HK1 | |
| hexokinase 1 | |
| length = 3962 | |
| aaaacatctatcttgctgtgtttggacaggccagcccctgaaacatcttgggcaatggag | SEQ ID NO: 156 |
| ggttaacttctcaaagtttaataggcaagaccagcaaccatgcaacaaggacttcaacta | |
| accaactaaagaactgttccccagagcattgttcctgagaaggaaaagagtccaaacacc | |
| tacccacacctgctttgtgccaagaatccacagttggattgcaaggacagtgtatgttgt | |
| ccttttggaaaaatgagagtgagcccaaatgaagaacaagcaaaggcgttcaagacccag | |
| ctgttgagagtagaaaagcagaagaaaggacccgaggtcagcaagtgccctccccacaat | |
| ggggcagatctgccagcgagaatcggctacagcagctgaaaaaccaaaacttcatctact | |
| tgctgaaagtgagcattgttgatgctcttgagagcatcagccaggacattaatgtgcacc | |
| actgtggtggcgtggaaagatggcaaaaagagccctgcgtgattttattgacaagtatct | |
| ctatgccatgcggctctccgatgaaactctcatagatatcatgactcgcttcaggaagga | |
| gatgaagaatggcctctcccgggattttaatccaacagccacagtcaagatgttgccaac | |
| attcgtaaggtccattcctgatggctctgaaaagggagatttcattgccctggatcttgg | |
| tgggtcttcctttcgaattctgcgggtgcaagtgaatcatgagaaaaaccagaatgttca | |
| catggagtccgaggtttatgacaccccagagaacatcgtgcacggcagtggaagccagct | |
| ttttgatcatgttgctgagtgcctgggagatttcatggagaaaaggaagatcaaggacaa | |
| gaagttacctgtgggattcacgttttcttttccttgccaacaatccaaaatagatgaggc | |
| catcctgatcacctggacaaagcgatttaaagcgagcggagtggaaggagcagatgtggt | |
| caaactgcttaacaaagccatcaaaaagcgaggggactatgatgccaacatcgtagctgt | |
| ggtgaatgacacagtgggcaccatgatgacctgtggctatgacgaccagcactgtgaagt | |
| cggcctgatcatcggcactggcaccaatgcttgctacatggaggaactgaggcacattga | |
| tctggtggaaggagacgaggggaggatgtgtatcaatacagaatggggagcctttggaga | |
| cgatggatcattagaagacatccggacagagtttgacagggagatagaccggggatccct | |
| caaccctggaaaacagctgtttgagaagatggtcagtggcatgtacttgggagagctggt | |
| tcgactgatcctagtcaagatggccaaggagggcctcttatttgaagggcggatcacccc | |
| ggagctgctcacccgagggaagtttaacaccagtgatgtgtcagccatcgaaaagaataa | |
| ggaaggcctccacaatgccaaagaaatcctgacccgcctgggagtggagccgtccgatga | |
| tgactgtgtctcagtccagcacgtttgcaccattgtctcatttcgctcagccaacttggt | |
| ggctgccacactgggcgccatcttgaaccgcctgcgtgataacaagggcacacccaggct | |
| gcggaccacggttggtgtcgacggatctctttacaagacgcacccacagtattcccggcg | |
| tttccacaagactctaaggcgcttggtgccagactccgatgtgcgcttcctcctctcgga | |
| gagtggcagcggcaagggggctgccatggtgacggcggtggcctaccgcttggccgagca | |
| gcaccggcagatagaggagaccctggctcatttccacctcaccaaagacatgctgctgga | |
| ggtgaagaagaggatgcgggccgagatggagctggggctgaggaagcagacgcacaacaa | |
| tgccgtggttaagatgctgccctccttcgtccggagaactcccgacgggaccgagaatgg | |
| tgacttcttggccctggatcttggaggaaccaatttccgtgtgctgctggtgaaaatccg | |
| tagtgggaaaaagagaacggtggaaatgcacaacaagatctacgccattcctattgaaat | |
| catgcagggcactggggaagagctgtttgatcacattgtctcctgcatctctgacttctt | |
| ggactacatggggatcaaaggccccaggatgcctctgggcttcacgttctcatttccctg | |
| ccagcagacgagtctggacgcgggaatcttgatcacgtggacaaagggttttaaggcaac | |
| agactgcgtgggccacgatgtagtcaccttactaagggatgcgataaaaaggagagagga | |
| atttgacctggacgtggtggctgtggtcaacgacacagtgggcaccatgatgacctgtgc | |
| ttatgaggagcccacctgtgaggttggactcattgttgggaccggcagcaatgcctgcta | |
| catggaggagatgaagaacgtggagatggtggagggggaccaggggcagatgtgcatcaa | |
| catggagtggggggcctttggggacaacgggtgtctggatgatatcaggacacactacga | |
| cagactggtggacgaatattccctaaatgctgggaaacaaaggtatgagaagatgatcag | |
| tggtatgtacctgggtgaaatcgtccgcaacatcttaatcgacttcaccaagaagggatt | |
| cctcttccgagggcagatctctgagacgctgaagacccggggcatctttgagaccaagtt | |
| tctctctcagatcgagagtgaccgattagcactgctccaggtccgggctatcctccagca | |
| gctaggtctgaatagcacctgcgatgacagtatcctcgtcaagacagtgtgcggggtggt | |
| gtccaggagggccgcacagctgtgtggcgcaggcatggctgcggttgtggataagatccg | |
| cgagaacagaggactggaccgtctgaatgtgactgtgggagtggacgggacactctacaa | |
| gcttcatccacacttctccagaatcatgcaccagacggtgaaggaactgtcaccaaaatg | |
| taacgtgtccttcctcctgtctgaggatggcagcggcaagggggccgccctcatcacggc | |
| cgtgggcgtgcggttacgcacagaggcaagcagctaagagtccgggatccccagcctact | |
| gcctctccagcacttctctcttcaagcggcgaccccctaccctcccagcgagttgcgctg | |
| ggagacgctggcgccagggcctgccggcgcggggaggaaagcaaaatccaactaatggta | |
| tatattgtagggtacagaatagagcgtgtgctgttgataatatctctcacccggatccct | |
| cctcacttgccctgccactttgcatggtttgattttgacctggtcccccacgtgtgaagt | |
| gtagtggcatccatttctaatgtatgcattcatccaacagagttatttattggctggaga | |
| tggaaaatcacaccacctgacaggccttctgggcctccaaagcccatccttggggttccc | |
| cctccctgtgtgaaatgtattatcaccagcagacactgccgggcctccctcccgggggca | |
| ctgcctgaaggcgagtgtgggcatagcattagctgcttcctcccctcctggcacccactg | |
| tggcctggcatcgcatcgtggtgtgtcaatgccacaaaatcgtgtgtccgtggaaccagt | |
| cctagccgcgtgtgacagtcttgcattctgtttgtctcgtggggggaggtggacagtcct | |
| gcggaaatgtgtcttgtcttccatttggataaaaggaaccaaccaacaaacaatgccatc | |
| actggaatttcccaccgctttgtgagccgtgtcgtatgacctagtaaactttgtaccaat | |
| tc | |

TABLE 166

| | |
|---|---|
| cbe_3670766 ACTC | |
| actin, alpha, cardiac muscle | |
| length = 1294 | |
| atgtgtgacgacgaggagaccaccgccctggtgtgcgacaacggctctgggctggtgaag | SEQ ID NO: 157 |
| gccggctttgcgggcgatgacgcgccccgcgctgtcttcccgtccatcgtgggccgcccg | |
| cggcaccagggagttatggtgggtatgggtcagaaggactcctacgtaggtgatgaagcc | |
| cagagcaagagaggcatcctgaccctgaagtatcccatcgagcatggtatcatcaccaac | |
| tgggacgacatggagaagatctggcaccacaccttctacaatgagctccgtgttgctccc | |
| gaggagcaccccaccctgctcacagaggccccgctgaaccccaaggccaaccgggagaag | |
| atgactcagatcatgtttgagaccttcaatgtccctgccatgtacgtggccatccaggca | |
| gtgctatccctgtatgcttctggccgtaccacaggcattgttctggactctggggatggt | |
| gtaactcacaatgtccccatctatgagggctacgctttgccccatgccatcatgcgtctg | |
| gatctggctggtcgggacctcactgactacctcatgaagatcctcactgagcgtggctac | |
| tcctttgtcaccactgctgaacgtgaaattgtccgtgacattaaagagaagctgtgctat | |
| gtcgccctggattttgagaatgagatggccacagctgcctcttcctcctccttggagaag | |
| agctatgaactgcctgatggccaagtcatcactatcggcaatgagcgcttccgctgtcct | |
| gagacactcttccagccctccttcattggtatggaatctgctggcatccatgaaacaact | |
| tacaatagcatcatgaagtgtgacattgatatccgcaaggacctgtatgccaacaatgtc | |
| ttatctggaggcaccactatgtaccctggtattgctgatcgtatgcagaaggaaatcact | |
| gctctggctcctagcaccatgaagattaagattattgctccccctgagcgtaaatactct | |
| gtctggattgggggctccatcttggcctctctgtccaccttccagcaaatgtggattagc | |
| aagcaagagtacgatgaggcaggcccatccattgtccaccgcaaatgcttctaagatgcc | |
| ttctctctccatctaccttccagtcaggatgacggtattatgcttcttggagtctcccaa | |
| accaccttccctcatctttcatcaatcattgtacagtttgtttacacacgtgcaatttgt | |
| ttgtgcttctaatatttattgctttataaataaa | |

TABLE 167

| | |
|---|---|
| cbe_3694420 ARHGEF2 | |
| rho/rac guanine nucleotide exchange factor (GEF) 2 | |
| length = 4093 | |
| gggatcccgggagggagcggagcggacctgggcttggtcgcctccaagccggcgggaccg | SEQ ID NO: 158 |
| agtgctttaggccgctcgaaagaaagttgctccgacccgggaaaaggagaagatgaagga | |
| agccaaggatgcccgctataccaatgggcacctcttcaccaccatttcagtttcaggcat | |
| gaccatgtgctatgcctgtaacaagagcatcacagccaaggaagccctcatctgcccaac | |
| ctgcaatgtgactatccacaaccgctgtaaagacaccctcgccaactgtaccaaggtcaa | |
| gcagaagcaacagaaagcggccctgctgaagaacaacaccgccttgcagtccgtttctct | |
| tcgaagtaagacaaccatccgggagcggccaagctcggccatctacccctccgacagctt | |
| ccggcagtccctcctgggctcccgccgtggccgctcctccttgtctttagccaagagtgt | |
| ttctaccaccaacattgctggacatttcaatgatgagtctcccctggggctgcgccggat | |
| cctctcacagtccacagactccctcaacatgcggaaccgaaccctatccgtggaatccct | |
| cattgacgaagaggtaatctacagtgagctgatgagtgactttgagatggatgagaagga | |
| ctttgcagctgactcttggagtcttgctgtggacagcagcttcctgcagcagcataaaaa | |
| ggaggtgatgaagcagcaagatgtcatctatgagctaatccagacagagctgcaccatgt | |
| gaggacactgaagatcatgacccgcctcttccgcacggggatgctggaagagctacactt | |
| ggagccaggagtggtccagggcctgttcccctgcgtggacgagctcagtgacatccatac | |
| acgcttcctcagccagctattagaacgccgacgccaggccctgtgccctggcagcacccg | |
| gaactttgtcatccatcgcttgggtgatctgctcatcagccagttctcaggtcctagtgc | |
| ggagcagatgtgtaagacctactcggagttctgcagccgccacagcaaggccttaaagct | |
| ctataaggagctgtacgcccgagacaaacgcttccagcaattcatccggaaagtgacccg | |
| ccccgccgtgctcaagcggcacggggtacaggagtgcatcctgctggtgactcagcgcat | |
| caccaagtacccgttactcatcagccgcatcctgcagcattcccacgggatcgaggagga | |
| gcgccaggacctgaccacagcactggggctagtgaaggagctgctgtccaatgtggacga | |
| gggtatttatcagctggagaaaggggcccgtctgcaggagatctacaaccgcatggaccc | |
| tcgggcccaaaccccagtgcctggcaagggcccctttggccgagaggaacttctgaggcg | |
| caaactcatccacgatggctgcctgctctggaagacagcgacggggcgcttcaaagatgt | |
| gctagtgctgctgatgacagatgtactggtgtttctccaggaaaaggaccagaagtacat | |
| ctttcctaccctggacaagccttcagtggtatcgctgcagaatctaatcgtacgagacat | |
| tgccaaccaggagaaagggatgtttctgatcagcgcagccccacctgagatgtacgaggt | |
| gcacacagcatcccgggatgaccggagcacctggatccgggtcattcagcagagcgtgcg | |
| cacatgcccatccagggaggacttccccctgattgagacagaggatgaggcttacctgcg | |
| gcgaattaagatggagttgcagcagaaggaccgggcactggtggagctgctgcgagagaa | |
| ggtcgggctgtttgctgagatgacccatttccaggccgaagaggatggtggcagtgggat | |
| ggccctgcccaccctgcccaggggccttttccgctctgagtcccttgagtcccctcgtgg | |
| cgagcggctgctgcaggatgccatccgtgaggtggagggtctgaaagacctgctggtggg | |
| gccaggagtggaactgctcttgacaccccgagagccagccctgcccttggaaccagacag | |
| cggtggtaacacgagtcctggggtcactgccaatggtgaggccagaaccttcaatggctc | |
| cattgaactctgcagagctgactcagactctagccagagggatcgaaatggaaatcagct | |
| gagatcaccgcaagaggaggcgttacagcgattggtcaatctctatggacttctacatgg | |
| cctacaggcagctgtggcccagcaggacactctgatggaagcccggttccctgagggccc | |
| tgagcggcgggagaagctgtgccgagccaactctcgggatggggaggctggcagggctgg | |
| ggctgcccctgtggcccctgaaaagcaggccacggaactggcattactgcagcggcaaca | |
| tgcgctgctgcaggaggagctacggcgctgccggcggctaggtgaagaacgggcaaccga | |
| agctggcagcctggaggcccggctccgggagagtgagcaggcccgggcactgctggagcg | |
| tgaggccgaagaggctcgaaggcagctggccgccctgggccagaccgagccactcccagc | |
| tgaggccccctgggcccgcagacctgtggatcctcggcggcgcagcctccccgcaggcga | |
| tgccctgtacttgagtttcaaccccccacagcccagccgaggcactgaccgcctggatct | |
| acctgtcactactcgctctgtccatcgaaactttgaggaccgagagaggcaggaactggg | |
| gagccccgaagagcggctgcaagacagcagtgaccctgacactggcagcgaggaggaagg | |
| tagcagccgtctgtctccgccccacagtccacgagactttaccagaatgcaggacatccc | |
| ggaggagacggagagccgcgacggggaggctgtagcctccgagagctaagggggcccctc | |
| ccccctgccccgtgccccactgaagaacattactgagggggctaaccttggggactccaa | |
| tttgccaatgatgagggaacatttgaaagaactgcaaattgtccttgccagctcttggga | |
| tccttggatacctggggccatttaagaagctaggggaattaggccacaacaccccctggg | |
| acatccgaaagctacaccacagatgccagtggttcatgccttcttcccgcaactttagga | |
| aaatttatttatttattgtttattagttatggggggagaggggagatttaaaggaccagg | |
| gacatgggaaccaagccatagggatcagagggccttgtccttgaacactactggggtata | |
| ttcaggctcatccacgcagctgctgggttcttgccctaacggccctcccctgcaacatcc | |
| gtcttggaggagaggctgcagccacagcaccctactgccctttaaataaaggagggctgt | |
| gggcagggccatgtccctttctcctctcccctcaacctcttactgctgttctccctttct | |
| ccgtccttcatggaagccctgggagataacctggcttcctggagttgatggaataaaggt | |
| tggggtggccataatggtttgttgggggtgagggaaaaaacccacagggaccagaatgtt | |
| ttgttgttcttttgttttcttttttgtaccaaagtcaactgcacgtgttttatattttta | |
| agagatcgtaggcaattagagatcgaagcctcctatctccacatctctgaagaagttgag | |
| gggtgggggagagaatgacttctgccttcatctgcagtaacggggggacctatactgacc | |
| tcttccccagccatttagaaacaagttctagggtgggttggaaaatctccaagagccctg | |
| acctcatcttccacctcagcaaccatgacctgaaacctcagcgtgaatttgggggatttt | |
| tcagtggaacccttgcccccaaatgtcgaccagcccccaaatgtcgaagaattttcttct | |
| tgccaattttgttgtttaaaaaaaaaattcagggaaaattaaaaacctggaactccaaaa | |
| aaaaaaaaaaaaa | |

TABLE 168

| | |
|---|---|
| cbe_3705944 TNFAIP1 | |
| tumor necrosis factor, alpha-induced protein 1 (endothelial) | |
| length = 3508 | |
| gccacccagctgagaggagaggcgcccccggggacgcactgagattatgaggctctggcc | SEQ ID NO: 159 |
| tccactggccactcactcgtgaccctttccaccacggcggagccttccaagcctacctcc | |
| tgccgtgtggtgatctacctgcagcgggagatgtcgggggacacctgcctgtgcccagcc | |
| tcaggggccaagcccaagctcagtggcttcaagggaggagggttgggcaacaagtatgtc | |
| cagctcaacgtgggcggctctctgtactacaccactgtgcgggccctgacccgccacgac | |
| accatgctcaaggccatgttcagtgggcgcatggaggtgctgaccgacaaagaaggctgg | |
| atcctcatagaccgttgtggaaagcactttggcaccattttgaattacctccgagatgac | |
| accatcaccctccctcagaaccggcaagaaatcaaggaattgatggctgaagcaaagtat | |
| tacctcatccaggggctggtgaatatgtgccagagtgccctgcaggacaagaaggactcc | |
| taccagcctgtgtgcaacatccccatcatcacatccctaaaggaggaggagcggctcatc | |
| gaatcctccaccaagcccgtggtgaagctgctgtacaacagaagcaacaacaagtattcc | |
| tacaccagcaactctgacgaccacctgctgaaaaacatcgagctgtttgacaagctctcc | |
| ctgcgcttcaacggccgcgtgctcttcatcaaggatgtcattggtgacgagatctgctgc | |
| tggtccttttatggccagggccgtaagctggcagaggtgtgctgtacctccatcgtgtat | |
| gccacggagaagaagcagaccaaggtggaattcccagaggcccgaatctatgaggagaca | |
| ctcaacgtcctactctatgagactccccgcgtccccgacaactccttgttggaggccaca | |
| agccgtagccgcagccaggcttcccccagtgaagatgaggagacctttgaactgcgggac | |
| cgtgtccgccgcatccacgtcaagcgctacagcacttacgatgaccggcagctcggccac | |
| cagtctacccatcgcgactgaccagaccctcagggagtcagggcacgggaggccctatct | |
| cccatcctgtggaacccgccccattggccaccccatgctgctgctgcctgggtctctgct | |
| ctagcacccagaggcatgacaggccctgctcagaggtcagagggtctgggcagaggaggg | |
| accacattcccctgccttgcccctgagcacttctggagactgcgtcctgtcctatctgct | |
| caccatcacccttcctgcccgacggagctgcttctgctccctggggcatatggactgacc | |
| cacctcctgctgagaaccttcccctaggccctgtgcagaaggctactgccccttaggcct | |
| cagctgggggaaaggcagttctggtgctgtagaggccctggtgcagaaagtgggacgtct | |
| tttttcctaaggtgtttaagcacaggcttgataagtttggtttttaaaaaataatctagg | |
| aaatgaataattctaaatctagtaatgaggaaactgagcatttcttttgccctccagggt | |
| gccaagaccctacatatgacagaacccttggcccttctccatgcctgtgggatctgtttc | |
| tttaaagcactttgtactgttattcaggaggttgataatctccttgacccatgtctttct | |
| accctaatccccacttccctgcagaatcaatctgagggaggggataaagaggaagcaata | |
| aaaaaaaaacatccgacagagcagctctggctttgcagcctggccagcagctcagagtgc | |
| accgaggagggaaggatggctaagctgggaccggcagtcctcacagggtgcctgtgagaa | |
| aggacattttacccccacatcatagtcacatcactgactcctaggtctagcacgactgct | |
| ctttgtgattctcttgagtacccttggcttccagccatgctgtcctcacatacggtaaag | |
| ccaaagagctgtcacatgggccagaaacatgagccacggcaggaagaccgtggagcccgt | |
| gggcactgcatggtgttggctggcatgcccatcagctgaggacagcaaactcccagcagc | |
| ccctacagaggtggcacatgcttggccacacatctactccctgcccacaccatctatgct | |
| cttggttggtgctggctgggatggcggttctgcccagtggtgtctctgagcgcgggatga | |
| caggagcaaccgaagcaccctgaaggccttcactccttgttgggtaactcagccatggag | |
| atgccaagcactagccaggaggtgagttcctctttagggctttggttttcattccttttt | |
| gtttggcttggccaaaccagaattcagcttatctgaattattttccaaaggaatgctgtc | |
| agggagggactgttctgccagcctaacaaagcaacgtagccacgtatagtacccactttc | |
| tgctctttggagagaacacaggttatcaagttcatctctcttgactactcttatgatagc | |
| tgatgccacagagcctatgggcaaatgccagacccagggttagacacaaggacctgaagt | |
| gacatgacggcgggacaggggaaatgtgactttctaattaggcattttatgttagtcaca | |
| gtcttgaatgtataaacagcactaagactctcaggtcaggtaccttggtgatcagctact | |
| agttcttccagccctcattgaggtaacaagataaagacaaatccacttctttggccaaat | |
| tcaggctttggctttatgactttcccacagagactggaatgcgtcagcctgagaccactg | |
| gcctattttctcagctgccctcttgaggtcctttaacactcaaattcccagctccccact | |
| gaggtgttgtgatgcttgccttttgacctccccatcccctttagtccctgcttactactt | |
| tgacattcacatcctcagtgtctcagtcttttttgccgagaaagcacagtagtctgggac | |
| tgggcatttatcttctctgactgaaaatctctccttggtcttaaggaaaatactaacatt | |
| gaactcactgacatgatcttagcttctttaatcagactttgtgacttaaaagtttggggg | |
| ttttctttgaaagtttccagccctattcagaaagcaactcttggctgtgtgcatttttca | |
| actccaagcagcccaggggtaagtaaacaaagtatggatgaaggtcagattttcttgtca | |
| gtttctgagaaacctggcagcctgctgttaacaacacaggccagtattgggttttattga | |
| atttggtatgtgaccaaggtcggcctaaaggatggcgcaggtcctgggcaggaaagaatt | |
| tttcctttatcacataactgtaatatttggttgctcagcataagtgatggaagcaaacac | |
| taatttctaataaaattgtgttaaactc | |

TABLE 169

| | |
|---|---|
| cbe_3714119 Prey1053021 | |
| Sequence 1 from Patent WO0131003. | |
| length = 3331 | |
| gtgattactcactatagggctcgagcggccgcccgggcaggtctatggctgagcctgggc | SEQ ID NO: 160 |
| acagccaccatctctccgccagagtcaggggaagaactgagaggcgcataccccggctgt | |
| ggcggctgctgctctgggctgggaccgccttccaggtgacccagggaacgggaccggagc | |
| ttcatgcctgcaaagagtctgagtaccactatgagtacacggcgtgtgacagcacgggtt | |
| ccaggtggagggtcgccgtgccgcataccccgggcctgtgcaccagcctgcctgaccccg | |
| tcaagggcaccgagtgctccttctcctgcaacgccggggagtttctggatatgaaggacc | |
| agtcatgtaagccatgcgctgagggccgctactccctcggcacaggcattcggtttgatg | |
| agtgggatgagctgccccatggctttgccagcctctcagccaacatggagctggatgaca | |
| gtgctgctgagtccaccgggaactgtacttcgtccaagtgggttccccggggcgactaca | |
| tcgcctccaacacggacgaatgcacagccacactgatgtacgccgtcaacctgaagcaat | |
| ctggcaccgttaacttcgaatactactatccagactccagcatcatctttgagtttttcg | |
| ttcagaatgaccagtgccagcccaatgcagatgactccaggtggatgaagaccacagaga | |
| aaggatgggaattccacagtgtggagctaaatcgaggcaataatgtcctctattggagaa | |
| ccacagccttctcagtatggaccaaagtacccaagcctgtgctggtgagaaacattgcca | |
| taacaggggtggcctacacttcagaatgcttcccctgcaaacctggcacgtatgcagaca | |
| agcagggctcctctttctgcaaactttgcccagccaactcttattcaaataaaggagaaa | |
| cttcttgccaccagtgtgaccctgacaaatactcagagaaaggatcttcttcctgtaacg | |
| tgcgcccagcttgcacagacaaagattatttctacacacacacggcctgcgatgccaacg | |
| gagagacacaactcatgtacaaatgggccaagccgaaaatctgtagcgaggaccttgagg | |
| gggcagtgaagctgcctgcctctggtgtgaagacccactgcccaccctgcaacccaggct | |
| tcttcaaaaccaacaacagcacctgccagccctgcccatatggtccctactccaatggct | |
| cagactgtacccgctgccctgcagggactgaacctgctgtgggatttgaatacaaatggt | |
| ggaacacgctgcccacaaacatggaaacgaccgttctcagtgggatcaacttcgagtaca | |
| agggcatgacaggctgggaggtggctggtgatcacatttacacagctgctggagcctcag | |
| acaatgacttcatgattctcactctggttgtgccaggatttagacctccgcagtcggtga | |
| tggcagacacagagaataaagaggtggccagaatcacatttgtctttgagaccctctgtt | |
| ctgtgaactgtgagctctacttcatggtgggtgtgaattctaggaccaacactcctgtgg | |
| agacgtggaaaggttccaaaggcaaacagtcctatacctacatcattgaggagaacacta | |
| ccacgagcttcacctgggccttccagaggaccacttttcatgaggcaagcaggaagtaca | |
| ccaatgacgttgccaagatctactccatcaatgtcaccaatgttatgaatggtgtggcct | |
| cctactgccgtccctgtgccctagaagcctctgatgtgggctcctcctgcacctcttgtc | |
| ctgctggttactatattgaccgagattcaggaacctgccactcctgcccccctaacacaa | |
| ttctgaaagcccaccagccttatggtgtccaggcctgtgtgccctgtggtccagggacca | |
| agaacaacaagatccactctctgtgctacaacgattgcaccttctcacgcaacactccaa | |
| ccaggactttcaactacaacttctccgctttggcaaacaccgtcactcttgctggagggc | |
| caagcttcacttccaaagggttgaaatacttccatcactttaccctcagtctctgtggaa | |
| accagggtaggaaaatgtctgtgtgcaccgacaatgtcactgacctccggattcctgagg | |
| gtgagtcagggttctccaaatctatcacagcctacgtctgccaggcagtcatcatccccc | |
| cagaggtgacaggctacaaggccggggtttcctcacagcctgtcagccttgctgatcgac | |
| ttattggggtgacaacagatatgactctggatggaatcacctccccagctgaacttttcc | |
| acctggagtccttgggaataccggacgtgatcttcttttataggtccaatgatgtgaccc | |
| agtcctgcagttctgggagatcaaccaccatccgcgtcaggtgcagtccacagaaaactg | |
| tccctggaagtttgctgctgccaggaacgtgctcagatgggacctgtgatggctgcaact | |
| tccacttcctgtgggagagcgcggctgcttgcccgctctgctcagtggctgactaccatg | |
| ctatcgtcagcagctgtgtggctgggatccagaagactacttacgtgtggcgagaaccca | |
| agctatgctctggtggcatttctctgcctgagcagagagtcaccatctgcaaaaccatag | |
| atttctggctgaaagtgggcatctctgcaggcacctgtactgccatcctgctcaccgtct | |
| tgacctgctacttttggaaaaagaatcaaaaactagagtacaagtactccaagctggtga | |
| tgaatgctactctcaaggactgtgacctgccagcagctgacagctgcgccatcatggaag | |
| gcgaggatgtagaggacgacctcatctttaccagcaagaagtcactctttgggaagatca | |
| aatcatttacctccaagaggactcctgatggatttgactcagtgccgctgaagacatcct | |
| caggaggccccgacatggacctgtgagaggcactgcctgcctcacctgcctcctcacctt | |
| gcatagcacctttgcaagcctgcggcgatttgggtgccagcatcctgcaacacccactgc | |
| tggaaatctcttcattgtggccttatcagatgtttgaatttcagatctttttttatagag | |
| tacccaaaccctcctttctgcttgcctcaaacctgccaaatatacccacactttgtttgt | |
| aaaaaaaaaaaaaaaaaaaaaaaaaaaaaaa | |

TABLE 170

| | |
|---|---|
| cbe_3753270 gbh_ac004503 | |
| Homo sapiens chromosome 5, P1 clone 1354A7 (LBNL H47), complete | |
| length = 2039 | |
| ccaggggcgcacgggttgggaggcgccaggcggcccgccctccttgcacgggccggccca | SEQ ID NO: 161 |
| gcttccccgcccctggcgtccgctccctcccgctcgcagcttacttaacctggcccgggc | |
| ggcggaggcgctctcacttccctggagccgcccgcttgcccgtcggtcgctagctcgctc | |
| ggtgcgcgtcgtcccgctccatggcgctcttcgtgcggctgctggctctcgccctggctc | |
| tggccctgggccccgccgcgaccctggcgggtcccgccaagtcgccctaccagctggtgc | |
| tgcagcacagcaggctccggggccgccagcacggccccaacgtgtgtgctgtgcagaagg | |
| ttattggcactaataggaagtacttcaccaactgcaagcagtggtaccaaaggaaaatct | |
| gtggcaaatcaacagtcatcatctacgagtgctgtcctggatatgaaaaggtccctgggg | |
| agaagggctgtccagcagccctaccactctcaaacctttacgagaccctgggagtcgttg | |
| gatccaccaccactcagctgtacacggaccgcacggagaagctgaggcctgagatggagg | |
| ggcccggcagcttcaccatcttcgcccctagcaacgaggcctgggcctccttgccagctg | |
| aagtgctggactccctggtcagcaatgtcaacattgagctgctcaatgccctccgctacc | |
| atatggtgggcaggcgagtcctgactgatgagctgaaacacggcatgaccctcacctcta | |
| tgtaccagaattccaacatccagatccaccactatcctaatgggattgtaactgtgaact | |
| gtgcccggctgctgaaagccgaccaccatgcaaccaacggggtggtgcacctcatcgata | |
| aggtcatctccaccatcaccaacaacatccagcagatcattgagatcgaggacacctttg | |
| agacccttcgggctgctgtggctgcatcagggctcaacacgatgcttgaaggtaacggcc | |
| agtacacgcttttggccccgaccaatgaggccttcgagaagatccctagtgagactttga | |
| accgtatcctgggcgacccagaagccctgagagacctgctgaacaaccacatcttgaagt | |
| cagctatgtgtgctgaagccatcgttgcggggctgtctgtagagaccctggagggcacga | |
| cactggaggtgggctgcagcggggacatgctcactatcaacgggaaggcgatcatctcca | |
| ataaagacatcctagccaccaacggggtgatccactacattgatgagctactcatcccag | |
| actcagccaagacactatttgaattggctgcagagtctgatgtgtccacagccattgacc | |
| ttttcagacaagccggcctcggcaatcatctctctggaagtgagcggttgaccctcctgg | |
| ctcccctgaattctgtattcaaagatggaacccctccaattgatgcccatacaaggaatt | |
| tgcttcggaaccacataattaaagaccagctggcctctaagtatctgtaccatggacaga | |
| ccctggaaactctgggcggcaaaaaactgagagtttttgtttatcgtaatagcctctgca | |
| ttgagaacagctgcatcgcggcccacggcaagagggggaggtacgggaccctgttcacga | |
| tggaccgggtgctgacccccccaatggggactgtcatggatgtcctgaagggagacaatc | |
| gctttaggtaattagttccatccccgggtggagcttctgcccagtggtcatgctggagtg | |
| ggatgtggggccccagctatttgtcaagctttcttctaccttggggattcaattaacact | |
| agcagtgcactgctgcgaccttccagacttgggatggggaaaaggcaagggtcgccttga | |
| aagcttacattgggaagaagggttacttctaagagtgtaatcttcacatgcatgggaagc | |
| agggaggggggactacatttttatgactgaagtgcaaggaaaacatcaccctctcattg | |

TABLE 171

| | |
|---|---|
| cbe_3760926 gbh_al359334 | |
| Homo sapiens EST from clone 628609, 5′ end. | |
| length = 881 | |
| tctagtctgtttgttttccttgtgtgtgttttctgttttcgggttggtctttttgtttgc | SEQ ID NO: 162 |
| tttttctcttttttccccctgtgtggttgcctttgtcttttttctcttttgtgtcgtgtt | |
| ctttgtttttgtttgtgttagaccctcatcaatagatggagacatacagaaatagtcaaa | |
| ccacatctacaaaatgccagtatcaggcggcggcttcgaagccaaagtgatgtttggatg | |
| taaagtgaaatattagttggcggatgaagcagatagtgaggaaagttgagccaataatga | |
| cgtgaagtccgtggaagcctgtggtacaaaaaatgttgagccgtagatgccgtcggaaat | |
| ggtgaagggagactcgaagtactctgaggcttgtaggagggtaaaatagagacccagtaa | |
| aattgtaataagcagtgcttgaattatttggtttcggttgttttctattagactatggtg | |
| agctcaggtgattgatactcctgatgcgagtaatacggatgtgtttaggagtgggacttc | |
| taggggatttagcggggtgatgcctgttgggggccagtgccctcctaattggggggtagg | |
| ggctaggctggagtggtaaaaggctcagaaaaatcctgcgaagaaaaaaacttctgaggt | |
| aataaataggattatcccgtatcgaaggcctttttggacaggtggtgtgtgggtggcctt | |
| ggtatgtgctttctcgtgttacatcgcgccatcattggtatatggttagtgtgttggtta | |
| gtaggcctagtatgaggagcgttatggagtggagtgaaatcacatggctaggccggaggt | |
| cattaggagggctgagagggctcctgttacggggtcatggg | |

TABLE 172

| | |
|---|---|
| cbe_3764052 MARK4 | |
| MAP/microtubule affinity-regulating kinase 4 | |
| length = 1620 | |
| tgggtcagtgtcacctccaggatacagacagccccccttcagcccagcccagccaggtct | SEQ ID NO: 163 |
| cctacaccgccaccatgccattcggtaacacccacaacaagttcaagctgaattacaagc | |
| ctgaggaggagtaccccgacctcagcaaacataacaaccacatggccaaggtactgaccc | |
| ttgaactctacaagaagctgcgggacaaggagactccatctggcttcactgtagacgatg | |
| tcatccagacaggagtggacaacccaggtcaccccttcatcatgaccgtgggctgcgtgg | |
| ctggtgatgaggagtcctacgaagttttcaaggaactctttgaccccatcatctcggatc | |
| gccacgggggctacaaacccactgacaagcacaagactgacctcaaccatgaaaacctca | |
| agggtggagacgacctggaccctaactacgtgctcagcagccgcgtccgcactggccgca | |
| gcatcaagggctacacgttgcccccacactgctcccgtggcgagcgccgggcggtggaga | |
| agctctctgtggaagctctcaacagcctgacgggcgagttcaaagggaagtactaccctc | |
| tgaagagcatgacggagaaggagcagcagcagctcatcgatgaccacttcctgttcgaca | |
| agcccgtgtccccgctgctgctggcctcaggcatggcccgcgactggcccgacgcccgtg | |
| gcatctggcacaatgacaacaagagcttcctggtgtgggtgaacgaggaggatcacctcc | |
| gggtcatctccatggagaaggggggcaacatgaaggaggttttccgccgcttctgcgtag | |
| ggctgcagaagattgaggagatctttaagaaagctggccaccccttcatgtggaaccagc | |
| acctgggctacgtgctcacctgcccatccaacctgggcactgggctgcgtggaggcgtgc | |
| atgtgaagctggcgcacctgagcaagcaccccaagttcgaggagatcctcacccgcctgc | |
| gtctgcagaagaggggtacaggtggcgtggacacagctgccgtgggctcagtatttgacg | |
| tgtccaacgctgatcggctgggctcgtccgaagtagaacaggtgcagctggtggtggatg | |
| gtgtgaagctcatggtggaaatggagaagaagttggagaaaggccagtccattgacgaca | |
| tgatccccgcccagaagtaggcgcctgcccacctgccaccgactgctggaacccagccag | |
| tgggagggcctggcccaccagagtcctgctccctcactcctcgccccgccccctgtccca | |
| gagtcccacctgggggctctctccacccttctcagagttccagtttcaaccagagttcca | |
| accaatgggctccatcctctggattctggccaatgaaatatctccctggcagggtcctct | |
| tcttttcccagagctccaccccaaccaggagctctagttaatggagagctcccagcacac | |
| tcggagcttgtgctttgtctccacgcaaagcgataaataaaagcattggtggccttaaaa | |
| aaaaaaaaaaaaaaaaaaaaaaaaaaaaaaaaaaaaaaaaaaaaaaaaaaaaaaaaaaaa | |

TABLE 173

| | |
|---|---|
| cbe_3770440 gbh_af217505 | |
| Homo sapiens uncharacterized bone marrow protein BM028 mRNA, | |
| length = 1725 | |
| cctcaaaagaagaagaaatgtgtcatatatcaatttggggatataagcaaatcctagtca | SEQ ID NO: 164 |
| cagtcgttttctaaaaagtattgttttctggaaaaacctagtttttaagaatgatgccac | |
| agcgaagttctattttaactttttaatttgatttttttttctttgttacagaagaagaca | |
| atgatgaaattaaggattggggacctcatgtaagaatgggaggggtgttccaaaggacat | |
| accaggggtctaggaggagtctaggaaggaagtcctgtgttatatcattctgggagtacc | |
| tattttccatgaggggatgaaatactggagctgttgtagaagaaaaacttctgattttaa | |
| tacattcttagcccaagagggctgtacaaaagggaaacacatgtggactaaaaaagatgc | |
| tgggaaaaaagttgttccatgtagacatgactggcatcagactggaggtgaagttaccat | |
| ttcagtatatgctaaaaactcacttccagaacttagccgagtagaagcaaatagcacatt | |
| gttaaatgtgcatattgtatttgaaggagagaaggaatttgatcaaaatgtgaaattatg | |
| gggtgtgattgatgtaaagcgaagttatgtaactatgactgcaacaaagattgaaatcac | |
| tatgagaaaagctgaaccgatgcagtgggcaagccttgaactgcctgcagctaaaaagca | |
| ggaaaaacaaaaagatgacacaacagattgagtgggagatggaaggaaggctattacatt | |
| atttccgaatttttaatactgtgtgaagtggtggcttgctgcgtgtaatcttttgttttg | |
| ttgttgtgttactgaatgtggcatttcagggttaacattaggttcttaaaagccaaagtc | |
| agtttgtctttttgtgcctctcatctttcttttgtgttatgtaagattgattattcattt | |
| ctccctactggtaggaaccatagttgtgtcctatacttgaagaggctggaaagtagccca | |
| taaccataattgcagtatttctttgtatttctctgttaagcaaagaaatattaaggaaca | |
| ttttttttatgtttttgtattattccataattagtaaagcaagatgaaatgtcaaatttt | |
| aatcagttttttcatgggatttgtgttcttacagtacttgaaaatatttaaggaagagat | |
| gaagctctgcagttttttctatgtgggatgattacttttttaaggaggattaattctgag | |
| gtagtatagtaactaaaggggaatatatgaattgtttaacaaattagaatttgtttacaa | |
| ctacttgaatttttaaattatgtcaaaacttacattacttgccaagcagtatgatgttat | |
| aggaaacataaataagattacagaggtatcaatttggttaaaattcaccattttataaga | |
| ctaagcaataatcttaacaacctctttcctgaatatttaaatgtgtttgtatggtgttat | |
| gactaattgttactgatttagagactaagccctcttaaaacctttagttaaatataaaaa | |
| gaaattatatatatcttgcctccctgatggaaaactatataaaattgtagacttaaaagg | |
| tttgtggaaatacattaggatatcagaaaactaaatatatggagttgctttatgactatt | |
| acatgttaaataaaaatagcttaattgttttggaaaaaaaaaaaa | |

TABLE 174

| | |
|---|---|
| cbe_3772244 PIGQ | |
| phosphatidylinositol glycan, class Q | |
| length = 2878 | |
| cagcgagcgccgtcgtctgcccgggcccgcccatcggggtccccaaccccatccggaccc | SEQ ID NO: 165 |
| cgccgcccgagcgcgcggccccggaagcacccgcctcccggcatggtgctcaaggccttc | |
| ttccccacgtgctgcgtctcgacggacagcgggctgctggtgggacggtgggtgccggag | |
| cagagcagcgccgtggtcctggcggtcctgcactttcccttcatccccatccaggtcaag | |
| cagctcctggcccaggtgcggcaggccagccaggtgggcgtggccgtgctgggcacctgg | |
| tgccactgccggcaggagcccgaggagagcctgggccgcttcctggagagcctgggtgct | |
| gtcttcccccatgagccctggctgcggctgtgccgggagagaggcggcacgttctggagc | |
| tgcgaggccacccaccggcaagcgcccactgcccccggtgcccctggtgaggaccaggtc | |
| atgctcatcttctatgaccagcgccaggtgttgctgtcacagctacacctgcccaccgtc | |
| ctgcccgaccgccaggctggagccaccactgccagcacggggggcctggctgccgtcttc | |
| gacacggtagcacgcagtgaggtgctcttccgcagtgaccgctttgatgagggccccgtg | |
| cggctgagccactggcagtcggagggcgtggaggccagcatcctcgcggagctggccagg | |
| cgagcctcgggacccatttgcctgctgttggccagcctgctgtcgctggtctcagctgtc | |
| agtgcctgccgagtgttcaagctctggcccctgtccttcctcgggagcaaactctccacg | |
| tgcgaacagctccggcaccggctggagcacctcacgctaatcttcagtacacggaaggcg | |
| gagaaccctgcccagctgatgaggaaggccaacacggtggcctctgtgctgctggacgtg | |
| gccctgggcctcatgctgctgtcctggctccacgggagaagccgcatcgggcatctggcc | |
| gacgccctcgttcctgtggctgaccacgtggccgaggagctccagcatctgctgcagtgg | |
| ctgatgggtgctcccgccgggctcaagatgaaccgtgcactggaccaggtgctgggccgc | |
| ttcttcctctaccacatccacctgtggatcagctacatccacctcatgtcccccttcgtg | |
| gagcacatcctttggcacgtgggcctctcggcctgcctgggcctgacggtggccctgtcc | |
| ctcctctcggacattatcgccctcctcaccttccacatctactgcttttacgtctatgga | |
| gccaggctgtactgcctgaagatccatggcctgtcctcactgtggcgtctgttccggggg | |
| aagaagtggaacgttctgcgccagcgcgtggactcctgttcctatgacctggaccagctg | |
| ttcatcgggactctgctcttcaccatcctgctcttcctcctgcctaccacagccctgtac | |
| tacctggtgttcaccctgctccggctcctggtggtcgccgtgcagggcctgatccatctg | |
| ctcgtggacctcatcaactccctgccgctgtactcactgggtcttcggctctgccggccc | |
| tacaggctggcggataaacccactgccctacagccgcgtggtgcacacctaccgcctccc | |
| cagctgtggctgccaccccaagcactcctggggcgccctgtgccgcaagctgttccttgg | |
| ggagctcatctacccctggaggcagagaggggacaagcaggactgagggaactgctggct | |
| cgcctggcaccaccacacggccacagccagccatctgctctgccagggtggcaccagctc | |
| agctggcgcatgtcctgtgctttgtggacgctgctgtgtgctcctgaacacggcaggccc | |
| tgctatcacaccttgggcttggaggtcattgggagtgagcagatgtgggggtggccagcc | |
| aggctggccgcactccatcactggcactgcctgccttgggacccgcttcccacctgctgc | |
| ggtcaccatggtggcgagcacagcaaccccaggtgtccagagcactgccccatgcccacc | |
| ctgtgtacccaggtccagagggtccgtccaccacagcagccccaggtggagggctggtct | |
| ccctgggggctccccagtggctctgccctggctgtgggggtggagggaccttgccaggat | |
| gaaccccccagtcccaggcaccctctagctccctcagccgaacagcaccctgcatctggg | |
| ggattgaagcagtcgctgacccccgtccccagcgggcccgggccctcactccctgaacca | |
| cacggggtttatttgcggatgttccctggagaggtcgctttgtgaagaaaccatcagcag | |
| gctgtgagcatcgccaggctgctgtgggggcgggagcagcctcagtgtcaagggcccgcc | |
| cactgacccagccgtacctattcgtccacggtgccccgtagcagcaggtcctgcggccaa | |
| atctgtctcccttcatgggcctcccagggaaggaggaagccctgctgtgcagacacctct | |
| gtggccccccaggagtgtgagtggcctggggagggggccgtggcactgaggccgaaagtg | |
| cctgccagacggcacggtctgggtgcgggtgttccctgtgagcccgagtccgcttcagga | |
| ggggagcctgcaggtgccggctggtgaggggatgacgcgctgtgggtgggaggaggcagc | |
| gcccatctcagcagcaccaggactgcctgggactccctggcaacccagcaccggggaagc | |
| cgtcagctgctgtgacaataaaacctgccccgtgtctggaaaaaaaaaaaaaaaaaaa | |

TABLE 175

| | |
|---|---|
| cbe_3773649 EEF1A1L14 | |
| eukaryotic translation elongation factor 1 alpha 1-like 14 | |
| length = 2106 | |
| gtatacgaaatcataaaatctcatagatgtatcctgagtagggcggggcccgtgaaaccc | SEQ ID NO: 166 |
| tctgaatctgcggccaccacccggtaaggctaaatactaatcagacaccgatagtgaact | |
| agtaccgtgagggaaaggtgaaaagaacccgagaggggagtgaaatagattctgaaacca | |
| tttacttacaagtggtccatttacttacaagtgtcagagcacgttaaagtgtgatggcgt | |
| acatcttgcagtatgggccggcgagttatgttaatatgcaaggttaagcagaaaaaagcg | |
| gagccgtagggaaaccgagtctgaatagggcgactttagtatattggcatatacccgaaa | |
| tcaggtgatctatccatgagcaggttgaagcttaggtaaaactaagtggaggaccgaacc | |
| gtagtacgctaaaaagtgcccggatggacttgtggatagtggtgaaattccaatcgaacc | |
| tggagatagctggttctcttcgaaatagctttagggctagcgtatagtattgtttaatgg | |
| gggtagagcactgaatgtggaatggcggcatctagctgtactgactataatcaaactccg | |
| aataccattaaaattaagctatgcagtcggaacgtggtatcaccattgatatctccttgt | |
| ggaaatttgagaccagcaagtactatgtgactatcattgatgccccaggacacagagact | |
| ttatccaaaacatgattacagggacctctcaggctgactgtgctgtcctgattgttgctg | |
| ctggtgttggtgaatttgaagctggtatctccaagaatgggcagacccgagagcatgccc | |
| ttctggcttacacactgggtgtgaaacaactaattgtcggtgttaacaaaatggattcca | |
| ctgagccaccctacagccagaagagatatgaggaaattgttaaggaagtcagcacttaca | |
| ttaagaaaattggctacaaccccgacacagtagcatttgtgccaatttctggttggaatg | |
| gtgacaacatgctggagccaagtgctaacatgccttggttcaagggatggaaagtcaccc | |
| gtaaggatggcaatgccagtggaaccacgctgcttgaggctctggactgcatcctaccac | |
| caactcgtccaactgacaagcccttgggcctgcctctccaggatgtctacaaaattggtg | |
| gtattggtactgttcctgttggccgagtggagactggtgttctcaaacccggtatggtgg | |
| tcacctttggtccagtcaacgttacaacggaagtaaaatctgtcgaaatgcaccatgaag | |
| ctttgggtgaagctcttcctggggacaatgtgggcttcaatgtcaagaatgtgtctgtca | |
| aggatgttcgtcgtggcaacgttgctggtgacagcaaaaatgacccaccaatggaagcag | |
| ctggcttccctgctcaggtgattatcctgaaccatccaggccaaataagcgccggctatg | |
| cccctgtattggattgccacacggctcacattgcatgcaagtttgctgagctgaaggaaa | |
| agattgatcgccgttctggtaaaaagctggaagatggccctaaattcttgaagtctggtg | |
| atgctgccattgttgatatggttcctggcaagcccatgtgtgttgagagcttctcagact | |
| atccacctttgggctgctttgctgttcgtgatatgagacagacagttgcggtgggtgtca | |
| tcaaagcagtggacaagaaggctgctggagctggcaaggtcaccaagtctgcccagaaag | |
| ctcagaaggctaaatgaatattatccctaatcctcccaccccactcttaatcagtggtgg | |
| aagaccggtctcagaactgtttgtttcaattgccatttaagtttagtagtaaaagactgg | |
| ttaatgataacaatgcatcgtaaaacctttcagaaggaaaggagaatgttttgtggacac | |
| gttggttttcttttttgcgtgtggcagttttagttattagtttttaaaatcagtactttt | |
| taatggaaacaacttgacccccaaatttgtcacagaattttgggacccattaaaaggtta | |
| actggg | |

TABLE 176

| | |
|---|---|
| cbe_3787129 LOC55828 | |
| zinc finger protein ZNF140-like protein | |
| length = 2978 | |
| ggaggccctgctgaggactccggcaagtgtgggtcgcggcgacggcggggctaaggccct | SEQ ID NO: 167 |
| gggtccgcgcgcggtttgaccacggccggggccttgggcatttcctggccttcctgttga | |
| gccgtgtaaacgcggggtgatgacggcgccgacctcttggcactgttgtgagagcgaagt | |
| gggcgcgagagcagacgccagctacagtttttttgggttatgtcgtcatgaagccggcgc | |
| tttcagttgtgcaaccttgaacaaatgggacactgcccatctctaagataagaacctgga | |
| aaggggactctgttggccattggaaattgcagaataatgtctcaggtgacatttagtgat | |
| gtggctatagacttctctcatgaagagtgggcatgcctagattctgctcagagggactta | |
| tacaaggatgtgatggtccagaattatgagaacctggtctctgtaggtctttccgtaact | |
| aagccatatgtgatcatgttattggaggatggaaaagagccctggatgatggagaaaaaa | |
| ctgtcaaaagcttacccatttcctttatcacactctgttcctgcttctgtgaactttgga | |
| ttctctgctctatttgagcattgttcagaagtcactgaaatatttgagttgtcagaacta | |
| tgtgttttctgggtgcttcatttcttatccaattctcctaattccactgtagaagctttt | |
| tcaagaagtaaaaaaaaaaaaaaaaaaaaaaaaaaaaggcagtgctttgctttcttgata | |
| tatttcagattgggaatcaagatgggaaaacaaggaattatcaacaaagaaggatattta | |
| tatgaagattcaccccaaccagtaacaatggaaaaagttgtaaaacaaagttatgaattt | |
| tcaaattctaataagaatttggaatatacagaatgcgacacatttagaagcacctttcat | |
| tcaaagtctactctttctgaaccacaaaacaattctgctgaagggaattcacacaaatat | |
| gatatattaaagaagaatttatcaaaaaagtcagttataaaaagtgagagaataaatggt | |
| ggaaagaaacttttaaattctaataaaagtggggcagccttcaaccagagcaaatctctt | |
| acccttccccagacttgtaatagagagaaaatctatacatgcagtgaatgtgggaaagcc | |
| tttggcaaacagtcaatcctcagtcgccactggagaattcatacaggagagaagccctat | |
| gaatgtcgtgaatgtgggaagacttttagccatggttcatcccttacacgacatcagata | |
| agccatagtggagagaaaccttacaaatgcattgaatgtgggaaggcctttagccatggc | |
| tcatcacttactaaccatcagagcactcacacgggagagaaaccgtatgaatgtatgaac | |
| tgtggaaagtcttttagtcgtgtgtcccttctcattcagcatctaagaattcatacgcaa | |
| gaaaaacgctatgagtgtcgtatatgtggaaaggccttcattcatagttcgtctctcatt | |
| caccatcagaaaagccatactggagagaagccttatgaatgtagagaatgtgggaaagct | |
| ttctgctgtagctcacaccttactcaacatcaaagaattcacagtatgaagaaaaaatat | |
| gaatgcaacaaatgtctcaaggtctttagtagcttctcatttcttgttcaacatcagagt | |
| attcatactgaagaaaaaccgtttgaagtttagaaatgcaggaaatccttcaaccagctt | |
| gaatcactgaatatgcatttgagaaatcacattagattgaaaccctacgaatgcagtata | |
| tgtgggaaagcctttagtcataggtcgtccctgcttcaacatcacagtattcatactgga | |
| gagaaaccttacgaatgtattaaatgtgggaagaccttcagctgtagttcaaaccttact | |
| gtacatcagagaattcatactggagaaaagccatataaatgtagtgagtgtgggaaagct | |
| tttagcaaaggctcgaatcttactgcccatcaaagagtacataatggagagaaacccaat | |
| agtgtggtaagtgtggaaaagcctttagatcatatgaatccctatacatgtgagaaatct | |
| tacagaagagaagcagtgtttatcacggtaaacttcattcatagatcctcccttatttaa | |
| catcagaaaaatgtatactggggaaaagttgtatgaaggtggtgaacatgggagactttt | |
| agcaatgatgcagatttttttattagagtttatactgtagagaaatcatatgaagtcaat | |
| aaatgtgggaaagcctttgtcagtattaatcccttaattgaccntaagtatactcacact | |
| aggaaaaatctgtgtacatgtagcaaatgtgggaaagactataggcaataggaatctcct | |
| gcaaactcctacaggagaaaagttgtatgaatgtggaaactttagaaattgaaggaattt | |
| ttccagttccaagtgcatcccttattctataggaaataaactggagacaaatctcattta | |
| agagatgcagcaaagtgttcactaagagtgtttatcttgccagacataagaagatgaatg | |
| gtagagcaacctgaaggatttagaaattacatataaatctttgcagttatgctatttgta | |
| aacaggattatataggagagcaaataaacataagtatgcatttcttagagcagtagcttg | |
| cagtttcagttgagttctacttagaaattctttttagctagtgggcatgtgaagatattt | |
| agtcacccagaggagccagtaaatgttataatgttaaaaattaaagctgcaaaagaaata | |
| aaatggtgttaataaaaattttggcatctaataaaatcattttgtatatcaaaaaaaaaa | |
| aaaaaaaaaaaaaaaaaaaaaaaaaaaaaaaaaaaaaa | |

TABLE 177

| | |
|---|---|
| cbe_3789063 AP2M1 | |
| adaptor-related protein complex 2, mu 1 subunit | |
| length = 1936 | |
| ccgaggcagcgggcagacgagcagggggcgggcggacatcttgggatccggagagtggcc | SEQ ID NO: 168 |
| gggccggcagagcagggggccgaggacaccaggtctgttctcagagcgatgggccgcgga | |
| gactgatctgccgccatgattggaggcttattcatctataatcacaagggggaggtgctc | |
| atctcccgagtctaccgagatgacatcgggaggaacgcagtggatgcctttcgggtcaat | |
| gttatccatgcccggcagcaggtgcgcagccccgtcaccaacattgctcgcaccagcttc | |
| ttccacgttaagcggtccaacatttggctggcagcagtcaccaagcagaatgtcaacgct | |
| gccatggtcttcgaattcctctataagatgtgtgacgtgatggctgcctactttggcaag | |
| atcagcgaggaaaacatcaagaacaattttgtgctcatatatgagctgctggatgagatt | |
| ctagactttggctacccacagaattccgagacaggcgcgctgaaaaccttcatcacgcag | |
| cagggcatcaagagtcagcatcagacaaaagaagagcagtcacagatcaccagccaggta | |
| actgggcagattggctggcggcgagagggtatcaagtatcgtcggaatgagctcttcctg | |
| gatgtgctggagagtgtgaacctgctcatgtccccacaagggcaggtgctgagtgcccat | |
| gtgtcgggccgggtggtgatgaagagctacctgagtggcatgcctgaatgcaagtttggg | |
| atgaatgacaagattgttattgaaaagcagggcaaaggcacagctgatgaaacaagcaag | |
| agcgggaagcaatcaattgccattgatgactgcaccttccaccagtgtgtgcgactcagc | |
| aagtttgactctgaacgcagcatcagctttatcccgccagatggagagtttgagcttatg | |
| aggtatcgcacaaccaaggacatcatccttcccttccgggtgatcccgctagtgcgagaa | |
| gtgggacgcaccaaactggaggtcaaggtggtcatcaagtccaactttaaaccctcactg | |
| ctggctcagaagatcgaggtgaggatcccaaccccactgaacacaagcggggtgcaggtg | |
| atctgcatgaaggggaaggccaagtacaaggccagcgagaatgccatcgtgtggaagatc | |
| aagcgcatggcaggcatgaaggaatcgcagatcagcgcagagattgagcttctgcctacc | |
| aacgacaagaagaaatgggctcgaccccccatttccatgaactttgaggtgccattcgcg | |
| ccctctggcctcaaggtgcgctacttgaaggtgtttgaaccgaagctgaactacagcgac | |
| catgatgtcatcaaatgggtgcgctacattggccgcagtggcatttatgaaactcgctgc | |
| tagctgccactaggcagctagcccacctccccagccaccctcctccacaggtccaggtgc | |
| cgctccctcccccaccacacatcagtgtctcctccctcctgctttgctgccttccctttg | |
| caccagcccgagtctaggtctgggccaagcacattacaagtgggaccggtggagcagccc | |
| ctgggctccctgggcaggggagttctgaggctcctgctctcccatccacctgtctgtcct | |
| ggcctaatgccaggctctgagttctgtgaccaaagccaggtgggttccctttccttccca | |
| cccctgtggccacagctctggagtgggagggttggttgcccctcacctcagagctccccc | |
| aaaggccagtaatggatccccggcctcagtccctactctgctttgggatagtgtgagctt | |
| cattttgtacacgtgtgacttcgtccagttacaaacccaataaactctgtagagtggaaa | |
| aaaaaaaaaaaaaaaa | |

TABLE 178

| | |
|---|---|
| cbe_3800240 IVD | |
| isovaleryl Coenzyme A dehydrogenase | |
| length = 2216 | |
| tttccgcagttaggggctgctatttcaacgcagggagataaaaagaaaaaaacacttgct | SEQ ID NO: 169 |
| cttctaccccgctaaaaacactcatcctagggagcacgccagcatttgcagcgttcgggg | |
| cagggccactcggcctgcggccgttgcactggctggaagctggcaggcgatcacggttga | |
| ttggctcgggtgcggtccaagggcagcaacgccttcggcgggccgcctagggtgattggc | |
| tgctgcagcccaccccctagccggtttggtgggcggcgaagcctggattggtggagctaa | |
| gagctggctcagtttcagcgctggctcttcgtgcatggcagagatggcgactgcgactcg | |
| gctgctggggtggcgtgtggcgagctggaggctgcggccgccgcttgccggcttcgtttc | |
| ccagcgggcccactcgcttttgcccgtggacgatgcaatcaatgggctaagcgaggagca | |
| gaggcagcttcgtcagaccatggctaagttccttcaggagcacctggcccccaaggccca | |
| ggagatcgatcgcagcaatgagttcaagaacctgcgagaattttggaagcagctggggaa | |
| cctgggcgtattgggcatcacagcccctgttcagtatggcggctccggcctgggctacct | |
| ggagcatgtgctggtgatggaggagatatcccgagcttccggagcagtggggctcagtta | |
| cggtgcccactccaacctctgcatcaaccagcttgtacgcaatgggaatgaggcccagaa | |
| agagaagtatctcccgaagctgatcagtggtgagtacatcggagccctggccatgagtga | |
| gcccaatgcaggctctgatgttgtctctatgaagctcaaagcggaaaagaaaggaaatca | |
| ctacatcctgaatggcaacaagttctggatcactaatggccctgatgctgacgtcctgat | |
| tgtctatgccaagacagatctggctgctgtgccagcttctcggggcatcacagccttcat | |
| tgtggagaagggtatgcctggctttagcacctctaagaagctggacaagctggggatgag | |
| gggctctaacacctgtgagctaatctttgaagactgcaagattcctgctgccaacatcct | |
| gggccatgagaataagggtgtctacgtgctgatgagtgggctggacctggagcggctggt | |
| gctggccggggggcctcttgggctcatgcaagcggtcctggaccacaccattccctacct | |
| gcacgtgagggaagcctttggccagaagatcggccacttccagttgatgcaggggaagat | |
| ggctgacatgtacacccgcctcatggcgtgtcggcagtatgtctacaatgtcgccaaggc | |
| ctgcgatgagggccattgcactgctaaggactgtgcaggtgtgattctttactcagctga | |
| gtgtgctacacaggtagccctggacggcattcagtgttttggtggcaatggctacatcaa | |
| tgactttcccatgggccgctttcttcgagatgccaagctgtatgagataggggctgggac | |
| cagcgaggtgaggcggctggtcatcggcagagccttcaatgcagactttcactagtcctg | |
| agacccttcgcccccttttcctgcacctagtggcctttcttgggaagtagagatgtggcg | |
| gctttcccaccctgcccacagcaggccctcctgcccagctgctcttgtcagccctctggc | |
| ctctggatgaggttgagttctccacaacagctcccaagcatcatgggcctcgcagccggg | |
| cctgtgccacggctagtgttgtgtgatttaaaatggactcagcaggaagcatattgtctg | |
| gggattgttgggacaggttttggtgactctgtgcccttgctctctaacttctgagcccac | |
| ctcccagggtaggcacctgggggcatgcaggtgcccacctcccagggtaggcacctgggg | |
| gcatgcaggtacccacctctttctcttgggtgaggctctggcaaggagatctctctgctc | |
| aagcacagcagaatcatggcccctctccatgaattggaacttggtacaggttaagtatcc | |
| ctaatcctgaaatctgaaacacttgtggttccaagcattttggataaggcaaattcaact | |
| ttcagtctcttttctgggggaaaaaaataataaacctagcctagccaggcgtggtg | |

TABLE 179

| | |
|---|---|
| cbe_3810032 gbh_af142421 | |
| Homo sapiens QUAKING isoform 5 (QUAKING) mRNA, complete cds. | |
| length = 1497 | |
| ccgggggcttctcagttttattttatcaatctatatgcatatccttatgccagtctcaca | SEQ ID NO: 170 |
| cagtgttaattactgtagctttgtagtcagttttgaaatcaagaagtatgagtccttcgt | |
| tcttatttttcaaaattatcttggctgttctgtgtcccgtgtgtttctagataaatttta | |
| ggatcatcttgtcaatttctggggatattggctgggttcttggtttacttccatgtgttc | |
| tctttaaatggctagactgggctttggttttttcagtatcgggttggaatctgagagggg | |
| aaatggaaacgaaggagaagccgaagcccaccccagattacctgatgcagctgatgaacg | |
| acaagaagctcatgagcagcctgcccaacttctgcgggatcttcaaccacctcgagcggc | |
| tgctggacgaagaaattagcagagtacggaaagacatgtacaatgacacattaaatggca | |
| gtacagagaaaaggagtgcagaattgcctgatgctgtgggacctattgttcagttacaag | |
| agaaactttatgtgcctgtaaaagaatacccagattttaattttgttgggagaatccttg | |
| gacctagaggacttacagccaaacaacttgaagcagaaaccggatgtaaaatcatggtcc | |
| gaggcaaaggctcaatgagggataaaaaaaaggaggagcaaaatagaggcaagcccaatt | |
| gggagcatctaaatgaagatttacatgtactaatcactttggaagatgctcagaacagag | |
| cagaaatcaaattgaagagagcagttgaagaagtgaagaaattattggtacctgcagcag | |
| aaggagaagacagcctgaagaagatgcagctgatggagcttgcgattctgaatggcacct | |
| acagagatgccgacattaaatcaccagcccttgccttttctcttgcagcaacagcccagg | |
| ctgctccaaggatcattactgggcctgcgccggttctcccaccagctgccctgcgtactc | |
| ctacgccagctggccctaccataatgcctttgatcagacaaatacagaccgctgtcatgc | |
| caaacggaactcctcacccaactgctgcaatagttcctccagggcccgaagctggtttaa | |
| tctatacaccctatgagtacccctacacattggcaccagctacatcaatccttgagtatc | |
| ctattgaacctagtggtgtattaggtgcggtggctactaaagttcgaaggcacgatatgc | |
| gtgtccatccttaccaaaggattgtgaccgcagaccgagccgccaccggcaactaaccta | |
| tgaccttctgacctctgaactcttcacccaatgatgacctgaccatgcctgcctgctgat | |
| cagttaactggtaatcgcctttgcttgcctgtcgtcagtgcagcgagctgaggcacttgt | |
| ccgttcgtcttaccatctaaccaaacaaaagacaaagaaattgttgtcctccaactc | |

TABLE 180

| | |
|---|---|
| cbe_3810791 EPHB1 | |
| EphB1 | |
| length = 3865 | |
| cacatgcacacccacacccacgcgcgcccgcaccgccccacgcgcacacactcctgccca | SEQ ID NO: 171 |
| cgcccacgcagcgctccgggaagtccggtccgggcgagagcgcgaaaggataccgagaag | |
| ccacccgcggagagcgcagcggcgccctgggacgcggcgctctcccggcgctgctgcctc | |
| ggcttggtctcggcctgcgggccgtcggccggcgatggccctggattatctactactgct | |
| cctcctggcatccgcagtggctgcgatggaagaaacgttaatggacaccagaacggctac | |
| tgcagagctgggctggacggccaatcctgcgtccgggtgggaagaagtcagtggctacga | |
| tgaaaacctgaacaccatccgcacctaccaggtgtgcaatgtcttcgagcccaaccagaa | |
| caattggctgctcaccaccttcatcaaccggcggggggcccatcgcatctacacagagat | |
| gcgcttcactgtgagagactgcagcagcctccctaatgtcccaggatcctgcaaggagac | |
| cttcaacttgtattactatgagactgactctgtcattgccaccaagaagtcagccttctg | |
| gtctgaggccccctacctcaaagtagacaccattgctgcagatgagagcttctcccaggt | |
| ggactttgggggaaggctgatgaaggtaaacacagaagtcaggagctttgggcctcttac | |
| tcggaatggtttttacctcgcttttcaggattatggagcctgtatgtctcttctttctgt | |
| ccgtgtcttcttcaaaaagtgtcccagcattgtgcaaaattttgcagtgtttccagagac | |
| tatgacaggggcagagagcacatctctggtgattgctcggggcacatgcatccccaacgc | |
| agaggaagtggacgtgcccatcaaactctactgcaacggggatggggaatggatggtgcc | |
| tattgggcgatgcacctgcaagcctggctatgagcctgagaacagcgtggcatgcaaggc | |
| ttgccctgcagggacattcaaggccagccaggaagctgaaggctgctcccactgcccctc | |
| caacagccgctcccctgcagaggcgtctcccatctgcacctgtcggaccggttattaccg | |
| agcggactttgaccctccagaagtggcatgcactagcgtcccatcaggtccccgcaatgt | |
| tatctccatcgtcaatgagacgtccatcattctggagtggcaccctccaagggagacagg | |
| tgggcgggatgatgtgacctacaacatcatctgcaaaaagtgccgggcagaccgccggag | |
| ctgctcccgctgtgacgacaatgtggagtttgtgcccaggcagctgggcctgacggagtg | |
| ccgcgtctccatcagcagcctgtgggcccacaccccctacacctttgacatccaggccat | |
| caatggagtctccagcaagagtcccttccccccacagcacgtctctgtcaacatcaccac | |
| aaaccaagccgccccctccaccgttcccatcatgcaccaagtcagtgccactatgaggag | |
| catcaccttgtcatggccacagccggagcagcccaatggcatcatcctggactatgagat | |
| ccggtactatgagaaggaacacaatgagttcaactcctccatggccaggagtcagaccaa | |
| cacagcaaggattgatgggctgcggcctggcatggtatatgtggtacaggtgcgtgcccg | |
| cactgttgctggctacggcaagttcagtggcaagatgtgcttccagactctgactgacga | |
| tgattacaagtcagagctgagggagcagctgcccctgattgctggctcggcagcggccgg | |
| ggtcgtgttcgttgtgtccttggtggccatctctatcgtctgtagcaggaaacgggctta | |
| tagcaaagaggctgtgtacagcgataagctccagcattacagcacaggccgaggctcccc | |
| agggatgaagatctacattgaccccttcacttatgaggatcccaacgaagctgtccggga | |
| gtttgccaaggagattgatgtatcttttgtgaaaattgaagaggtcatcggagcagggga | |
| gtttggagaagtgtacaaggggcgtttgaaactgccaggcaagagggaaatctacgtggc | |
| catcaagaccctgaaggcagggtactcggagaagcagcgtcgggactttctgagtgaggc | |
| gagcatcatgggccagttcgaccatcctaacatcattcgcctggagggtgtggtcaccaa | |
| gagtcggcctgtcatgatcatcacagagttcatggagaatggtgcattggattctttcct | |
| caggcaaaatgacgggcagttcaccgtgatccagcttgtgggtatgctcaggggcatcgc | |
| tgctggcatgaagtacctggctgagatgaattatgtgcatcgggacctggctgctaggaa | |
| cattctggtcaacagtaacctggtgtgcaaggtgtccgactttggcctctcccgctacct | |
| ccaggatgacacctcagatcccacctacaccagctccttgggagggaagatccctgtgag | |
| atggacagctccagaggccatcgcctaccgcaagttcacttcagccagcgacgtttggag | |
| ctatgggatcgtcatgtgggaagtcatgtcatttggagagagaccctattgggatatgtc | |
| caaccaagatgtcatcaatgccatcgagcaggactaccggctgcccccacccatggactg | |
| tccagctgctctacaccagctcatgctggactgttggcagaaggaccggaacagccggcc | |
| ccggtttgcggagattgtcaacaccctagataagatgatccggaacccggcaagtctcaa | |
| gactgtggcaaccatcaccgccgtgccttcccagcccctgctcgaccgctccatcccaga | |
| cttcacggcctttaccaccgtggatgactggctcagcgccatcaaaatggtccagtacag | |
| ggacagcttcctcactgctggcttcacctccctccagctggtcacccagatgacatcaga | |
| agacctcctgagaataggcatcaccttggcaggccatcagaagaagatcctgaacagcat | |
| tcattctatgagggtccagataagtcagtcaccaacggcaatggcatgagaactcttgtt | |
| tcttggggaaggagaggagggaaaaggaccagggtcaagggggaccagaggttgaccact | |
| gtggaatgtactggagagactggcttctcagctgaggaatgcatttccatcagtgaagaa | |
| tcaaccggacctgttgctagcaggcaatctccatttctcagtgacagaagcatgtttgag | |
| atgccgtgggaaaccaaatatataataataaaaatataaaaaggtgatgttcaacagaag | |
| tgaagacaaaacaatatgcatcaggagaacaagagtaaacccagctcccactctcagtgg | |
| gctgcagttgcccaaccacaggaagaaagggaaggaggtagagggaagaaacagaagcag | |
| tgttccattttcttcctcaccaatgacattcttttcttttctcctttcgtactcctccct | |
| gagagtcccctcccttctcccacactcgtttccctttgctcatgactcctgtagggaagt | |
| ttcttcaaacaaaacccagctcctgagtctccagatgttgttctgtcagttgccaaagga | |
| ctttgctgaccactgcatggggatccaaccaattcaattaatgtcttcatattgaagaag | |
| agatgtaccttcaattgaaaacctcgtttttcttttgtttgcattttctgcaaaaaggaa | |
| aaagaaaccacaaattggggaattc | |

TABLE 181

| | |
|---|---|
| cbe_3820530 PGAM1 | |
| phosphoglycerate mutase 1 (brain) | |
| length = 1709 | |
| gggcggggtgccgcatccccagcccgccgccatggccgcctacaaactggtgctgatccg | SEQ ID NO: 172 |
| gcacggcgagagcgcatggaacctggagaaccgcttcagcggctggtacgacgccgacct | |
| gagcccggcgggccacgaggaggcgaagcgcggcgggcaggcgctacgagatgctggcta | |
| tgagtttgacatctgcttcacctcagtgcagaagagagcgatccggaccctctggacagt | |
| gctagatgccattgatcagatgtggctgccagtggtgaggacttggcgcctcaatgagcg | |
| gcactatgggggtctaaccggtctcaataaagcagaaactgctgcaaagcatggtgaggc | |
| ccaggtgaagatctggaggcgctcctatgatgtcccaccacctccgatggagcccgacca | |
| tcctttctacagcaacatcagtaaggatcgcaggtatgcagacctcacagaagatcagct | |
| accctcctgtgagagtctgaaggatactattgccagagctctgcccttctggaatgaaga | |
| aatagttccccagatcaaggaggggaaacgtgtactgattgcagcccatggcaacagcct | |
| ccggggcattgtcaagcatctggagggtctctctgaagaggctatcatggagctgaacct | |
| gccgactggtattcccattgtctatgaattggacaagaacttgaagcctatcaagcccat | |
| gcagtttctgggggatgaagagacggtgcgcaaagccatggaagctgtggctgcccaggg | |
| caaggccaagaagtgaaggccggcggggaggatactgtccccaggagcaccctccctgcc | |
| cgtcttgtccctctgcccctcccacctgcacatgtcacactgaccacatctgtagacatc | |
| ttgagttgtagctgcagacggggaccagtggctcccattttcattttagccattttgtcg | |
| cctgcacccactcccttcatacaatctagtcagaatagcagttctagagcacaggttctc | |
| agtctaagctatggaaaagctccccttatccaacagagtttaaaagtagtgacttgggtt | |
| tttgcgagtgctttgtttactaaggactttggggaggaaccatgctaagccatgaccagt | |
| gaggagaagcaacagagcctgtctgtccccatgagcggagtctgtcctctgctcttctgc | |
| agtcaggtcactgcctactgcctgggggctctagtcattccagtggaagacgaatgtaac | |
| ctgcgtggtgatgtgacaactgtttcctccctgaccccagaggatctggctctaggttgg | |
| gatcaatcctgaatttcgttatgtgttaatttacttttattaaaaaagtatagtatatat | |
| aatacaaaacaataacccttctggggtttcttgtggcggttgaaatagtcccacatgtgg | |
| tcatcagaaatagcattcctcataccaatataggatcagctccttgacctctgaggggtc | |
| aggagtgcttcctggtgtgtgtattagaatcccttcctgccttgtttcatggcagtgaaa | |
| tgcctcttggtcctgtccagtgtatctttcactgatttctgaatcatgttctagttgctt | |
| gaccctgccacatgggtccagtgttcatctgagcataactgtactaaatcctttttccat | |
| atcagtataataaaggagtgatgtgcaat | |

TABLE 182

| | |
|---|---|
| cbe_3825897 POLR2L | |
| polymerase (RNA) II (DNA directed) polypeptide L, 7.6 kDa | |
| length = 392 | |
| agtctgggacgcgccgccgccatgatcatccctgtacgctgcttcacttgtggcaagatc | SEQ ID NO: 173 |
| gtcggcaacaagtgggaggcttacctggggctgctgcaggccgagtacaccgagggggac | |
| gcgctggatgccctgggcctgaagcgctactgctgccgccggatgctgctggcccacgtg | |
| gacctgatcgagaagctgctcaattatgcacccctggagaagtgaccacgctgaaaccca | |
| cccacccgctgtgctgaccatgggccctgagcgtcctaccccgaattcacgaggctgagg | |
| catccgggagctggcgtaatgcctggccgcagtgtgtgtgtatcccataccccactctgg | |
| aaggaaccatccagtaaaggtctttcagaacc | |

TABLE 183

| | |
|---|---|
| cbe_3826540 NYD-TSP1 | |
| testis-specific protein NYD-TSP1 | |
| length = 1880 | |
| ctttcccacccttcctccccacaaggctgtctgagatgccaccttccacctaaacatcat | SEQ ID NO: 174 |
| ctcccaaattttaatccttaactttggtctctgacttctgcttgattccacagtctccac | |
| ccacatttgcacaaaggaccatcacctgtccttcctggaatctctaaagttttctgcttt | |
| ccttctgtcctgatggccagctctgctaagtcagctgagatgcccaccatctccaaaacc | |
| cttaaccctactcctgatcctcatcaagaatatctggaccctaggattaccattgcctta | |
| ttcgaaattggatcacattccccttcctcctggggctctctccctttcctaaagaatagc | |
| agccatcaagttacagaacaacagactgcacagaagtttaacaatctcttaaaagaaatt | |
| aaagatattcttaaaaatatggcaggttttgaagagaagatcacagaagcaaaagaactt | |
| tttgaggaaaccaatattactgaggatgtgtcagcccacaaagaaaatatcagaggactt | |
| gacaaaatcaatgaaatgttatcaacaaacctgcctgttagtttagccccagagaaagaa | |
| gacaatgaaaagaaacaggagatgatattggaaaccaatattactgaggatgtgtcagcc | |
| cacaaagaaaatatcagaggacttgacaaaatcaatgaaatgttatcaacaaacctgcct | |
| gttagtttagccccagagaaagaagacaatgaaaagaaacagcagatgataatggaaaac | |
| cagaactctgagaacaccgcacaagtttttgcaagagatttggtaaatcgtttagaagaa | |
| aaaaaagtccttaacgaaactcaacaaagtcaggaaaaagcaaaaaacagacttaatgtt | |
| caagaagaaactatgaaaattaggaacaacatggagcagttactacaggaagcagaacac | |
| tggagtaaacaacatactgagctcagtaaactgataaaatcctatcagaaatctcagaaa | |
| gacataagtgaaactcttggaaataatggagtcggtttccaaacccagccaaataatgaa | |
| gtgtcggctaagcatgagctggaggaacaggtgaagaaactgagccatgacacctattca | |
| ttgcagttgatggcagctttgctagagaatgaatgccaaatcttacagcagagagtagag | |
| attctcaaggaactccatcatcagaaacagggaactctgcaagagaagccaattcagata | |
| aactataaacaggacaagaaaaatcagaagccatcagaagcaaagaaagtagaaatgtat | |
| aagcagaacaagcaagcaatgaagggtacattttggaaaaaagacagatcctgtagaagc | |
| ctggatgtttgtcttaataagaaagcttgcaatacccagttcaatattcatgttgcaaga | |
| aaagctcttaggggaaaaatgaggtcagctagcagcctaagatagaaaataccaaaagca | |
| gatgaaaaggtgaatccttaaaaaactacatctgttacctccaacttgtcagtcatgatc | |
| aatcatcaaaccttgggcagatctgctggttcaaccaagaaagagctatagaataaggag | |
| attgacacttatattactcattacttcagcagttatgtaagtctggtctttaatgaccat | |
| tgtgtctacatcaagttcataacttttcacctttacaatctgcttgttttttttgtaatt | |
| attagatagacagcctagagtgagaagttaaaggtccaattaagttaagatctaaataaa | |
| aaccagaatacaagggaaaaaaaaaaaaaaaaaaaaaaaaaaaaaaaaaaaaaaaaaaaa | |
| aaaaaaaaaaaaaaaaaaaa | |

TABLE 184

| | |
|---|---|
| cbe_3833533 TRIM29 | |
| tripartite motif-containing 29 | |
| length = 3018 | |
| ctcctcacaggtgtgtctctagtcctcgtggttgcctgccccactccctgccgagacgcc | SEQ ID NO: 175 |
| tgccagaaaggtcacctatcctgaaccccagcaagcctgaaacagctcagccaagcaccc | |
| tgcgatggaagctgcagatgcctccaggagcaacgggtcgagcccagaagccagggatgc | |
| ccggagcccgtcgggccccagtggcagcctggagaatggcaccaaggctgacggcaagga | |
| tgccaagaccaccaacgggcacggcggggaggcagctgagggcaagagcctgggcagcgc | |
| cctgaagccaggggaaggtaggagcgccctgttcgcgggcaatgagtggcggcgacccat | |
| catccagtttgtcgagtccggggacgacaagaactccaactacttcagcatggactctat | |
| ggaaggcaagaggtcgccgtacgcagggctccagctgggggctgccaagaagccacccgt | |
| tacctttgccgaaaagggcgagctgcgcaagtccattttctcggagtcccggaagcccac | |
| ggtgtccatcatggagcccggggagacccggcggaacagctacccccgggccgacacggg | |
| ccttttttcacggtccaagtccggctccgaggaggtgctgtgcgactcctgcatcggcaa | |
| caagcagaaggcggtcaagtcctgcctggtgtgccaggcctccttctgcgagctgcatct | |
| caagccccacctggagggcgccgccttccgagaccaccagctgctcgagcccatccggga | |
| ctttgaggcccgcaagtgtcccgtgcatggcaagacgatggagctcttctgccagaccga | |
| ccagacctgcatctgctacctttgcatgttccaggagcacaagaatcatagcaccgtgac | |
| agtggaggaggccaaggccgagaaggagacggagctgtcactgcaaaaggagcagctgca | |
| gctcaagatcattgagattgaggatgaagctgagaagtggcagaaggagaaggaccgcat | |
| caagagcttcaccaccaatgagaaggccatcctggagcagaacttccgggacctggtgcg | |
| ggacctggagaagcaaaaggaggaagtgagggctgcgctggagcagcgggagcaggatgc | |
| tgtggaccaagtgaaggtgatcatggatgctctggatgagagagccaaggtgctgcatga | |
| ggacaagcagacccgggagcagctgcatagcatcagcgactctgtgttgtttctgcagga | |
| atttggtgcattgatgagcaattactctctccccccacccctgcccacctatcatgtcct | |
| gctggagggggagggcctgggacagtcactaggcaacttcaaggacgacctgctcaatgt | |
| atgcatgcgccacgttgagaagatgtgcaaggcggacctgagccgtaacttcattgagag | |
| gaaccacatggagaacggtggtgaccatcgctatgtgaacaactacacgaacagcttcgg | |
| gggtgagtggagtgcaccggacaccatgaagagatactccatgtacctgacacccaaagg | |
| tggggtccggacatcataccagccctcgtctcctggccgcttcaccaaggagaccaccca | |
| gaagaatttcaacaatctctatggcaccaaaggtaactacacctcccgggtctgggagta | |
| ctcctccagcattcagaactctgacaatgacctgcccgtcgtccaaggcagctcctcctt | |
| ctccctgaaaggctatccctccctcatgcggagccaaagccccaaggcccagccccagac | |
| ttggaaatctggcaagcagactatgctgtctcactaccggccattctacgtcaacaaagg | |
| caacgggattgggtccaacgaagccccatgagctcctggcggaaggaacgaggcgccaca | |
| cccctgctcttcctcctgaccctgctgctcttgccttctaagctactgtgcttgtctggg | |
| tgggagggagcctggtcctgcacctgccctctgcagccctctgccagcctcttgggggca | |
| gttccggcctctccgacttccccactggccacactccattcagactcctttcctgccttg | |
| tgacctcagatggtcaccatcattcctgtgctcagaggccaacccatcacaggggtgaga | |
| taggttggggcctgccctaacccgccagcctcctcctctcgggctggatctgggggctag | |
| cagtgagtacccgcatggtatcagcctgcctctcccgcccacgccctgctgtctccaggc | |
| ctatagacgtttctctccaaggccctatcccccaatgttgtcagcagatgcctggacagc | |
| acagccacccatctcccattcacatggcccacctcctgcttcccagaggactggccctac | |
| gtgctctctctcgtcctacctatcaatgcccagcatggcagaacctgcagcccttggcca | |
| ctgcagatggaaacctctcagtgtcttgacatcaccctacccaggcggtgggtctccacc | |
| acagccactttgagtctgtggtccctggagggtggcttctcctgactggcaggatgacct | |
| tagccaagatattcctctgttccctctgctgagataaagaattcccttaacatgatataa | |
| tccacccatgcaaatagctactggcccagctaccatttaccatttgcctacagaatttca | |
| ttcagtctacactttggcattctctctggcgatggagtgtggctgggctgaccgcaaaag | |
| gtgccttacacactgcccccaccctcagccgttgccccatcagaggctgcctcctccttc | |
| tgattaccccccatgttgcatatcagggtgctcaaggattggagaggagacaaaaccagg | |
| agcagcacagtggggacatctcccgtctcaacagccccaggcctatgggggctctggaag | |
| gatgggccagcttgcaggggttggggagggagacatccagcttgggctttcccctttgga | |
| ataaaccattggtctgtc | |

TABLE 185

| | |
|---|---|
| cbe_3834015 TFPI | |
| tissue factor pathway inhibitor (lipoprotein-associated coagulation inhibitor) | |
| length = 915 | |
| atgatttacacaatgaagaaagtacatgcactttgggcttctgtatgcctgctgcttaat | SEQ ID NO: 176 |
| cttgcccctgcccctcttaatgctgattctgaggaagatgaagaacacacaattatcaca | |
| gatacggagttgccaccactgaaacttatgcattcattttgtgcattcaaggcggatgat | |
| ggcccatgtaaagcaatcatgaaaagatttttcttcaatattttcactcgacagtgcgaa | |
| gaatttatatatgggggatgtgaaggaaatcagaatcgatttgaaagtctggaagagtgc | |
| aaaaaaatgtgtacaagagataatgcaaacaggattataaagacaacattgcaacaagaa | |
| aagccagatttctgctttttggaagaagatcctggaatatgtcgaggttatattaccagg | |
| tatttttataacaatcagacaaaacagtgtgaacgtttcaagtatggtggatgcctgggc | |
| aatatgaacaattttgagacactggaagaatgcaagaacatttgtgaagatggtccgaat | |
| ggtttccaggtggataattatggaacccagctcaatgctgtgaataactccctgactccg | |
| caatcaaccaaggttcccagcctttttgaatttcacggtccctcatggtgtctcactcca | |
| gcagacagaggattgtgtcgtgccaatgagaacagattctactacaattcagtcattggg | |
| aaatgccgcccatttaagtacagtggatgtgggggaaatgaaaacaattttacttccaaa | |
| caagaatgtctgagggcatgtaaaaaaggtttcatccaaagaatatcaaaaggaggccta | |
| attaaaaccaaaagaaaaagaaagaagcagagagtgaaaatagcatatgaagaaattttt | |
| gttaaaaatatgtga | |

TABLE 186

| | |
|---|---|
| cbe_3842044 PSMB3 | |
| proteosome (prosome, macropain) subunit, beta type, 3 | |
| length = 784 | |
| gagcggttgcgcagtgaaggctagacccggtttactggaattgctctggcgatcgagggg | SEQ ID NO: 177 |
| tcctagtacaccgcaatcatgtctattatgtcctataacggaggggccgtcatggccatg | |
| aaggggaagaactgtgtggccatcgctgcagacaggcgcttcgggatccaggcccagatg | |
| gtgaccacggacttccagaagatctttcccatgggtgaccggctgtacatcggtctggcc | |
| gggctcgccactgacgtccagacagttgcccagcgcctcaagttccggctgaacctgtat | |
| gagttgaaggaaggtcggcagatcaaaccttataccctcatgagcatggtggccaacctc | |
| ttgtatgagaaacggtttggcccttactacactgagccagtcattgccgggttggacccg | |
| aagacctttaagcccttcatttgctctctagacctcatcggctgccccatggtgactgat | |
| gactttgtggtcagtggcacctgcgccgaacaaatgtacggaatgtgtgagtccctctgg | |
| gagcccaacatggatccggatcacctgtttgaaaccatctcccaagccatgctgaatgct | |
| gtggaccgggatgcagtgtcaggcatgggagtcattgtccacatcatcgagaaggacaaa | |
| atcaccaccaggacactgaaggcccgaatggactaaccctgttcccagagcccacttttt | |
| tttctttttttgaaataaaatagcctgtctttcaaaaaaaaaaaaaaaaaaaaaaaaaaa | |
| aaaa | |

TABLE 187

| | |
|---|---|
| cbe_3845697 Hs. 191063 | |
| Human DNA sequence from clone RP1-63M2 on chromosome 20 Contains the gene for | |
| CBFA2T2 (core-binding factor, runt domain, alpha subunit 2; translocated to, | |
| 2), part of a gene for a protein similar to ACTIN, the E2F1 (E2F transcription | |
| factor 1) gene, a nov | |
| length = 532 | |
| tttggcatggacacaagtggggcatcacctttattgccataaagtagggccctgtgggta | SEQ ID NO: 178 |
| ggcagctggactggcattctctgaactggcagtggttggggagattcaaactgcagttca | |
| aaatgagggtcccatgggtcccaaataccttctcctggcccagaaagggtatgggggtga | |
| cccatccagtcagctccaacctccaggaggaccctgccagctccaaacacttgactcttt | |
| agatagagagctgtcccccaacgcagtgccaccccccccagtccagcctgactcagttga | |
| acacatcgtacagcagcctggagccacactcctggtacatggcccgagttatccagcggc | |
| gctggaaggagtgcagggaggccaccatggagccgccggtccacacagccatgccacgcc | |
| catgcttggccaccaggtggggccgcagggccgcgtagccgtgccgccggcactgcgcct | |
| ncagctccttgtccaggcgctcggcgaagccaggaaacagtgtggagccgcc | |

TABLE 188

| | |
|---|---|
| cbe_3849002 CKLF1 | |
| chemokine-like factor 1 | |
| length = 689 | |
| gcaagagagcgggaagccgagctgggcgagaagtaggggagggcggtgctccgccgcggt | SEQ ID NO: 179 |
| ggcggttgctatcgcttcgcagaacctactcaggcagccagctgagaagagttgagggaa | |
| agtgctgctgctgggtctgcagacgcgatggataacgtgcagccgaaaataaaacatcgc | |
| cccttctgcttcagtgtgaaaggccacgtgaagatgctgcggctggcactaactgtgaca | |
| tctatgaccttttttatcatcgcacaagcccctgaaccatatattgttatcactggattt | |
| gaagtcaccgttatcttatttttcatacttttatatgtactcagacttgatcgattaatg | |
| aagtggttattttggcctttgcttgatattatcaactcactggtaacaacagtattcatg | |
| ctcatcgtatctgtgttggcactgataccagaaaccacaacattgacagttggtggaggg | |
| gtgtttgcacttgtgacagcagtatgctgtcttgccgacggggcccttatttaccggaag | |
| cttctgttcaatcccagcggtccttaccagaaaaagcctgtgcatgaaaaaaaagaagtt | |
| ttgtaattttatattactttttagtttgatactaagtattaaacatatttctgtattctt | |
| ccaaaaaaaaaaaaaaaaaaaaaaaaaaa | |

TABLE 189

| | |
|---|---|
| cbe_3880186 FLJ20343 | |
| hypothetical protein FLJ20343 | |
| length = 2784 | |
| gatgccccgcccgcgcccatggcgcagccacacacgacctcggtcccgtacttcgcgcgc | SEQ ID NO: 180 |
| tctcctgcaccgccgccgccatctcgctcaggagctcctccacaaccgccggcaacacta | |
| cggccatcgcgccgcaggacacgccctccacgaccggcggaccgccgcgacgctccagct | |
| gactgcgcctacctgtggaggatcctgaccccccgccggggcagggcgagacggagtgac | |
| gtcggggcgcgtcatcgcgcgtgcggacgcagggatgtcctgctcagtcgtcagggcccg | |
| gccaatccggagggcgctcggcgcgtggtcgggggccaggagcgcgtctggcctgcagtg | |
| cgcagaggacgcggcgggagcatgttccggctcctgagctggagcctgggccgaggcttc | |
| ctgcgggccgcggggcggcggtgccggggctgctccgcgcgcctgctcccggggctggca | |
| ggaggtccggggcccgaggtgcaggtgccgccatcccgagtcgcgccgcacggccggggc | |
| ccaggcctgctgccgctgctggcagcgctcgcctggttctcgaggcccgctgcggcagag | |
| gaggaggagcagcagggagccgacggggccgctgccgaggacggggcggacgaggccgag | |
| gcagagatcatccagctgctgaagcgagccaagttgagcattatgaaagatgagccagaa | |
| gaggctgagttaattttgcatgacgctcttcgtctcgcctatcagactgataacaagaag | |
| gccatcacttacacttatgatttgatggccaacttagcatttatacggggtcagcttgaa | |
| aatgctgaacaactttttaaagcaacaatgagttacctccttggagggggcatgaagcag | |
| gaggacaatgcaataattgaaatttccctaaagctggccagtatctatgctgcgcagaac | |
| agacaggaatttgctgttgctggctatgaattctgcatttcaactctagaggaaaaaatt | |
| gaaagagaaaaggaattagcagaagacattatgtcagtggaagagaaagccaatacccac | |
| ctcctcttgggcatgtgcttagacgcctgtgctcgctaccttctgttctccaagcagccg | |
| tcacaggcacaaaggatgtatgaaaaagctctgcagatttctgaagaaatacaaggagaa | |
| agacacccacagaccattgtgctgatgagtgacctggctactaccctggatgcacagggc | |
| cgctttgatgaggcctatatttatatgcaaagggcatcagatctggcaagacagataaat | |
| catcctgagctacacatggtactcagtaatctagctgcagttttgatgcacagagaacga | |
| tatacacaagcaaaagagatctaccaggaagcactgaagcaagcaaagctgaaaaaagat | |
| gaaatttctgtacaacacatcagggaagagttggctgagctgtcaaagaaaagtagacct | |
| ttgacaaattctgtcaagctctaaatccatttttgtgtagggagaataatgtctagtaat | |
| gtggaagaatagctatcattcctgtctctgtggcacccgatcaatggcttaaatctgtcg | |
| tttttgatattcaggtttcctcaatttagccttagtgaaggaggggttgtacacactgcc | |
| atttttgtattttaaaggaaaaatgactttcattcccaactgattatgacctttcaggat | |
| gtcgtcaagtgatgctttcagttgtaacacgtgacttggtgctgtccctgctggtctaag | |
| tagaactgtagattcatatgggctggtgttcctgtgcgctgtgggtgtggtgattcagcc | |
| tggcatttctaccataagtttttggtctgctgatttgctgccctgtcttctcttacttta | |
| ctttatcaatacctggcaaactgaccagaattaccttcctcatggcgaagggggattatg | |
| gtgaattgttgttcttatagtctgtttcatgaagcacaagtggaatttaatacataaaag | |
| agaaaaatatcttagtttgctaccagcatccagcatgaagttgtaaagtggggattaggc | |
| acgtgacagtatagcacccatttgaatttaaataaaagtgaaccatatttatctggttat | |
| ataaaactaaaaatgggggtgtttatataaaactaaaaactaagaatgatgtaacctttt | |
| gtctgtgttatctgaacactctacttcctttgcagccttagtcacacaactgagtcatct | |
| caagtactctttaaggacacacagcccaggctgttctgagtcagaataggcccctacagg | |
| tatattttaaaactcttcgtaattctaatgtgtactgctggtatagctgaactactgacc | |
| tggatcttagtcctagcctttttgcttttgcaatttcagtatcttcatctctaaactagg | |
| gaaacactgggattctttcttagctgtgggggaaggtatttggttagatgactttgaatg | |
| aatagactgctgtgctgaaagagctttatcacactgtctcaaagtatgtaaagatacata | |
| ggtggatgctcttactgcagcagtcatgaatacatttttagccatttacctaaggaaaaa | |
| gacagtttttctaggtaccatgaaggaagattgaccctgttggtatgcctgtgggggtgg | |
| gatgtgagtgggactgataaactgatacttttggttcgtatgtacatactggaagaatct | |
| tcataataaatgagactacacaac | |

TABLE 190

| | |
|---|---|
| cbe_3884811 RCP | |
| Rab coupling protein | |
| length = 2112 | |
| cccgcttctggagtgttatcgtcaccatgtccctaatggtctcggctggccggggcctgg | SEQ ID NO: 181 |
| gggccgtgtggtccccaacccacgtgcaggtgacggtgctgcaggcgcggggcctgcggg | |
| ccaagggccccgggggcacgagcgacgcgtacgcggtgatccaggtgggcaaggagaagt | |
| acgccacctccgtgtcggagcgcagcctgggcgcgcccgtgtggcgcgaggaggccacct | |
| tcgagctgccatcgctgctgtcctccggacccgcggccgccgccaccctgcagctcaccg | |
| tgctgcaccgcgcgctgctcggcctcgacaagttcctgggccgcgccgaggtggacctgc | |
| gggatctgcaccgcgaccagggccgcaggaagacgcagtggtataagttgaaatccaaac | |
| caggaaagaaggacaaggagcgaggagaaattgaggttgacatccagtttatgagaaaca | |
| acatgactgccagcatgtttgacctttctatgaaagacaagtctcggaatccatttggaa | |
| agctgaaggacaagatcaaggggaagaataaggacagtgggtcagacaccgcctccgcca | |
| tcatccctagcacgacaccttcggtcgacagtgatgatgagtctgtggttaaagacaaga | |
| aaaagaaatcaaagatcaagaccttactttccaagtcaaatttgcagaagacgcctcttt | |
| cccagtccatgtctgtcctgccgacttcaaagccagaaaaagtgctgcttcgtcccggag | |
| actttcagtcccagtgggatgaagatgacaatgaggatgagtcctcctcggcctcggatg | |
| tcatgtctcacaagagaacagcgagtacggatcttaagcaactgaaccaggtcaacttta | |
| cccttcccaagaaggaaggactttcctttcttggtggccttcggtctaagaatgatgtcc | |
| tttcccgctctaatgtctgcatcaatgggaaccatgtttacctggagcagccccaaccca | |
| ccggtgagatcaaggatagcagcccgtcctcctccccatcccccaagggtttcagaaaga | |
| aaacattgttctcttctacagagaacctggcggctgggtcttggaaggagcctgctgaag | |
| gaggtgggctgtctactgacagggatgtctccgaatcttccaccaaggactccttgaagt | |
| ctatgaccttgccgacctaccgacctgccccactgatcagtggggacctcagggaaaaaa | |
| tggcccccgcaaactcagaggccacaaaagaagctaaggagagcaagaagccagagagca | |
| ggaggtcctctttgctgtctctgatgacggggaagaaggatgtggctaagggcagtgaag | |
| gtgaaaaccttctcacggtcccagggagggagaaggaaggcatgctgatgggggttaagc | |
| cgggggaggacgcatcggggcctgctgaagaccttgtgagaagatctgagaaagatactg | |
| cagctgttgtctccagacagggcagctccctgaacctctttgaagatgtgcagatcacag | |
| aaccagaagctgagccagagtccaagtctgaaccgagacctccaatttcctctccgaggg | |
| ctccccagaccagagctgtcaagccccgacttcatcctgtgaagccaatgaatgccacgg | |
| ccaccaaggttgctaactgcagcttgggaactgccaccatcatcagtgagaacttgaaca | |
| atgaggtcatgatgaagaaatacagcccctcggaccctgcatttgcatatgcgcagctga | |
| cccacgatgagctgattcagctggtcctcaaacagaaggaaacgataagcaagaaggagt | |
| tccaggtccgcgagctggaagactacattgacaacctgcttgtcagggtcatggaagaaa | |
| cccccaatatcctccgcatcccgactcaggttggcaaaaaagcaggaaagatgtaaatca | |
| gcagaaaaaaaacaccgagacgtttctgtgacttcactttcacctgctccaggggtcaag | |
| gacttgccttgcctgataaccagccagcaggctccgaatcaccatctccctcacatgtta | |
| tccggcaagagt | |

TABLE 191

| | |
|---|---|
| cbe_4138433 KIFAP3 | |
| kinesin-associated protein 3 | |
| length = 2997 | |
| caagaggtgcagagcaagcgcatgcgtcgtgacggcccggcttaggcgactctgggcggg | SEQ ID NO: 182 |
| tctgggccgctccagtgttttggggcacagaagctgtgggaggagctggaggcttcaccg | |
| tggtaaccacagcgccgctgctgccccgccttgcaggcctcaggactgtcatcgcctctg | |
| ggtgtgagggtactttggccaccgtccccggaaataaccgcgcctgcctctcaagatacc | |
| ccatcctctccacgccgctgccgctgccgccatgcaaggggaggacgccagatacctcaa | |
| aaggaaagttaaaggagggaatatagatgtacatccatcagaaaaagcactcattgttca | |
| ctatgaagtggaagctaccattcttggagaaatgggggaccccatgttgggagaacgaaa | |
| agaatgtcaaaaaatcattcgacttaagagtctcaatgccaacacagatataacttccct | |
| ggcaaggaaggtggttgaagaatgtaaactcattcatccttcaaaactaaatgaggtaga | |
| acagctgttgtactatctacagaaccgccgtgattcattgtcaggaaaagagaaaaaaga | |
| aaaatcaagcaagcctaaagatccacctccttttgaaggaatggagattgatgaagttgc | |
| taacattaatgacatggatgaatatattgagttattatatgaagatattcctgacaaagt | |
| tcggggttctgctttgatcctgcagcttgctcgaaatcctgataacttggaagaactact | |
| attgaatgaaactgcccttggtgcattagcaagggtcctgagagaagactggaagcaaag | |
| tgtcgagttagctacaaacataatttacatctttttttgtttctccagcttttctcaatt | |
| tcatggacttattactcactataaaattggagctctgtgtatgaatattattgatcatga | |
| gttaaaaagacatgagctttggcaagaagaactctcaaagaagaagaaagctgttgatga | |
| agaccctgaaaaccaaaccttgagaaaggattatgaaaaaacctttaaaaagtaccaggg | |
| gcttgtggtaaaacaggaacagctattacgagttgctctttatttgcttctgaatcttgc | |
| tgaggatactcgtaccgaactgaaaatgaggaacaagaacatagttcacatgttggtgaa | |
| agcccttgatcgggacaattttgagctgctaattttagttgtgtcattcttgaagaaact | |
| cagcatttttatggagaataaaaatgatatggtggaaatggatattgttgaaaaactggt | |
| gaaaatgataccttgtgagcatgaagacctgctgaatatcaccctccgacttttactaaa | |
| cctatcctttgacacaggactgaggaataagatggtacaagttggactgcttcccaagct | |
| cactgcactcctaggcaatgacaactacaaacaaatagcaatgtgtgttctttaccacat | |
| aagcatggatgaccgctttaaatcaatgtttgcatacactgactgtataccacagttaat | |
| gaagatgctgtttgaatgttcagatgaacgaattgacttggaactcatttctttctgcat | |
| taatcttgctgctaacaaaagaaatgtacagcttatctgtgaaggaaatgggctgaagat | |
| gctcatgaagagggctctgaagtttaaggatccattgctgatgaaaatgattagaaacat | |
| ttctcagcatgatggaccaactaaaaatctgtttattgattatgttggggaccttgcagc | |
| ccagatctctaatgatgaagaagaggagtttgtgattgaatgtttgggaactcttgcaaa | |
| cttgaccattccagacttagactgggaattggttcttaaagaatataagttggttccata | |
| cctcaaggataaactaaaaccaggtgctgcagaagatgatcttgttttagaagtggttat | |
| aatgattggaactgtatccatggatgactcttgtgctgcattgctagccaaatctggcat | |
| aatccctgcactcattgaattgctaaatgctcaacaagaagatgatgaatttgtgtgtca | |
| gataatttatgtcttctaccagatggttttccaccaagccacaagagacgtcataatcaa | |
| ggaaacacaggctccagcatatctcatagacctaatgcatgataagaataatgaaatccg | |
| aaaggtctgtgataatacattagatattatagcggaatatgatgaagaatgggctaagaa | |
| aattcagagtgaaaagtttcgctggcataactctcagtggctggagatggtagagagtcg | |
| tcagatggatgagagtgagcagtacttgtatggtgatgatcgaattgagccatacattca | |
| tgaaggagatattctcgaaagacctgaccttttctacaactcagatggattaattgcctc | |
| tgaaggagccataagtcccgatttcttcaatgattaccaccttcaaaatggagatgttgt | |
| tgggcagcattcatttcctggcagccttggaatggatggctttggccaaccagttggcat | |
| tcttggacgccctgccacagcatatggattccgccctgatgaaccttactactatggcta | |
| tggatcttgataaagtatctgtttccatgtgtaatctcagcttagaagaaatctgtgtgg | |
| gttgggttaattttggatctttgcctaataatgcatgttgatgttattgtgggtctgtgt | |
| ttgtttttatttttatatgttgttagctgcagattaaccccagcccctctgtcttctgtt | |
| aagtacagttgatactgacattgttcactcatcaaaccacatcttgatgctaagtaacat | |
| ttcccatgagcctcaaaactgaatgctgaaaagctactagactggaaaacaaacactgca | |
| ttatgtatgttaagtgactaatttaatttcaattaaaaagcgtaaagtgaaaatgaa | |

TABLE 192

| | |
|---|---|
| cbe_422204 Prey2335 | |
| PathCalling Prey Sequence 2335 | |
| length = 394 | |
| aaggaaggaaggaaggaaggaaggaaggaaggaaagaaagaaattcatccagcatgtccc | SEQ ID NO: 183 |
| tttcttctcctatgggatttaccaactgacaaattctagtgctcctgccatcacccacac | |
| atgtgcagcaaagcctccacatttgcaaaatctaagtcaatctctaaaagatgcttcgag | |
| gaagcacccagttccttctgctaataaccgcagtgtgtcatgtgacacctccccctcccc | |
| caaccagttctctctgaaaactacccacccaccccaccaataaagtagcatgacgtctgt | |
| ccttcagctgccaatatctacaatcaaccaacaagtcattaaaaaaaaaaaaaaaaaaaa | |
| aaaaaaaaaaaaaaaaaaaaaaaaaaaaaaagaa | |

TABLE 193

| | |
|---|---|
| cbe_422227 Prey277610 | |
| PathCalling Prey Sequence 277610 | |
| length = 140 | |
| gtctgagtgctactgaagaggacgtcgtcacttctggaccaagccctgcccaacgacgtc | SEQ ID NO: 184 |
| ctcttcagtagcacgtacccttctctccccaagtcgatcccgttgaggaggccttcttac | |
| accttaggaatgaaatcgct | |

TABLE 194

| | |
|---|---|
| cbe_422236 BCR1 | |
| PathCalling Prey Sequence 1759 | |
| length = 553 | |
| ggcagaggggttacaactcaggttcccacacctcgcttttcacagcctccgagggcagca | SEQ ID NO: 185 |
| gtgcacatgggggagacatctcccacgaggccaaggcctccagtgctcaccgatgccctc | |
| tgtgaagttggggtagaactcgtcagtgaaaaggggctcgggcagctcacggaagtacag | |
| cttcagcatgcctgcgatggcgttcacgtccatctcgctcatcatcaccgacacgtcctt | |
| gttatctggaaagagcatggaaatgcagcggcctccttgaagatcctgataaatgaagaa | |
| aagacacagagaaggatgtgacatgcctgggccgtggagcactctgagatctcatcgcgg | |
| acaccactgcccacacctccatcccgtcctgcgcaggccggcactcactgacgttgaagc | |
| ctgccttcagtgcctggatgtccgtggccaccccagacatgcggtagatgcccacctcct | |
| ccatgcctcggcgcttgatctcctcacacactggcgcacgatgtagggcacttggacctc | |
| tctctcctgcgga | |

TABLE 195

| | |
|---|---|
| cbe_4279962 DKKL1-pending | |
| soggy-1 gene | |
| length = 908 | |
| aaaattcggcacgaggccgggctgtggtctagcataaaggcggagcccagaagaaggggc | SEQ ID NO: 186 |
| ggggtatgggagaagcctccccacctgcccccgcaaggcggcatctgctggtcctgctgc | |
| tgctcctctctaccctggtgatcccctccgctgcagctcctatccatgatgctgacgccc | |
| aagagagctccttgggtctcacaggcctccagagcctactccaaggcttcagccgacttt | |
| tcctgaaaggtaacctgcttcggggcatagacagcttattctctgcccccatggacttcc | |
| ggggcctccctgggaactaccacaaagaggagaaccaggagcaccagctggggaacaaca | |
| ccctctccagccacctccagatcgacaagatgaccgacaacaagacaggagaggtgctga | |
| tctccgagaatgtggtggcatccattcaaccagcggaggggagcttcgagggtgatttga | |
| aggtacccaggatggaggagaaggaggccctggtacccatccagaaggccacggacagct | |
| tccacacagaactccatccccgggtggccttctggatcattaagctgccacggcggaggt | |
| cccaccaggatgccctggagggcggccactggctcagcgagaagcgacaccgcctgcagg | |
| ccatccgggatggactccgcaaggggacccacaaggacgtcctagaagaggggaccgaga | |
| gctcctcccactccaggctgtccccccgaaagacccacttactgtacatcctcaggccct | |
| ctcggcagctgtaggggtggggaccggggagcacctgcctgtagcccccatcagaccctg | |
| ccccaagcaccatatggaaataaagttctttcttacatctaaaaaaaaaaaaaaaaaaaa | |
| aaaaaaaa | |

TABLE 196

| | |
|---|---|
| cbe_4281271 CRYAB | |
| crystallin, alpha B | |
| length = 691 | |
| gacccctcacactcacctagccaccatggacatcgccatccaccacccctggatccgccg | SEQ ID NO: 187 |
| ccccttctttcctttccactcccccagccgcctctttgaccagttcttcggagagcacct | |
| gttggagtctgatcttttcccgacgtctacttccctgagtcccttctaccttcggccacc | |
| ctccttcctgcgggcacccagctggtttgacactggactctcagagatgcgcctggagaa | |
| ggacaggttctctgtcaacctggatgtgaagcacttctccccagaggaactcaaagttaa | |
| ggtgttgggagatgtgattgaggtgcatggaaaacatgaagagcgccaggatgaacatgg | |
| tttcatctccagggagttccacaggaaataccggatcccagctgatgtagaccctctcac | |
| cattacttcatccctgtcatctgatggggtcctcactgtgaatggaccaaggaaacaggt | |
| ctctggccctgagcgcaccattcccatcacccgtgaagagaagcctgctgtcaccgcagc | |
| ccccaagaaatagatgccctttcttgaattgcattttttaaaacaagaaagtttccccac | |
| cagtgaatgaaagtcttgtgactagtgctgaagcttattaatgctaagggcaggcccaaa | |
| ttatcaagctaataaaatatcattcagcaac | |

TABLE 197

| | |
|---|---|
| cbe_4344185 SRA1 | |
| steroid receptor RNA activator 1 | |
| length = 2069 | |
| ttatagcaaaatcagtgcaaataaaaatccctcagtgacctcactggatgtgagtatatt | SEQ ID NO: 188 |
| gggcctgggacagggctgggggctaacaccctgtgtgagatgagtgtctttgtgtctgtg | |
| cttgatgttggtggctctctgtagtcacatgacagcatgggtgtgatggagatctgactt | |
| cattcaacaaacatattttctaaggagttccctgtgccaggcactaagctgggcactggg | |
| aatgtaataaaatagtcaaggtcccaccttctaagactgtccgacagggaaacgaacaag | |
| agtcaaataaggcagaagatgtgatgtaatacacctacgaaatctcagagggttgtaggg | |
| tcgtgggagctcaagtgagacacttaacctggcctgagacattccagaaggcctcctgaa | |
| gaactgacatctgaactgagaactgaaggaagatgagtactagtgaggctaccggacgtg | |
| aatgtggagattgtgcagggcaatgcaagaggaggctgtagaagtcaacctggctagatc | |
| acagcggggtgtatgtggggcaggagcttctttgtttgaatttgctcctgagaggatgag | |
| gcctcctagagcactggctcctggacagcaacctcctttggtgccttgtgaccagggccc | |
| tgatggttcattagatggagccttcgagtcttagggagttgccgcagggtccccacagcg | |
| gctcccgacggttgtgaaccagcatccattctccacggattccggcaacccgcctggccc | |
| tggacgtgtctcaactggcccgcgtgaggggccgccccggaaatgacgcgctgccccgct | |
| ggccaagcggaagtggagatggcggagctgtacgtgaagccgggcaacaaggaacgcggc | |
| tggaacgacccgccgcagttctcatacgggctgcagacccaggccggcggacccaggcgc | |
| tcgctgcttaccaagagggtagccgcaccccaggatggatcccccagagtccccgcatca | |
| gagacttctcctgggcctcccccaatggggcctccacctccttcaagtaaggctcccagg | |
| tccccacctgtggggagtggtcctgcctctggcgtggagcccacaagtttcccagtcgag | |
| tctgaggctcgactgatggaggatgtgctgagacctttggaacaggcattggaagactgc | |
| cgtggccacacaaggaagcaggtatgtgatgacatcagccgacgcctggcactgctgcag | |
| gaacagtgggctggaggaaagttgtcaatacctgtaaagaagagaatggctctactggtg | |
| caagagctttcaagccaccggtgggacgcagcagatgacatccaccgctccctcatggtt | |
| gaccatgtgactgaggtcagtcagtggatggtaggagttaaaagattaattgcagaaaag | |
| aggagtctgttttcagaggaggcagccaatgaagagaaatctgcagccacagctgagaag | |
| aaccataccataccaggcttccagcaggcttcataatcctcggttccccagactcaccgg | |
| acaccatcccctatgccttggagaccttctgtcacttggctcccttcttaccaccaccaa | |
| gactgtcccactgggcctgacccacctatgagggaagaagtcccacctgggccagaggga | |
| gttcatgtgttactcataacatgcatttcaataaaaacatctctgcggtgggccttgggt | |
| aggagagatgaacccttccggtgccaagctagtcccctctggtgtcctcgactgccctgc | |
| tccctgtgtatctgcaaacctctgttctcccttctccattcatcaggaagggatctgctg | |
| ggtaaagtcagactactgcctaccactttttcccaaagtagactgaaagcacatcctgtg | |
| ctgggcggagcagctgtgtttggatggtttcatttcagcatgagaacagactcaaataga | |
| acggggagacttttccctcaacaaaaggaaagacagtcctatttgcactgtatcaccctt | |
| gagatactactgttacagagattagaacc | |

TABLE 198

| | |
|---|---|
| cbe_4383836 KIAA0905 | |
| yeast Sec31p homolog | |
| length = 4129 | |
| gacgagcgctgcactaacgcaggatccggctgccgaaggtcctcgccagcaggatgaagt | SEQ ID NO: 189 |
| taaaggaagtagatcgtacagccatgcaggcatggagccctgcccagaatcaccccattt | |
| acctagcaacaggaacatctgctcagcaattggatgcaacatttagtacgaatgcttccc | |
| ttgagatatttgaattagacctctctgatccatccttggatatgaaatcttgtgccacat | |
| tctcctcttctcacaggtaccacaagttgatttgggggccttataaaatggattccaaag | |
| gagatgtctctggagttctgattgcaggtggtgaaaatggaaatattattctctatgatc | |
| cttctaaaattatagctggagacaaggaagttgtgattgcccagaatgacaagcatactg | |
| gcccagtgagagccttggatgtgaacattttccagactaatctggtagcttctggtgcta | |
| atgaatctgaaatctacatatgggatctaaataattttgcaaccccaatgacaccaggag | |
| ccaaaacacagccgccagaagatatcagctgcattgcatggaacagacaagttcagcata | |
| ttttagcatcagccagtcccagtggccgggccactgtatgggatcttagaaaaaatgagc | |
| caatcatcaaagtcagtgaccatagtaacagaatgcattgttctgggttggcatggcatc | |
| ctgatgttgctactcagatggtccttgcctccgaggatgaccggttaccagtgatccaga | |
| tgtgggatcttcgatttgcttcctctccacttcgtgtcctggaaaaccatgccaggggga | |
| ttttggcaattgcttggagcatggcagatcctgaattgttactgagctgtggaaaagatg | |
| ctaagattctctgctccaatccaaacacaggagaggtgttatatgaacttcccaccaaca | |
| cacagtggtgcttcgatattcagtggtgtccccgaaatcctgctgtcttatcagctgctt | |
| cgtttgatgggcgtatcagtgtttattctatcatgggaggaagcacagatggtttaagac | |
| agaaacaagttgacaagctttcatcatcttttgggaatcttgatccctttggcacaggac | |
| agccccttcctccgttacaaattccacagcagactgctcagcatagtatagtgctgcctc | |
| tgaagaagccgcccaagtggattcgaaggcctgttggtgcttctttttcatttggaggca | |
| aactggttacgtttgagaatgtcagaatgccttctcatcagggagctgagcagcagcagc | |
| agcagcaccatgtgttcattagtcaggttgtaacagaaaaggagttcctcagccgatcag | |
| accaacttcagcaggctgtgcagtcacaaggatttatcaattattgccaaaaaaaaattg | |
| atgcttctcagactgaatttgagaaaaatgtgtggtcctttttgaaggtaaactttgagg | |
| atgattctcgtggaaaataccttgaacttctaggatacagaaaagaagatctaggaaaga | |
| agattgctttggccttgaacaaagtggatggagccaatgtggctcttaaagactctgacc | |
| aagtagcacagagtgatggggaggagagccctgctgctgaagagcagctcttgggagagc | |
| acattaaagaggaaaaagaagaatctgaatttctaccctcatctggaggaacatttaata | |
| tctctgtcagtggggacattgatggtttaattactcaggctttgctgacgggcaattttg | |
| agagtgctgttgacctttgtttacatgataaccgcatggccgatgccattatattggcca | |
| tagcaggtggacaagaactcttggctcgaacccagaaaaaatacttcgcaaaatcccaaa | |
| gcaaaattaccaggctcatcactgcagtggtgatgaagaactggaaagagattgttgagt | |
| cttgtgatcttaaaaattggagagaggctttagctgcagtattgacttatgcaaagccgg | |
| atgaattttcagccctttgtgatcttttgggaaccaggcttgaaaatgaaggagatagcc | |
| tcctgcagactcaagcatgtctctgctatatttgtgcagggaatgtagagaaattagttg | |
| catgttggactaaagctcaagatggaagccaccctttgtcacttcaggatctgattgaga | |
| aagttgtcatcctgcgaaaagctgtgcaactcactcaagccatggacactagtactgtag | |
| gagttctcttggctgcgaagatgagtcagtatgccaatttgttggcagctcagggcagta | |
| ttgctgcagccttggcttttcttcctgacaacaccaaccagccaaatatcatgcagcttc | |
| gtgacagactttgtagagcacaaggagagcctgtagcaggacatgaatcacctaaaattc | |
| cgtacgagaaacagcagctccccaagggcaggcctggaccagttgctggccaccaccaga | |
| tgccaagagttcaaactcaacaatattatccccatggagaaaatcctccacctccgggtt | |
| tcataatgcatggaaatgttaatccaaatgcttctggtcagcttcccacatctccaggtc | |
| atatgcacacccaggtaccaccttatccacagccacagccttatcaaccagcccagccgt | |
| atcccttcggaacaggggggtcagcaatgtatcgacctcagcagcctgttgctcctccta | |
| cttcaaacgcttaccctaacaccccttacatatcttctgcttcttcctatactgggcagt | |
| ctcagctgtacgcagcacagcaccaggcctcttcacctacctccagccctgctacttctt | |
| tccctcctcccccttcctctggagcatccttccagcatggcggaccaggagctccaccat | |
| catcttcagcttatgcactgcctcctggaacaacaggtacactgcctgctgccagtgagc | |
| tgcctgcgtcccaaagaacaggtcctcagaatggttggaatgaccctccagctttgaaca | |
| gagtacccaaaaagaagaagatgcctgaaaacttcatgcctcctgttcccatcacatcac | |
| caatcatgaacccgttgggtgacccccagtcacaaatgctgcagcaacagccttcagctc | |
| cagtaccactgtcaagccagtcttcattcccacagccacatcttccaggtggccagccct | |
| tccatggcgtacagcaacctcttggtcaaacaggcatgccaccatctttttcaaagccca | |
| atattgaaggtgccccaggggctcctattggaaataccttccagcatgtgcagtctttgc | |
| caacaaaaaaaattaccaagaaacctattccagatgagcacctcattctaaagaccacat | |
| ttgaggatcttattcagcgctgcctttcttcagcaacagaccctcaaaccaagaggaagc | |
| tagatgatgccagcaaacgtttggagtttctgtatgataaacttagggaacagacacttt | |
| caccaacaatcaccagtggtttacacaacattgcaaggagcattgaaactcgaaactact | |
| cagaaggattgaccatgcatacccacatagttagcaccagcaacttcagtgagacctctg | |
| ctttcatgccagttctcaaagttgttctcacccaggccaataagctgggtgtctaaaagg | |
| acagcttctcttccactcaatattgccatttttccaaagaaacatgttaaaaaaaaaaat | |
| tataagacatggactagtcctcattagcatgtttgcatagcaaccagtcaagagcattta | |
| cactatttctgctgatatactcaccttagaactgctcagaaccctggtgctttatttttg | |
| ttttaatcttttgttgccagtgatgattttcctattctgcaaatagtgtatttcctggat | |
| tacacatagtatggtttcctgaagtattctgataaatgtgtttttttaaaacctcaatat | |
| actttttagaaaaggagcatctggttatgcataaagcagagctaaaactaaatttctttc | |
| atgtcctccctacttcctcagtgtcaatcagattaaagtgtgtaatcct | |

TABLE 199

| | |
|---|---|
| cbe_4465094 Prey1053092 | |
| PathCalling Prey Sequence 1053092 | |
| length = 452 | |
| TCACAAACAAGCCTTCAGAATCAGATGAATGGAGGACCTTTTTATAGCCAGAATCCAGTT | SEQ ID NO: 190 |
| TCAGATACACCACCTCCACCGCCACCTGTGGAAGAACCAGTCTTTGATGAGTCTCCCCCA | |
| CCTCCTCCTCCTCCAGAAGATTACGAAGAGGAGGAAGCTGCTGTGGTTGAGTATAGTGAT | |
| CCTTATGCTGAAGAGGACCCACCGTGGGCTCCACGTTCTTACTTGGAAAAGGTTGTGGCA | |
| ATTTATGACTATACAAAAGACAAGGAAGATGAGCTGTCCTTTCAGGAAGGAGCCATTATT | |
| TATGTCATCAAGAAGAATCACGATGTATTAAAAACAAAGCAAGCTGAGTCTGAACAAATG | |
| GATCTTTCTGCCATCATTTGTACAATCCTGAGCTGTCTGGATTGAAATAAAATGACCATT | |
| TTTATGTATGTCAAAAAAAAAAAAAAAAACCC | |

TABLE 200

| | |
|---|---|
| cbe_4540466 PUM1 | |
| pumilio homolog 1 (Drosophila) | |
| length = 5390 | |
| gggggtgaaaggtaagggggagcgagagcgccagagagagaagatcggggggctgaaatc | SEQ ID NO: 191 |
| catcttcatcctaccgctccgcccgtgttggtggaatgagcgttgcatgtgtcttgaaga | |
| gaaaagcagtgctttggcaggactctttcagcccccacctgaaacatcaccctcaagaac | |
| cagctaatcccaacatgcctgttgttttgacatctggaacagggtcgcaagcgcagccac | |
| aaccagctgcaaatcaggctcttgcagctgggactcactccagccctgtcccaggatcta | |
| taggagttgcaggccgttcccaggacgacgctatggtggactacttctttcagaggcagc | |
| atggtgagcagcttgggggaggaggaagtggaggaggcggctataataatagcaaacatc | |
| gatggcctactggggataacattcatgcagaacatcaggtgcgttccatggatgaactga | |
| atcatgattttcaagcacttgctctggagggaagagcgatgggagagcagctcttgccag | |
| gtaaaaagttttgggaaacagatgaatccagcaaagatggaccaaaaggaatattcctgg | |
| gtgatcaatggcgagacagtgcctggggaacatcagatcattcagtttcccagccaatca | |
| tggtgcagagaagacctggtcagagtttccatgtgaacagtgaggtcaattctgtactgt | |
| ccccacgatcggagagtgggggactaggcgttagcatggtggagtatgtgttgagctcat | |
| ccccgggcgattcctgtctaagaaaaggaggatttggcccaagggatgcagacagtgatg | |
| aaaacgacaaaggtgaaaagaagaacaagggtacgtttgatggagataagctaggagatt | |
| tgaaggaggagggtgatgtgatggacaagaccaatggtttaccagtgcagaatgggattg | |
| atgcagacgtcaaagattttagccgtacccctggtaattgccagaactctgctaatgaag | |
| tggatcttctgggtccaaaccagaatggttctgagggcttagcccagctgaccagcacca | |
| atggtgccaagcctgtggaggatttctccaacatggagtcccagagtgtccccttggacc | |
| ccatggaacatgtgggcatggagcctcttcagtttgattattcaggcacgcaggtacctg | |
| tggactcagcagcagcaactgtgggactttttgactacaattctcaacaacagctgttcc | |
| aaagacctaatgcgcttgctgtccagcagttgacagctgctcagcagcagcagtatgcac | |
| tggcagctgctcatcagccgcacatcggtttagctcccgctgcgtttgtccccaatccat | |
| acatcatcagcgctgctcccccagggacggacccctacacagctggattggctgcagcag | |
| cgacactaggcccagctgtggtccctcaccagtattatggagttactccctggggagtct | |
| accctgccagtcttttccagcagcaagctgccgctgccgctgcagcaactaattcagcta | |
| atcaacagaccaccccacaggctcagcaaggacagcagcaggttctccgtggaggagcca | |
| gccaacgtcctttgaccccaaaccagaaccagcagggacagcaaacggatccccttgtgg | |
| cagctgcagcagtgaattctgcccttgcatttggacaaggtctggcagcaggcatgccag | |
| gttatccggtgttggctcctgctgcttactatgaccaaactggtgcccttgtagtgaatg | |
| caggcgcgagaaatggtcttggagctcctgttcgacttgtagctcctgccccagtcatca | |
| ttagttcctcagctgcacaagcagctgttgcagcagccgcagcttcagcaaatggagcag | |
| ctggtggtcttgctggaacaacaaatggaccatttcgccctttaggaacacagcagcctc | |
| agccccagccccagcagcagcccaataacaacctggcatccagttctttctacggcaaca | |
| actctctgaacagcaattcacagagcagctccctcttctcccagggctctgcccagcctg | |
| ccaacacatccttgggattcggaagtagcagttctctcggcgccaccctgggatccgccc | |
| ttggagggtttggaacagcagttgcaaactccaacactggcagtggctcccgccgtgact | |
| ccctgactggcagcagtgacctttataagaggacatcgagcagcttgacccccattggac | |
| acagtttttataacggccttagcttttcctcctctcctggacccgtgggcatgcctctcc | |
| ctagtcagggaccaggacattcacagacaccacctccttccctctcttcacatggatcct | |
| cttcaagcttaaacctgggaggactcacgaatggcagtggaagatacatctctgctgctc | |
| caggcgctgaagccaagtaccgcagtgcaagcagcgcctccagcctcttcagcccgagca | |
| gcactcttttctcttcctctcgtttgcgatatggaatgtctgatgtcatgccttctggca | |
| ggagcaggcttttggaagattttcgaaacaaccggtaccccaatttacaactgcgggaga | |
| ttgctggacatataatggaattttcccaagaccagcatgggtccagattcattcagctga | |
| aactggagcgtgccacaccagctgagcgccagcttgtcttcaatgaaatcctccaggctg | |
| cctaccaactcatggtggatgtgtttggtaattacgtcattcagaagttctttgaatttg | |
| gcagtcttgaacagaagctggctttggcagaacggattcgaggccacgtcctgtcattgg | |
| cactacagatgtatggctgccgtgttatccagaaagctcttgagtttattccttcagacc | |
| agcagaatgagatggttcgggaactagatggccatgtcttgaagtgtgtgaaagatcaga | |
| atggcaatcacgtggttcagaaatgcattgaatgtgtacagccccagtctttgcaattta | |
| tcatcgatgcgtttaagggacaggtatttgccttatccacacatccttatggctgccgag | |
| tgattcagagaatcctggagcactgtctccctgaccagacactccctattttagaggagc | |
| ttcaccagcacacagagcagcttgtacaggatcaatatggaaattatgtaatccaacatg | |
| tactggagcacggtcgtcctgaggataaaagcaaaattgtagcagaaatccgaggcaatg | |
| tacttgtattgagtcagcacaaatttgcaagcaatgttgtggagaagtgtgttactcacg | |
| cctcacgtacggagcgcgctgtgctcatcgatgaggtgtgcaccatgaacgacggtcccc | |
| acagtgccttatacaccatgatgaaggaccagtatgccaactacgtggtccagaagatga | |
| ttgacgtggcggagccaggccagcggaagatcgtcatgcataagatccggccccacatcg | |
| caactcttcgtaagtacacctatggcaagcacattctggccaagctggagaagtactaca | |
| tgaagaacggtgttgacttagggcccatctgtggcccccctaatggtatcatctgaggca | |
| gtgtcacccgctgttccctcattcccgctgacctcactggcccactggcaaatccaacca | |
| gcaaccagaaatgttctagtgtagagtctgagacgggcaagtggttgctccaggattact | |
| ccctcctccaaaaaaggaatcaaatccacgagtggaaaagcctttgtaaatttaatttta | |
| ttacacataacatgtactattttttttaattgactaattgccctgctgttttactggtgt | |
| ataggatacttgtacataggtaaccaatgtacatgggaggccacatattttgttcactgt | |
| tgtatctatatttcacatgtggaaactttcagggtggttggtttaacaaaaaaaaaaagc | |
| tttaaaaaaaaaagaaaaaaaggaaaaggtttttagctcatttgcctggccggcaagttt | |
| tgcaaatagctcttccccacctcctcattttagtaaaaaacaaacaaaaacaaaaaaacc | |
| tgagaagtttgaattgtagttaaatgaccccaaactggcatttaacactgtttataaaaa | |
| atatatatatatatatatatatataatgaaaaaggtttcagagttgctaaagcttcagtt | |
| tgtgacattaagtttatgaaattctaaaaaatgccttttttggagactatattatgctga | |
| agaaggctgttcgtgaggaggagatgcgagcacccagaacgtcttttgaggctgggcggg | |
| tgtgattgtttactgcctactggatttttttctattaacattgaaaggtaaaatctgatt | |
| atttagcatgagaaaaaaaaatccaactctgcttttggtcttgcttctataaatatatag | |
| tgtatacttggtgtagactttgcatatatacaaatttgtagtattttcttgttttgatgt | |
| ctaatctgtatctataatgtaccctagtagtcgaacatacttttgattgtacaattgtac | |
| atttgtatacctgtaatgtaaatgtggagaagtttgaatcaacataaacacgttttttgg | |
| taagaaaagagaattagccagccctgtgcattcagtgtatattctcaccttttatggtcg | |
| tagcatatagtgttgtatattgtaaattgtaatttcaaccagaagtaaatttttttcttt | |
| tgaaggaataaatgttctttatacagcctagttaatgtttaaaaagaaaaaaatagcttg | |
| gttttatttgtcatctagtctcaagtatagcgagattctttctaaatgttattcaagatt | |
| gagttctcactagtgtttttttaatcctaaaaaagtaatgttttgattttgtgacagtca | |
| aaaggacgtgcaaaagtctagccttgcccgagctttccttacaatcagagcccctctcac | |
| cttgtaaagtgtgaatcgcccttcccttttgtacagaagatgaactgtattttgcatttt | |
| gtctacttgtaagtgaatgtaacatactgtcaattttccttgtttgaatatagaattgta | |
| acactacacggtgtacatttccagagccttgtgtatatttccaatgaacttttttgcaag | |
| cacacttgtaaccatatgtgtataattaacaaacctgtgtatgcttatgcctgggcaact | |
| attttttgtaactcttgtgtagattgtctctaaacaatgtgtgatctttattttgaaaaa | |
| tacagaactttggaatctgaaaaaaaaaaaaaaaaaaaaaaaaaaaaaaa | |

TABLE 201

| | |
|---|---|
| cbe_4567083 LOC157378 | |
| hypothetical protein BC017881 | |
| length = 2604 | |
| ggctgggcacgcaccccaagaaggagcccatggaggcgctgaacacggcgcagggcgcgc | SEQ ID NO: 192 |
| gcgacttcatctacagcctgcactccacggagaggagctgcctgctcaaagagctgcacc | |
| gcttcgagtctattgccattgcccaagaaaaattggaagctccaccacccaccccaggac | |
| agctgagatatgtattcatccacaatgcgatacctttcatagggtttggctttttggata | |
| atgcaattatgattgttgctggaacccatattgaaatgtctattggaattattttgggaa | |
| tttcaactatggcagctgctgctttgggaaatcttgtgtcagatctagctggacttggac | |
| ttgcaggctacgttgaagcattggcttccaggttaggcctgtcaattcctgatctcacac | |
| caaagcaagttgacatgtggcaaacacgtcttagtacacatttgggcaaagctgttgggg | |
| tgactattggctgcattctaggaatgtttcctttaattttctttggaggaggtgaagaag | |
| atgaaaaactggaaacgaaaagttaatcctcttagaatacctataaaaagatgtaaacta | |
| atgtacctcagtaattaaatatgctgtcacaacatttaggaattaagacagtaacagtat | |
| agatatgggatcaaataatttagcatgtattatggaaaacactaacttattgtggcttga | |
| tcttcttaggacatcttttttaaaaagctgtttagtatcattttgtgtatattgttgaaa | |
| tgctttttcatcaatagcagtcaacattttatcctttctttttatattcataatgttatt | |
| taagtgtcattgatgtactgtattgacttggggtttgcttatttgttacttaacatgtgt | |
| acatgcatgaaagcatttttcgttgttccctgatagttacatttcaaccttgggattttt | |
| ccaaattacttaagatgtttaatgtcagttaaagatttttttaccctctttttgggaaca | |
| tcaattttgtactgttatgcagtaaacatttataataatataatttcagtcatttcttaa | |
| ctgtcacatctattgaaaatggatatagatacaggttttaagtattttaagtatatatta | |
| cttattttaattttctgactttactattttaagggccagagggttaatcacaaagagcaa | |
| ttatgtggtctccctgctacatgaaaccgtgtatactaacaagcgtacaatttttagttg | |
| attttttttaaccttttagtttcccagttttgaataattacatggtggattctgactttt | |
| gaggggaagcaaatgattattttagagtctttgaaatggggattgtggaattagattgaa | |
| ctaagggatttaacatgatgcttggaaattaagagactaaagctttttttaaaaaaaggt | |
| ggaaaataggaactgtcaagaaggtttatggtataaatgatgaagttgaagtgatgtttg | |
| aaagattaatgagatacaatttatattatttggtaaggtttttttttttccctccaaaga | |
| tgtcatcttctcatctgaatggaataagtctgaataccccatattcatactcctaatctc | |
| attatatcttatttagtgaattttatttatgaataatttctgttgaagtgaaaactagat | |
| atttaatattttgcttttttgctacatagtctactcaaaaattacatgaggagaaatcct | |
| ttttccctttgtttttcttttttcttcttttgtggtatttaaagcatattttaggttgaa | |
| gttacttatttctagtcttgtacttctggcttaagtataaccatgtaagaattataaatt | |
| ttagttttctgaacccttaaacttttttagcatgtggtctgttacacatgctaaaaaatt | |
| agtcttacttgtaacagcgtaattaaacacatcatggaggagagaaacttaaaatagtat | |
| tttggcttttgaagttatttgtgttgctaaatagatgcagaggttttacagcagtttatt | |
| ttaaggttttatttatacataattactttgaactcttcagagtagatattttttcacaag | |
| gcagtttgtcataaattctctcatgcgcttcagaatgcataagtgccatcctttaatcat | |
| agactttgaggagagaaagcataaaaatatagcatataatctaaaaataaatatataaca | |
| tgcacaaataatgtgacattcttactgaatcaaatcatgattctagaacttgagatctta | |
| aatagaattccggtttgtatcttccatataataaccacacacagataaccacacaaaaaa | |
| tcctttgtaaaatttctgattgataggattagagtgcttaaatttttggggggggaaggg | |
| tggggtaaagtgtaagtgctttcttttgtccctaacttgtgtattgatggcagtccactc | |
| tgttttctaaaaatgtattttactgtggtgcttaacttcttattaattaaatcccgtatc | |
| agaaaccttgaaaaaaaaaaaaaaaaaaaaaaaaaaaaaaaaaaaaaaaaaaaaaaaaaa | |
| aaaaaaaaaaaaaaaaaaaaaaaa | |

TABLE 202

| | |
|---|---|
| cbe_552305 Prey2354 | |
| PathCalling Prey Sequence 2354 | |
| length = 241 | |
| aaggaaggaaggaaggaaggaaggaaggaaggaaagaaagaaattcatccagcatgtccc | SEQ ID NO: 193 |
| tttcttctcctatgggatttaccaactgacaaattctagtgctcctgccatcacccacac | |
| atgtgcagcaaagcctccacatttgcaaaatctaagtcaatctctaaaagatgcttcgag | |
| gaagcacccagttccttctgctaataaccgcagtgtgtcatgtgacacctccccctcccc | |
| g | |

TABLE 203

| | |
|---|---|
| cbe_552975 HPV 1a E1 | |
| Human Papillomavirus 1a E1 | |
| length = 1839 | |
| atggcagataataaaggtactgaaaacgattggtttttggtggaggcgacagattgtgag | SEQ ID NO: 194 |
| gaaacgttagaggaaacctcacttggtgacctagataatgtttcttgtgttagcgactta | |
| tctgatttattagacgaggcgccgcaaagccaggggaattccctggaattgttccacaag | |
| caagaatcgctggaaagcgaacaggaacttaatgctttaaaacgaaagttactttacagt | |
| cctcaggcgagaagcgcggacgaaacagacattgctagcattagtcctagattagaaact | |
| atttctattacaaagcaagacaaaaaaaggtatcgaaggcaactgttttctcaggatgat | |
| agtggtttagagctatcgctgcttcaggatgaaactgaaaatattgatgaatcgacacag | |
| gtagatcaacagcagaaagaacatactggggaagttggggccgctggggtgaacattttg | |
| aaagctagtaatatccgcgccgcattattaagcagatttaaagatacggctggcgtcagt | |
| tttacagacctgacgcggtcgtacaagagcaacaaaacctgttgtggagattgggttttg | |
| gcagtttggggtgtccgtgaaaatttaattgacagtgtaaaagaattattgcaaacccat | |
| tgtgtgtatattcaattggaacatgcagtaactgaaaaaaatagatttttatttttattg | |
| gtacgatttaaagcccagaaaagtagagagactgtgataaaacttataaccacaattctt | |
| ccagttgatgctagctatattttgtctgagcctccaaaatcaagaagtgtggctgctgca | |
| ttattttggtataaaagatctatgtcttcaactgtttttacatggggtacaactttggag | |
| tggattgcacagcaaacccttattaatcatcagttagattccgaaagtccctttgagctt | |
| tgtaaaatggttcagtgggcctatgataatggacatacagaagagtgtaaaattgcatat | |
| tattatgctgttttagcagatgaggatgaaaatgcaagggcatttctaagctctaattca | |
| caggcaaaatatgtgaaagactgtgcacaaatggtaagacactatttacgtgctgagatg | |
| gcacaaatgtctatgtcagagtggatttttagaaaactagataatgtagaaggttctggt | |
| aattggaaagaaattgtaagatttttaagatttcaagaagttgaatttataagctttatg | |
| attgcatttaaagatttgttatgtggtaagccaaagaaaaactgtttgttaatatttgga | |
| cctccaaatacaggaaaatcaatgttttgtacaagtttattaaagttgttaggagggaaa | |
| gtgatttcatactgtaacagtaaaagtcagttttggttgcagcctctggctgatgctaag | |
| atagggctattagatgatgcaacaaagccatgttgggattatatggacatttatatgaga | |
| aatgcattggatggtaacactatttgtattgatttaaaacatagagctcctcaacaaatt | |
| aaatgcccacctttacttattactagtaatattgatgttaaatcagatacctgttggatg | |
| tatttgcatagtagaatatcagcttttaaatttgctcatgagtttccatttaaagacaat | |
| ggtgatccaggattttccttaacagacgaaaattggaaatctttctttgaaaggttttgg | |
| caacagttagaattaagtgaccaagaagacgagggaaacgatggaaaacctcagcagtcg | |
| cttagacttactgcaagagcagctaatgaacctatatga | |

TABLE 204

| | |
|---|---|
| cbe_552976 HPV 11 L2 | |
| Human papillomavirus 11 L2 | |
| length = 1368 | |
| atgaaacctagggcacgcagacgtaaacgtgcgtcagccacacaactatatcaaacatgc | SEQ ID NO: 195 |
| aaggccactggtacatgtcccccagatgtaattcctaaagttgaacatactactattgca | |
| gatcaaatattaaaatggggaagcttaggggttttttttggtgggttaggtattggtaca | |
| ggggctggtagtggcggtcgtgcagggtatatacccttgggaagctctcccaagcctgct | |
| attactggggggccagcagcacgtccgccagtgcttgtggagcctgttgccccttccgat | |
| ccctccattgtgtccttaattgaggagtctgctattattaatgctggtgcacctgaggtg | |
| gtaccccctacacagggtggctttactataacatcatctgaatcgactacacctgctatt | |
| ttagatgtgtctgttaccaatcacactaccactagtgtgtttcaaaatcccctgtttaca | |
| gaaccgtctgtaatacagccccaaccacctgtggaggccagtggtcacatacttatatct | |
| gccccaacaataacatcccaacatgtagaagacattccactagacacttttgttgtatcc | |
| tctagtgatagtggacctacatccagtactcctcttcctcgtgcttttcctcggcctcgg | |
| gtgggtttgtatagtcgtgccttacagcaggtacaggttacggaccccgcgtttttgtcc | |
| acgccacagcgattggtaacttatgacaaccctgtctatgaaggagaagatgtaagttta | |
| caatttacccatgagtctatccacaatgcacctgatgaagcatttatggatattattaga | |
| ctacatagaccagctataacgtccagacggggtcttgtgcgttttagtcgcattgggcaa | |
| cgggggtccatgtacacacgcagtggacaacatataggtgcccgcatacattattttcag | |
| gacatttcaccagttacacaagctgcagaggaaatagaactgcaccctctagtggctgca | |
| gaaaatgacacgtttgatatttatgctgaaccatttgaccctatccctgaccctgtccaa | |
| cattctgttacacagtcttatcttacctccacacctaataccctttcacaatcgtggggt | |
| aataccacagtcccattgtcaatccctagtgactggtttgtgcagtctgggcctgacata | |
| acttttcctactgcatctatgggaacaccctttagtcctgtaactcctgctttacctaca | |
| ggccctgtttttattacaggttctgacttctatttgcatcctacatggtactttgcacgc | |
| agacgccgtaaacgtattcccttattttttacagatgtggcggcctag | |

TABLE 205

| | |
|---|---|
| cbe_6464 Hs. 19954 | |
| 603029812F1 Homo sapiens cDNA, 5′ end | |
| length = 959 | |
| ctggcggcggcggccactctaaccagcgcaaaatgtccctggaacaggaggaggaaacgc | SEQ ID NO: 196 |
| aacctgggcggctcctaggacgcagagacgccgtccccgccttcattgagcccaacgtgc | |
| gcttctggatcaccgagcgccaatcctttattcgacgatttcttcaatggacagaattat | |
| tagatcctacaaatgtgttcatttcagttgaaagtatagaaaactcgaggcaactattgt | |
| gcacaaatgaagatgtttccagccctgcctcggcggaccaaaggatacaagaagcttgga | |
| agcggagtcttgcaacagtgcatcccgacagcagcaacctgatccccaagctttttcgac | |
| ctgcagcgttcctgcctttcatggcgcccacggtatttttgtcaatgacgccactgaaag | |
| ggatcaagtccgtgattttacctcaggttttcctctgtgcctacatggcagcgttcaaca | |
| gcatcaatggaaacagaagttacacttgtaagccactagaaagatcattactaatggcgg | |
| gagccgttgcttcttcaactttcttaggagtaatccctcagtttgtccagatgaagtatg | |
| gcctgactggcccttggattaaaagactcttacctgtgatcttcctcgtgcaagccagtg | |
| gaatgaatgtctacatgtcccgaagtcttgaatccattaaggggattgcggtcatggaca | |
| aggaaggcattgtcctgggtcattcccagaattgctgggacaaaggctgttagagaaaac | |
| gctagccatccagaaatagtgcatgtttgggaccctcagttcttgattcctgaagttctt | |
| caacctaactttttaaaaggacccagtatttcaggaaaaacccaggtcatgtggatttga | |
| aactgtcttgtaactgtcctggcatggaactgatggtggcatttcttttaatttttcaa | |

TABLE 206

| | |
|---|---|
| cbe_71919 FLJ13236 | |
| hypothetical protein FLJ13236 | |
| length = 1854 | |
| ctccaaccctctgctcccgtttctccctctggttcatagcccgcgggacagagcagccct | SEQ ID NO: 197 |
| ggattgggaggctcctcaggtctcaggctcaggtgcagaagcagagcccctaactggaag | |
| gctcggaggtgggccggtgaggcaagcaagccctgtggggggttgaggacagcttgggtt | |
| ggatgtccctcactgcagcctgtccgtggatgtctctgccttaagggaattggaggtgga | |
| accatccagtcttgaaggtcaacggagcgtgaagagaggagcgtgcagcttgagggtcta | |
| aggataactctggggccatgacagccatgagtctcacagaggaccccgagacttctgtgc | |
| tgcgagtaaccggacttgttctgagacctttgccctagaggatggccaaggggctcctgg | |
| tgacctatgccctctgggctgtggggggccctgctgggctccaccacctgtacctgggaa | |
| gggacagccacgccctgctctggatgctgaccctggggggaggtgggctgggctggctct | |
| gggagttctggaagctcccaagctttgtagctcaggccaacagagcccagggacagaggc | |
| agagccccagaggggtgacaccccctctgagtcccattcgctttgctgcccaggtgatag | |
| ttggcatctattttggccttgtggcactgattagcctttcttccatggtcaacttctata | |
| ttgtggccctcccactggcagttggcttaggggtcttgctggtggctgctgttggcaacc | |
| agacctcagactttaagaacactctggggtcagcatttctcacttcacctatcttctatg | |
| gccgccccatagccatactgcccattagcgtggccgccagcattacagctcagaggcatc | |
| gccgctacaaagctttggtggcatcagagccgctcagtgtgcggctctatcgtctgggct | |
| tggcttaccttgctttcacaggcccactggcatacagtgccctctgcaacacagctgcca | |
| ccctcagctatgtggcagaaacctttggctccttcttgaattggttcagcttcttccccc | |
| ttcttggccgcctcatggagtttgtcctccttctgccttaccggatctggaggctactga | |
| tgggggagactggcttcaacagcagctgctttcaggagtgggcgaagctctatgagtttg | |
| ttcacagttttcaggatgagaagcgtcagctggcttaccaggttttgggcctctcagaag | |
| gggcaacaaatgaagaaatacatcggagttaccaggagctagtgaaggtctggcacccag | |
| accacaacctggaccagacagaggaggcacagaggcacttcctggagatccaggctgcgt | |
| atgaagtcctgagtcaacccaggaagccctggggatcccggaggtgaaaagaaacttccc | |
| cctgaggactgactcttcctagcagagctgggcaacttgtcccaaatctagctttgccca | |
| cgaatggcatcccaacagagttaaagaaactgcttgcaggggctgggcgtggtggctcat | |
| gcctgtaatcccggcactttgggaggccgaggcgggcagatcacgaggtcaggagttcga | |
| gaccagcagcctggcdaatatggtgaaacttcgtctctactaaaaatacaaaaaattggc | |
| caggagtggtggcaggcacctgtagtcccagctacttgggaggctgaggcaggagaatca | |
| cctgaacctgagaggcggaggttgcagtgagccgagatcacgccactgcactccagcctg | |
| ggcaacaagagtgaaactccgtctcaaaaaaaaaaaaaaaaaaaaaaaaaaaaa | |

TABLE 207

| | |
|---|---|
| cbe_92628 FTHP1 | |
| ferritin, heavy polypeptide pseudogene 1 | |
| length = 2083 | |
| catcaaaagaatgaagaacataagaaaaggtaactacatcagtaaatatattgctttttt | SEQ ID NO: 198 |
| cttcttatttaaatgctttaaaagagaattgattgtttaaataaaaatatcaatgtaggc | |
| tacatgcagtggctcacacctataatcccagcactttgggaggctgaggtgggtggatca | |
| cttgaggtcagaatgttcaagaccagcctggccaacatggtgatctttgtgtctattaaa | |
| aaatacaaaaataagccaggcgtggtggcgggcacctgtaatccagctactctaaggctg | |
| aggcaagagatcacttgaatccaggaggcagaggctgcagtcagccaaaattactccact | |
| gccctccagcctaggcaacagagtgagaatctgtctcaaaaataaaaataaaaataaatc | |
| aatgtagtatcagacttatatgtagaaattaaatatttgacaaaataacatgaaattata | |
| aggagagaaattaaaatttattattttaaggttcttaatatacaaactagtatattatca | |
| gttgaagatagtctgcaataatttaaagctatatactataaacactaaagcaaccattaa | |
| tatagcaaaattgaaaatatgaagttaatagggcaacaaaggagataaaatgtaatttta | |
| agaaatatacaaataattacaaaaagacagcataatagtaaaagagaatgaacagataaa | |
| acaattataaaacaaacaccaagctgagagacttaaacctaatcacacgataatcacact | |
| aaatgtaaatggtccaaatacccccatcaaaaaggagagactttcaaatagataatgaaa | |
| attcaagacctaaccacaaaaaaaacacacttagaatataaagtcacagatagggtaaaa | |
| gtggaagaatgacaaaagaagagacattcttcaccaagagtcctcggggtttcctgcttc | |
| agcagtgcttggacggaacccggcacttgtcccccacccaggccagctgcccatagccag | |
| ccctccgtcacgtcttcactgcaccctcagaccaccccaaggcccccaccgccgctccag | |
| cgccacgcagccaccgccgccgcagcctctccttagtcattgccatgatgactgcatcca | |
| actcgcaggtgcgccagaactaccaccaggactcagaggccgccatcaaccgccagatca | |
| acctggagctctacgcctcctactttaacctctccatgtcttactactttgaccgtgatg | |
| atgcggctttgaagaactttgccaaatactttcttcaccaatctcatgaggagagggagc | |
| atgctgagaaactgatgaaactgcagaaccaacaaggtggctgaattttccttcaggata | |
| tcaagaaaccagactgatgactgggatagtgggctgaatgtgatagagtgtgcattacat | |
| ttggaaaaaaatgtgaatcagtcactactggaactgcacaaactggccactgacaaaaat | |
| gacccccatttgtgtgacttcattgagacacattacctgaatgagcaggtgaaagccatc | |
| aaagaattgggtgaacgtgaccaacttgcacaagatgggagcacccgaatctggcttggc | |
| agaatatctctttgacaagcacaccctgggagaaagtgataatgaaagctaagcctcagg | |
| ctaatttccccatagctgtggggtgacttccctggtcaccaaggcagtgcatgcatgttg | |
| gggtttcctttaccttttctataagttgtaccaaaacatccacttaagttcttttgattt | |
| gtaccattccttcaaataaagaaatctggtacccccccccaaaaaagaatgacaaaagat | |
| agcacatgtgaatacaaataaaaagtaagctactgtggctacattcatgtcagacaaagt | |
| aaatttcagagtagagaatattaccagtaataaatgattatttcatagtgataaaggggt | |
| caattcatgaaaaagacataatcctaaacctaatacaacagttcaaaataaatgcagcaa | |
| aaactggaaggagaaagaaacagatcctctagagtcgacctgc | |

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

### REFERENCES

Ahmed, S., J. Lee, et al. (1994). “Breakpoint cluster region gene product-related domain of n-chimaerin. Discrimination between Rac-binding and GTPase-activating residues by mutational analysis.” J Biol Chem 269(26): 17642-8.
Boonstra, J., P. Rijken, et al. (1995). “The epidermal growth factor.” Cell Biol Int 19(5): 413-30.
Cannistra, S. A. and J. M. Niloff (1996). “Cancer of the uterine cervix.” N Engl J Med 334(16): 1030-8.
Dyson, N., P. M. Howley, et al. (1989). “The human papilloma virus-16 E7 oncoprotein is able to bind to the retinoblastoma gene product.” Science 243(4893): 934-7.
Gillison, M. L. and K. V. Shah (2001). “Human papillomavirus-associated head and neck squamous cell carcinoma: mounting evidence for an etiologic role for human papillomavirus in a subset of head and neck cancers.” Curr Opin Oncol 13(3): 183-8.
Hwang, E. S., T. Nottoli, et al. (1995). “The HPV16 E5 protein: expression, detection, and stable complex formation with transmembrane proteins in COS cells.” Virology 211(1): 227-33.
Johnson, C. V., D. E. Cool, et al. (1993). “Isolation and mapping of human T-cell protein tyrosine phosphatase sequences: localization of genes and pseudogenes discriminated using fluorescence hybridization with genomic versus cDNA probes.” Genomics 16(3): 619-29.
Kiyono, T., S. A. Foster, et al. (1998). “Both Rb/p16INK4a inactivation and telomerase activity are required to immortalize human epithelial cells.” Nature 396(6706): 84-8.
Koutsky, L. A., K. A. Ault, et al. (2002). “A controlled trial of a human papillomavirus type 16 vaccine.” N Engl J Med 347(21): 1645-51.
McDougall, J. K. (1994). “Immortalization and transformation of human cells by human papillomavirus.” Curr Top Microbiol Immunol 186: 101-19.
Munger, K., W. C. Phelps, et al. (1989). “The E6 and E7 genes of the human papillomavirus type 16 together are necessary and sufficient for transformation of primary human keratinocytes.” J Virol 63(10): 4417-21.
Munoz, N. (2000). “Human papillomavirus and cancer: the epidemiological evidence.” J Clin Virol 19(1-2): 1-5.
Nicoll, D., S. McPhee, et al. (2001). Pocket Guide to Diagnostic Tests, Lange/McGraw-Hill. Schiffman, M. H., N. B. Kiviat, et al. (1995). “Accuracy and interlaboratory reliability of human papillomavirus DNA testing by hybrid capture.” J Clin Microbiol 33(3): 545-50.
Takai, Y., T. Sasaki, et al. (2001). “Small GTP-binding proteins.” Physiol Rev 81(1): 153-208.
Tindle, R. W. (2002). “Immune evasion in human papillomavirus-associated cervical cancer.” Nature Rev Cancer 2(1): 59-65.
Walboomers, J. M., M. V. Jacobs, et al. (1999). “Human papillomavirus is a necessary cause of invasive cervical cancer worldwide.” J Pathol 189(1): 12-9.
Werness, B. A., A. J. Levine, et al. (1990). “Association of human papillomavirus types 16 and 18 E6 proteins with p53.” Science 248(4951): 76-9.
Wright, T. C., Jr., J. T. Cox, et al. (2002). “2001 Consensus Guidelines for the management of women with cervical cytological abnormalities.” Jama 287(16): 2120-9.
zur Hausen, H. (1996). “Papillomavirus infections—a major cause of human cancers.” Biochim Biophys Acta 1288(2): F55-78.
zur Hausen, H. (2002). “Papillomaviruses and cancer: from basic studies to clinical application.” Nat Rev Cancer 2(5): 342-50.

1. A purified complex comprising a first polypeptide and a second polypeptide, wherein said first polypeptide comprises an amino acid sequence of a polypeptide selected from the group consisting of the polypeptides recited in Table 1 through 7, column 2, and wherein said second polypeptide comprises an amino acid sequence of the corresponding polypeptide recited in Tables 1 through 7, column 5, wherein said first polypeptide binds said second polypeptide.

2. The complex of claim 1, wherein said first polypeptide is labeled with a detectable substance.

3. The complex of claim 1, wherein said second polypeptide is labeled is labeled with a detectable substance.

4. A chimeric polypeptide comprising six or more amino acids of the first polypeptide of claim 1 covalently linked to six or more amino acids of the second polypeptide of claim 1.

5. A nucleic acid encoding the chimeric polypeptide of claim 4.

6. A vector comprising the nucleic acid of claim 5.

7. A cell comprising the vector of claim 8.

8. An antibody which specifically binds the complex of claim 1.

9. The antibody of claim 8, wherein said antibody binds to the complex of claim 1 with higher affinity than it binds to said first or second polypeptide when said polypeptides are not complexed.

10. A pharmaceutical composition comprising the complex of claim 1.

11. A kit comprising a reagent which can specifically detect the complex of claim 1.

12. The kit of claim 11, wherein said reagent is selected from the group consisting of an antibody specific for said complex, an antibody specific for said first polypeptide, and an antibody specific for said second polypeptide.

13. A method of identifying an agent which disrupts a polypeptide complex, the method comprising:
(a) providing the complex of claim 1;
(b) contacting the complex with a test agent; and
(c) detecting the presence of a polypeptide displaced from said complex, wherein the presence of displaced polypeptide indicates said agent disrupts said complex.

14. A method for identifying an agent which disrupts a polypeptide complex comprising at least one HPV-associated protein or HPV interactor protein, the method comprising:
(a) providing the complex of claim 1;
(b) contacting said complex with a test agent; and
(c) detecting the presence of a polypeptide displaced from said complex, wherein the presence of displaced polypeptide indicates said agent disrupts said complex.

15. A method of identifying a polypeptide complex in a subject, the method comprising:
(a) providing a biological sample from said subject; and
(b) detecting, if present, the polypeptide complex of claim 1 in said sample, thereby identifying said complex.

16. A method of detecting a polypeptide in a biological sample, the method comprising:
(a) providing a biological sample comprising the first polypeptide of claim 1;
(b) contacting said biological sample with the second polypeptide of claim 1 under conditions suitable for formation of a complex comprising said first and second polypeptides; and
(c) detecting the presence of a complex of said first and second polypeptide, wherein the presence of said complex indicates the presence of said first polypeptide in said sample.

17. A method of detecting a polypeptide in a biological sample, the method comprising:
(a) providing a biological sample comprising the second polypeptide of claim 1;
(b) contacting said biological sample with the first polypeptide of claim 1 under conditions suitable for formation of a complex comprising said first and second polypeptides; and
(c) detecting the presence of a complex of said first and second polypeptide, wherein the presence of said complex indicates the presence of said second polypeptide in said sample.

18. A method of removing a polypeptide from a biological sample, the method comprising:
(a) providing a biological sample comprising the first polypeptide of claim 1;
(b) contacting said biological sample with the second polypeptide of claim 1 under conditions suitable for formation of a complex comprising said first and second polypeptide; and
(c) removing said complex from said sample, thereby removing said first polypeptide from said sample.

19. A method of determining altered expression of a polypeptide in a subject, the method comprising:
(a) providing a biological sample from said subject,
(b) measuring the level of the complex of claim 1 in said sample; and
(c) comparing the level of said complex from step (b) to the level of said complex in a reference sample whose level of the complex of claim 1 is known, thereby determining whether said subject has altered expression of said first or second polypeptide.

20. A method of treating or preventing a disease or disorder involving altered levels of the complex of claim 1, the method comprising:
administering a therapeutically-effective amount of least one molecule that modulates the function of said complex to a subject in need thereof.

21. A method of screening for pre-cancerous cervical lesions comprising contacting a patient derived cervical cell with a molecule that binds specifically to HPV E6 or E7 protein, detecting binding of said molecule, thereby identifying pre-cancerous cervical lesions.

22. The method of claim 21, wherein said E6 or E7 protein are proteins from HPV types selected from the group consisting of HPV 1a, HPV 11 and HPV 16.

23. A method of classifying an HPV infection in a patient, comprising contacting a patient derived cell with a molecule that binds specifically to a subset of HPV E6 or E7 proteins, said molecule selected from a keratinocyte library, detecting binding of said molecule thereby classifying an HPV infection in said patient.

24. The method of claim 23, wherein the cell is obtained from the site of HPV infection.

25. The method of claim 23, wherein the cell is selected from head, neck, larynx, skin, anogenital, epithelial, mucosal or cervical tissue.
